(19) United States
(12) Patent Application Publication
(10) Pub. No.: US 2022/0170057 A1
(43) Pub. Date: Jun. 2, 2022

(54) MICROORGANISMS AND METHODS FOR THE FERMENTATION OF CANNABINOIDS

(71) Applicant: ELESZTO GENETIKA, INC., Budapest (HU)

(72) Inventors: Bela Krisztian SZAMECZ, Budapest (HU); Szilvia VARSZEGI, Kalosca (HU); Attila NEMETH, Budapest (HU); Lorand SZABO, Szentendre (HU)

(21) Appl. No.: 17/594,266

(22) PCT Filed: Apr. 11, 2020
(86) PCT No.: PCT/IB2020/000241

§ 371 Date: Oct. 8, 2021

Related U.S. Application Data
(60) Provisional application No. 62/832,852, filed on Apr. 11, 2019.
(60) Provisional application No. 62/861,667, filed on Jun. 14, 2019.
(60) Provisional application No. 62/861,992, filed on Jun. 14, 2019.
(60) Provisional application No. 62/899,378, filed on Sep. 12, 2019.

(51) Int. Cl. C12P 7/42; C12N 1/18; C12N 15/52; C12N 9/02; C12N 9/10; C12P 7/22

(52) U.S. Cl. CPC C12P 7/42; C12N 1/18; C12N 15/52; C12N 9/001; C12N 9/1029; C12N 9/1085; C12Y 205/0101; C12Y 103/03006; C12Y 121/03007; C12Y 121/03008; C12Y 203/01206; C12Y 205/01102; C12P 7/22

(57) ABSTRACT

Disclosed herein are microorganism and methods that can be used for the synthesis of cannabigerolic acid (CBGA) and cannabinoids. The methods disclosed can be used to produce CBGA, Δ⁹-tetrahydrocannabinolic acid (THCA), cannabidiolic acid (CBDA), cannabichromenic acid (CBGA), Δ⁹-tetrahydrocannabivarinic acid (THCVA), cannabidivarinic acid (CBDVA), cannabichromevarinic acid (CBCVA), Δ⁹-tetrahydrocannabinol (THC), cannabidiol (CBD), cannabichromene (CBC). Enzymes useful for the synthesis of CBGA and cannabinoids, include but are not limited to acyl activating enzyme (AAE1), polyketide synthase (PKS), olivetolic acid cyclase (OAC), prenyltransferase (PT), THCA synthase (THCAS), CBDA synthase (CBDAS), CBC A synthase (CBCAS), HMG-Co reductase (HMG1), and/or famesyl pyrophosphate synthetase (ERG20). The microorganisms can also have one or more genes disrupted, such as gene that that controls beta oxidation of long chain fatty acids.

### CROSS-REFERENCE TO RELATED APPLICATIONS

This application claims priority to U.S. Provisional Patent Application No. 62/899,378, filed Sep. 12, 2019, U.S. Provisional Patent Application No. 62/861,992, filed Jun. 14, 2019, U.S. Provisional Patent Application No. 62/861,667, filed Jun. 14, 2019, and U.S. Provisional Patent Application No. 62/832,852, filed Apr. 11, 2019, all of which are incorporated herein by reference in their entireties.

### SEQUENCE LISTING

The instant application contains a Sequence Listing which has been submitted electronically in ASCII format and is hereby incorporated by reference in its entirety. Said ASCII file, created on Apr. 10, 2020, is named 35066-002WO_SL.txt and is 1,684,311 bytes in size.

### BACKGROUND OF THE DISCLOSURE

Cannabis sativa (marijuana, hemp; Cannabaceae) is a medicinal and psychoactive herbal drug. Its unique effects are believed to be caused by cannabinoids, which include Δ⁹-tetrahydrocannabinol (THC) and more than 80 related metabolites. Medical marijuana and cannabinoid drugs are increasingly used to treat a range of diseases and conditions such as multiple sclerosis and chronic pain.

Currently, the production of cannabinoids for pharmaceutical or other use is through the extraction of cannabinoids from plants, for example Cannabis sativa, or by chemical synthesis.

There are several drawbacks of the natural production and extraction of cannabinoids from plants. It is often difficult to reproduce identical cannabinoid profiles in plants using an extraction process. In addition, extraction from Cannabis sativa produces a mixture of cannabinoids, which can be difficult to purify to provide a single compound needed for pharmaceutical applications.

The chemical synthesis of various cannabinoids is a costly process compared to extraction, but it provides the final product as single pure product, which is often required for pharmaceutical use.

The microbial fermentation-based production of cannabigerolic acid (“CBGA”) or cannabinoids can be more economical, more robust, scalable, and can provide specific cannabinoid products for simplified isolation and purification versus current routes.

There are some known microbial fermentation processes. For example, WO 2016/010827 A1 and WO 2011/017798 A1 describe several processes. However, attempts at reproducing the methods disclosed therein, were unsuccessful: CBGA was not produced.

The methods described in WO 2019/071000 A1, incorporated herein by reference, were successful at producing CGBA, however higher titers of CBGA and various cannabinoids are desired. The inventors have discovered ways to produce cannabinoids as described herein.

### INCORPORATION BY REFERENCE

All publications, patents, and patent applications herein are incorporated by reference in their entireties to the same extent as if each individual publication, patent, or patent application was specifically and individually indicated to be incorporated by reference in its entirety. In the event of a conflict between a term herein and a term in an incorporated reference, the term herein controls.

### SUMMARY

This application discloses microorganisms that are capable of producing CBGA and cannabinoids (e.g., THC), in an efficient manner, as well as methods of increasing the efficiency of CBGA and cannabinoid, cannabinoid intermediate, or cannabinoid precursor synthesis (“cannabinoid,” “cannabinoid intermediate,” and “cannabinoid precursor” used interchangibly herein). The products that can be made by the processes and microorganism described herein can include, but are not limited to CBGA, Δ⁹-tetrahydrocannabinolic acid (THCA), cannabidiolic acid (CBDA), cannabichromenic acid (CBCA), Δ⁹-tetrahydrocannabinol (THC), cannabidiol (CBD), cannabichromene (CBC), Δ⁹-tetrahydrocannabivarinic acid (THCVA), cannabidivarinic acid (CBDVA), and cannabichromevarinic acid (CBCVA), as described in WO 2019/071000 (herein incorporated by reference) and as described herein. In some cases, a combination of mutants, deletions and yeast strains may be used as described or in various combinations.

In an embodiment a genetically modified microorganism comprises at least three polynucleotides that encode for: a) an amino acid sequence that is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO: 27; b) an amino acid sequence that is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO: 32; or c) combinations thereof. In an embodiment, the genetically modified microorganism of claim 1 comprises a polynucleotide that encodes for an amino acid sequence that is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO: 27. In an embodiment, the genetically modified microorganism comprises a polynucleotide that encodes for an amino acid sequence that is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO: 32.

In an embodiment, the genetically modified microorganism comprises at least two polynucleotides that encode for an amino acid sequence that is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO: 32.

In an embodiment, the at least three polynucleotides encode for proteins having prenyltransferase activity.

In an embodiment, the microorganism comprises at least one polynucleotide that encodes a F96W mutant of Saccharomyces cerevisiae ERG20. In an embodiment, the microorganism comprises at least one polynucleotide that encodes an N127W mutant of Saccharomyces cerevisiae ERG20. In an embodiment, at least one of the microorganism's engodenous genes is disrupted; preferably wherein the engodenous genes is deleted. In an embodiment, the microorganism further comprises the polynucleotide sequence of the Saccharomyces cerevisiae GAL1/GAL10 promoter. In an embodiment, the GAL1/GAL10 promoter is inserted into the microorganism's native LPP1 locus. In an embodiment, the microorganism's native LPP1 open reading frame is deleted. In an embodiment, the microorganism further comprises at least one polynucleotide that encodes for an amino acid sequence that is substantially identical to a truncated amino acid sequence of the Saccharomyces cerevisiae HMG1, wherein the first 530 amino acids of the HMG1 are truncated.

In an embodiment, the genetically modified microorganism further comprises at least one polynucleotide encoding at least one polypeptide with acyl activating activity; polyketide synthase activity; olivetol synthase activity; tetraketide synthase activity; olivetolic acid cyclase activity; THCA synthase activity; CBDA synthase activity; CBCA synthase activity; HMG-Co reductase activity; farnesyl pyrophosphate synthetase activity; or any combination thereof. In an embodiment, the genetically modified microorganism further comprises at least one polynucleotide encoding an acyl activating enzyme (AAE1); a polyketide synthase (PKS), such as a tetraketide synthase (TKS); an olivetolic acid cyclase (OAC); a THCA synthase (THCAS); a CBDA synthase (CBDAS); a CBCA synthase (CBCAS); a HMG-Co reductase (HMG1); a farnesyl pyrophosphate synthetase (ERG20); or any combination thereof; preferably wherein the AAE1 is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO: 14; preferably wherein the TKS is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO: 41; preferably wherein the OAC is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO: 8; preferably wherein the THCAS is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to a sequence chosen from SEQ ID NO: 10 or SEQ ID NO: 120; preferably wherein the THCAS is a T446A mutant of SEQ ID NO: 120, a T446V mutant of SEQ ID NO: 120, or a T446I mutant of SEQ ID NO: 120, or a combination thereof; preferably wherein the polynucleotide encodes a THCAS signal sequence substantially identical to, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to a sequence chosen from SEQ ID NO: 121 to SEQ ID NO: 138; preferably wherein the CBDAS is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO: 12; preferably wherein the CBCAS is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO: 18; preferably wherein the HMG1 is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO: 20 or SEQ ID NO: 22; preferably wherein the ERG20 is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO: 24.

In an embodiment, the genetically modified microorganism comprises at least one polynucleotide encoding an enzyme that is capable of converting olivetolic acid to cannabigerolic acid (“CBGA”). In an embodiment, the genetically modified microorganism further comprises at least one polynucleotide encoding an enzyme that is capable of converting butyric acid to cannabigerolic acid (“CBGVA”).

In an embodiment, the genetically modified microorganism further comprises a polynucleotide that encodes for an amino acid sequence that is substantially identical to, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO: 5.

In an embodiment, the genetically modified microorganism further comprises a polynucleotide that is substantially identical to, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO: 6.

In an embodiment, the genetically modified microorganism further comprises a polynucleotide that encodes for an amino acid sequence that is substantially identical to, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO: 7.

In an embodiment, the genetically modified microorganism further comprises a polynucleotide that is substantially identical to, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO:8.

In an embodiment, the genetically modified microorganism further comprises a polynucleotide that encodes for an amino acid sequence that is substantially identical to, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO: 13.

In an embodiment, the genetically modified microorganism further comprises a polynucleotide that is substantially identical to, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO:14.

In an embodiment, the genetically modified microorganism comprises at least two polynucleotides encoding a protein with AAE1 activity. In an embodiment, the genetically modified microorganism comprises at least three polynucleotides encoding a protein with AAE1 activity. In an embodiment, genetically modified microorganism comprises at least two polynucleotides encoding a protein with TKS activity. In an embodiment, the genetically modified microorganism comprises at least three polynucleotides encoding a protein with TKS activity. In an embodiment, the genetically modified microorganism comprises at least two polynucleotides encoding a protein with OAC activity. In an embodiment, the genetically modified microorganism comprises at least three polynucleotides encoding a protein with OAC activity. In an embodiment, the genetically modified microorganism comprises at least three polynucleotides encoding a protein with AAE1 activity; at least three polynucleotides encoding a protein with TKS activity; and at least three polynucleotides encoding a protein with OAC activity.

In an embodiment, the genetically modified microorganism further comprises one or more polynucleotides encoding proteins with Hydroxymethylglutaryl-CoA synthase activity; Hydroxymethylglutaryl-CoA reductase activity; tHMG1 activity; Acetyl-CoA C-acetyltransferase activity; or any combination thereof. In an embodiment, the genetically modified microorganism further comprises one or more polynucleotides encoding a Hydroxymethylglutaryl-CoA synthase (ERG13); a Hydroxymethylglutaryl-CoA reductase (HMG1); a tHMG1; a Acetyl-CoA C-acetyltransferase (ERG10); or any combination thereof. In an embodiment, the genetically modified microorganism further comprises a polynucleotide encoding an ERG13; a polynucleotide encoding a HGM1 and a polynucleotide encoding an amino acid sequence that is substantially identical to, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical SEQ ID NO: 32. In an embodiment, the genetically modified microorganism further comprises a polynucleotide encoding a tHMG1; a polynucleotide encoding an ERG10 and a polynucleotide encoding an EGR13. In an embodiment, the genetically modified microorganism further comprises a polynucleotide encoding a tHMG1; a polynucleotide encoding an ERG13 and a polynucleotide encoding an AAE1. In an embodiment, the genetically modified microorganism further comprises a polynucleotide encoding an enzyme with CBDA synthase activity, a polynucleotide encoding an enzyme with CBCA synthase, a polynucleotide encoding an enzyme with CBCA and CBDA synthase activity, or a combination thereof, preferably wherein the the enzyme with CBDA synthase activity is substantially identical to, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to a sequence chosen from SEQ ID NO; 43 or SEQ ID NO: 153 to SEQ ID NO: 287; preferably wherein the polynucleotide encodes a CBDA synthase signal sequence substantially identical to, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to a sequence chosen from SEQ ID NO: 44 to SEQ ID NO: 73 or SEQ ID NO: 104 to SEQ ID NO: 110, preferably wherein the enzyme with CBCA synthase activity is substantially identical to, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to a sequence chosen from SEQ ID NO: 288 to SEQ ID NO: 297 or SEQ ID NO: 305 to SEQ ID NO: 318.

In an embodiment, the genetically modified microorganism further comprises the bCBGA1854 plasmid of SEQ ID No.: 435.

In an embodiment, the genetically modified microorganism further comprises a polynucleotiode encoding a protein with PKS activity, a polynucleotiode encoding a protein with OAC activity, and a polynucleotiode encoding a protein with AAE1 activity.

In an embodiment, the genetically modified microorganism further comprises a polynucleotide encoding a PIR3-CBDA of SEQ ID NO: 302.

In an embodiment, the genetically modified microorganism further comprises a signal peptide corresponding to 0253/asn053-2.

In an embodiment, the genetically modified microorganism's engodenous VPS10 gene is disrupted; preferably wherein the sequence of the disrupted gene is SEQ ID NO: 300. In an embodiment, the coding sequence of the microorganism's engodenous VPS10 gene is deleted

In an embodiment, the microorganism is capable of producing cannabigerolic acid. In an embodiment, the microorganism is capable of producing a cannabinoid. In an embodiment, the cannabinoid is selected from Δ9-tetrahydrocannabinolic acid (THCA), cannabidiolic acid (CBDA), cannabichromenic acid (CBCA), Δ9-tetrahydrocannabinol (THC), cannabidiol (CBD), cannabichromene (CBC), Δ⁹-tetrahydrocannabivarinic acid (THCVA), cannabidivarinic acid (CBDVA), cannabichromevarinic acid (CBCVA), or any combination thereof.

In an embodiment, the genetically modified microorganism comprises one or more endogenous genes is from a pathway that controls beta oxidation of long chain fatty acids. In an embodiment, the at least one endogenous gene is FOX1, FAA1, FAA4, FAT1, PXA1, PXA2, and/or PEX11. In an embodiment, the at least one endogenous gene is FOX1. In an embodiment, the one or more gene is disrupted using a CRISPR/Cas system.

In an embodiment, the genetically modified microorganism is a bacterium or a yeast. In an embodiment, said microorganism is a yeast. In an embodiment, said yeast is from the genus Saccharomyces. In an embodiment, wherein said yeast is from the species Saccharomyces cerevisiae.

In an embodiment, the genetically modified microorganism comprises at least two polynucleotides that encode for amino acid sequences that are substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO: 27, SEQ ID NO: 32, or combinations thereof.

In an embodiment, the genetically modified microorganism comprises

genetically modified microorganism comprises at least three polynucleotides that encode for a protein with acyl activating activity, at least three polynucleotides that encode for a protein with polyketide synthase activity, at least three polynucleotides that encode for a protein with olivetolic acid cyclase activity.

In an embodiment, the genetically modified microorganism comprises a polynucleotide that encodes for a Saccharomyces cerevisiae TKS with a mutation at Ala125. In an embodiment, the mutation is Ala125Ser.

In an embodiment, the organism comprises a polynucleotide sequence encoding at least one amino acid sequence substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to a sequence chosen from SEQ ID NO: 153 to SEQ ID NO: 287; preferably wherein the polynucleotide further encodes a CBDA synthase signal sequence substantially identical to, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to a sequence chosen from SEQ ID NO: 44 to SEQ ID NO: 73 or SEQ ID NO: 104 to SEQ ID NO: 110.

In an embodiment, the genetically modified organism comprises a polynucleotide sequence encoding at least one amino acid sequence substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to a sequence chosen from SEQ ID NO: 288 to SEQ ID NO: 297 or SEQ ID NO: 305 to SEQ ID NO: 318.

In an embodiment the modified organism comprises a polypeptide comprising an amino acid sequence substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO: 27.

In an embodiment the modified organism comprises a polynucleotide sequence encoding at least one amino acid sequence substantially identical to, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID: No. 120; preferably wherein the THCAS is a T446A mutant of SEQ ID NO: 120; a T446V mutant of SEQ ID NO: 120; or a T446I mutant of SEQ ID NO: 120, or a combination thereof.

In an embodiment, the genetically modified microorganism comprises at least one polynucleotide encoding for an amino acid sequence that is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to a sequence chosen from SEQ ID NO: 320 to SEQ ID NO: 379, a K239A+I240V+L241A combination mutant of SEQ ID NO: 320; a N242D mutant of SEQ ID NO: 320; a N24Q mutant of SEQ ID NO: 320; a G244L+H245K mutant of SEQ ID NO: 320; a K249R mutant of SEQ ID NO: 320; a C264S mutant of SEQ ID NO: 320; a F272I mutant of SEQ ID NO: 320; a R275P mutant of SEQ ID NO: 320; a R275K mutant of SEQ ID NO: 320; a M283I mutant of SEQ ID NO: 320; a M283C+W284F mutant of SEQ ID NO: 320; a F287L mutant of SEQ ID NO: 320; a S295C mutant of SEQ ID NO: 320; a F298G mutant of SEQ ID NO: 320; a F309I mutant of SEQ ID NO: 320; a I314V mutant of SEQ ID NO: 320; a S323A mutant of SEQ ID NO: 320; a S323T mutant of SEQ ID NO: 320; a M326I mutant of SEQ ID NO: 320, a E329Q mutant of SEQ ID NO: 320; a I333L mutant of SEQ ID NO: 320; a L343F mutant of SEQ ID NO: 320; a K348G mutant of SEQ ID NO: 320; a K350N mutant of SEQ ID NO: 320; a L354F mutant of SEQ ID NO: 320; a L354V+F355Y+V356I mutant of SEQ ID NO: 320; a F357Y mutant of SEQ ID NO: 320; a I360C mutant of SEQ ID NO: 320, a F361L mutant of SEQ ID NO: 320; a I363L mutant of SEQ ID NO: 320; a I374L mutant of SEQ ID NO: 320; a Q378K mutant of SEQ ID NO: 320; a T382A mutant of SEQ ID NO: 320; a S398V mutant of SEQ ID NO: 320, a S398V mutant of SEQ ID NO: 320; a T402S mutant of SEQ ID NO: 320; a S417T mutant of SEQ ID NO: 320; a A421L mutant of SEQ ID NO: 320; a M426F mutant of SEQ ID NO: 320, a M428L mutant of SEQ ID NO: 320; a V447+V450I mutant of SEQ ID NO: 320; a S448T mutant of SEQ ID NO: 320; a V450L mutant of SEQ ID NO: 320; a T460S+F461W+V462L mutant of SEQ ID NO: 320, a V473A mutant of SEQ ID NO: 320; a S476L mutant of SEQ ID NO: 320; a W481M mutant of SEQ ID NO: 320; a V484A mutant of SEQ ID NO: 320; a V484L+I489V+I491V mutant of SEQ ID NO: 320, a N488S mutant of SEQ ID NO: 320; a I489V mutant of SEQ ID NO: 320; a S493A mutant of SEQ ID NO: 320; a A495I mutant of SEQ ID NO: 320; a F499S mutant of SEQ ID NO: 320, a C500S mutant of SEQ ID NO: 320; a F503Y mutant of SEQ ID NO: 320; a L510K mutant of SEQ ID NO: 320, a Q520S mutant of SEQ ID NO: 320; a I525L mutant of SEQ ID NO: 320; a L527I mutant of SEQ ID NO: 320; or combinations thereof. In an embodiment, a polynucleotide encoding can encode at least one amino acid sequence disclosed herein. In an embodiment, a vector can comprise a polynucleotide disclosed herein. And in an embodiment, a polypeptide can comprise an amino acid sequence disclosed herein.

In an embodiment, the genetically modified microorganism may comprise at least one polynucleotide encoding for an amino acid sequence that is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to a sequence chosen from SEQ ID NO: 153 to SEQ ID NO: 287, or combinations thereof; preferably wherein the at least one polynucleotide further encodes a CBDA synthase signal sequence substantially identical to, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to a sequence chosen from SEQ ID NO: 44 to SEQ ID NO: 73 or SEQ ID NO: 104 to SEQ ID NO: 110. In an embodiment, a polynucleotide encoding can encode at least one amino acid sequence disclosed herein. In an embodiment, a vector can comprise a polynucleotide disclosed herein. And in an embodiment, a polypeptide can comprise an amino acid sequence disclosed herein.

In an embodiment, the genetically modified microorganism may comprise at least one polynucleotide encoding for an amino acid sequence that is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to a sequence chosen from SEQ ID NO: 288 to SEQ ID NO: 297 or SEQ ID NO: 305 to SEQ ID NO: 318, or combinations thereof. In an embodiement, a polynucleotide encoding can encode at least one amino acid sequence disclosed herein. In an embodiment, a vector can comprise a polynucleotide disclosed herein. And in an embodiment, a polypeptide can comprise an amino acid sequence disclosed herein.

In an embodiment, the genetically modified microorganism may comprise at least one polynucleotide encoding for an amino acid sequence that is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to a sequence chosen from THCAS is a T446A mutant of SEQ ID NO: 120; a T446V mutant of SEQ ID NO: 120; or a T446I mutant of SEQ ID NO: 120, or combinations thereof; preferably wherein the at least one polynucleotide encodes a THCAS signal sequence substantially identical to, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to a sequence chosen from SEQ ID NO: 121 to SEQ ID NO: 138. In an embodiement, a polynucleotide encoding can encode at least one amino acid sequence disclosed herein. In an embodiment, a vector can comprise a polynucleotide disclosed herein. And in an embodiment, a polypeptide can comprise an amino acid sequence disclosed herein.

In an embodiment, a method of producing a cannabinoid comprises (a) contacting a carbon substrate with a genetically modified microorganism disclosed herein (b) growing said genetically modified microorganism to produce a cannabinoid; and optionally (c) isolating the cannabinoid from the genetically modified organism. In an embodiment, the carbon substrate is a sugar, alcohol, and/or fatty acid. In an embodiment, the carbon substrate is selected from hexanoic acid, glucose, fructose, xylose, sucrose, dextrins, starch, xylan, cellulose, hemicellulose, arabinose, glycerol, ethanol, butanol, methanol, or any combination thereof. In an embodiment, the carbon substrate is hexanoic acid. In an embodiment, the cannabinoid is converted to Δ⁹-tetrahydrocannabinol (THC), cannabidiol (CBD), cannabichromene (CBC), or any combination thereof. In an embodiment the conversion is completed outside the microorganism. In an embodiment, the conversion is a non-enzymatic conversion. In an embodiment, the conversion is an enzymatic conversion.

Further embodiments disclose a use of a cannabinoid produced by any one of the disclosed methods for the manufacture of a medicament for the treatment of a disease or a symptom of a disease. In an embodiment, the disease or the symptom of a disease is anorexia, multiple sclerosis, neurodegenerative disorders, epilepsy, glaucoma, osteoporosis, schizophrenia, bipolar disorder, post-traumatic stress disorder (PTSD), asthma, cardiovascular disorders, cancer, obesity, metabolic syndrome-related disorders, depression, anxiety, insomnia, emesis, pain, or inflammation.

Further embodiments disclose a medicament comprising a cannabinoid made by any one of the disclosed methods and a pharmaceutically acceptable excipient. Further embodiments disclose a method of treating a disease or a symptom of a disease comprising administering a subject in need thereof the cannabinoid made by any one of the disclosed methods. In an embodiment, the disease or a symptom of a disease is anorexia, multiple sclerosis, neurodegenerative disorders, epilepsy, glaucoma, osteoporosis, schizophrenia, bipolar disorder, post-traumatic stress disorder (PTSD), asthma, cardiovascular disorders, cancer, obesity, metabolic syndrome-related disorders, depression, anxiety, insomnia, emesis, pain, or inflammation. Further embodiments disclose a method of treating a disease or a symptom of a disease comprising administering a subject in need thereof the disclosed medicament. Further embodiments disclose a use of a cannabinoid produced by any one of the disclosed microorganisms or methods for the manufacture of a medicament for recreational use. In an embodiment, the medicament or cannabinoid is delivered through inhalation, intravenously, oral, or topical application. In an embodiment, the delivery is inhalation and completed through a vaporizer. In an embodiment, the delivery is intravenous and the medicament is delivered through a saline solution. In an embodiment, the delivery is oral and the medicament is delivered with food. In an embodiment, the delivery is oral and the medicament is delivered through drink. In an embodiment, the delivery is topical and the medicament is delivered through a patch. In an embodiment, the delivery is topical and the medicament is delivered through a lotion.

Disclosed herein is a genetically modified microorganism comprising a polynucleotide that is at least 60%, 70%, 80%, 85%, 90%, 95%, 96%, 97%, 98%, or 99% identical to SEQ ID NO: 2. The polynucleotide can encode an amino acid sequence that is at least 60%, 70%, 80%, 85%, 90%, 95%, 96%, 97%, 98%, or 99% identical to SEQ ID NO: 1.

Disclosed herein is also a genetically modified microorganism comprising a polynucleotide that is at least 60%, 70%, 80%, 85%, 90%, 95%, 96%, 97%, 98%, or 99% identical to SEQ ID NO: 26. The polynucleotide can encode an amino acid sequence that is at least 60% identical to SEQ ID NO: 27.

Disclosed herein is also a genetically modified microorganism comprising a polynucleotide that is at least 60%, 70%, 80%, 85%, 90%, 95%, 96%, 97%, 98%, or 99% identical to SEQ ID NO: 31. The polynucleotide can encode an amino acid sequence that is at least 60% identical to SEQ ID NO: 32.

Disclosed herein is also a genetically modified microorganism comprising a polynucleotide that is at least 60%, 70%, 80%, 85%, 90%, 95%, 96%, 97%, 98%, or 99% identical to SEQ ID NO: 37. The polynucleotide can encode an amino acid sequence that is at least 60% identical to SEQ ID NO: 38.

In some cases, the polynucleotide can encode for an enzyme that is capable of converting olivetolic acid to cannabigerolic acid. In other cases, the polynucleotide can encode for a protein having prenyltransferase activity.

In some cases, the genetically modified microorganism can further comprise one or more nucleic acids encoding for acyl activating enzyme (AAE1); polyketide synthase (PKS); tetraketide synthase (TKS) (also referred to as olivetol synthase (OS)); olivetolic acid cyclase (OAC); THCA synthase (THCAS); CBDA synthase (CBDAS); CBCA synthase (CBCAS); HMG-Co reductase (HMG1); farnesyl pyrophosphate synthetase (ERG20); or any combination thereof. For example, if the microorganism comprises an AAE1, the AAE1 can be encoded by a polynucleotide sequence that is substantially identical to SEQ ID NO: 14. If the microorganism comprises a PKS, the PKS can be encoded by a polynucleotide sequence that is substantially identical, 60%, 70%, 80%, 85%, 90%, 95%, 96%, 97%, 98%, or 99% identical to SEQ ID NO: 6. If the microorganism comprises an OAC, the OAC can be encoded by a polynucleotide sequence that is substantially identical, 60%, 70%, 80%, 85%, 90%, 95%, 96%, 97%, 98%, or 99% identical to SEQ ID NO: 8. If the microorganism comprises a THCAS, the THCAS can be encoded by a polynucleotide sequence that is substantially identical, 60%, 70%, 80%, 85%, 90%, 95%, 96%, 97%, 98%, or 99% identical to SEQ ID NO: 10. If the microorganism comprises a CBDAS, the CBDAS can be encoded by a polynucleotide sequence that is substantially identical, 60%, 70%, 80%, 85%, 90%, 95%, 96%, 97%, 98%, or 99% identical to SEQ ID NO: 12. If the microorganism comprises a CBCAS, the CBCAS can be encoded by a polynucleotide sequence that is substantially identical, 60%, 70%, 80%, 85%, 90%, 95%, 96%, 97%, 98%, or 99% identical to SEQ ID NO: 18. If the microorganism comprises a HMG1, the HMG1 can be encoded by a polynucleotide sequence that is at least 90%, 95%, 96%, 97%, 98%, or 99% identical to SEQ ID NO: 20 or 22. If the microorganism comprises an ERG20, the ERG20 can be encoded by a polynucleotide sequence that is at least 90%, 95%, 96%, 97%, 98%, or 99% identical to SEQ ID NO: 24.

Disclosed herein is a method of making CBGA comprising (a) contacting a carbon substrate with a genetically modified microorganism, where the genetically modified microorganism comprises one or more polynucleotides encoding for i) acyl activating enzyme (AAE1); ii) a polyketide synthase (PKS), iii) a olivetolic acid cyclase (OAC), and iv) a prenyltransferase that comprises an amino acid sequence that is at least 60%, 70%, 80%, 85%, 90%, 95%, 96%, 97%, 98%, or 99% identical to SEQ ID NO: 1; and (b) growing the genetically modified microorganism to make CBGA.

Disclosed herein is a method of making CBGA comprising (a) contacting a carbon substrate with a genetically modified microorganism, where the genetically modified microorganism comprises one or more polynucleotides encoding for i) acyl activating enzyme (AAE1); ii) a polyketide synthase (PKS), iii) a olivetolic acid cyclase (OAC), and iv) a prenyltransferase that comprises an amino acid sequence that is at least 60% identical to SEQ ID NO: 27; and (b) growing the genetically modified microorganism to make CBGA.

Disclosed herein is a method of making CBGA comprising (a) contacting a carbon substrate with a genetically modified microorganism, where the genetically modified microorganism comprises one or more polynucleotides encoding for i) acyl activating enzyme (AAE1); ii) a polyketide synthase (PKS), iii) a olivetolic acid cyclase (OAC), and iv) a prenyltransferase that comprises an amino acid sequence that is at least 60%, 70%, 80%, 85%, 90%, 95%, 96%, 97%, 98%, or 99% identical to SEQ ID NO: 32; and (b) growing the genetically modified microorganism to make CBGA.

Disclosed herein is a method of making CBGA comprising (a) contacting a carbon substrate with a genetically modified microorganism, where the genetically modified microorganism comprises one or more polynucleotides encoding for i) acyl activating enzyme (AAE1); ii) a polyketide synthase (PKS), iii) a olivetolic acid cyclase (OAC), and iv) a prenyltransferase that comprises an amino acid sequence that is at least 60%, 70%, 80%, 85%, 90%, 95%, 96%, 97%, 98%, or 99% identical to SEQ ID NO: 38; and (b) growing the genetically modified microorganism to make CBGA.

The methods can also further comprise isolating the CBGA from (b). The method can also further comprise converting CBGA into CBG, Δ⁹-tetrahydrocannabinolic acid; THC; cannabidiolic acid; CBD; cannabichromenic acid; CBC; Δ⁹-tetrahydrocannabivarinic acid;

THCVA; cannabidivarinic acid; CBDVA; cannabichromevarinic acid; CBCVA; other cannabinoid; or any combination thereof. This CBGA conversion can be completed outside the microorganism. In some cases, the conversion is a non-enzymatic conversion. In other cases, the conversion is an enzymatic conversion.

Also disclosed herein is a method of making a cannabinoid comprising (a) contacting a carbon substrate with a genetically modified microorganism, where the genetically modified microorganism comprises one or more polynucleotides encoding for i) acyl activating enzyme (AAE1); ii) a polyketide synthase (PKS), iii) a olivetolic acid cyclase (OAC), iv) a prenyltransferase that comprises an amino acid sequence that is at least 60%, 70%, 80%, 85%, 90%, 95%, 96%, 97%, 98%, or 99% identical to SEQ ID NO: 1, and (v) a THCA synthase (THCAS); CBDA synthase (CBDAS), CBCA synthase (CBCAS), or any combination thereof; and (b) growing the genetically modified microorganism to make a cannabinoid.

Also disclosed herein is a method of making a cannabinoid comprising (a) contacting a carbon substrate with a genetically modified microorganism, where the genetically modified microorganism comprises one or more polynucleotides encoding for i) acyl activating enzyme (AAE1); ii) a polyketide synthase (PKS), iii) a olivetolic acid cyclase (OAC), iv) a prenyltransferase that comprises an amino acid sequence that is at least 60%, 70%, 80%, 85%, 90%, 95%, 96%, 97%, 98%, or 99% identical to SEQ ID NO: 27, and (v) a THCA synthase (THCAS); CBDA synthase (CBDAS), CBCA synthase (CBCAS), or any combination thereof; and (b) growing the genetically modified microorganism to make a cannabinoid.

Also disclosed herein is a method of making a cannabinoid comprising (a) contacting a carbon substrate with a genetically modified microorganism, where the genetically modified microorganism comprises one or more polynucleotides encoding for i) acyl activating enzyme (AAE1); ii) a polyketide synthase (PKS), iii) a olivetolic acid cyclase (OAC), iv) a prenyltransferase that comprises an amino acid sequence that is at least 60%, 70%, 80%, 85%, 90%, 95%, 96%, 97%, 98%, or 99% identical to SEQ ID NO: 32, and (v) a THCA synthase (THCAS); CBDA synthase (CBDAS), CBCA synthase (CBCAS), or any combination thereof; and (b) growing the genetically modified microorganism to make a cannabinoid.

Also disclosed herein is a method of making a cannabinoid comprising (a) contacting a carbon substrate with a genetically modified microorganism, where the genetically modified microorganism comprises one or more polynucleotides encoding for i) acyl activating enzyme (AAE1); ii) a polyketide synthase (PKS), iii) a olivetolic acid cyclase (OAC), iv) a prenyltransferase that comprises an amino acid sequence that is at least 60%, 70%, 80%, 85%, 90%, 95%, 96%, 97%, 98%, or 99% identical to SEQ ID NO: 38, and (v) a THCA synthase (THCAS); CBDA synthase (CBDAS), CBCA synthase (CBCAS), or any combination thereof; and (b) growing the genetically modified microorganism to make a cannabinoid.

The methods can further comprise isolating the cannabinoid from (b).

The carbon substrate used in the methods can be a sugar, alcohol, and/or fatty acid. For example, the sugar, alcohol or fatty acid can include without limitation hexanoic acid, butyric acid, glucose, fructose, xylose, sucrose, dextrins, starch, xylan, cellulose, hemicellulose, arabinose, glycerol, ethanol, butanol, methanol, or any combination thereof. In some cases, the carbon substrate is hexanoic acid. In other cases, the carbon substrate is butyric acid.

The methods can use the same or similar genetically modified microorganism described throughout. For example, if the microorganism comprises an AAE1, the AAE1 can be encoded by a polynucleotide sequence that is substantially identical or at least 60%, 70%, 80%, 85%, 90%, 95%, 96%, 97%, 98%, or 99% identical to SEQ ID NO: 14. If the microorganism comprises a PKS, the PKS can be encoded by a polynucleotide sequence that is substantially identical or at least 60%, 70%, 80%, 85%, 90%, 95%, 96%, 97%, 98%, or 99% identical to SEQ ID NO: 6. If the microorganism comprises an OAC, the OAC can be encoded by a polynucleotide sequence that is substantially identical or at least 60%, 70%, 80%, 85%, 90%, 95%, 96%, 97%, 98%, or 99% identical to SEQ ID NO: 8.

The methods can use a microorganism that can further comprise one or more nucleic acids encoding for THCA synthase (THCAS); CBDA synthase (CBDAS), CBCA synthase (CBCAS); or any combination thereof. If the microorganism comprises a THCAS, the THCAS can be encoded by a polynucleotide sequence that is substantially identical or at least 60%, 70%, 80%, 85%, 90%, 95%, 96%, 97%, 98%, or 99% identical to SEQ ID NO: 10. If the microorganism comprises a CBDAS, the CBDAS can be encoded by a polynucleotide sequence that is substantially identical or at least 60%, 70%, 80%, 85%, 90%, 95%, 96%, 97%, 98%, or 99% identical to SEQ ID NO: 12. If the microorganism comprises a CBCAS, the CBCAS can be encoded by a polynucleotide sequence that is substantially identical or at least 60%, 70%, 80%, 85%, 90%, 95%, 96%, 97%, 98%, or 99% identical to SEQ ID NO: 18. If the microorganism comprises an HMG1, the HMG1 can be encoded by a polynucleotide sequence that is substantially identical or at least 60%, 70%, 80%, 85%, 90%, 95%, 96%, 97%, 98%, or 99% identical to SEQ ID NO: 20 or 22. If the microorganism comprises an ERG20, the ERG20 can be encoded by a polynucleotide sequence that is substantially identical or at least 60%, 70%, 80%, 85%, 90%, 95%, 96%, 97%, 98%, or 99% identical to SEQ ID NO: 24. One or more of these enzymes can be present outside of a microorganism.

The methods can use a microorganism that can further comprise one or more genes that are disrupted. For example, the one or more genes that are disrupted can be from a pathway that controls beta oxidation of long chain fatty acids. In some cases, the one or more genes can be endogenous to the microorganism. In some cases, the one or more genes can comprise FOX1, FAA1, FAA4, FAT1, PXA1, PXA2, and/or PEX11.

Disclosed herein is a vector comprising a polynucleotide that is at least 60% identical or at least 60%, 70%, 80%, 85%, 90%, 95%, 96%, 97%, 98%, or 99% identical to SEQ ID NO: 2 and a promoter suitable for expression in a yeast host. Also disclosed herein is a vector comprising a polynucleotide that is at least 60%, 70%, 80%, 85%, 90%, 95%, 96%, 97%, 98%, or 99% identical to SEQ ID NO: 36 and a promoter suitable for expression in a yeast host. Also disclosed herein is a vector comprising a polynucleotide that is at least 60%, 70%, 80%, 85%, 90%, 95%, 96%, 97%, 98%, or 99% identical to SEQ ID NO: 31 and a promoter suitable for expression in a yeast host. Also disclosed herein is a vector comprising a polynucleotide that is at least 60%, 70%, 80%, 85%, 90%, 95%, 96%, 97%, 98%, or 99% identical to SEQ ID NO: 37 and a promoter suitable for expression in a yeast host.

Also disclosed herein is an isolated polynucleotide comprising a nucleotide sequence that is at least 60%, 70%, 80%, 85%, 90%, 95%, 96%, 97%, 98%, or 99% identical to SEQ ID NO: 2. Also disclosed herein is an isolated polynucleotide comprising a nucleotide sequence that is at least 60%, 70%, 80%, 85%, 90%, 95%, 96%, 97%, 98%, or 99% identical to SEQ ID NO: 26.

Also disclosed herein is an isolated polynucleotide comprising a nucleotide sequence that is at least 60%, 70%, 80%, 85%, 90%, 95%, 96%, 97%, 98%, or 99% identical to SEQ ID NO: 31.

Also disclosed herein is an isolated polynucleotide comprising a nucleotide sequence that is at least 60%, 70%, 80%, 85%, 90%, 95%, 96%, 97%, 98%, or 99% identical to SEQ ID NO: 37.

Further disclosed herein is a method of making a genetically modified microorganism capable of synthesizing CBGA comprising (a) contacting a microorganism with a polynucleotide that is at least 60%, 70%, 80%, 85%, 90%, 95%, 96%, 97%, 98%, or 99% identical to SEQ ID NO: 2; and (b) growing the microorganism so that the polynucleotide is inserted into the microorganism.

Also disclosed herein is a method of making a genetically modified microorganism capable of synthesizing CBGA comprising (a) contacting a microorganism with a polynucleotide that is at least 60%, 70%, 80%, 85%, 90%, 95%, 96%, 97%, 98%, or 99% identical to SEQ ID NO: 26; and (b) growing the microorganism so that the polynucleotide is inserted into the microorganism.

Also disclosed herein is a method of making a genetically modified microorganism capable of synthesizing CBGA comprising (a) contacting a microorganism with a polynucleotide that is at least 60%, 70%, 80%, 85%, 90%, 95%, 96%, 97%, 98%, or 99% identical to SEQ ID NO: 31; and (b) growing the microorganism so that the polynucleotide is inserted into the microorganism.

Also disclosed herein is a method of making a genetically modified microorganism capable of synthesizing CBGA comprising (a) contacting a microorganism with a polynucleotide that is at least 60%, 70%, 80%, 85%, 90%, 95%, 96%, 97%, 98%, or 99% identical to SEQ ID NO: 37; and (b) growing the microorganism so that the polynucleotide is inserted into the microorganism.

Also disclosed herein is a method of making a genetically modified microorganism capable of synthesizing cannabinoid comprising (a) contacting a microorganism with a polynucleotide that is at least 60%, 70%, 80%, 85%, 90%, 95%, 96%, 97%, 98%, or 99% identical to SEQ ID NO: 2; and (b) growing the microorganism so that the polynucleotide is inserted into the microorganism.

Also disclosed herein is a method of making a genetically modified microorganism capable of synthesizing cannabinoid comprising (a) contacting a microorganism with a polynucleotide that is at least 60%, 70%, 80%, 85%, 90%, 95%, 96%, 97%, 98%, or 99% identical to SEQ ID NO: 26; and (b) growing the microorganism so that the polynucleotide is inserted into the microorganism.

Also disclosed herein is a method of making a genetically modified microorganism capable of synthesizing cannabinoid comprising (a) contacting a microorganism with a polynucleotide that is at least 60%, 70%, 80%, 85%, 90%, 95%, 96%, 97%, 98%, or 99% identical to SEQ ID NO: 31; and (b) growing the microorganism so that the polynucleotide is inserted into the microorganism.

Also disclosed herein is a method of making a genetically modified microorganism capable of synthesizing cannabinoid comprising (a) contacting a microorganism with a polynucleotide that is at least 60%, 70%, 80%, 85%, 90%, 95%, 96%, 97%, 98%, or 99% identical to SEQ ID NO: 37; and (b) growing the microorganism so that the polynucleotide is inserted into the microorganism.

In some cases, the microorganism can translate the polynucleotide into an amino acid sequence that is at least 60%, 70%, 80%, 85%, 90%, 95%, 96%, 97%, 98%, or 99% identical to SEQ ID NO: 1. In some cases, the microorganism can translate the polynucleotide into an amino acid sequence that is at least 60%, 70%, 80%, 85%, 90%, 95%, 96%, 97%, 98%, or 99% identical to SEQ ID NO: 27. In some cases, the microorganism can translate the polynucleotide into an amino acid sequence that is at least 60%, 70%, 80%, 85%, 90%, 95%, 96%, 97%, 98%, or 99% identical to SEQ ID NO: 32. In some cases, the microorganism can translate the polynucleotide into an amino acid sequence that is at least 60%, 70%, 80%, 85%, 90%, 95%, 96%, 97%, 98%, or 99% identical to SEQ ID NO: 38. The polynucleotide can encode for a protein having prenyltransferase activity.

In some cases, the microorganism can be a bacterium or a yeast. If a yeast, the yeast can be from the genus Saccharomyces.

The microorganism can also comprise one or more additional polynucleotides that encodes for acyl activating enzyme (AAE1); polyketide synthase (PKS); olivetolic acid cyclase (OAC); THCA synthase (THCAS); CBDA synthase (CBDAS), CBCA synthase (CBCAS); HMG-Co reductase (HMG1); farnesyl pyrophosphate synthetase (ERG20); or any combination thereof.

In some cases, the method can comprise a genetically modified microorganism that comprises a polynucleotide encoding for an acyl activating enzyme (AAE1); polyketide synthase (PKS); olivetolic acid cyclase (OAC); and a prenyltransferase that is at least 60%, 70%, 80%, 85%, 90%, 95%, 96%, 97%, 98%, or 99% identical to SEQ ID NO: 2.

The methods can result in a cannabinoid, where the cannabinoid is Δ⁹-tetrahydrocannabinolic acid (THCA), cannabidiolic acid (CBDA), cannabichromenic acid (CBCA), Δ⁹-tetrahydrocannabinol (THC), cannabidiol (CBD), cannabichromene (CBC), Δ⁹-tetrahydrocannabivarinic acid (THCVA), cannabidivarinic acid (CBDVA), cannabichromevarinic acid (CBCVA) or any combination thereof.

Further disclosed is the use of a cannabinoid made by any one of the microorganisms or methods disclosed throughout for the manufacture of a medicament for recreational use. In some cases, the recreational use is delivered through inhalation, intravenously, oral, or topical. In some cases, the delivery is inhalation and completed through a vaporizer. In some cases, the delivery is intravenous and completed through a saline solution. In some cases, the delivery is oral and completed through food. In some cases, the delivery is oral and completed through drink. In some cases, the delivery is topical and completed through a patch. In some cases, the delivery is topical and completed through a lotion.

Further disclosed herein is a genetically modified microorganism that is capable of making a CBGA, which comprising a disruption of an endogenous gene that is FOX1. Further disclosed herein is a genetically modified microorganism that is capable of making a CBGA or CBGVA, which comprises a disruption of an endogenous gene that is VPS10. Further disclosed herein is a genetically modified microorganism of claims wherein FOX1 and VPS10 genes are deleted.

Further disclosed herein is a genetically modified microorganism comprising a polynucleotide that is at least 60% identical to the sequences depicted in FIG. 6A or 6B or 7.

Further disclosed is a genetically modified microorganism comprising a polynucleotide that is at least 60%, 70%, 80%, 85%, 90%, 95%, 96%, 97%, 98%, or 99% identical to the sequence depicted in Table 1.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows the synthesis pathway from hexanoyl-CoA to CBGA. From CBGA, various cannabinoids can be made including but not limited to THC, CBD, CBC, and CBG.

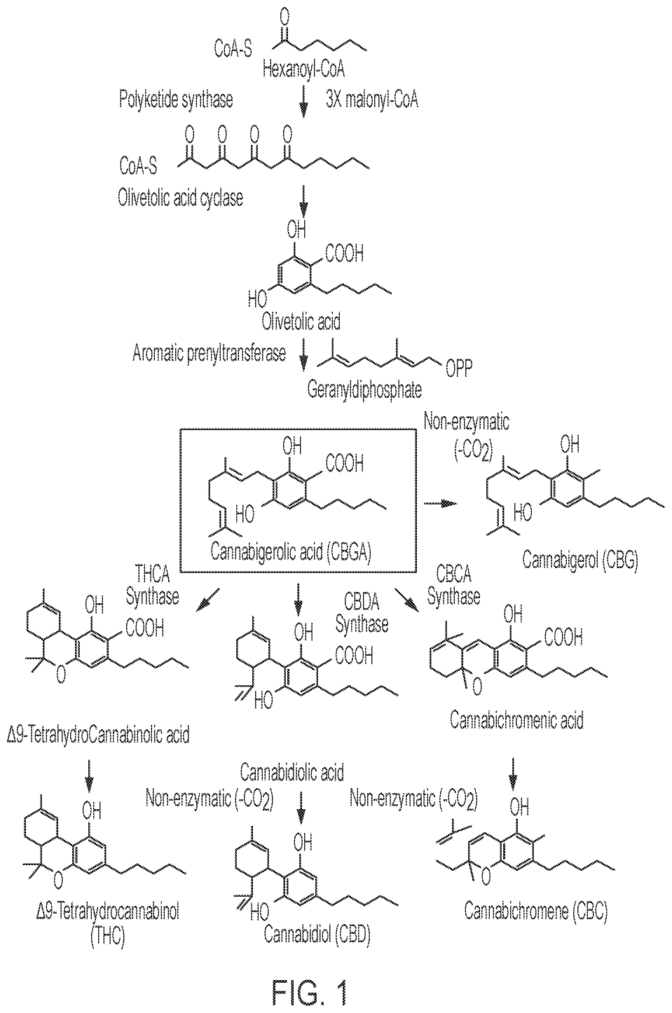

FIG. 2 shows a representative chromatogram of one sample compared to a CBGA standard. This indicates that our strains produce CBGA, since our sample and the CBGA standard overlap.

FIG. 3 shows a representative MRM chromatogram of a THCA containing sample produced by the microorganism described throughout.

FIG. 4 shows a representative UV chromatogram of a THCA containing sample produced by the microorganism described throughout.

FIG. 5 shows the ability of two different yeast strains to produce CBGA, olivetolic acid, and olivetol. yCBGA_0373 strain with a knocked out FOX1 gene produced more CBGA, olivetolic acid, and olivetol compared to its parental yCBGA_0326 strain with wild type FOX1 gene. Error bars show standard deviation of the four replicates measured.

FIG. 6 depicts improved yields on various steps of the high throughput process for production of CBDA.

FIG. 7 depicts a yield of 700 mg/L CBGA using the yCBGA0513 strain in stirred tank fermenter in 3 days from 1 g/L HXA feed.

FIG. 8 depicts the ATG26 locus of the yCBGA0513 strain and the corresponding locuses in the yCBGA0520 strain, the yCBGA0523 strain, and the yCBGA0526 strain which replaced the ATG26 locus. All promoters are labeled beginning with “p.” All coding regions are labeled beginning with “cds.” All terminators are labeled beginning with “t.” The scale ruler (or graphic bar scale) at the top of the figure is in base pairs. Each increment denotes 500 base pairs.

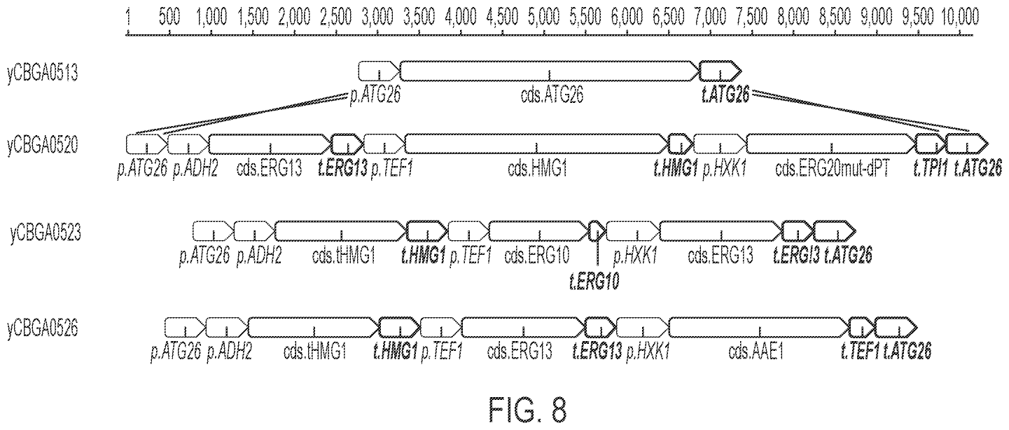

### DETAILED DESCRIPTION OF THE DISCLOSURE

The following description and examples illustrate embodiments of the invention in detail. It is to be understood that this invention is not limited to the particular embodiments described herein and as such can vary. Those of skill in the art will recognize that there are numerous variations and modifications of this invention, which are encompassed within its scope.

The cannabinoid biosynthetic pathway starts with acyl activating enzyme (AAE1) (also known hexanoyl-CoA synthetase) which converts hexanoic acid to hexanoyl-CoA, which is used as a substrate for a reaction involving two enzymes, polyketide synthase (PKS) and olivetolic acid cyclase (OAC), to form olivetolic acid. Olivetolic acid is then geranylated by a prenyltransferase enzyme (PT) to form cannabigerolic acid (CBGA), a branch-point intermediate that is converted by oxidocyclase enzymes to Δ⁹-tetrahydrocannabinolic acid (THCA), cannabidiolic acid (CBDA), and cannabichromenic acid (CBCA). These compounds undergo nonenzymatic decarboxylation to their neutral forms, THC and cannabidiol (CBD) and cannabichromene (CBC), respectively. CBGA is a key pathway intermediate that is an important compound for the preparation of both known, commercialized cannabinoids and compounds in development. In some cases, butyric acid is used as a substrate for cannabinoid biosynthesis.

Described herein are genetically modified microorganisms, enzymes, polynucleotides, and methods to more efficiently produce CBGA or cannabinoids, including, THCA, CBDA, CBCA, THC, CBC and CBD.

### Definitions

The term “about” in relation to a reference numerical value and its grammatical equivalents as used herein can include the numerical value itself and a range of values plus or minus 10% from that numerical value. For example, the amount “about 10” includes 10 and any amounts from 9 to 11. For example, the term “about” in relation to a reference numerical value can also include a range of values plus or minus 10%, 9%, 8%, 7%, 6%, 5%, 4%, 3%, 2%, or 1% from that value. In some cases, the numerical disclosed throughout can be “about” that numerical value even without specifically mentioning the term “about.”

The term “genetic modification” or “genetically modified” and their grammatical equivalents as used herein can refer to one or more alterations of a nucleic acid, e.g., the nucleic acid within a microorganism's genome. For example, genetic modification can refer to alterations, additions, and/or deletion of nucleic acid (e.g., whole genes or fragments of genes).

The term “disrupting” and its grammatical equivalents as used herein can refer to a process of altering a gene, e.g., by deletion, insertion, mutation, rearrangement, or any combination thereof. For example, a gene can be disrupted by knockout. Disrupting a gene can be partially reducing or completely suppressing expression (e.g., mRNA and/or protein expression) of the gene. Disrupting can also include inhibitory technology, such as shRNA, siRNA, microRNA, dominant negative, or any other means to inhibit functionality or expression of a gene or protein.

The term “gene editing” and its grammatical equivalents as used herein can refer to genetic engineering in which one or more nucleotides are inserted, replaced, or removed from a genome. For example, gene editing can be performed using a nuclease (e.g., a natural-existing nuclease or an artificially engineered nuclease).

The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.”

The term “sugar” and its grammatical equivalents as used herein can include, but are not limited to (i) simple carbohydrates, such as monosaccharides (e.g., glucose fructose, galactose, ribose); disaccharides (e.g., maltose, sucrose, lactose); oligosaccharides (e.g., raffinose, stachyose); or (ii) complex carbohydrates, such as starch (e.g., long chains of glucose, amylose, amylopectin); glycogen; fiber (e.g., cellulose, hemicellulose, pectin, gum, mucilage).

The term “alcohol” and its grammatical equivalents as used herein can include, but are not limited to any organic compound in which the hydroxyl functional group (—OH) is bound to a saturated carbon atom. For example, the term alcohol can include i) monohydric alcohols (e.g., methanol, ethanol, isopropyl alcohol, butanol, pentanol, cetyl alcohol); ii) polyhydric alcohols (e.g., ethylene glycol, propylene glycol, glycerol, erythritol, threitol, xylitol, mannitol, sorbitol, volemitol); iii) unsaturated aliphatic alcohols (e.g., allyl alcohol, geraniol, propargyl alcohol); or iv) alicyclic alcohols (e.g., inositol, menthol).

The term “fatty acid” and its grammatical equivalents as used herein can include, but are not limited to, a carboxylic acid with a long aliphatic chain, which is either saturated or unsaturated. Some examples of unsaturated fatty acids include but are not limited to myristoleic acid, sapienic acid; linoelaidic acid; α-linolenic acid; stearidonic acid; eicosapentaenoic acid; docosahexaenoic acid; linoleic acid; γ-linolenic acid; dihomo-γ-linolenic acid; arachidonic acid; docosatetraenoic acid; palmitoleic acid; vaccenic acid; paullinic acid; oleic acid; elaidic acid; gondoic acid; erucic acid; nervonic acid; and mead acid. Some examples of saturated fatty acids include but are not limited to propionic acid, butyric acid, valeric acid, hexanoic acid, enanthic acid, caprylic acid, pelargonic acid, capric acid, undecylic acid, lauric acid, tridecylic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, hexatriacontylic acid, heptatriacontanoic acid, and octatriacontanoic acid.

The term “substantially pure” and its grammatical equivalents as used herein can mean that a particular substance does not contain a majority of another substance. For example, “substantially pure CBGA” can mean at least 90% CBGA. In some instances, “substantially pure CBGA” can mean at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, 99.99%, 99.999%, or 99.9999% CBGA. For example, substantially pure CBGA can mean at least 70% CBGA. In some cases, substantially pure CBGA can mean at least 75% CBGA. In some cases, substantially pure CBGA can mean at least 80% CBGA. In some cases, substantially pure CBGA can mean at least 85% CBGA. In some cases, substantially pure CBGA can mean at least 90% CBGA. In some cases, substantially pure CBGA can mean at least 91% CBGA. In some cases, substantially pure CBGA can mean at least 92% CBGA. In some cases, substantially pure CBGA can mean at least 93% CBGA. In some cases, substantially pure CBGA can mean at least 94% CBGA. In some cases, substantially pure CBGA can mean at least 95% CBGA. In some cases, substantially pure CBGA can mean at least 96% CBGA. In some cases, substantially pure CBGA can mean at least 97% CBGA. In some cases, substantially pure CBGA can mean at least 98% CBGA. In some cases, substantially pure CBGA can mean at least 99% CBGA. In some cases, substantially pure CBGA can mean at least 99.9% CBGA. In some cases, substantially pure CBGA can mean at least 99.99% CBGA. In some cases, substantially pure CBGA can mean at least 99.999% CBGA. In some cases, substantially pure CBGA can mean at least 99.9999% CBGA.

The term “heterologous” and its grammatical equivalents as used herein can mean “derived from a different species.” For example, a “heterologous gene” can mean a gene that is from a different species. In some instances, as “a yeast comprising a heterologous gene” can mean that the yeast contains a gene that is not from the same yeast. The gene can be from a different microorganism such as bacterium or from a different species such as a different yeast species.

The term “substantially identical” and its grammatical equivalents in reference to another sequence as used herein can mean at least 50% identical. In some instances, the term substantially identical refers to a sequence that is 55% identical. In some instances, the term substantially identical refers to a sequence that is 60% identical. In some instances, the term substantially identical refers to a sequence that is 65% identical. In some instances, the term substantially identical refers to a sequence that is 70% identical. In some instances, the term substantially identical refers to a sequence that is 75% identical. In some instances, the term substantially identical refers to a sequence that is 80% identical. In other instances, the term substantially identical refers to a sequence that is 81% identical. In other instances, the term substantially identical refers to a sequence that is 82% identical. In other instances, the term substantially identical refers to a sequence that is 83% identical. In other instances, the term substantially identical refers to a sequence that is 84% identical. In other instances, the term substantially identical refers to a sequence that is 85% identical. In other instances, the term substantially identical refers to a sequence that is 86% identical. In other instances, the term substantially identical refers to a sequence that is 87% identical. In other instances, the term substantially identical refers to a sequence that is 88% identical. In other instances, the term substantially identical refers to a sequence that is 89% identical. In some instances, the term substantially identical refers to a sequence that is 90% identical. In some instances, the term substantially identical refers to a sequence that is 91% identical. In some instances, the term substantially identical refers to a sequence that is 92% identical. In some instances, the term substantially identical refers to a sequence that is 93% identical. In some instances, the term substantially identical refers to a sequence that is 94% identical. In some instances, the term substantially identical refers to a sequence that is 95% identical. In some instances, the term substantially identical refers to a sequence that is 96% identical. In some instances, the term substantially identical refers to a sequence that is 97% identical. In some instances, the term substantially identical refers to a sequence that is 98% identical. In some instances, the term substantially identical refers to a sequence that is 99% identical. In order to determine the percentage of identity between two sequences, the two sequences are aligned, using for example the alignment method of Needleman and Wunsch (J. Mol. Biol., 1970, 48: 443), as revised by Smith and Waterman (Adv. Appl. Math., 1981, 2: 482) so that the highest order match is obtained between the two sequences and the number of identical amino acids/nucleotides is determined between the two sequences. For example, methods to calculate the percentage identity between two amino acid sequences are generally art recognized and include, for example, those described by Carillo and Lipton (SIAM J. Applied Math., 1988, 48:1073) and those described in Computational Molecular Biology, Lesk, e.d. Oxford University Press, New York, 1988, Biocomputing: Informatics and Genomics Projects. Generally, computer programs will be employed for such calculations. Computer programs that can be used in this regard include, but are not limited to, GCG (Devereux et al., Nucleic Acids Res., 1984, 12: 387) BLASTP, BLASTN and FASTA (Altschul et al., J. Molec. Biol., 1990:215:403). A particularly preferred method for determining the percentage identity between two polypeptides involves the Clustal W algorithm (Thompson, J D, Higgines, D G and Gibson T J, 1994, Nucleic Acid Res 22(22): 4673-4680 together with the BLOSUM 62 scoring matrix (Henikoff S & Henikoff, J G, 1992, Proc. Natl. Acad. Sci. USA 89: 10915-10919 using a gap opening penalty of 10 and a gap extension penalty of 0.1, so that the highest order match obtained between two sequences wherein at least 50% of the total length of one of the two sequences is involved in the alignment.

The term “polyketide synthase”, “PKS”, “tetraketide synthase”, “olivetol synthase”, “OLS”, “OS” and their grammatical equivalents can be interchangeably used, as they refer to the same enzyme.

### General

A cannabinoid is one of a class of diverse chemical compounds that acts on cannabinoid receptors. Cannabinoids can alter neurotransmitter release in the brain. Ligands for these receptor proteins include the endocannabinoids (produced naturally in the body by animals), the phytocannabinoids (found in cannabis and some other plants), and synthetic cannabinoids (manufactured artificially). The most notable cannabinoid is the phytocannabinoid tetrahydrocannabinol (THC), the primary psychoactive compound in cannabis. Cannabidiol (CBD) is another major constituent of the plant. There are at least 113 different cannabinoids isolated from cannabis, exhibiting varied effects.

Cannabinoids can be useful in treating the side effects of cancer and cancer treatment. For example, one of the severe side effects of chemotherapy is loss of appetite. Marinol (containing delta-9-THC API) has been used to effectively treat this side effect. Other medical uses of cannabinoids include but are not limited to anti-inflammatory activity, blocking cell growth, preventing the growth of blood vessels that supply tumors, antiviral activity, and relieving muscle spasms caused by multiple sclerosis.

Disclosed herein are microorganisms and methods of making CBGA or cannabinoids.

### Microrganisms Used in the Synthesis of Cannabinoids

### Cell-Types

The cells that can be used include but are not limited to plant or animal cells, fungus, yeast, algae, or bacterium. The cells can be prokaryotes or in some cases can be eukaryotes. For example, the cell can be a Saccharomyces cerevisiae, Yarrowia lipolytica, or Escherichia coli, or any other cell disclosed throughout.

In certain cases, the cells are not naturally capable of producing CBGA or cannabinoids (e.g., THC, CBD, CBC, THCVA, CBDVA, CBCVA). In some cases, the cells are able to produce CBGA or cannabinoids but at a low level. By implementation of the methods described herein, the cells can be modified such that the level of CBGA or cannabinoids in the cells is higher relative to the level of CBGA or the same cannabinoid produced in the unmodified cells.

In some cases, the modified cell is capable of producing a substrate capable of being converted into a CBGA or a cannabinoid, however, the cells is not capable of naturally producing a cannabinoid. The genetically modified microorganisms in some cases are unable to produce a substrate capable of being converted into a CBGA or a cannabinoid (for example, hexanoic acid), and the substrate capable of being converted into a CBGA or a cannabinoid is provided to the cells as part of the cell's growth medium. In this case, the genetically modified microorganism can process the substrate into a desired product such as CBGA, THC, CBD, or CBC.

The cell can naturally comprise one or more enzyme capable of catalyzing one or more of the reactions: Hexanoyl-CoA to Olivetolic Acid; Olivetolic Acid to CBGA; CBGA to THCA; CBGA to CBDA; CBGA to CBCA; THCA to THC; CBDA to CBD; or CBCA to CBC.

The cell can naturally comprise one or more enzyme capable of catalyzing one or more of the reactions from a substrate such as butyric acid: CBGVA to THCVA; CBGVA to CBDVA; CBGVA to CBCVA; THCVA to THCV; CBDVA to CBDV; or CBCVA to CBCV.

In some cases, the modified cell is capable of producing a substrate capable of being converted into a CBGVA or a cannabinoid, however, the cells is not capable of naturally producing a cannabinoid. The genetically modified microorganisms in some cases are unable to produce a substrate capable of being converted into a CBGVA or a cannabinoid (for example, butyric acid), and the substrate capable of being converted into a CBGVA or a cannabinoid is provided to the cells as part of the cell's growth medium. In this case, the genetically modified microorganism can process the substrate into a desired product such as THCVA, CBDVA, or CBCVA.

Enzymes

The cells disclosed can be genetically modified with one or more enzymes that are capable of producing CBGA or CBGVA or a cannabinoid, and other pathway intermediates such as olivetolic acid. The cells disclosed can also be genetically modified with one or more enzymes that are capable of assisting in or enhancing the ability of the cell to produce CBGA or CBGVA or a cannabinoid, and other pathway intermediate (as disclosed throughout).

The cell can be modified to include an enzyme that can perform any one of the following reactions: hexanoic acid to hexanoyl-CoA, hexanoyl-CoA to olivetolic Acid; olivetolic Acid to CBGA; CBGA to THCA; CBGA to CBDA; CBGA to CBCA; THCA to THC; CBDA to CBD; or CBCA to CBC. For example, the cell can be modified with one or more of the following enzymes: polyketide synthase (PKS); olivetolic acid cyclase (OAC); prenyltransferase (PT); THCA synthase (THCAS); CBDA synthase (CBDAS), CBCA synthase (CBCAS); or any combination thereof. Additional enzymes that can be included include but are not limited to HMG-CoA reductase, ERG20 reductase, or both. These enzymes can either be endogenous to the cell or heterologous. However, in some cases, even if the enzyme is endogenous, it can be made to be overexpressed. The heterologous enzymes can also be overexpressed.

In some cases, two or more consecutive enzymes in the pathway from a carbon substrate (e.g., sugar) to any of the cannabinoids described throughout (e.g., THCA, CBDA, CBCA, THC, CBD, CBC, CBGVA, THCVA, CBDVA, CBCVA) can be used. In some cases, three or more consecutive enzymes in the pathway can be used. In some cases, four or more consecutive enzymes in the pathway can be used. In some cases, five or more consecutive enzymes in the pathway can be used. In some cases, six or more consecutive enzymes in the pathway can be used. In some cases, seven or more consecutive enzymes in the pathway can be used. In some cases, eight or more consecutive enzymes in the pathway can be used. In some cases, nine or more consecutive enzymes in the pathway can be used. In some cases, ten or more consecutive enzymes in the pathway can be used.

In some cases, when an acyl activating enzyme (AAE1) is desired, the AAE1 can be encoded by an amino acid sequence that is substantially identical to SEQ ID NO: 13. In some cases, the AAE1 can be encoded by an amino acid sequence that is at least 50% identical to SEQ ID NO: 13. In some cases, the AAE1 can be encoded by an amino acid sequence that is at least 55% identical to SEQ ID NO: 13. In some cases, the AAE1 can be encoded by an amino acid sequence that is at least 60% identical to SEQ ID NO: 13. In some cases, the AAE1 can be encoded by an amino acid sequence that is at least 65% identical to SEQ ID NO: 13. In some cases, the AAE1 can be encoded by an amino acid sequence that is at least 70% identical to SEQ ID NO: 13. In some cases, the AAE1 can be encoded by an amino acid sequence that is at least 75% identical to SEQ ID NO: 13. In some cases, the AAE1 can be encoded by an amino acid sequence that is at least 80% identical to SEQ ID NO: 13. In some cases, the AAE1 can be encoded by an amino acid sequence that is at least 81% identical to SEQ ID NO: 13. In some cases, the AAE1 can be encoded by an amino acid sequence that is at least 82% identical to SEQ ID NO: 13. In some cases, the AAE1 can be encoded by an amino acid sequence that is at least 83% identical to SEQ ID NO: 13. In some cases, the AAE1 can be encoded by an amino acid sequence that is at least 84% identical to SEQ ID NO: 13. In some cases, the AAE1 can be encoded by an amino acid sequence that is at least 85% identical to SEQ ID NO: 13. In some cases, the AAE1 can be encoded by an amino acid sequence that is at least 86% identical to SEQ ID NO: 13. In some cases, the AAE1 can be encoded by an amino acid sequence that is at least 87% identical to SEQ ID NO: 13. In some cases, the AAE1 can be encoded by an amino acid sequence that is at least 88% identical to SEQ ID NO: 13. In some cases, the AAE1 can be encoded by an amino acid sequence that is at least 89% identical to SEQ ID NO: 13. In some cases, the AAE1 can be encoded by an amino acid sequence that is at least 90% identical to SEQ ID NO: 13. In some cases, the AAE1 can be encoded by an amino acid sequence that is at least 91% identical to SEQ ID NO: 13. In some cases, the AAE1 can be encoded by an amino acid sequence that is at least 92% identical to SEQ ID NO: 13. In some cases, the AAE1 can be encoded by an amino acid sequence that is at least 93% identical to SEQ ID NO: 13. In some cases, the AAE1 can be encoded by an amino acid sequence that is at least 94% identical to SEQ ID NO: 13. In some cases, the AAE1 can be encoded by an amino acid sequence that is at least 95% identical to SEQ ID NO: 13. In some cases, the AAE1 can be encoded by an amino acid sequence that is at least 96% identical to SEQ ID NO: 13. In some cases, the AAE1 can be encoded by an amino acid sequence that is at least 97% identical to SEQ ID NO: 13. In some cases, the AAE1 can be encoded by an amino acid sequence that is at least 98% identical to SEQ ID NO: 13. In some cases, the AAE1 can be encoded by an amino acid sequence that is at least 99% identical to SEQ ID NO: 13. In some cases, the AAE1 can be encoded by an amino acid sequence that is identical to SEQ ID NO: 13. In some cases, the amino acid sequence can be optimized to correspond to amino acid usage within a specific host organism/cell.

In some cases when a polyketide synthase (PKS) is desired, the PKS can be encoded by an amino acid sequence that is substantially identical to SEQ ID NO: 5. In some cases, the PKS can be encoded by an amino acid sequence that is at least 50% identical to SEQ ID NO: 5. In some cases, the PKS can be encoded by an amino acid sequence that is at least 55% identical to SEQ ID NO: 5. In some cases, the PKS can be encoded by an amino acid sequence that is at least 60% identical to SEQ ID NO: 5. In some cases, the PKS can be encoded by an amino acid sequence that is at least 65% identical to SEQ ID NO: 5. In some cases, the PKS can be encoded by an amino acid sequence that is at least 70% identical to SEQ ID NO: 5. In some cases, the PKS can be encoded by an amino acid sequence that is at least 75% identical to SEQ ID NO: 5. In some cases, the PKS can be encoded by an amino acid sequence that is at least 80% identical to SEQ ID NO: 5. In some cases, the PKS can be encoded by an amino acid sequence that is at least 81% identical to SEQ ID NO: 5. In some cases, the PKS can be encoded by an amino acid sequence that is at least 82% identical to SEQ ID NO: 5. In some cases, the PKS can be encoded by an amino acid sequence that is at least 83% identical to SEQ ID NO: 5. In some cases, the PKS can be encoded by an amino acid sequence that is at least 84% identical to SEQ ID NO: 5. In some cases, the PKS can be encoded by an amino acid sequence that is at least 85% identical to SEQ ID NO: 5. In some cases, the PKS can be encoded by an amino acid sequence that is at least 86% identical to SEQ ID NO: 5. In some cases, the PKS can be encoded by an amino acid sequence that is at least 87% identical to SEQ ID NO: 5. In some cases, the PKS can be encoded by an amino acid sequence that is at least 88% identical to SEQ ID NO: 5. In some cases, the PKS can be encoded by an amino acid sequence that is at least 89% identical to SEQ ID NO: 5. In some cases, the PKS can be encoded by an amino acid sequence that is at least 90% identical to SEQ ID NO: 5. In some cases, the PKS can be encoded by an amino acid sequence that is at least 91% identical to SEQ ID NO: 5. In some cases, the PKS can be encoded by an amino acid sequence that is at least 92% identical to SEQ ID NO: 5. In some cases, the PKS can be encoded by an amino acid sequence that is at least 93% identical to SEQ ID NO: 5. In some cases, the PKS can be encoded by an amino acid sequence that is at least 94% identical to SEQ ID NO: 5. In some cases, the PKS can be encoded by an amino acid sequence that is at least 95% identical to SEQ ID NO: 5. In some cases, the PKS can be encoded by an amino acid sequence that is at least 96% identical to SEQ ID NO: 5. In some cases, the PKS can be encoded by an amino acid sequence that is at least 97% identical to SEQ ID NO: 5. In some cases, the PKS can be encoded by an amino acid sequence that is at least 98% identical to SEQ ID NO: 5. In some cases, the PKS can be encoded by an amino acid sequence that is at least 99% identical to SEQ ID NO: 5. In some cases, the PKS can be encoded by an amino acid sequence that is identical to SEQ ID NO: 5. In some cases, the amino acid sequence can be optimized to correspond to amino acid usage within a specific host organism/cell.

In some cases when an olivetolic acid cyclase (OAC) is desired, the OAC can be encoded by an amino acid sequence that is substantially identical to SEQ ID NO: 7. In some cases, the OAC can be encoded by an amino acid sequence that is at least 50% identical to SEQ ID NO: 7. In some cases, the OAC can be encoded by an amino acid sequence that is at least 55% identical to SEQ ID NO: 7. In some cases, the OAC can be encoded by an amino acid sequence that is at least 60% identical to SEQ ID NO: 7. In some cases, the OAC can be encoded by an amino acid sequence that is at least 65% identical to SEQ ID NO: 7. In some cases, the OAC can be encoded by an amino acid sequence that is at least 70% identical to SEQ ID NO: 7. In some cases, the OAC can be encoded by an amino acid sequence that is at least 75% identical to SEQ ID NO: 7. In some cases, the OAC can be encoded by an amino acid sequence that is at least 80% identical to SEQ ID NO: 7. In some cases, the OAC can be encoded by an amino acid sequence that is at least 81% identical to SEQ ID NO: 7. In some cases, the OAC can be encoded by an amino acid sequence that is at least 82% identical to SEQ ID NO: 7. In some cases, the OAC can be encoded by an amino acid sequence that is at least 83% identical to SEQ ID NO: 7. In some cases, the OAC can be encoded by an amino acid sequence that is at least 84% identical to SEQ ID NO: 7. In some cases, the OAC can be encoded by an amino acid sequence that is at least 85% identical to SEQ ID NO: 7. In some cases, the OAC can be encoded by an amino acid sequence that is at least 86% identical to SEQ ID NO: 7. In some cases, the OAC can be encoded by an amino acid sequence that is at least 87% identical to SEQ ID NO: 7. In some cases, the OAC can be encoded by an amino acid sequence that is at least 88% identical to SEQ ID NO: 7. In some cases, the OAC can be encoded by an amino acid sequence that is at least 89% identical to SEQ ID NO: 7. In some cases, the OAC can be encoded by an amino acid sequence that is at least 90% identical to SEQ ID NO: 7. In some cases, the OAC can be encoded by an amino acid sequence that is at least 91% identical to SEQ ID NO: 7. In some cases, the OAC can be encoded by an amino acid sequence that is at least 92% identical to SEQ ID NO: 7. In some cases, the OAC can be encoded by an amino acid sequence that is at least 93% identical to SEQ ID NO: 7. In some cases, the OAC can be encoded by an amino acid sequence that is at least 94% identical to SEQ ID NO: 7. In some cases, the OAC can be encoded by an amino acid sequence that is at least 95% identical to SEQ ID NO: 7. In some cases, the OAC can be encoded by an amino acid sequence that is at least 96% identical to SEQ ID NO: 7. In some cases, the OAC can be encoded by an amino acid sequence that is at least 97% identical to SEQ ID NO: 7. In some cases, the OAC can be encoded by an amino acid sequence that is at least 98% identical to SEQ ID NO: 7. In some cases, the OAC can be encoded by an amino acid sequence that is at least 99% identical to SEQ ID NO: 7. In some cases, the OAC can be encoded by an amino acid sequence that is identical to SEQ ID NO: 7. In some cases, the amino acid sequence can be optimized to correspond to amino acid usage within a specific host organism/cell.

In some cases when a prenyltransferase (PT) is desired, the PT can be encoded by an amino acid sequence that is substantially identical to any one of SEQ ID NOs: 1, 27, 32, 38 or 320-379. In some cases, the amino acid sequence encoding a prenyltransferase can be at least 50% identical to any one of SEQ ID NOs: 1, 27, 32, 38 or 320-379. In some cases, the amino acid sequence encoding a prenyltransferase can be at least 55% identical to any one of SEQ ID NOs: 1, 27, 32, 38 or 320-379. In some cases, the amino acid sequence encoding a prenyltransferase can be at least 60% identical to any one of SEQ ID NOs: 1, 27, 32, 38 or 320-379. In some cases, the amino acid sequence encoding a prenyltransferase can be at least 65% identical to any one of SEQ ID NOs: 1, 27, 32, 38 or 320-379. In some cases, the amino acid sequence encoding a prenyltransferase can be at least 70% identical to any one of SEQ ID NOs: 1, 27, 32, 38 or 320-379. In some cases, the amino acid sequence encoding a prenyltransferase can be at least 75% identical to any one of SEQ ID NOs: 1, 27, 32, 38 or 320-379. In some cases, the amino acid sequence encoding a prenyltransferase can be at least 80% identical to any one of SEQ ID NOs: 1, 27, 32, 38 or 320-379. In some cases, the amino acid sequence encoding a prenyltransferase can be at least 81% identical to any one of SEQ ID NOs: 1, 27, 32, 38 or 320-379. In some cases, the amino acid sequence encoding a prenyltransferase can be at least 82% identical to any one of SEQ ID NOs: 1, 27, 32, 38 or 320-379. In some cases, the amino acid sequence encoding a prenyltransferase can be at least 83% identical to any one of SEQ ID NOs: 1, 27, 32, 38 or 320-379. In some cases, the amino acid sequence encoding a prenyltransferase can be at least 84% identical to any one of SEQ ID NOs: 1, 27, 32, 38 or 320-379. In some cases, the amino acid sequence encoding a prenyltransferase can be at least 85% identical to any one of SEQ ID NOs: 1, 27, 32, 38 or 320-379. In some cases, the amino acid sequence encoding a prenyltransferase can be at least 86% identical to any one of SEQ ID NOs: 1, 27, 32, 38 or 320-379. In some cases, the amino acid sequence encoding a prenyltransferase can be at least 87% identical to any one of SEQ ID NOs: 1, 27, 32, 38 or 320-379. In some cases, the amino acid sequence encoding a prenyltransferase can be at least 88% identical to any one of SEQ ID NOs: 1, 27, 32, 38 or 320-379. In some cases, the amino acid sequence encoding a prenyltransferase can be at least 89% identical to any one of SEQ ID NOs: 1, 27, 32, 38 or 320-379. In some cases, the amino acid sequence encoding a prenyltransferase can be at least 90% identical to any one of SEQ ID NOs: 1, 27, 32, 38 or 320-379. In some cases, the amino acid sequence encoding a prenyltransferase can be at least 91% identical to any one of SEQ ID NOs: 1, 27, 32, 38 or 320-379. In some cases, the amino acid sequence encoding a prenyltransferase can be at least 92% identical to any one of SEQ ID NOs: 1, 27, 32, 38 or 320-379. In some cases, the amino acid sequence encoding a prenyltransferase can be at least 93% identical to any one of SEQ ID NOs: 1, 27, 32, 38 or 320-379. In some cases, the amino acid sequence encoding a prenyltransferase can be at least 94% identical to any one of SEQ ID NOs: 1, 27, 32, 38 or 320-379. In some cases, the amino acid sequence encoding a prenyltransferase can be at least 95% identical to any one of SEQ ID NOs: 1, 27, 32, 38 or 320-379. In some cases, the amino acid sequence encoding a prenyltransferase can be at least 96% identical to any one of SEQ ID NOs: 1, 27, 32, 38 or 320-379. In some cases, the amino acid sequence encoding a prenyltransferase can be at least 97% identical to any one of SEQ ID NOs: 1, 27, 32, 38 or 320-379. In some cases, the amino acid sequence encoding a prenyltransferase can be at least 98% identical to any one of SEQ ID NOs: 1, 27, 32, 38 or 320-379. In some cases, the amino acid sequence encoding a prenyltransferase can be at least 99% identical to any one of SEQ ID NOs: 1, 27, 32, 38 or 320-379. In some cases, the amino acid sequence encoding a prenyltransferase can be identical to any one of SEQ ID NOs: 1, 27, 32, 38 or 320-379. In some cases, the amino acid sequence can be optimized to correspond to amino acid usage within a specific host organism/cell.

Additionally, other enzymes can be used to make different products. These enzymes can include a THCA synthase (THCAS); CBDA synthase (CBDAS), CBCA synthase (CBCAS), or any combination thereof.

In some cases, when a THCA synthase (THCAS) is desired, the THCAS can be encoded by an amino acid sequence that is substantially identical to SEQ ID NO: 9. In some cases, the amino acid sequence encoding a THCAS can be at least 50% identical to SEQ ID NO: 9. In some cases, the amino acid sequence encoding a THCAS can be at least 55% identical to SEQ ID NO: 9. In some cases, the amino acid sequence encoding a THCAS can be at least 60% identical to SEQ ID NO: 9. In some cases, the amino acid sequence encoding a THCAS can be at least 65% identical to SEQ ID NO: 9. In some cases, the amino acid sequence encoding a THCAS can be at least 70% identical to SEQ ID NO: 9. In some cases, the amino acid sequence encoding a THCAS can be at least 75% identical to SEQ ID NO: 9. In some cases, the amino acid sequence encoding a THCAS can be at least 80% identical to SEQ ID NO: 9. In some cases, the amino acid sequence encoding a THCAS can be at least 81% identical to SEQ ID NO: 9. In some cases, the amino acid sequence encoding a THCAS can be at least 82% identical to SEQ ID NO: 9. In some cases, the amino acid sequence encoding a THCAS can be at least 83% identical to SEQ ID NO: 9. In some cases, the amino acid sequence encoding a THCAS can be at least 84% identical to SEQ ID NO: 9. In some cases, the amino acid sequence encoding a THCAS can be at least 85% identical to SEQ ID NO: 9. In some cases, the amino acid sequence encoding a THCAS can be at least 86% identical to SEQ ID NO: 9. In some cases, the amino acid sequence encoding a THCAS can be at least 87% identical to SEQ ID NO: 9. In some cases, the amino acid sequence encoding a THCAS can be at least 88% identical to SEQ ID NO: 9. In some cases, the amino acid sequence encoding a THCAS can be at least 89% identical to SEQ ID NO: 9. In some cases, the amino acid sequence encoding a THCAS can be at least 90% identical to SEQ ID NO: 9. In some cases, the amino acid sequence encoding a THCAS can be at least 91% identical to SEQ ID NO: 9. In some cases, the amino acid sequence encoding a THCAS can be at least 92% identical to SEQ ID NO: 9. In some cases, the amino acid sequence encoding a THCAS can be at least 93% identical to SEQ ID NO: 9. In some cases, the amino acid sequence encoding a THCAS can be at least 94% identical to SEQ ID NO: 9. In some cases, the amino acid sequence encoding a THCAS can be at least 95% identical to SEQ ID NO: 9. In some cases, the amino acid sequence encoding a THCAS can be at least 96% identical to SEQ ID NO: 9. In some cases, the amino acid sequence encoding a THCAS can be at least 97% identical to SEQ ID NO: 9. In some cases, the amino acid sequence encoding a THCAS can be at least 98% identical to SEQ ID NO: 9. In some cases, the amino acid sequence encoding a THCAS can be at least 99% identical to SEQ ID NO: 9. In some cases, the amino acid sequence encoding a THCAS can be identical to SEQ ID NO: 9. In some cases, the amino acid sequence can be optimized to correspond to amino acid usage within a specific host organism/cell. The use of a THCAS, in some cases, can result in the enzymatic synthesis of Δ9-tetrahydrocannabinol (THC) and the accumulation of THC within the cell or culture medium.

In some cases, when a CBDA synthase (CBDAS) is desired, the CBDAS can be encoded by an amino acid sequence that is substantially identical to SEQ ID NO: 11. In some cases, the amino acid sequence encoding a CBDAS can be at least 50% identical to SEQ ID NO: 11. In some cases, the amino acid sequence encoding a CBDAS can be at least 55% identical to SEQ ID NO: 11. In some cases, the amino acid sequence encoding a CBDAS can be at least 60% identical to SEQ ID NO: 11. In some cases, the amino acid sequence encoding a CBDAS can be at least 65% identical to SEQ ID NO: 11. In some cases, the amino acid sequence encoding a CBDAS can be at least 70% identical to SEQ ID NO: 11. In some cases, the amino acid sequence encoding a CBDAS can be at least 75% identical to SEQ ID NO: 11. In some cases, the amino acid sequence encoding a CBDAS can be at least 80% identical to SEQ ID NO: 11. In some cases, the amino acid sequence encoding a CBDAS can be at least 81% identical to SEQ ID NO: 11. In some cases, the amino acid sequence encoding a CBDAS can be at least 82% identical to SEQ ID NO: 11. In some cases, the amino acid sequence encoding a CBDAS can be at least 83% identical to SEQ ID NO: 11. In some cases, the amino acid sequence encoding a CBDAS can be at least 84% identical to SEQ ID NO: 11. In some cases, the amino acid sequence encoding a CBDAS can be at least 85% identical to SEQ ID NO: 11. In some cases, the amino acid sequence encoding a CBDAS can be at least 86% identical to SEQ ID NO: 11. In some cases, the amino acid sequence encoding a CBDAS can be at least 87% identical to SEQ ID NO: 11. In some cases, the amino acid sequence encoding a CBDAS can be at least 88% identical to SEQ ID NO: 11. In some cases, the amino acid sequence encoding a CBDAS can be at least 89% identical to SEQ ID NO: 11. In some cases, the amino acid sequence encoding a CBDAS can be at least 90% identical to SEQ ID NO: 11. In some cases, the amino acid sequence encoding a CBDAS can be at least 91% identical to SEQ ID NO: 11. In some cases, the amino acid sequence encoding a CBDAS can be at least 92% identical to SEQ ID NO: 11. In some cases, the amino acid sequence encoding a CBDAS can be at least 93% identical to SEQ ID NO: 11. In some cases, the amino acid sequence encoding a CBDAS can be at least 94% identical to SEQ ID NO: 11. In some cases, the amino acid sequence encoding a CBDAS can be at least 95% identical to SEQ ID NO: 11. In some cases, the amino acid sequence encoding a CBDAS can be at least 96% identical to SEQ ID NO: 11. In some cases, the amino acid sequence encoding a CBDAS can be at least 97% identical to SEQ ID NO: 11. In some cases, the amino acid sequence encoding a CBDAS can be at least 98% identical to SEQ ID NO: 11. In some cases, the amino acid sequence encoding a CBDAS can be at least 99% identical to SEQ ID NO: 11. In some cases, the amino acid sequence encoding a CBDAS can be identical to SEQ ID NO: 11. In some cases, the amino acid sequence can be optimized to correspond to amino acid usage within a specific host organism/cell. The use of a CBDAS in some cases can result in the enzymatic synthesis of cannabidiol (CBD) and the accumulation of CBD within the cell or culture medium.

In some cases, when a CBCA synthase (CBCAS) is desired, the CDCS can be encoded by an amino acid sequence that is substantially identical to SEQ ID NO: 17. In some cases, the amino acid sequence encoding a CBCAS can be at least 50% identical to SEQ ID NO: 17. In some cases, the amino acid sequence encoding a CBCAS can be at least 55% identical to SEQ ID NO: 17. In some cases, the amino acid sequence encoding a CBCAS can be at least 60% identical to SEQ ID NO: 17. In some cases, the amino acid sequence encoding a CBCAS can be at least 65% identical to SEQ ID NO: 17. In some cases, the amino acid sequence encoding a CBCAS can be at least 70% identical to SEQ ID NO: 17. In some cases, the amino acid sequence encoding a CBCAS can be at least 75% identical to SEQ ID NO: 17. In some cases, the amino acid sequence encoding a CBCAS can be at least 80% identical to SEQ ID NO: 17. In some cases, the amino acid sequence encoding a CBCAS can be at least 81% identical to SEQ ID NO: 17. In some cases, the amino acid sequence encoding a CBCAS can be at least 82% identical to SEQ ID NO: 17. In some cases, the amino acid sequence encoding a CBCAS can be at least 83% identical to SEQ ID NO: 17. In some cases, the amino acid sequence encoding a CBCAS can be at least 84% identical to SEQ ID NO: 17. In some cases, the amino acid sequence encoding a CBCAS can be at least 85% identical to SEQ ID NO: 17. In some cases, the amino acid sequence encoding a CBCAS can be at least 86% identical to SEQ ID NO: 17. In some cases, the amino acid sequence encoding a CBCAS can be at least 87% identical to SEQ ID NO: 17. In some cases, the amino acid sequence encoding a CBCAS can be at least 88% identical to SEQ ID NO: 17. In some cases, the amino acid sequence encoding a CBCAS can be at least 89% identical to SEQ ID NO: 17. In some cases, the amino acid sequence encoding a CBCAS can be at least 90% identical to SEQ ID NO: 17. In some cases, the amino acid sequence encoding a CBCAS can be at least 91% identical to SEQ ID NO: 17. In some cases, the amino acid sequence encoding a CBCAS can be at least 92% identical to SEQ ID NO: 17. In some cases, the amino acid sequence encoding a CBCAS can be at least 93% identical to SEQ ID NO: 17. In some cases, the amino acid sequence encoding a CBCAS can be at least 94% identical to SEQ ID NO: 17. In some cases, the amino acid sequence encoding a CBCAS can be at least 95% identical to SEQ ID NO: 17. In some cases, the amino acid sequence encoding a CBCAS can be at least 96% identical to SEQ ID NO: 17. In some cases, the amino acid sequence encoding a CBCAS can be at least 97% identical to SEQ ID NO: 17. In some cases, the amino acid sequence encoding a CBCAS can be at least 98% identical to SEQ ID NO: 17. In some cases, the amino acid sequence encoding a CBCAS can be at least 99% identical to SEQ ID NO: 17. In some cases, the amino acid sequence encoding a CBCAS can be identical to SEQ ID NO: 17. In some cases, the amino acid sequence can be optimized to correspond to amino acid usage within a specific host organism/cell. The use of a CBCAS in some cases can result in the enzymatic synthesis of cannabichromene (CBC) and the accumulation of CBC within the cell or culture medium.

The various combinations of enzymes can be used to make a desired product such as olivetolic acid; CBGA; THCA; CBDA; CBCA; THC; CBD; CBC, or any combination thereof.

The enzymes disclosed throughout can be from a plant. For example, the enzymes can be from a plant that is from the genus Cannabis. More specifically, Cannabis plants that can be used include, but are not limited to Cannabis sativa, Cannabis indica, and Cannabis ruderalis. Other plants that can be used can be from the genus Echinacea, Acmella (e.g., Acmella oleracea), Helichrysum (e.g., Helichrysum umbraculigerum), Radula (e.g., Radula marginata), Theobroma (e.g., Theobroma cacao), and/or Piper (e.g., Piper nigrum).

Additional enzymes can be added in order to improve the production of CBGA or cannabinoids. For example, a gene encoding an HMG-CoA reductase, such as HMG1, can be used to increase cannabinoid titers. In some instances, the titer of CBGA can be increased by expressing HMG1. Additionally, HMG1 can be in different forms. For example, a truncated form of HMG1 can be used to increase cannabinoid titers. Other enzymes such as Farnesyl pyrophosphate synthetase, which is encoded by the gene ERG20 can be used to increase cannabinoid/CBGA titers. Additionally, ERG20 can be in different forms, such as mutant forms.

In cases where a HMG-CoA reductase (HMG1) is desired, the HMG1 can be encoded by an amino acid sequence that is substantially identical to SEQ ID NO: 19 or 21. For example, the HMG1 can be encoded by an amino acid sequence that is at least 50%, 60%, 70%, 80%, 85%, 90%, 95%, 96%, 97%, 98% or 99% identical to any one of SEQ ID NO: 19 or 21. In some cases, the HMG1 can be at least 50% identical to SEQ ID NO: 19 or 21. In some cases, the HMG1 can be at least 60% identical to SEQ ID NO: 19 or 21. In some cases, the HMG1 can be at least 65% identical to SEQ ID NO: 19 or 21. In some cases, the HMG1 can be at least 70% identical to SEQ ID NO: 19 or 21. In some cases, the HMG1 can be at least 75% identical to SEQ ID NO: 19 or 21. In some cases, the HMG1 can be at least 80% identical to SEQ ID NO: 19 or 21. In some cases, the HMG1 can be at least 81% identical to SEQ ID NO: 19 or 21. In some cases, the HMG1 can be at least 82% identical to SEQ ID NO: 19 or 21. In some cases, the HMG1 can be at least 83% identical to SEQ ID NO: 19 or 21. In some cases, the HMG1 can be at least 84% identical to SEQ ID NO: 19 or 21. In some cases, the HMG1 can be at least 85% identical to SEQ ID NO: 19 or 21. In some cases, the HMG1 can be at least 86% identical to SEQ ID NO: 19 or 21. In some cases, the HMG1 can be at least 87% identical to SEQ ID NO: 19 or 21. In some cases, the HMG1 can be at least 88% identical to SEQ ID NO: 19 or 21. In some cases, the HMG1 can be at least 89% identical to SEQ ID NO: 19 or 21. In some cases, the HMG1 can be at least 90% identical to SEQ ID NO: 19 or 21. In some cases, the HMG1 can be at least 91% identical to SEQ ID NO: 19 or 21. In some cases, the HMG1 can be at least 92% identical to SEQ ID NO: 19 or 21. In some cases, the HMG1 can be at least 93% identical to SEQ ID NO: 19 or 21. In some cases, the HMG1 can be at least 94% identical to SEQ ID NO: 19 or 21. In some cases, the HMG1 can be at least 95% identical to SEQ ID NO: 19 or 21. In some cases, the HMG1 can be at least 96% identical to SEQ ID NO: 19 or 21. In some cases, the HMG1 can be at least 97% identical to SEQ ID NO: 19 or 21. In some cases, the HMG1 can be at least 98% identical to SEQ ID NO: 19 or 21. In some cases, the HMG1 can be at least 99% identical to SEQ ID NO: 19 or 21. In some cases, the HMG1 can be identical to SEQ ID NO: 19 or 21. Further, codon optimized polynucleotides (for a particular host cell/organism) for the above referenced sequences can be used herein.

In cases where a farnesyl pyrophosphate synthetase (ERG20) is used, the ERG20 can be encoded by a nucleic acid sequence that is substantially identical to SEQ ID NO: 23. For example, the ERG20 can be encoded by an amino acid sequence that is at least 50%, 60%, 70%, 80%, 85%, 90%, 95%, 96%, 97%, 98% or 99% identical to any one of SEQ ID NO: 23. In some cases, the ERG20 can be at least 50% identical to SEQ ID NO: 23. In some cases, the ERG20 can be at least 60% identical to SEQ ID NO: 23. In some cases, the ERG20 can be at least 65% identical to SEQ ID NO: 23. In some cases, the ERG20 can be at least 70% identical to SEQ ID NO: 23. In some cases, the ERG20 can be at least 75% identical to SEQ ID NO: 23. In some cases, the ERG20 can be at least 80% identical to SEQ ID NO: 23. In some cases, the ERG20 can be at least 81% identical to SEQ ID NO: 23. In some cases, the ERG20 can be at least 82% identical to SEQ ID NO: 23. In some cases, the ERG20 can be at least 83% identical to SEQ ID NO: 23. In some cases, the ERG20 can be at least 84% identical to SEQ ID NO: 23. In some cases, the ERG20 can be at least 85% identical to SEQ ID NO: 23. In some cases, the ERG20 can be at least 86% identical to SEQ ID NO: 23. In some cases, the ERG20 can be at least 87% identical to SEQ ID NO: 23. In some cases, the ERG20 can be at least 88% identical to SEQ ID NO: 23. In some cases, the ERG20 can be at least 89% identical to SEQ ID NO: 23. In some cases, the ERG20 can be at least 90% identical to SEQ ID NO: 23. In some cases, the ERG20 can be at least 91% identical to SEQ ID NO: 23. In some cases, the ERG20 can be at least 92% identical to SEQ ID NO: 23. In some cases, the ERG20 can be at least 93% identical to SEQ ID NO: 23. In some cases, the ERG20 can be at least 94% identical to SEQ ID NO: 23. In some cases, the ERG20 can be at least 95% identical to SEQ ID NO: 23. In some cases, the ERG20 can be at least 96% identical to SEQ ID NO: 23. In some cases, the ERG20 can be at least 97% identical to SEQ ID NO: 23. In some cases, the ERG20 can be at least 98% identical to SEQ ID NO: 23. In some cases, the ERG20 can be at least 99% identical to SEQ ID NO: 23. In some cases, the ERG20 can be identical to SEQ ID NO: 23. Further, codon optimized polynucleotides (for a particular host cell/organism) for the above referenced sequences can be used herein.

In some cases, the enzymes described herein can be a fragment thereof. The fragment can still retain its respective biological activity. For example, a fragment of the prenyltransferase can be used as long as the activity of the fragment retains its biological activity.

The enzymes or fragments thereof described throughout can also be in some cases can be fused or linked together. Any fragment linker can be used to link the two or more of the enzymes or fragments thereof together. In some cases, the linker can be any random array of amino acid sequences. In some cases, linkers such as the T2A linker (SEQ ID NO: 15 (amino acid) or 16 (nucleic acid)) can be used.

The fused or linked enzymes can be two or more of any of the enzymes described throughout. For example, the disclosed prenyltransferase can be linked with a CBDA synthase. The resulting fused or linked enzyme can produce increased cannabidiol titers compared to separate enzymes that are not linked or fused. Additionally, other enzymes such as prenyltransferase and THCA synthase can be fused or linked. The resulting fused or linked enzyme can produce increased THC titers compared to separate enzymes that are not linked or fused. Enzymes that can catalyze the product of another enzyme can be fused or linked. For example AAE1 can be fused or linked to PKS. In some cases, OAC can be fused or linked to PKS. This can in some cases, increase the speed of two or more enzymatic conversions due to the proximity of the enzymatic substrates/products.

Vectors

Polynucleotide constructs prepared for introduction into a prokaryotic or eukaryotic host can typically, but not always, comprise a replication system (i.e. vector) recognized by the host, including the intended polynucleotide fragment encoding the desired polypeptide, and can but not necessarily, also include transcription and translational initiation regulatory sequences operably linked to the polypeptide-encoding segment. Expression systems (such as expression vectors) can include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, mRNA stabilizing sequences, nucleotide sequences homologous to host chromosomal DNA, and/or a multiple cloning site. Signal peptides can also be included where appropriate, for example from secreted polypeptides of the same or related species, which allow the protein to cross and/or lodge in cell membranes or be secreted from the cell.

The vectors can be constructed using standard methods (see, e.g., Sambrook et al., Molecular Biology: A Laboratory Manual, Cold Spring Harbor, N.Y. 1989; and Ausubel, et al., Current Protocols in Molecular Biology, Greene Publishing, Co. N.Y, 1995).

The manipulation of polynucleotides that encode the enzymes disclosed herein is typically carried out in recombinant vectors. Numerous vectors are publicly available, including bacterial plasmids, bacteriophage, artificial chromosomes, episomal vectors and gene expression vectors, which can all be employed. A vector can be selected to accommodate a polynucleotide encoding a protein of a desired size. Following recombinant modification of a selected vector, a suitable host cell (e.g., the microorganisms described herein) is transfected or transformed with the vector. Each vector contains various functional components, which generally include a cloning site, an origin of replication and at least one selectable marker gene. A vector can additionally possess one or more of the following elements: an enhancer, promoter, and transcription termination and/or other signal sequences. Such sequence elements can be optimized for the selected host species. Such sequence elements can be positioned in the vicinity of the cloning site, such that they are operatively linked to the gene encoding a preselected enzyme.

Vectors, including cloning and expression vectors, can contain nucleic acid sequences that enable the vector to replicate in one or more selected microorganisms. For example, the sequence can be one that enables the vector to replicate independently of the host chromosomal DNA and can include origins of replication or autonomously replicating sequences. Such sequences are well known for a variety of bacteria, yeast and viruses. For example, the origin of replication from the plasmid pBR322 is suitable for most Gram-negative bacteria, the 2 micron plasmid origin is suitable for yeast, and various viral origins (e.g. SV40, adenovirus) are useful for cloning vectors.

A cloning or expression vector can contain a selection gene (also referred to as a selectable marker). This gene encodes a protein necessary for the survival or growth of transformed microorganisms in a selective culture medium. Microorganisms not transformed with the vector containing the selection gene will therefore not survive in the culture medium. Typical selection genes encode proteins that confer resistance to antibiotics and other toxins, e.g. ampicillin, neomycin, methotrexate, hygromycin, thiostrepton, apramycin or tetracycline, complement auxotrophic deficiencies, or supply critical nutrients not available in the growth media.

The replication of vectors can be performed in E. coli. An E. coli-selectable marker, for example, the β-lactamase gene that confers resistance to the antibiotic ampicillin, can be of use. These selectable markers can be obtained from E. coli plasmids, such as pBR322 or a pUC plasmid such as pUC18 or pUC19, or pUC119.

Some exemplary vectors that can be used in the methods and microorganisms/cells are SEQ ID NO: 3 and 4. SEQ ID NO: 3 is also called the RUNM000898_511.1 vector, which comprises a Saccharomyces cerevisiae 2μ replication origin, a URA3 gene as an auxotrophic marker and the PKS and OAC genes under the regulation of the bidirectional GAL1/GAL10 promoter. SEQ ID NO: 4 is the bCBGA0098 vector that comprises a Saccharomyces cerevisiae 2μ replication origin, a LEU2 gene as an auxotrophic marker, and the AAE1 and PT genes under the regulation of the bidirectional GAL1/GAL10 promoter.

SEQ ID NO: 25 is a bCBGA0306 is a vector that comprises the Saccharomyces cerevisiae 2μ replication origin, the LEU2 gene as an auxotrophic marker and the PT gene under the regulation of the bidirectional GAL1/GAL10 promoter.

SEQ ID NO: 34 is the RUNM001233_51.1 vector comprising the Saccharomyces cerevisiae 2μ replication origin, the URA3 gene as an auxotrophic marker and the THCA synthase gene under the regulation of the bidirectional GAL1/GAL10 promoter.

SEQ ID NO: 35 is the RUNM001210_96.1 vector comprising the Saccharomyces cerevisiae 2μ replication origin, the URA3 gene as an auxotrophic marker, the PKS and OAC genes under the regulation of the bidirectional GAL1/GAL10 promoter and the AAE1 gene under the regulation of the STE5 promoter.

SEQ ID NO: 36 is the bCBGA0409 vector comprising the Saccharomyces cerevisiae 2μ replication origin, the LEU2 gene as an auxotrophic marker, the THCA synthase and PT genes under the regulation of the bidirectional GAL1/GAL10 promoter.

SEQ ID NO: 29 is the bCBGA0385 vector comprising the Saccharomyces cerevisiae 2μ replication origin, the LEU2 gene as an auxotrophic marker and the GFP-dPT gene under the regulation of the bidirectional GAL1/GAL10 promoter.

SEQ ID NO: 30 is the bCBGA0305 vector comprising the Saccharomyces cerevisiae 2μ replication origin, the TRP1 gene as an auxotrophic marker and the AAE1 gene under the regulation of the bidirectional GAL1/GAL10 promoter.

SEQ ID NO: 33 is the bCBGA0559 vector comprising the Saccharomyces cerevisiae 2μ replication origin, the LEU2 gene as an auxotrophic marker and the ERG20mut-dPT gene under the regulation of the bidirectional GAL1/GAL10 promoter.

Promoters

Vectors can contain a promoter that is recognized by the host microorganism. The promoter can be operably linked to a coding sequence of interest. Such a promoter can be inducible or constitutive. Polynucleotides are operably linked when the polynucleotides are in a relationship permitting them to function in their intended manner.

Different promoters can be used to drive the expression of the genes. For example, if temporary gene expression (i.e., non-constitutively expressed) is desired, expression can be driven by inducible promoters.

In some cases, some of the genes disclosed can be expressed temporarily. In other words, the genes are not constitutively expressed. The expression of the genes can be driven by inducible or repressible promoters. For example, the inducible or repressible promoters that can be used include but are not limited to: (a) sugars such as arabinose and lactose (or non metabolizable analogs, e.g., isopropyl β-D-1-thiogalactopyranoside (IPTG)); (b) metals such as lanthanum, copper, calcium; (c) temperature; (d) Nitrogen-source; (e) oxygen; (f) cell state (growth or stationary); (g) metabolites such as phosphate; (h) CRISPRi; (i) jun; (j) fos, (k) metallothionein and/or (l) heat shock.

Constitutively expressed promoters can also be used in the vector systems herein. For example, the expression of some of the genes disclosed throughout can be controlled by constitutively active promoters. For examples, the promoters that can be used include but are not limited to p.Bba.J23111, J23111, and J23100.

Promoters suitable for use with prokaryotic hosts can, for example, include but are not limited to the a-lactamase and lactose promoter systems, alkaline phosphatase, the tryptophan (trp) promoter system, the erythromycin promoter, apramycin promoter, hygromycin promoter, methylenomycin promoter and hybrid promoters such as the tac promoter. Promoters for use in bacterial systems will also generally contain a Shine-Dalgarno sequence operably linked to the coding sequence.

Generally, a strong promoter can be employed to provide for high level transcription and expression of the desired product.

One or more promoters of a transcription unit can be an inducible promoter. For example, a GFP can be expressed from a constitutive promoter while an inducible promoter drives transcription of a gene coding for one or more enzymes as disclosed herein and/or the amplifiable selectable marker.

Some vectors can contain prokaryotic sequences that facilitate the propagation of the vector in bacteria. Thus, the vectors can have other components such as an origin of replication (e.g., a nucleic acid sequence that enables the vector to replicate in one or more selected microorganisms), antibiotic resistance genes for selection in bacteria, and/or an amber stop codon which can permit translation to read through the codon. Additional selectable gene(s) can also be incorporated. Generally, in cloning vectors the origin of replication is one that enables the vector to replicate independently of the host chromosomal DNA, and includes origins of replication or autonomously replicating sequences. Such sequences can include the ColEl origin of replication in bacteria or other known sequences.

Genes

The genetically modified microorganisms can comprise a nucleic acid sequence encoding for one or more enzymes that are capable of catalyzing one or more of the following reactions: hexanoic acid to hexanoyl-CoA; hexanoyl-CoA to olivetolic Acid; olivetolic Acid to CBGA; CBGA to THCA; CBGA to CBDA; CBGA to CBCA; THCA to THC; CBDA to CBD; or CBCA to CBC. For example, the genetically modified microorganism can comprise a nucleic acid sequence encoding for one or more of the following enzymes: acyl activating enzyme (AAE1); polyketide synthase (PKS); olivetolic acid cyclase (OAC); prenyltransferase (PT); THCA synthase (THCAS); CBDA synthase (CBDAS), CBCA synthase (CBCAS); or any combination thereof. The nucleic acid sequence in some cases can be within a vector. In some cases, the nucleic acid sequences do not need to be within a vector but rather integrated into the microorganism's genome or isolated. In some cases, the isolated nucleic acids can be inserted into the genome of the cell/microorganism used. In some cases, the isolated nucleic acid is inserted into the genome at a specific locus, where the isolated nucleic acid can be expressed in sufficient amounts.

In some cases, two or more genes encoding for consecutive enzymes in the pathway from a carbon substrate (e.g., sugar) to any of the cannabinoids described throughout (e.g., THCA, CBDA, CBCA, THC, CBD, or CBC) can be used. In some cases, three or more genes encoding for consecutive enzymes in the pathway can be used. In some cases, four or more genes encoding for consecutive enzymes in the pathway can be used. In some cases, five or more genes encoding for consecutive enzymes in the pathway can be used. In some cases, six or more genes encoding for consecutive enzymes in the pathway can be used. In some cases, seven or more genes encoding for consecutive enzymes in the pathway can be used. In some cases, eight or more genes encoding for consecutive enzymes in the pathway can be used. In some cases, nine or more genes encoding for consecutive enzymes in the pathway can be used. In some cases, ten or more genes encoding for consecutive enzymes in the pathway can be used.

In some cases, when an acyl activating enzyme (AAE1) is desired, the AAE1 can be encoded by a nucleic acid sequence that is substantially identical to SEQ ID NO: 14. For example, the AAE1 can be encoded by a polynucleotide that is at least 50%, 60%, 70%, 80%, 85%, 90%, 95%, 96%, 97%, 98% or 99% identical to SEQ ID NO: 14. In some cases, the AAE1 can be at least 50% identical to SEQ ID NO: 14. In some cases, the AAE1 can be at least 60% identical to SEQ ID NO: 14. In some cases, the AAE1 can be at least 65% identical to SEQ ID NO: 14. In some cases, the AAE1 can be at least 70% identical to SEQ ID NO: 14. In some cases, the AAE1 can be at least 75% identical to SEQ ID NO: 14. In some cases, the AAE1 can be at least 80% identical to SEQ ID NO: 14. In some cases, the AAE1 can be at least 81% identical to SEQ ID NO: 14. In some cases, the AAE1 can be at least 82% identical to SEQ ID NO: 14. In some cases, the AAE1 can be at least 83% identical to SEQ ID NO: 14. In some cases, the AAE1 can be at least 84% identical to SEQ ID NO: 14. In some cases, the AAE1 can be at least 85% identical to SEQ ID NO: 14. In some cases, the AAE1 can be at least 86% identical to SEQ ID NO: 14. In some cases, the AAE1 can be at least 87% identical to SEQ ID NO: 14. In some cases, the AAE1 can be at least 88% identical to SEQ ID NO: 14. In some cases, the AAE1 can be at least 89% identical to SEQ ID NO: 14. In some cases, the AAE1 can be at least 90% identical to SEQ ID NO: 14. In some cases, the AAE1 can be at least 91% identical to SEQ ID NO: 14. In some cases, the AAE1 can be at least 92% identical to SEQ ID NO: 14. In some cases, the AAE1 can be at least 93% identical to SEQ ID NO: 14. In some cases, the AAE1 can be at least 94% identical to SEQ ID NO: 14. In some cases, the AAE1 can be at least 95% identical to SEQ ID NO: 14. In some cases, the AAE1 can be at least 96% identical to SEQ ID NO: 14. In some cases, the AAE1 can be at least 97% identical to SEQ ID NO: 14. In some cases, the AAE1 can be at least 98% identical to SEQ ID NO: 14. In some cases, the AAE1 can be at least 99% identical to SEQ ID NO: 14. In some cases, the AAE1 can be identical to SEQ ID NO: 14. Further, codon optimized polynucleotides (for a particular host cell/organism) for the above referenced sequences can be used herein.

In cases where a polyketide synthase (PKS) is used, the PKS can be encoded by a nucleic acid sequence that is substantially identical to SEQ ID NO: 6. For example, the PKS can be encoded by a polynucleotide that is at least 50%, 60%, 70%, 80%, 85%, 90%, 95%, 96%, 97%, 98% or 99% identical to SEQ ID NO: 6. In some cases, the PKS can be at least 50% identical to SEQ ID NO: 6. In some cases, the PKS can be at least 60% identical to SEQ ID NO: 6. In some cases, the PKS can be at least 65% identical to SEQ ID NO: 6. In some cases, the PKS can be at least 70% identical to SEQ ID NO: 6. In some cases, the PKS can be at least 75% identical to SEQ ID NO: 6. In some cases, the PKS can be at least 80% identical to SEQ ID NO: 6. In some cases, the PKS can be at least 81% identical to SEQ ID NO: 6. In some cases, the PKS can be at least 82% identical to SEQ ID NO: 6. In some cases, the PKS can be at least 83% identical to SEQ ID NO: 6. In some cases, the PKS can be at least 84% identical to SEQ ID NO: 6. In some cases, the PKS can be at least 85% identical to SEQ ID NO: 6. In some cases, the PKS can be at least 86% identical to SEQ ID NO: 6. In some cases, the PKS can be at least 87% identical to SEQ ID NO: 6. In some cases, the PKS can be at least 88% identical to SEQ ID NO: 6. In some cases, the PKS can be at least 89% identical to SEQ ID NO: 6. In some cases, the PKS can be at least 90% identical to SEQ ID NO: 6. In some cases, the PKS can be at least 91% identical to SEQ ID NO: 6. In some cases, the PKS can be at least 92% identical to SEQ ID NO: 6. In some cases, the PKS can be at least 93% identical to SEQ ID NO: 6. In some cases, the PKS can be at least 94% identical to SEQ ID NO: 6. In some cases, the PKS can be at least 95% identical to SEQ ID NO: 6. In some cases, the PKS can be at least 96% identical to SEQ ID NO: 6. In some cases, the PKS can be at least 97% identical to SEQ ID NO: 6. In some cases, the PKS can be at least 98% identical to SEQ ID NO: 6. In some cases, the PKS can be at least 99% identical to SEQ ID NO: 6. In some cases, the PKS can be identical to SEQ ID NO: 6. Further, codon optimized polynucleotides (for a particular host cell/organism) for the above referenced sequences can be used herein.

In cases where an olivetolic acid cyclase (OAC) is used, the OAC can be encoded by a nucleic acid sequence that is substantially identical to SEQ ID NO: 8. For example, the OAC can be encoded by a polynucleotide that is at least 50%, 60%, 70%, 80%, 85%, 90%, 95%, 96%, 97%, 98% or 99% identical to SEQ ID NO: 8. In some cases, the OAC can be at least 50% identical to SEQ ID NO: 8. In some cases, the OAC can be at least 60% identical to SEQ ID NO: 8. In some cases, the OAC can be at least 65% identical to SEQ ID NO: 8. In some cases, the OAC can be at least 70% identical to SEQ ID NO: 8. In some cases, the OAC can be at least 75% identical to SEQ ID NO: 8. In some cases, the OAC can be at least 80% identical to SEQ ID NO: 8. In some cases, the OAC can be at least 81% identical to SEQ ID NO: 8. In some cases, the OAC can be at least 82% identical to SEQ ID NO: 8. In some cases, the OAC can be at least 83% identical to SEQ ID NO: 8. In some cases, the OAC can be at least 84% identical to SEQ ID NO: 8. In some cases, the OAC can be at least 85% identical to SEQ ID NO: 8. In some cases, the OAC can be at least 86% identical to SEQ ID NO: 8. In some cases, the OAC can be at least 87% identical to SEQ ID NO: 8. In some cases, the OAC can be at least 88% identical to SEQ ID NO: 8. In some cases, the OAC can be at least 89% identical to SEQ ID NO: 8. In some cases, the OAC can be at least 90% identical to SEQ ID NO: 8. In some cases, the OAC can be at least 91% identical to SEQ ID NO: 8. In some cases, the OAC can be at least 92% identical to SEQ ID NO: 8. In some cases, the OAC can be at least 93% identical to SEQ ID NO: 8. In some cases, the OAC can be at least 94% identical to SEQ ID NO: 8. In some cases, the OAC can be at least 95% identical to SEQ ID NO: 8. In some cases, the OAC can be at least 96% identical to SEQ ID NO: 8. In some cases, the OAC can be at least 97% identical to SEQ ID NO: 8. In some cases, the OAC can be at least 98% identical to SEQ ID NO: 8. In some cases, the OAC can be at least 99% identical to SEQ ID NO: 8. In some cases, the OAC can be identical to SEQ ID NO: 8. Further, codon optimized polynucleotides (for a particular host cell/organism) for the above referenced sequences can be used herein.

In cases where a prenyltransferase (PT) is used, the PT can be encoded by a nucleic acid sequence that is substantially identical to any one of SEQ ID NOs: 2, 26, 31, or 37. For example, the PT can be encoded by a polynucleotide that is at least 50%, 60%, 70%, 80%, 85%, 90%, 95%, 96%, 97%, 98% or 99% identical to any one of SEQ ID NOs: 2, 26, 31, or 37. In some cases, the PT can be at least 50% identical to any one of SEQ ID NOs: 2, 26, 31, or 37. In some cases, the PT can be at least 60% identical to any one of SEQ ID NOs: 2, 26, 31, or 37. In some cases, the PT can be at least 65% identical to any one of SEQ ID NOs: 2, 26, 31, or 37. In some cases, the PT can be at least 70% identical to any one of SEQ ID NOs: 2, 26, 31, or 37. In some cases, the PT can be at least 75% identical to any one of SEQ ID NOs: 2, 26, 31, or 37. In some cases, the PT can be at least 80% identical to any one of SEQ ID NOs: 2, 26, 31, or 37. In some cases, the PT can be at least 81% identical to any one of SEQ ID NOs: 2, 26, 31, or 37. In some cases, the PT can be at least 82% identical to any one of SEQ ID NOs: 2, 26, 31, or 37. In some cases, the PT can be at least 83% identical to any one of SEQ ID NOs: 2, 26, 31, or 37. In some cases, the PT can be at least 84% identical to any one of SEQ ID NOs: 2, 26, 31, or 37. In some cases, the PT can be at least 85% identical to any one of SEQ ID NOs: 2, 26, 31, or 37. In some cases, the PT can be at least 86% identical to any one of SEQ ID NOs: 2, 26, 31, or 37. In some cases, the PT can be at least 87% identical to any one of SEQ ID NOs: 2, 26, 31, or 37. In some cases, the PT can be at least 88% identical to any one of SEQ ID NOs: 2, 26, 31, or 37. In some cases, the PT can be at least 89% identical to any one of SEQ ID NOs: 2, 26, 31, or 37. In some cases, the PT can be at least 90% identical to any one of SEQ ID NOs: 2, 26, 31, or 37. In some cases, the PT can be at least 91% identical to any one of SEQ ID NOs: 2, 26, 31, or 37. In some cases, the PT can be at least 92% identical to any one of SEQ ID NOs: 2, 26, 31, or 37. In some cases, the PT can be at least 93% identical to any one of SEQ ID NOs: 2, 26, 31, or 37. In some cases, the PT can be at least 94% identical to any one of SEQ ID NOs: 2, 26, 31, or 37. In some cases, the PT can be at least 95% identical to any one of SEQ ID NOs: 2, 26, 31, or 37. In some cases, the PT can be at least 96% identical to any one of SEQ ID NOs: 2, 26, 31, or 37. In some cases, the PT can be at least 97% identical to any one of SEQ ID NOs: 2, 26, 31, or 37. In some cases, the PT can be at least 98% identical to any one of SEQ ID NOs: 2, 26, 31, or 37. In some cases, the PT can be at least 99% identical to any one of SEQ ID NOs: 2, 26, 31, or 37. In some cases, the PT can be identical to any one of SEQ ID NOs: 2, 26, 31, or 37. Further, codon optimized polynucleotides (for a particular host cell/organism) for the above referenced sequences can be used herein.

In cases where a THCA synthase (THCAS) is used, the THCAS can be encoded by a nucleic acid sequence that is substantially identical to SEQ ID NO: 10. For example, the THCAS can be encoded by a polynucleotide that is at least 50%, 60%, 70%, 80%, 85%, 90%, 95%, 96%, 97%, 98% or 99% identical to SEQ ID NO: 10. In some cases, the THCAS can be at least 50% identical to SEQ ID NO: 10. In some cases, the THCAS can be at least 60% identical to SEQ ID NO: 10. In some cases, the THCAS can be at least 65% identical to SEQ ID NO: 10. In some cases, the THCAS can be at least 70% identical to SEQ ID NO: 10. In some cases, the THCAS can be at least 75% identical to SEQ ID NO: 10. In some cases, the THCAS can be at least 80% identical to SEQ ID NO: 10. In some cases, the THCAS can be at least 81% identical to SEQ ID NO: 10. In some cases, the THCAS can be at least 82% identical to SEQ ID NO: 10. In some cases, the THCAS can be at least 83% identical to SEQ ID NO: 10. In some cases, the THCAS can be at least 84% identical to SEQ ID NO: 10. In some cases, the THCAS can be at least 85% identical to SEQ ID NO: 10. In some cases, the THCAS can be at least 86% identical to SEQ ID NO: 10. In some cases, the THCAS can be at least 87% identical to SEQ ID NO: 10. In some cases, the THCAS can be at least 88% identical to SEQ ID NO: 10. In some cases, the THCAS can be at least 89% identical to SEQ ID NO: 10. In some cases, the THCAS can be at least 90% identical to SEQ ID NO: 10. In some cases, the THCAS can be at least 91% identical to SEQ ID NO: 10. In some cases, the THCAS can be at least 92% identical to SEQ ID NO: 10. In some cases, the THCAS can be at least 93% identical to SEQ ID NO: 10. In some cases, the THCAS can be at least 94% identical to SEQ ID NO: 10. In some cases, the THCAS can be at least 95% identical to SEQ ID NO: 10. In some cases, the THCAS can be at least 96% identical to SEQ ID NO: 10. In some cases, the THCAS can be at least 97% identical to SEQ ID NO: 10. In some cases, the THCAS can be at least 98% identical to SEQ ID NO: 10. In some cases, the THCAS can be at least 99% identical to SEQ ID NO: 10. In some cases, the THCAS can be identical to SEQ ID NO: 10. Further, codon optimized polynucleotides (for a particular host cell/organism) for the above referenced sequences can be used herein.

In cases where a CBDA synthase (CBDAS) is used, the CBDAS can be encoded by a nucleic acid sequence that is substantially identical to SEQ ID NO: 12. For example, the CBDAS can be encoded by a polynucleotide that is at least 50%, 60%, 70%, 80%, 85%, 90%, 95%, 96%, 97%, 98% or 99% identical to SEQ ID NO: 12. In some cases, the CBDAS can be at least 50% identical to SEQ ID NO: 12. In some cases, the CBDAS can be at least 60% identical to SEQ ID NO: 12. In some cases, the CBDAS can be at least 65% identical to SEQ ID NO: 12. In some cases, the CBDAS can be at least 70% identical to SEQ ID NO: 12. In some cases, the CBDAS can be at least 75% identical to SEQ ID NO: 12. In some cases, the CBDAS can be at least 80% identical to SEQ ID NO: 12. In some cases, the CBDAS can be at least 81% identical to SEQ ID NO: 12. In some cases, the CBDAS can be at least 82% identical to SEQ ID NO: 12. In some cases, the CBDAS can be at least 83% identical to SEQ ID NO: 12. In some cases, the CBDAS can be at least 84% identical to SEQ ID NO: 12. In some cases, the CBDAS can be at least 85% identical to SEQ ID NO: 12. In some cases, the CBDAS can be at least 86% identical to SEQ ID NO: 12. In some cases, the CBDAS can be at least 87% identical to SEQ ID NO: 12. In some cases, the CBDAS can be at least 88% identical to SEQ ID NO: 12. In some cases, the CBDAS can be at least 89% identical to SEQ ID NO: 12. In some cases, the CBDAS can be at least 90% identical to SEQ ID NO: 12. In some cases, the CBDAS can be at least 91% identical to SEQ ID NO: 12. In some cases, the CBDAS can be at least 92% identical to SEQ ID NO: 12. In some cases, the CBDAS can be at least 93% identical to SEQ ID NO: 12. In some cases, the CBDAS can be at least 94% identical to SEQ ID NO: 12. In some cases, the CBDAS can be at least 95% identical to SEQ ID NO: 12. In some cases, the CBDAS can be at least 96% identical to SEQ ID NO: 12. In some cases, the CBDAS can be at least 97% identical to SEQ ID NO: 12. In some cases, the CBDAS can be at least 98% identical to SEQ ID NO: 12. In some cases, the CBDAS can be at least 99% identical to SEQ ID NO: 12. In some cases, the CBDAS can be identical to SEQ ID NO: 12. Further, codon optimized polynucleotides (for a particular host cell/organism) for the above referenced sequences can be used herein.

In cases where a CBCA synthase (CBCAS) is used, the CBCAS can be encoded by a nucleic acid sequence that is substantially identical to SEQ ID NO: 18. For example, the CBCAS can be encoded by a polynucleotide that is at least 50%, 60%, 70%, 80%, 85%, 90%, 95%, 96%, 97%, 98% or 99% identical to SEQ ID NO: 18. In some cases, the CBCAS can be at least 50% identical to SEQ ID NO: 18. In some cases, the CBCAS can be at least 60% identical to SEQ ID NO: 18. In some cases, the CBCAS can be at least 65% identical to SEQ ID NO: 18. In some cases, the CBCAS can be at least 70% identical to SEQ ID NO: 18. In some cases, the CBCAS can be at least 75% identical to SEQ ID NO: 18. In some cases, the CBCAS can be at least 80% identical to SEQ ID NO: 18. In some cases, the CBCAS can be at least 81% identical to SEQ ID NO: 18. In some cases, the CBCAS can be at least 82% identical to SEQ ID NO: 18. In some cases, the CBCAS can be at least 83% identical to SEQ ID NO: 18. In some cases, the CBCAS can be at least 84% identical to SEQ ID NO: 18. In some cases, the CBCAS can be at least 85% identical to SEQ ID NO: 18. In some cases, the CBCAS can be at least 86% identical to SEQ ID NO: 18. In some cases, the CBCAS can be at least 87% identical to SEQ ID NO: 18. In some cases, the CBCAS can be at least 88% identical to SEQ ID NO: 18. In some cases, the CBCAS can be at least 89% identical to SEQ ID NO: 18. In some cases, the CBCAS can be at least 90% identical to SEQ ID NO: 18. In some cases, the CBCAS can be at least 91% identical to SEQ ID NO: 18. In some cases, the CBCAS can be at least 92% identical to SEQ ID NO: 18. In some cases, the CBCAS can be at least 93% identical to SEQ ID NO: 18. In some cases, the CBCAS can be at least 94% identical to SEQ ID NO: 18. In some cases, the CBCAS can be at least 95% identical to SEQ ID NO: 18. In some cases, the CBCAS can be at least 96% identical to SEQ ID NO: 18. In some cases, the CBCAS can be at least 97% identical to SEQ ID NO: 18. In some cases, the CBCAS can be at least 98% identical to SEQ ID NO: 18. In some cases, the CBCAS can be at least 99% identical to SEQ ID NO: 18. In some cases, the CBCAS can be identical to SEQ ID NO: 18. Further, codon optimized polynucleotides (for a particular host cell/organism) for the above referenced sequences can be used herein.

The genetically modified microorganism can also further comprise one or more nucleic acids encoding for enzymes (in some cases heterologous enzymes), including but not limited to HMG1, ERG20, and/or isoforms and mutants thereof.

In cases where a HMG-CoA reductase (HMG1) is used, the HMG1 can be encoded by a nucleic acid sequence that is substantially identical to SEQ ID NO: 20 or 22. For example, the HMG1 can be encoded by a polynucleotide that is at least 50%, 60%, 70%, 80%, 85%, 90%, 95%, 96%, 97%, 98% or 99% identical to SEQ ID NO: 20 or 22. In some cases, the HMG1 can be at least 50% identical to SEQ ID NO: 20 or 22. In some cases, the HMG1 can be at least 60% identical to SEQ ID NO: 20 or 22. In some cases, the HMG1 can be at least 65% identical to SEQ ID NO: 20 or 22. In some cases, the HMG1 can be at least 70% identical to SEQ ID NO: 20 or 22. In some cases, the HMG1 can be at least 75% identical to SEQ ID NO: 20 or 22. In some cases, the HMG1 can be at least 80% identical to SEQ ID NO: 20 or 22. In some cases, the HMG1 can be at least 81% identical to SEQ ID NO: 20 or 22. In some cases, the HMG1 can be at least 82% identical to SEQ ID NO: 20 or 22. In some cases, the HMG1 can be at least 83% identical to SEQ ID NO: 20 or 22. In some cases, the HMG1 can be at least 84% identical to SEQ ID NO: 20 or 22. In some cases, the HMG1 can be at least 85% identical to SEQ ID NO: 20 or 22. In some cases, the HMG1 can be at least 86% identical to SEQ ID NO: 20 or 22. In some cases, the HMG1 can be at least 87% identical to SEQ ID NO: 20 or 22. In some cases, the HMG1 can be at least 88% identical to SEQ ID NO: 20 or 22. In some cases, the HMG1 can be at least 89% identical to SEQ ID NO: 20 or 22. In some cases, the HMG1 can be at least 90% identical to SEQ ID NO: 20 or 22. In some cases, the HMG1 can be at least 91% identical to SEQ ID NO: 20 or 22. In some cases, the HMG1 can be at least 92% identical to SEQ ID NO: 20 or 22. In some cases, the HMG1 can be at least 93% identical to SEQ ID NO: 20 or 22. In some cases, the HMG1 can be at least 94% identical to SEQ ID NO: 20 or 22. In some cases, the HMG1 can be at least 95% identical to SEQ ID NO: 20 or 22. In some cases, the HMG1 can be at least 96% identical to SEQ ID NO: 20 or 22. In some cases, the HMG1 can be at least 97% identical to SEQ ID NO: 20 or 22. In some cases, the HMG1 can be at least 98% identical to SEQ ID NO: 20 or 22. In some cases, the HMG1 can be at least 99% identical to SEQ ID NO: 20 or 22. In some cases, the HMG1 can be identical to SEQ ID NO: 20 or 22. Further, codon optimized polynucleotides (for a particular host cell/organism) for the above referenced sequences can be used herein.

In cases where a farnesyl pyrophosphate synthetase (ERG20) is used, the ERG20 can be encoded by a nucleic acid sequence that is substantially identical to SEQ ID NO: 24. For example, the ERG20 can be encoded by a polynucleotide that is at least 50%, 60%, 70%, 80%, 85%, 90%, 95%, 96%, 97%, 98% or 99% identical to SEQ ID NO: 24. In some cases, the ERG20 can be at least 50% identical to SEQ ID NO: 24. In some cases, the ERG20 can be at least 60% identical to SEQ ID NO: 24. In some cases, the ERG20 can be at least 65% identical to SEQ ID NO: 24. In some cases, the ERG20 can be at least 70% identical to SEQ ID NO: 24. In some cases, the ERG20 can be at least 75% identical to SEQ ID NO: 24. In some cases, the ERG20 can be at least 80% identical to SEQ ID NO: 24. In some cases, the ERG20 can be at least 81% identical to SEQ ID NO: 24. In some cases, the ERG20 can be at least 82% identical to SEQ ID NO: 24. In some cases, the ERG20 can be at least 83% identical to SEQ ID NO: 24. In some cases, the ERG20 can be at least 84% identical to SEQ ID NO: 24. In some cases, the ERG20 can be at least 85% identical to SEQ ID NO: 24. In some cases, the ERG20 can be at least 86% identical to SEQ ID NO: 24. In some cases, the ERG20 can be at least 87% identical to SEQ ID NO: 24. In some cases, the ERG20 can be at least 88% identical to SEQ ID NO: 24. In some cases, the ERG20 can be at least 89% identical to SEQ ID NO: 24. In some cases, the ERG20 can be at least 90% identical to SEQ ID NO: 24. In some cases, the ERG20 can be at least 91% identical to SEQ ID NO: 24. In some cases, the ERG20 can be at least 92% identical to SEQ ID NO: 24. In some cases, the ERG20 can be at least 93% identical to SEQ ID NO: 24. In some cases, the ERG20 can be at least 94% identical to SEQ ID NO: 24. In some cases, the ERG20 can be at least 95% identical to SEQ ID NO: 24. In some cases, the ERG20 can be at least 96% identical to SEQ ID NO: 24. In some cases, the ERG20 can be at least 97% identical to SEQ ID NO: 24. In some cases, the ERG20 can be at least 98% identical to SEQ ID NO: 24. In some cases, the ERG20 can be at least 99% identical to SEQ ID NO: 24. In some cases, the ERG20 can be identical to SEQ ID NO: 24. Further, codon optimized polynucleotides (for a particular host cell/organism) for the above referenced sequences can be used herein.

### Modifying Endogenous Gene Expression

The genetically modified microorganisms disclosed herein can have their endogenous genes regulated. This can be useful, for example, when there is negative feedback to the expression of a desired polypeptide, such as any of the enzymes described throughout including but not limited to acyl activating enzyme (AAE1); polyketide synthase (PKS); olivetolic acid cyclase (OAC); prenyltransferase (PT); THCA synthase (THCAS); CBDA synthase (CBDAS), CBCA synthase (CBCAS); HMG-CoA reductase (HMG1); farnesyl pyrophosphate synthetase (ERG20); or any combination thereof. Modifying one or more negative regulator can lead to increased expression of a desired polypeptide, and in some cases, increase the production level of the cannabinoids.

Modifying the expression of endogenous genes can be achieved in a variety of ways. For example, antisense or RNA interference approaches can be used to down-regulate expression of the polynucleotides of the present disclosure, e.g., as a further mechanism for modulating cellular phenotype. That is, antisense sequences of the polynucleotides of the present disclosure, or subsequences thereof, can be used to block expression of naturally occurring homologous polynucleotide sequences. In particular, constructs comprising a desired polypeptide coding sequence, including fragments thereof, in antisense orientation, or combinations of sense and antisense orientation, can be used to decrease or effectively eliminate the expression of the desired polypeptide in a cell or plant and obtain an improvement in shelf life as is described herein. Accordingly, this can be used to “knock-out” the desired polypeptide or homologous sequences thereof. A variety of sense and antisense technologies, e.g., as set forth in Lichtenstein and Nellen (Antisense Technology: A Practical Approach IRL Press at Oxford University, Oxford, England, 1997), can be used. Sense or antisense polynucleotide can be introduced into a cell, where they are transcribed. Such polynucleotides can include both simple oligonucleotide sequences and catalytic sequences such as ribozymes.

Other methods for a reducing or eliminating expression (i.e., a “knock-out” or “knockdown”) of a desired polypeptide in a transgenic cell or plant can be done by introduction of a construct which expresses an antisense of the desired polypeptide coding strand or fragment thereof. For antisense suppression, the desired polypeptide cDNA or fragment thereof is arranged in reverse orientation (with respect to the coding sequence) relative to the promoter sequence in the expression vector. Further, the introduced sequence need not always correspond to the full length cDNA or gene, and need not be identical to the cDNA or gene found in the cell or plant to be transformed.

Additionally, the antisense sequence need only be capable of hybridizing to the target gene or RNA of interest. Thus, where the introduced polynucleotide sequence is of shorter length, a higher degree of homology to the endogenous transcription factor sequence will be needed for effective antisense suppression. While antisense sequences of various lengths can be utilized, in some embodiments, the introduced antisense polynucleotide sequence in the vector is at least 10, 20, 30, 40, 50, 100 or more nucleotides in length in certain embodiments. Transcription of an antisense construct as described results in the production of RNA molecules that comprise a sequence that is the reverse complement of the mRNA molecules transcribed from the endogenous gene to be repressed.

Other methods for a reducing or eliminating expression can be done by introduction of a construct that expresses siRNA that targets a desired polypeptide (e.g., CBGA synthesis polypeptide). In certain embodiments, siRNAs are short (20 to 24-bp) double-stranded RNA (dsRNA) with phosphorylated 5′ ends and hydroxylated 3′ ends with two overhanging nucleotides.

Other methods for a reducing or eliminating expression can be done by insertion mutagenesis using the T-DNA of Agrobacterium tumefaciens or a selection marker cassette or any other non-sense DNA fragments. After generating the insertion mutants, the mutants can be screened to identify those containing the insertion in the CBGA synthesis polypeptide (or other desired polypeptide) gene. Plants containing one or more transgene insertion events at the desired gene can be crossed to generate homozygous plant for the mutation, as described in Koncz et al., (Methods in Arabidopsis Research; World Scientific, 1992).

Suppression of gene expression can also be achieved using a ribozyme. Ribozymes are RNA molecules that possess highly specific endoribonuclease activity. The production and use of ribozymes are disclosed in U.S. Pat. Nos. 4,987,071 and 5,543,508. Synthetic ribozyme sequences including antisense RNAs can be used to confer RNA cleaving activity on the antisense RNA, such that endogenous mRNA molecules that hybridize to the antisense RNA are cleaved, which in turn leads to an enhanced antisense inhibition of endogenous gene expression.

A cell or plant gene can also be modified by using the Cre-lox system (for example, as described in U.S. Pat. No. 5,658,772). A cellular or plant genome can be modified to include first and second lox sites that are then contacted with a Cre recombinase. If the lox sites are in the same orientation, the intervening DNA sequence between the two sites is excised. If the lox sites are in the opposite orientation, the intervening sequence is inverted.

In addition, silencing approach using short hairpin RNA (shRNA) system, and complementary mature CRISPR RNA (crRNA) by CRISPR/Cas system, and virus inducing gene silencing (VIGS) system can also be used to make down regulated or knockout of synthase mutants. Dominant negative approaches can also be used to make down regulated or knockout of desired polypeptides.

The RNA-guided endonuclease can be derived from a clustered regularly interspersed short palindromic repeats (CRISPR)/CRISPR-associated (Cas) system. The CRISPR/Cas system can be a type I, a type II, or a type III system. Non-limiting examples of suitable CRISPR/Cas proteins include Cas3, Cas4, Cas5, Cas5e (or CasD), Cas6, Cas6e, Cas6f, Cas7, Cas8a1, Cas8a2, Cas8b, Cas8c, Cas9, Cask), Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cul966.

In general, CRISPR/Cas proteins comprise at least one RNA recognition and/or RNA binding domain. RNA recognition and/or RNA binding domains interact with guide RNAs. CRISPR/Cas proteins can also comprise nuclease domains (i.e., DNase or RNase domains), DNA binding domains, helicase domains, RNAse domains, protein-protein interaction domains, dimerization domains, as well as other domains.

The CRISPR/Cas-like protein can be a wild type CRISPR/Cas protein, a modified CRISPR/Cas protein, or a fragment of a wild type or modified CRISPR/Cas protein. The CRISPR/Cas-like protein can be modified to increase nucleic acid binding affinity and/or specificity, alter an enzyme activity, and/or change another property of the protein. For example, nuclease (i.e., DNase, RNase) domains of the CRISPR/Cas-like protein can be modified, deleted, or inactivated. Alternatively, the CRISPR/Cas-like protein can be truncated to remove domains that are not essential for the function of the fusion protein. The CRISPR/Cas-like protein can also be truncated or modified to optimize the activity of the effector domain of the fusion protein.

One method to silence a desired gene (or a CBGA synthesis polypeptide gene) is virus induced gene silencing (known to the art as VIGS). In general, in plants infected with unmodified viruses, the viral genome is targeted. However, when viral vectors have been modified to carry inserts derived from host genes (e.g. portions of sequences encoding a desired polypeptide such as CBGA synthesis polypeptide), the process is additionally targeted against the corresponding mRNAs. Thus disclosed is a method of producing a plant expressing reduced levels of a desired gene (such as CBGA synthesis polypeptide) or other desired gene(s), the method comprising (a) providing a plant expressing a desired gene (e.g., a CBGA synthesis polypeptide); and (b) reducing expression of the desired gene in the plant using virus induced gene silencing.

In some cases, one or more genes can be disrupted. In some cases, the one or more genes can be from the pathway that controls beta oxidation of long chain fatty acids. For example, in some cases, the one or more genes that can be disrupted can be any one of FOX1, FAA1, FAA4, FAT1, PXA1, PXA2, and/or PEX11. Any of the methods described throughout, can be used to disrupt one or more of the genes.

In some cases, the one or more genes that can be disrupted can comprise FOX1. For example, a sequence that is substantially identical to SEQ ID NO: 39 can be targeted for disruption. Any of the methods described throughout, can be used to disrupt the FOX1 gene, for example, but use of the CRISPR/Cas system or the use of RNAi technology. As few as a single nucleotide needs to be altered to have a disruptive effect to FOX1 or other genes that are targeted for disruption.

### Isolated Polynucleic Acids

The genes described throughout can be in the form of an isolated polynucleic acid. In other words, the genes can be in forms that do not exist in nature, isolated from a chromosome. The isolated polynucleic acids can comprise a nucleic acid sequence of one or more genes encoding a: (i) acyl activating enzyme (AAE1); (ii) polyketide synthase (PKS); (iii) olivetolic acid cyclase (OAC); (iv) prenyltransferase (PT); (v) THCA synthase (THCAS); (vi) CBDA synthase (CBDAS); and/or (vii) CBCA synthase (CBCAS). For example, the isolated polynucleic acid can comprise a PKS gene. The isolated polynucleic acid can comprise an OAC gene. The isolated polynucleic acid can comprise a PT gene. The isolated polynucleic acid can comprise a THCAS gene. The isolated polynucleic acid can comprise a CBDAS gene. The isolated polynucleic acid can comprise a CBCAS gene. The isolated polynucleic acid can comprise an AAE1 gene.

In some cases, the isolated polynucleic acid can encode an acyl activating enzyme (AAE1). For example, the isolated polynucleic acid can comprise a nucleotide sequence that is substantially identical to SEQ ID NO: 14. In some cases, the isolated polynucleic acid can comprise a nucleotide sequence that is at least 60%, 65%, 70%, 75%, 80%, 85%, 90%, 91%, 92%, 93%, 94%, 95%, 96%, 97%, 98%, 99%, 99.9%, 99.99%, 99.999%, or 99.9999% identical to SEQ ID NO: 14. In some cases, the isolated polynucleic acid can comprise a nucleotide sequence that is at least 60% identical to SEQ ID NO: 14. In some cases, the isolated polynucleic acid can comprise a nucleotide sequence that is at least 65% identical to SEQ ID NO: 14. In some cases, the isolated polynucleic acid can comprise a nucleotide sequence that is at least 70% identical to SEQ ID NO: 14. In some cases, the isolated polynucleic acid can comprise a nucleotide sequence that is at least 75% identical to SEQ ID NO: 14. In some cases, the isolated polynucleic acid can comprise a nucleotide sequence that is at least 80% identical to SEQ ID NO: 14. In some cases, the isolated polynucleic acid can comprise a nucleotide sequence that is at least 81% identical to SEQ ID NO: 14. In some cases, the isolated polynucleic acid can comprise a nucleotide sequence that is at least 82% identical to SEQ ID NO: 14. In some cases, the isolated polynucleic acid can comprise a nucleotide sequence that is at least 83% identical to SEQ ID NO: 14. In some cases, the isolated polynucleic acid can comprise a nucleotide sequence that is at least 84% identical to SEQ ID NO: 14. In some cases, the isolated polynucleic acid can comprise a nucleotide sequence that is at least 85% identical to SEQ ID NO: 14. In some cases, the isolated polynucleic acid can comprise a nucleotide sequence that is at least 86% identical to SEQ ID NO: 14. In some cases, the isolated polynucleic acid can comprise a nucleotide sequence that is at least 87% identical to SEQ ID NO: 14. In some cases, the isolated polynucleic acid can comprise a nucleotide sequence that is at least 88% identical to SEQ ID NO: 14. In some cases, the isolated polynucleic acid can comprise a nucleotide sequence that is at least 89% identical to SEQ ID NO: 14. In some cases, the isolated polynucleic acid can comprise a nucleotide sequence that is at least 90% identical to SEQ ID NO: 14. In some cases, the isolated polynucleic acid can comprise a nucleotide sequence that is at least 91% identical to SEQ ID NO: 14. In some cases, the isolated polynucleic acid can comprise a nucleotide sequence that is at least 92% identical to SEQ ID NO: 14. In some cases, the isolated polynucleic acid can comprise a nucleotide sequence that is at least 93% identical to SEQ ID NO: 14. In some cases, the isolated polynucleic acid can comprise a nucleotide sequence that is at least 94% identical to SEQ ID NO: 14. In some cases, the isolated polynucleic acid can comprise a nucleotide sequence that is at least 95% identical to SEQ ID NO: 14. In some cases, the isolated polynucleic acid can comprise a nucleotide sequence that is at least 96% identical to SEQ ID NO: 14. In some cases, the isolated polynucleic acid can comprise a nucleotide sequence that is at least 97% identical to SEQ ID NO: 14. In some cases, the isolated polynucleic acid can comprise a nucleotide sequence that is at least 98% identical to SEQ ID NO: 14. In some cases, the isolated polynucleic acid can comprise a nucleotide sequence that is at least 99% identical to SEQ ID NO: 14. In some cases, the isolated polynucleic acid can comprise a nucleotide sequence that is identical to SEQ ID NO: 14.

In some cases, the isolated polynucleic acid can encode a polyketide synthase (PKS). For example, the isolated polynucleic acid can comprise a nucleotide sequence that is substantially identical to SEQ ID NO: 6. In some cases, the isolated polynucleic acid can comprise a nucleotide sequence that is at least 60%, 65%, 70%, 75%, 80%, 85%, 90%, 91%, 92%, 93%, 94%, 95%, 96%, 97%, 98%, 99%, 99.9%, 99.99%, 99.999%, or 99.9999% identical to SEQ ID NO: 6. In some cases, the isolated polynucleic acid can comprise a nucleotide sequence that is at least 60% identical to SEQ ID NO: 6. In some cases, the isolated polynucleic acid can comprise a nucleotide sequence that is at least 65% identical to SEQ ID NO: 6. In some cases, the isolated polynucleic acid can comprise a nucleotide sequence that is at least 70% identical to SEQ ID NO: 6. In some cases, the isolated polynucleic acid can comprise a nucleotide sequence that is at least 75% identical to SEQ ID NO: 6. In some cases, the isolated polynucleic acid can comprise a nucleotide sequence that is at least 80% identical to SEQ ID NO: 6. In some cases, the isolated polynucleic acid can comprise a nucleotide sequence that is at least 81% identical to SEQ ID NO: 6. In some cases, the isolated polynucleic acid can comprise a nucleotide sequence that is at least 82% identical to SEQ ID NO: 6. In some cases, the isolated polynucleic acid can comprise a nucleotide sequence that is at least 83% identical to SEQ ID NO: 6. In some cases, the isolated polynucleic acid can comprise a nucleotide sequence that is at least 84% identical to SEQ ID NO: 6. In some cases, the isolated polynucleic acid can comprise a nucleotide sequence that is at least 85% identical to SEQ ID NO: 6. In some cases, the isolated polynucleic acid can comprise a nucleotide sequence that is at least 86% identical to SEQ ID NO: 6. In some cases, the isolated polynucleic acid can comprise a nucleotide sequence that is at least 87% identical to SEQ ID NO: 6. In some cases, the isolated polynucleic acid can comprise a nucleotide sequence that is at least 88% identical to SEQ ID NO: 6. In some cases, the isolated polynucleic acid can comprise a nucleotide sequence that is at least 89% identical to SEQ ID NO: 6. In some cases, the isolated polynucleic acid can comprise a nucleotide sequence that is at least 90% identical to SEQ ID NO: 6. In some cases, the isolated polynucleic acid can comprise a nucleotide sequence that is at least 91% identical to SEQ ID NO: 6. In some cases, the isolated polynucleic acid can comprise a nucleotide sequence that is at least 92% identical to SEQ ID NO: 6. In some cases, the isolated polynucleic acid can comprise a nucleotide sequence that is at least 93% identical to SEQ ID NO: 6. In some cases, the isolated polynucleic acid can comprise a nucleotide sequence that is at least 94% identical to SEQ ID NO: 6. In some cases, the isolated polynucleic acid can comprise a nucleotide sequence that is at least 95% identical to SEQ ID NO: 6. In some cases, the isolated polynucleic acid can comprise a nucleotide sequence that is at least 96% identical to SEQ ID NO: 6. In some cases, the isolated polynucleic acid can comprise a nucleotide sequence that is at least 97% identical to SEQ ID NO: 6. In some cases, the isolated polynucleic acid can comprise a nucleotide sequence that is at least 98% identical to SEQ ID NO: 6. In some cases, the isolated polynucleic acid can comprise a nucleotide sequence that is at least 99% identical to SEQ ID NO: 6. In some cases, the isolated polynucleic acid can comprise a nucleotide sequence that is identical to SEQ ID NO: 6.

In some cases, the isolated polynucleic acid can encode an olivetolic acid cyclase (OAC). For example, the isolated polynucleic acid can comprise a nucleotide sequence that is substantially identical to SEQ ID NO: 8. In some cases, the isolated polynucleic acid can comprise a nucleotide sequence that is at least 60%, 65%, 70%, 75%, 80%, 85%, 90%, 91%, 92%, 93%, 94%, 95%, 96%, 97%, 98%, 99%, 99.9%, 99.99%, 99.999%, or 99.9999% identical to SEQ ID NO: 8. In some cases, the isolated polynucleic acid can comprise a nucleotide sequence that is at least 60% identical to SEQ ID NO: 8. In some cases, the isolated polynucleic acid can comprise a nucleotide sequence that is at least 65% identical to SEQ ID NO: 8. In some cases, the isolated polynucleic acid can comprise a nucleotide sequence that is at least 70% identical to SEQ ID NO: 8. In some cases, the isolated polynucleic acid can comprise a nucleotide sequence that is at least 75% identical to SEQ ID NO: 8. In some cases, the isolated polynucleic acid can comprise a nucleotide sequence that is at least 80% identical to SEQ ID NO: 8. In some cases, the isolated polynucleic acid can comprise a nucleotide sequence that is at least 81% identical to SEQ ID NO: 8. In some cases, the isolated polynucleic acid can comprise a nucleotide sequence that is at least 82% identical to SEQ ID NO: 8. In some cases, the isolated polynucleic acid can comprise a nucleotide sequence that is at least 83% identical to SEQ ID NO: 8. In some cases, the isolated polynucleic acid can comprise a nucleotide sequence that is at least 84% identical to SEQ ID NO: 8. In some cases, the isolated polynucleic acid can comprise a nucleotide sequence that is at least 85% identical to SEQ ID NO: 8. In some cases, the isolated polynucleic acid can comprise a nucleotide sequence that is at least 86% identical to SEQ ID NO: 8. In some cases, the isolated polynucleic acid can comprise a nucleotide sequence that is at least 87% identical to SEQ ID NO: 8. In some cases, the isolated polynucleic acid can comprise a nucleotide sequence that is at least 88% identical to SEQ ID NO: 8. In some cases, the isolated polynucleic acid can comprise a nucleotide sequence that is at least 89% identical to SEQ ID NO: 8. In some cases, the isolated polynucleic acid can comprise a nucleotide sequence that is at least 90% identical to SEQ ID NO: 8. In some cases, the isolated polynucleic acid can comprise a nucleotide sequence that is at least 91% identical to SEQ ID NO: 8. In some cases, the isolated polynucleic acid can comprise a nucleotide sequence that is at least 92% identical to SEQ ID NO: 8. In some cases, the isolated polynucleic acid can comprise a nucleotide sequence that is at least 93% identical to SEQ ID NO: 8. In some cases, the isolated polynucleic acid can comprise a nucleotide sequence that is at least 94% identical to SEQ ID NO: 8. In some cases, the isolated polynucleic acid can comprise a nucleotide sequence that is at least 95% identical to SEQ ID NO: 8. In some cases, the isolated polynucleic acid can comprise a nucleotide sequence that is at least 96% identical to SEQ ID NO: 8. In some cases, the isolated polynucleic acid can comprise a nucleotide sequence that is at least 97% identical to SEQ ID NO: 8. In some cases, the isolated polynucleic acid can comprise a nucleotide sequence that is at least 98% identical to SEQ ID NO: 8. In some cases, the isolated polynucleic acid can comprise a nucleotide sequence that is at least 99% identical to SEQ ID NO: 8. In some cases, the isolated polynucleic acid can comprise a nucleotide sequence that is identical to SEQ ID NO: 8.

In some cases, the isolated polynucleic acid can encode a prenyltransferase (PT). For example, the isolated polynucleic acid can comprise a nucleotide sequence that is substantially identical to any one of SEQ ID NOs: 2, 26, 31, or 37. In some cases, the isolated polynucleic acid can comprise a nucleotide sequence that is at least 60%, 65%, 70%, 75%, 80%, 85%, 90%, 91%, 92%, 93%, 94%, 95%, 96%, 97%, 98%, 99%, 99.9%, 99.99%, 99.999%, or 99.9999% identical to any one of SEQ ID NOs: 2, 26, 31, or 37. In some cases, the isolated polynucleic acid can comprise a nucleotide sequence that is at least 60% identical to any one of SEQ ID NOs: 2, 26, 31, or 37. In some cases, the isolated polynucleic acid can comprise a nucleotide sequence that is at least 65% identical to any one of SEQ ID NOs: 2, 26, 31, or 37. In some cases, the isolated polynucleic acid can comprise a nucleotide sequence that is at least 70% identical to any one of SEQ ID NOs: 2, 26, 31, or 37. In some cases, the isolated polynucleic acid can comprise a nucleotide sequence that is at least 75% identical to any one of SEQ ID NOs: 2, 26, 31, or 37. In some cases, the isolated polynucleic acid can comprise a nucleotide sequence that is at least 80% identical to any one of SEQ ID NOs: 2, 26, 31, or 37. In some cases, the isolated polynucleic acid can comprise a nucleotide sequence that is at least 81% identical to any one of SEQ ID NOs: 2, 26, 31, or 37. In some cases, the isolated polynucleic acid can comprise a nucleotide sequence that is at least 82% identical to any one of SEQ ID NOs: 2, 26, 31, or 37. In some cases, the isolated polynucleic acid can comprise a nucleotide sequence that is at least 83% identical to any one of SEQ ID NOs: 2, 26, 31, or 37. In some cases, the isolated polynucleic acid can comprise a nucleotide sequence that is at least 84% identical to any one of SEQ ID NOs: 2, 26, 31, or 37. In some cases, the isolated polynucleic acid can comprise a nucleotide sequence that is at least 85% identical to any one of SEQ ID NOs: 2, 26, 31, or 37. In some cases, the isolated polynucleic acid can comprise a nucleotide sequence that is at least 86% identical to any one of SEQ ID NOs: 2, 26, 31, or 37. In some cases, the isolated polynucleic acid can comprise a nucleotide sequence that is at least 87% identical to any one of SEQ ID NOs: 2, 26, 31, or 37. In some cases, the isolated polynucleic acid can comprise a nucleotide sequence that is at least 88% identical to any one of SEQ ID NOs: 2, 26, 31, or 37. In some cases, the isolated polynucleic acid can comprise a nucleotide sequence that is at least 89% identical to any one of SEQ ID NOs: 2, 26, 31, or 37. In some cases, the isolated polynucleic acid can comprise a nucleotide sequence that is at least 90% identical to any one of SEQ ID NOs: 2, 26, 31, or 37. In some cases, the isolated polynucleic acid can comprise a nucleotide sequence that is at least 91% identical to any one of SEQ ID NOs: 2, 26, 31, or 37. In some cases, the isolated polynucleic acid can comprise a nucleotide sequence that is at least 92% identical to any one of SEQ ID NOs: 2, 26, 31, or 37. In some cases, the isolated polynucleic acid can comprise a nucleotide sequence that is at least 93% identical to any one of SEQ ID NOs: 2, 26, 31, or 37. In some cases, the isolated polynucleic acid can comprise a nucleotide sequence that is at least 94% identical to any one of SEQ ID NOs: 2, 26, 31, or 37. In some cases, the isolated polynucleic acid can comprise a nucleotide sequence that is at least 95% identical to any one of SEQ ID NOs: 2, 26, 31, or 37. In some cases, the isolated polynucleic acid can comprise a nucleotide sequence that is at least 96% identical to any one of SEQ ID NOs: 2, 26, 31, or 37. In some cases, the isolated polynucleic acid can comprise a nucleotide sequence that is at least 97% identical to any one of SEQ ID NOs: 2, 26, 31, or 37. In some cases, the isolated polynucleic acid can comprise a nucleotide sequence that is at least 98% identical to any one of SEQ ID NOs: 2, 26, 31, or 37. In some cases, the isolated polynucleic acid can comprise a nucleotide sequence that is at least 99% identical to any one of SEQ ID NOs: 2, 26, 31, or 37. In some cases, the isolated polynucleic acid can comprise a nucleotide sequence that is identical to any one of SEQ ID NOs: 2, 26, 31, or 37.

In some cases, the isolated polynucleic acid can encode a THCA synthase (THCAS). For example, the isolated polynucleic acid can comprise a nucleotide sequence that is substantially identical to SEQ ID NO: 10. In some cases, the isolated polynucleic acid can comprise a nucleotide sequence that is at least 60%, 65%, 70%, 75%, 80%, 85%, 90%, 91%, 92%, 93%, 94%, 95%, 96%, 97%, 98%, 99%, 99.9%, 99.99%, 99.999%, or 99.9999% identical to SEQ ID NO: 10. In some cases, the isolated polynucleic acid can comprise a nucleotide sequence that is at least 60% identical to SEQ ID NO: 10. In some cases, the isolated polynucleic acid can comprise a nucleotide sequence that is at least 65% identical to SEQ ID NO: 10. In some cases, the isolated polynucleic acid can comprise a nucleotide sequence that is at least 70% identical to SEQ ID NO: 10. In some cases, the isolated polynucleic acid can comprise a nucleotide sequence that is at least 75% identical to SEQ ID NO: 10. In some cases, the isolated polynucleic acid can comprise a nucleotide sequence that is at least 80% identical to SEQ ID NO: 10. In some cases, the isolated polynucleic acid can comprise a nucleotide sequence that is at least 81% identical to SEQ ID NO: 10. In some cases, the isolated polynucleic acid can comprise a nucleotide sequence that is at least 82% identical to SEQ ID NO: 10. In some cases, the isolated polynucleic acid can comprise a nucleotide sequence that is at least 83% identical to SEQ ID NO: 10. In some cases, the isolated polynucleic acid can comprise a nucleotide sequence that is at least 84% identical to SEQ ID NO: 10. In some cases, the isolated polynucleic acid can comprise a nucleotide sequence that is at least 85% identical to SEQ ID NO: 10. In some cases, the isolated polynucleic acid can comprise a nucleotide sequence that is at least 86% identical to SEQ ID NO: 10. In some cases, the isolated polynucleic acid can comprise a nucleotide sequence that is at least 87% identical to SEQ ID NO: 10. In some cases, the isolated polynucleic acid can comprise a nucleotide sequence that is at least 88% identical to SEQ ID NO: 10. In some cases, the isolated polynucleic acid can comprise a nucleotide sequence that is at least 89% identical to SEQ ID NO: 10. In some cases, the isolated polynucleic acid can comprise a nucleotide sequence that is at least 90% identical to SEQ ID NO: 10. In some cases, the isolated polynucleic acid can comprise a nucleotide sequence that is at least 91% identical to SEQ ID NO: 10. In some cases, the isolated polynucleic acid can comprise a nucleotide sequence that is at least 92% identical to SEQ ID NO: 10. In some cases, the isolated polynucleic acid can comprise a nucleotide sequence that is at least 93% identical to SEQ ID NO: 10. In some cases, the isolated polynucleic acid can comprise a nucleotide sequence that is at least 94% identical to SEQ ID NO: 10. In some cases, the isolated polynucleic acid can comprise a nucleotide sequence that is at least 95% identical to SEQ ID NO: 10. In some cases, the isolated polynucleic acid can comprise a nucleotide sequence that is at least 96% identical to SEQ ID NO: 10. In some cases, the isolated polynucleic acid can comprise a nucleotide sequence that is at least 97% identical to SEQ ID NO: 10. In some cases, the isolated polynucleic acid can comprise a nucleotide sequence that is at least 98% identical to SEQ ID NO: 10. In some cases, the isolated polynucleic acid can comprise a nucleotide sequence that is at least 99% identical to SEQ ID NO: 10. In some cases, the isolated polynucleic acid can comprise a nucleotide sequence that is identical to SEQ ID NO: 10.

In some cases, the isolated polynucleic acid can encode a CBDA synthase (CBDAS). For example, the isolated polynucleic acid can comprise a nucleotide sequence that is substantially identical to SEQ ID NO: 12. In some cases, the isolated polynucleic acid can comprise a nucleotide sequence that is at least 60%, 65%, 70%, 75%, 80%, 85%, 90%, 91%, 92%, 93%, 94%, 95%, 96%, 97%, 98%, 99%, 99.9%, 99.99%, 99.999%, or 99.9999% identical to SEQ ID NO: 12. In some cases, the isolated polynucleic acid can comprise a nucleotide sequence that is at least 60% identical to SEQ ID NO: 12. In some cases, the isolated polynucleic acid can comprise a nucleotide sequence that is at least 65% identical to SEQ ID NO: 12. In some cases, the isolated polynucleic acid can comprise a nucleotide sequence that is at least 70% identical to SEQ ID NO: 12. In some cases, the isolated polynucleic acid can comprise a nucleotide sequence that is at least 75% identical to SEQ ID NO: 12. In some cases, the isolated polynucleic acid can comprise a nucleotide sequence that is at least 80% identical to SEQ ID NO: 12. In some cases, the isolated polynucleic acid can comprise a nucleotide sequence that is at least 81% identical to SEQ ID NO: 12. In some cases, the isolated polynucleic acid can comprise a nucleotide sequence that is at least 82% identical to SEQ ID NO: 12. In some cases, the isolated polynucleic acid can comprise a nucleotide sequence that is at least 83% identical to SEQ ID NO: 12. In some cases, the isolated polynucleic acid can comprise a nucleotide sequence that is at least 84% identical to SEQ ID NO: 12. In some cases, the isolated polynucleic acid can comprise a nucleotide sequence that is at least 85% identical to SEQ ID NO: 12. In some cases, the isolated polynucleic acid can comprise a nucleotide sequence that is at least 86% identical to SEQ ID NO: 12. In some cases, the isolated polynucleic acid can comprise a nucleotide sequence that is at least 87% identical to SEQ ID NO: 12. In some cases, the isolated polynucleic acid can comprise a nucleotide sequence that is at least 88% identical to SEQ ID NO: 12. In some cases, the isolated polynucleic acid can comprise a nucleotide sequence that is at least 89% identical to SEQ ID NO: 12. In some cases, the isolated polynucleic acid can comprise a nucleotide sequence that is at least 90% identical to SEQ ID NO: 12. In some cases, the isolated polynucleic acid can comprise a nucleotide sequence that is at least 91% identical to SEQ ID NO: 12. In some cases, the isolated polynucleic acid can comprise a nucleotide sequence that is at least 92% identical to SEQ ID NO: 12. In some cases, the isolated polynucleic acid can comprise a nucleotide sequence that is at least 93% identical to SEQ ID NO: 12. In some cases, the isolated polynucleic acid can comprise a nucleotide sequence that is at least 94% identical to SEQ ID NO: 12. In some cases, the isolated polynucleic acid can comprise a nucleotide sequence that is at least 95% identical to SEQ ID NO: 12. In some cases, the isolated polynucleic acid can comprise a nucleotide sequence that is at least 96% identical to SEQ ID NO: 12. In some cases, the isolated polynucleic acid can comprise a nucleotide sequence that is at least 97% identical to SEQ ID NO: 12. In some cases, the isolated polynucleic acid can comprise a nucleotide sequence that is at least 98% identical to SEQ ID NO: 12. In some cases, the isolated polynucleic acid can comprise a nucleotide sequence that is at least 99% identical to SEQ ID NO: 12. In some cases, the isolated polynucleic acid can comprise a nucleotide sequence that is identical to SEQ ID NO: 12.

In some cases, the isolated polynucleic acid can encode a CBCA synthase (CBCAS). For example, the isolated polynucleic acid can comprise a nucleotide sequence that is substantially identical to SEQ ID NO: 18. In some cases, the isolated polynucleic acid can comprise a nucleotide sequence that is at least 60%, 65%, 70%, 75%, 80%, 85%, 90%, 91%, 92%, 93%, 94%, 95%, 96%, 97%, 98%, 99%, 99.9%, 99.99%, 99.999%, or 99.9999% identical to SEQ ID NO: 18. In some cases, the isolated polynucleic acid can comprise a nucleotide sequence that is at least 60% identical to SEQ ID NO: 18. In some cases, the isolated polynucleic acid can comprise a nucleotide sequence that is at least 65% identical to SEQ ID NO: 18. In some cases, the isolated polynucleic acid can comprise a nucleotide sequence that is at least 70% identical to SEQ ID NO: 18. In some cases, the isolated polynucleic acid can comprise a nucleotide sequence that is at least 75% identical to SEQ ID NO: 18. In some cases, the isolated polynucleic acid can comprise a nucleotide sequence that is at least 80% identical to SEQ ID NO: 18. In some cases, the isolated polynucleic acid can comprise a nucleotide sequence that is at least 81% identical to SEQ ID NO: 18. In some cases, the isolated polynucleic acid can comprise a nucleotide sequence that is at least 82% identical to SEQ ID NO: 18. In some cases, the isolated polynucleic acid can comprise a nucleotide sequence that is at least 83% identical to SEQ ID NO: 18. In some cases, the isolated polynucleic acid can comprise a nucleotide sequence that is at least 84% identical to SEQ ID NO: 18. In some cases, the isolated polynucleic acid can comprise a nucleotide sequence that is at least 85% identical to SEQ ID NO: 18. In some cases, the isolated polynucleic acid can comprise a nucleotide sequence that is at least 86% identical to SEQ ID NO: 18. In some cases, the isolated polynucleic acid can comprise a nucleotide sequence that is at least 87% identical to SEQ ID NO: 18. In some cases, the isolated polynucleic acid can comprise a nucleotide sequence that is at least 88% identical to SEQ ID NO: 18. In some cases, the isolated polynucleic acid can comprise a nucleotide sequence that is at least 89% identical to SEQ ID NO: 18. In some cases, the isolated polynucleic acid can comprise a nucleotide sequence that is at least 90% identical to SEQ ID NO: 18. In some cases, the isolated polynucleic acid can comprise a nucleotide sequence that is at least 91% identical to SEQ ID NO: 18. In some cases, the isolated polynucleic acid can comprise a nucleotide sequence that is at least 92% identical to SEQ ID NO: 18. In some cases, the isolated polynucleic acid can comprise a nucleotide sequence that is at least 93% identical to SEQ ID NO: 18. In some cases, the isolated polynucleic acid can comprise a nucleotide sequence that is at least 94% identical to SEQ ID NO: 18. In some cases, the isolated polynucleic acid can comprise a nucleotide sequence that is at least 95% identical to SEQ ID NO: 18. In some cases, the isolated polynucleic acid can comprise a nucleotide sequence that is at least 96% identical to SEQ ID NO: 18. In some cases, the isolated polynucleic acid can comprise a nucleotide sequence that is at least 97% identical to SEQ ID NO: 18. In some cases, the isolated polynucleic acid can comprise a nucleotide sequence that is at least 98% identical to SEQ ID NO: 18. In some cases, the isolated polynucleic acid can comprise a nucleotide sequence that is at least 99% identical to SEQ ID NO: 18. In some cases, the isolated polynucleic acid can comprise a nucleotide sequence that is identical to SEQ ID NO: 18.

In some cases, the isolated polynucleic acid can encode a HMG-CoA reductase (HMG1). For example, the isolated polynucleic acid can comprise a nucleotide sequence that is substantially identical to SEQ ID NO: 20 or 22. In some cases, the isolated polynucleic acid can comprise a nucleotide sequence that is at least 50%, 60%, 70%, 80%, 85%, 90%, 95%, 96%, 97%, 98% or 99% identical to SEQ ID NO: 20 or 22. In some cases, the isolated polynucleic acid can comprise a nucleotide sequence that is at least 50% identical to SEQ ID NO: 20 or 22. In some cases, the isolated polynucleic acid can comprise a nucleotide sequence that is at least 60% identical to SEQ ID NO: 20 or 22. In some cases, the isolated polynucleic acid can comprise a nucleotide sequence that is at least 65% identical to SEQ ID NO: 20 or 22. In some cases, the isolated polynucleic acid can comprise a nucleotide sequence that is at least 70% identical to SEQ ID NO: 20 or 22. In some cases, the isolated polynucleic acid can comprise a nucleotide sequence that is at least 75% identical to SEQ ID NO: 20 or 22. In some cases, the isolated polynucleic acid can comprise a nucleotide sequence that is at least 80% identical to SEQ ID NO: 20 or 22. In some cases, the isolated polynucleic acid can comprise a nucleotide sequence that is at least 81% identical to SEQ ID NO: 20 or 22. In some cases, the isolated polynucleic acid can comprise a nucleotide sequence that is at least 82% identical to SEQ ID NO: 20 or 22. In some cases, the isolated polynucleic acid can comprise a nucleotide sequence that is at least 83% identical to SEQ ID NO: 20 or 22. In some cases, the isolated polynucleic acid can comprise a nucleotide sequence that is at least 84% identical to SEQ ID NO: 20 or 22. In some cases, the isolated polynucleic acid can comprise a nucleotide sequence that is at least 85% identical to SEQ ID NO: 20 or 22. In some cases, the isolated polynucleic acid can comprise a nucleotide sequence that is at least 86% identical to SEQ ID NO: 20 or 22. In some cases, the isolated polynucleic acid can comprise a nucleotide sequence that is at least 87% identical to SEQ ID NO: 20 or 22. In some cases, the isolated polynucleic acid can comprise a nucleotide sequence that is at least 88% identical to SEQ ID NO: 20 or 22. In some cases, the isolated polynucleic acid can comprise a nucleotide sequence that is at least 89% identical to SEQ ID NO: 20 or 22. In some cases, the isolated polynucleic acid can comprise a nucleotide sequence that is at least 90% identical to SEQ ID NO: 20 or 22. In some cases, the isolated polynucleic acid can comprise a nucleotide sequence that is at least 91% identical to SEQ ID NO: 20 or 22. In some cases, the isolated polynucleic acid can comprise a nucleotide sequence that is at least 92% identical to SEQ ID NO: 20 or 22. In some cases, the isolated polynucleic acid can comprise a nucleotide sequence that is at least 93% identical to SEQ ID NO: 20 or 22. In some cases, the isolated polynucleic acid can comprise a nucleotide sequence that is at least 94% identical to SEQ ID NO: 20 or 22. In some cases, the isolated polynucleic acid can comprise a nucleotide sequence that is at least 95% identical to SEQ ID NO: 20 or 22. In some cases, the isolated polynucleic acid can comprise a nucleotide sequence that is at least 96% identical to SEQ ID NO: 20 or 22. In some cases, the isolated polynucleic acid can comprise a nucleotide sequence that is at least 97% identical to SEQ ID NO: 20 or 22. In some cases, the isolated polynucleic acid can comprise a nucleotide sequence that is at least 98% identical to SEQ ID NO: 20 or 22. In some cases, the isolated polynucleic acid can comprise a nucleotide sequence that is at least 99% identical to SEQ ID NO: 20 or 22. In some cases, the isolated polynucleic acid can comprise a nucleotide sequence that is identical to SEQ ID NO: 20 or 22.

In some cases, the isolated polynucleic acid can encode a farnesyl pyrophosphate synthetase (ERG20). For example, the isolated polynucleic acid can comprise a nucleotide sequence that is substantially identical to SEQ ID NO: 24. In some cases, the isolated polynucleic acid can comprise a nucleotide sequence that is at least 50%, 60%, 70%, 80%, 85%, 90%, 95%, 96%, 97%, 98% or 99% identical to SEQ ID NO: 24. In some cases, the isolated polynucleic acid can comprise a nucleotide sequence that is at least 50% identical to SEQ ID NO: 24. In some cases, the isolated polynucleic acid can comprise a nucleotide sequence that is at least 60% identical to SEQ ID NO: 24. In some cases, the isolated polynucleic acid can comprise a nucleotide sequence that is at least 65% identical to SEQ ID NO: 24. In some cases, the isolated polynucleic acid can comprise a nucleotide sequence that is at least 70% identical to SEQ ID NO: 24. In some cases, the isolated polynucleic acid can comprise a nucleotide sequence that is at least 75% identical to SEQ ID NO: 24. In some cases, the isolated polynucleic acid can comprise a nucleotide sequence that is at least 80% identical to SEQ ID NO: 24. In some cases, the isolated polynucleic acid can comprise a nucleotide sequence that is at least 81% identical to SEQ ID NO: 24. In some cases, the isolated polynucleic acid can comprise a nucleotide sequence that is at least 82% identical to SEQ ID NO: 24. In some cases, the isolated polynucleic acid can comprise a nucleotide sequence that is at least 83% identical to SEQ ID NO: 24. In some cases, the isolated polynucleic acid can comprise a nucleotide sequence that is at least 84% identical to SEQ ID NO: 24. In some cases, the isolated polynucleic acid can comprise a nucleotide sequence that is at least 85% identical to SEQ ID NO: 24. In some cases, the isolated polynucleic acid can comprise a nucleotide sequence that is at least 86% identical to SEQ ID NO: 24. In some cases, the isolated polynucleic acid can comprise a nucleotide sequence that is at least 87% identical to SEQ ID NO: 24. In some cases, the isolated polynucleic acid can comprise a nucleotide sequence that is at least 88% identical to SEQ ID NO: 24. In some cases, the isolated polynucleic acid can comprise a nucleotide sequence that is at least 89% identical to SEQ ID NO: 24. In some cases, the isolated polynucleic acid can comprise a nucleotide sequence that is at least 90% identical to SEQ ID NO: 24. In some cases, the isolated polynucleic acid can comprise a nucleotide sequence that is at least 91% identical to SEQ ID NO: 24. In some cases, the isolated polynucleic acid can comprise a nucleotide sequence that is at least 92% identical to SEQ ID NO: 24. In some cases, the isolated polynucleic acid can comprise a nucleotide sequence that is at least 93% identical to SEQ ID NO: 24. In some cases, the isolated polynucleic acid can comprise a nucleotide sequence that is at least 94% identical to SEQ ID NO: 24. In some cases, the isolated polynucleic acid can comprise a nucleotide sequence that is at least 95% identical to SEQ ID NO: 24. In some cases, the isolated polynucleic acid can comprise a nucleotide sequence that is at least 96% identical to SEQ ID NO: 24. In some cases, the isolated polynucleic acid can comprise a nucleotide sequence that is at least 97% identical to SEQ ID NO: 24. In some cases, the isolated polynucleic acid can comprise a nucleotide sequence that is at least 98% identical to SEQ ID NO: 24. In some cases, the isolated polynucleic acid can comprise a nucleotide sequence that is at least 99% identical to SEQ ID NO: 24. In some cases, the isolated polynucleic acid can be identical to SEQ ID NO: 24.

### Methods of Making Genetically Modified Microorganisms

Disclosed herein is a method of making a genetically modified microorganism capable of converting a carbon substrate into CBGA. Also disclosed herein is a method of making a genetically modified microorganism capable of converting a carbon substrate into a cannabinoid.

In some cases, the microorganism can be made by contacting the microorganism with one or more polynucleotides. The polynucleotides can be a vector. The polynucleotides can also comprise one or more genes encoding for an enzymes.

In some cases, the microorganism can be grown so that the polynucleotides are inserted into the microorganism. In some cases, the insertion can be done any method, e.g., transfections, transformation, etc. The insertion of the microorganism can be by plasmid or in some cases the insertion can lead to a stable integration of the plasmid into the chromosome of the microorganism.

The genes encoding for an enzymes can include (i) acyl activating enzyme (AAE1); (ii) polyketide synthase (PKS); (iii) olivetolic acid cyclase (OAC); (iv) prenyltransferase (PT); (v) THCA synthase (THCAS); (vi) CBDA synthase (CBDAS); and/or (vii) CBCA synthase (CBCAS). In some further cases, the genes encoding for an enzyme can include (viii) a HMG-Co reductase (HMG1) and/or (ix) a farnesyl pyrophosphate synthetase (ERG20).

In some cases, the microorganism can be contacted with a polynucleotide that encodes for a prenyltransferase (PT). In some cases, the PT can be encoded by a nucleotide sequence that is substantially identical to SEQ ID NO: 2. In some cases, the polynucleotide can be translated into an amino acid sequence that is substantially identical to SEQ ID NO: 1. In some cases, the PT can be encoded by a nucleotide sequence that is substantially identical to SEQ ID NO: 26. In some cases, the polynucleotide can be translated into an amino acid sequence that is substantially identical to SEQ ID NO: 27. In some cases, the PT can be encoded by a nucleotide sequence that is substantially identical to SEQ ID NO: 31. In some cases, the polynucleotide can be translated into an amino acid sequence that is substantially identical to SEQ ID NO: 32. In some cases, the PT can be encoded by a nucleotide sequence that is substantially identical to SEQ ID NO: 37. In some cases, the polynucleotide can be translated into an amino acid sequence that is substantially identical to SEQ ID NO: 38.

In some cases, the microorganism can also be contacted with a polynucleotide that encodes for an AAE1. In some cases, the AAE1 is encoded by a polynucleotide sequence that is substantially identical to SEQ ID NO: 14. In some cases, the polynucleotide can be translated into an amino acid sequence that is substantially identical to SEQ ID NO: 13.

In some cases, the microorganism can also be contacted with a polynucleotide that encodes for a PKS. In some cases, the PKS encoded by a polynucleotide sequence that is substantially identical to SEQ ID NO: 6. In some cases, the polynucleotide can be translated into an amino acid sequence that is substantially identical to SEQ ID NO: 5.

In some cases, the microorganism can also be contacted with a polynucleotide that encodes for an OAC. In some cases, the OAC encoded by a polynucleotide sequence that is substantially identical to SEQ ID NO: 8. In some cases, the polynucleotide can be translated into an amino acid sequence that is substantially identical to SEQ ID NO: 7.

In some cases, the microorganism can also be contacted with a polynucleotide that encodes for a THCAS. In some cases, the THCAS encoded by a polynucleotide sequence that is substantially identical to SEQ ID NO: 10. In some cases, the polynucleotide can be translated into an amino acid sequence that is substantially identical to SEQ ID NO: 9.

In some cases, the microorganism can also be contacted with a polynucleotide that encodes for a CBDAS. In some cases, the CBDAS encoded by a polynucleotide sequence that is substantially identical to SEQ ID NO: 12. In some cases, the polynucleotide can be translated into an amino acid sequence that is substantially identical to SEQ ID NO: 11.

In some cases, the microorganism can also be contacted with a polynucleotide that encodes for a CBCAS. In some cases, the CBCAS encoded by a polynucleotide sequence that is substantially identical to SEQ ID NO: 18. In some cases, the polynucleotide can be translated into an amino acid sequence that is substantially identical to SEQ ID NO: 17.

In some cases, the microorganism can also be contacted with a polynucleotide that encodes for an HMG-Co reductase (HMG1). In some cases, the HMG1 encoded by a polynucleotide sequence that is substantially identical to SEQ ID NO: 20 or 22. In some cases, the polynucleotide can be translated into an amino acid sequence that is substantially identical to SEQ ID NO: 19 or 21.

In some cases, the microorganism can also be contacted with a polynucleotide that encodes for a farnesyl pyrophosphate synthetase (ERG20). In some cases, the ERG20 encoded by a polynucleotide sequence that is substantially identical to SEQ ID NO: 24. In some cases, the polynucleotide can be translated into an amino acid sequence that is substantially identical to SEQ ID NO: 23.

The microorganism can be any type of microorganism that is disclosed throughout. For example, the microorganism can be a bacterium or a yeast.

The cannabinoid that can be made can be one or more of the following: cannabinoid is Δ⁹-tetrahydrocannabinolic acid (THCA), cannabidiolic acid (CBDA), cannabichromenic acid (CBCA), Δ⁹-tetrahydrocannabinol (THC), cannabidiol (CBD), cannabichromene (CBC), Δ⁹-tetrahydrocannabivarinic acid (THCVA), cannabidivarinic acid (CBDVA), cannabichromevarinic acid (CBCVA), or any combination thereof.

### Exemplary Genetically Modified Microorganisms

Disclosed herein is a genetically modified microorganism capable of converting a carbon substrate into CBGA, CBGVA or a cannabinoid.

The genetically modified microorganism can comprise a heterologous polynucleotide encoding an acyl activating enzyme (AAE1); polyketide synthase (PKS); olivetolic acid cyclase (OAC); and/or prenyltransferase (PT). In some cases, two or more polynucleotides encoding AAE1, PKS, OAC, and/or PT can be present within the genetically modified microorganism. In some cases, three of the polynucleotides encoding AAE1, PKS, OAC, and/or PT can be present within the genetically modified microorganism. In some cases, all four of the polynucleotides encoding AAE1, PKS, OAC, and PT can be present within the genetically modified microorganism.

Additionally, the genetically modified microorganism can further comprise polynucleotides encoding for a THCA synthase (THCAS); a CBDA synthase (CBDAS), a CBCA synthase (CBCAS), an HMG-Co reductase (HMG1) and/or a farnesyl pyrophosphate synthetase (ERG20). In some cases, the polynucleotides can be heterologous. In some cases, two or more polynucleotides encoding THCAS, CBDAS, CBCAS, HMG1, and/or ERG20 can be present within the genetically modified microorganism. In some cases, three or more of the polynucleotides encoding THCAS, CBDAS, CBCAS, HMG1, and/or ERG20 can be present within the genetically modified microorganism.

Should an AAE1 be present within the genetically modified microorganism, the AAE1 can be encoded by an amino acid sequence that is substantially identical to SEQ ID NO: 13. In some cases, the AAE1 can be encoded by a polynucleotide sequence that is substantially identical to SEQ ID NO: 14.

Should a PKS be present within the genetically modified microorganism, the PKS can be encoded by an amino acid sequence that is substantially identical to SEQ ID NO: 5. In some cases, the PKS can be encoded by a polynucleotide sequence that is substantially identical to SEQ ID NO: 6.

Should an OAC be present within the genetically modified microorganism, the OAC can be encoded by an amino acid sequence that is substantially identical to SEQ ID NO: 7. In some cases, the OAC can be encoded by a polynucleotide sequence that is substantially identical to SEQ ID NO: 8.

Should a PT be present within the genetically modified microorganism, the PT can be encoded by an amino acid sequence that is substantially identical to any one of SEQ ID NOs: 1, 27, 32, or 38. In some cases, the PT can be encoded by a polynucleotide sequence that is substantially identical to any one of SEQ ID NOs: 2, 26, 31, or 37.

Should a THCAS be present within the genetically modified microorganism, the THCAS can be encoded by an amino acid sequence that is substantially identical to SEQ ID NO: 9. In some cases, the THCAS can be encoded by a polynucleotide sequence that is substantially identical to SEQ ID NO: 10.

Should a CBDAS be present within the genetically modified microorganism, the CBDAS can be encoded by an amino acid sequence that is substantially identical to SEQ ID NO: 11. In some cases, the CBDAS can be encoded by a polynucleotide sequence that is substantially identical to SEQ ID NO: 12.

Should a CBCAS be present within the genetically modified microorganism, the CBCAS can be encoded by an amino acid sequence that is substantially identical to SEQ ID NO: 17. In some cases, the CBCAS can be encoded by a polynucleotide sequence that is substantially identical to SEQ ID NO: 18.

Should a HMG1 be present within the genetically modified microorganism, the CBCAS can be encoded by an amino acid sequence that is substantially identical to SEQ ID NO: 19 or 21. In some cases, the CBCAS can be encoded by a polynucleotide sequence that is substantially identical to SEQ ID NO: 20 or 22.

Should an ERG20 be present within the genetically modified microorganism, the ERG20 can be encoded by an amino acid sequence that is substantially identical to SEQ ID NO: 23. In some cases, the ERG20 can be encoded by a polynucleotide sequence that is substantially identical to SEQ ID NO: 24.

Should a TKS (OS) be present within the genetically modified microorganism, the TKS can be encoded by an amino acid sequence that is substantially identical to SEQ ID NO:41. In some cases, the TKS can be encoded by a polynucleotide sequence that is substantially identical to SEQ ID NO: 40.

In certain cases, the genetically modified microorganism can be a yeast or bacterium. Should the genetically modified microorganism be a yeast, the yeast can be from the genus Saccharomyces. More specifically, the yeast can be from the species Saccharomyces cerevisiae. Should the genetically modified microorganism be a bacterium, the bacterium can be from the genus Escherichia, e.g., Escherichia coli.

The genetically modified microorganism can use hexanoic acid and/or butyric acid. In some cases, the genetically modified microorganism can use sugar as a substrate. In some cases, the genetically modified microorganism can make a CBGA, CBGVA, THCA, CBDA, CBCA or a cannabinoid. If a cannabinoid is made, in some cases, the cannabinoid can be 49-tetrahydrocannabinol (THC), cannabidiol (CBD), cannabichromene (CBC), Δ⁹-tetrahydrocannabivarinic acid (THCVA), cannabidivarinic acid (CBDVA), and/or cannabichromevarinic acid (CBCVA).

### Fermentation Methods and Processes

In general, the microorganisms disclosed herein should be placed in fermentation conditions that are appropriate to convert a carbon source (such as a sugar, alcohol, and/or fatty acid) to CBGA, CBGVA or a cannabinoid (e.g., THC, CBD, CBC, THCVA, CBDVA, CBCVA). Reaction conditions that should be considered include temperature, media flow rate, pH, media redox potential, agitation rate (if using a continuous stirred tank reactor), inoculum level, maximum substrate concentrations and rates of introduction of the substrate to the bioreactor to ensure that substrate level does not become limiting, and maximum product concentrations to avoid product inhibition.

In some cases, non-genetically modified microorganisms can be used to increase CBGA or cannabinoid production. For example, cells taken from organisms that naturally produce cannabinoids can be used. These cells can be isolated and once isolated they can be used in a fermentation process.

### Fermentation Conditions

The fermentation conditions described herein are applicable to any and all methods disclosed throughout the application.

pH can have a profound effect on overall CBGA, CBGVA or cannabinoid production. Therefore, pH adjustments should be made in some cases.

In some cases, the pH during fermentation can vary from 4 to 10. In some instances, the pH can be from 5 to 9; 6 to 8; 6.1 to 7.9; 6.2 to 7.8; 6.3 to 7.7; 6.4 to 7.6; or 6.5 to 7.5. For example, the pH can be from 6.6 to 7.4. In some instances, the pH can be from 5 to 9. In some instances, the pH can be from 6 to 8. In some instances, the pH can be from 6.1 to 7.9. In some instances, the pH can be from 6.2 to 7.8. In some instances, the pH can be from 6.3 to 7.7. In some instances, the pH can be from 6.4 to 7.6. In some instances, the pH can be from 5.5 to 7.5. In some instances, the pH can be from 6.5 to 7.5. In some instances the pH used for the fermentation can be greater than 6. In some instances the pH used for the fermentation can be lower than 10.

Temperature

Temperature can also be adjusted based on cell, microorganism, or enzyme sensitivity. For example, the temperature used during fermentation, can from 27° C. to 45° C. In other instances, the temperature of the fermentation can be from 27° C. to 45° C.; 28° C. to 44° C.; 29° C. to 43° C.; 30° C. to 42° C.; 31° C. to 41° C.; 32° C. to 40° C. For example, the temperature can be from 36° C. to 39° C. (e.g., 36° C., 37° C., 38° C., or 39° C. In some instances, the temperature can be from 27° C. to 45° C. In some instances, the temperature can be from 28° C. to 44° C. In some instances, the temperature can be from 29° C. to 43° C. In some instances, the temperature can be from 30° C. to 42° C. In some instances, the temperature can be from 31° C. to 41° C. In some instances, the temperature can be from 32° C. to 40° C.

Gases

Availability of oxygen and other gases such as gaseous CO₂ can affect yield and fermentation rate. For example, when considering oxygen availability, the percent of dissolved oxygen (DO) within the fermentation media can be from 1% to 40%. In certain instances, the DO concentration can be from 1.5% to 35%; 2% to 30%; 2.5% to 25%; 3% to 20%; 4% to 19%; 5% to 18%; 6% to 17%; 7% to 16%; 8% to 15%; 9% to 14%; 10% to 13%; or 11% to 12%. For example, in some cases the DO concentration can be from 2% to 30%. In other cases, the DO can be from 3% to 20%. In some instances, the DO can be from 4% to 10%. In some cases, the DO can be from 1.5% to 35%. In some cases, the DO can be from 2.5% to 25%. In some cases, the DO can be from 4% to 19%. In some cases, the DO can be from 5% to 18%. In some cases, the DO can be from 6% to 17%. In some cases, the DO can be from 7% to 16%. In some cases, the DO can be from 8% to 15%. In some cases, the DO can be from 9% to 14%. In some cases, the DO can be from 10% to 13%. In some cases, the DO can be from 11% to 12%.

For example, when considering atmospheric CO₂, the percent of atmospheric CO₂ within an incubator can be from 0% to 10%. In some cases, atmospheric CO₂ can help to control the pH within cell culture medium. pH contain within cell culture media is dependent on a balance of dissolved CO₂ and bicarbonate (HCO₃). Changes in atmospheric CO₂ can alter the pH of the medium. In certain instances, the atmospheric CO₂ can be from 0% to 10%; 0.01% to 9%; 0.05% to 8%; 0.1% to 7%; 0.5% to 6%; 1% to 5%; 2% to 4%; 3% to 6%; 4% to 7%; 2% to 6%; or 5% to 10%. For example, in some cases the atmospheric CO₂ can be from 0% to 10%. In other cases, the atmospheric CO₂ can be from 0.01% to 9%. In some instances, the atmospheric CO₂ can be from 0.05% to 8%. In some cases, the atmospheric CO₂ can be from 0.1% to 7%. In some cases, the atmospheric CO₂ can be from 0.5% to 6%. In some cases, the atmospheric CO₂ can be from 1% to 5%. In some cases, the atmospheric CO₂ can be from 2% to 4%. In some cases, the atmospheric CO₂ can be from 3% to 6%. In some cases, the atmospheric CO₂ can be from 4% to 7%. In some cases, the atmospheric CO₂ can be from 2% to 6%. In some cases, the atmospheric CO₂ can be from 5% to 10%.

Bioreactors

Fermentation reactions can be carried out in any suitable bioreactor. In some embodiments of the invention, the bioreactor can comprise a first, growth reactor in which the microorganisms or cells are cultured, and a second, fermentation reactor, to which broth from the growth reactor is fed and in which most of the fermentation product (for example, CBGA or cannabinoids) is produced.

Media

The medium used to ferment CBGA, CBGVA or cannabinoid with the microorganisms described throughout can include hexanoic acid and/or butyric acid. For example, in some cases, the media can comprise a combination of hexanoic acid, yeast extract, peptone, and glucose. In other cases, the media can comprise a combination of butyric acid, yeast extract, peptone, and glucose. In certain cases, the media can comprise 10 g/L of yeast extract, 20 g/L peptone, 20 g/L glucose and 100 mg/L hexanoic acid or butyric acid. In some cases, hexanoic acid or butyric acid can be used in an amount of 1 mg/L to 1 g/L. In some cases, hexanoic acid or butyric acid can be used in an amount of 10 mg/to 900 mg/L. In some cases, hexanoic acid or butyric acid can be used in an amount of 25 mg/to 800 mg/L. In some cases, hexanoic acid or butyric acid can be used in an amount of 50 mg/to 700 mg/L. In some cases, hexanoic acid or butyric acid can be used in an amount of 75 mg/to 600 mg/L. In some cases, hexanoic acid or butyric acid can be used in an amount of 100 mg/to 500 mg/L. In some cases, hexanoic acid or butyric acid can be used in an amount of 125 mg/to 400 mg/L. In some cases, hexanoic acid or butyric acid can be used in an amount of 150 mg/to 300 mg/L. In some cases, hexanoic acid or butyric acid can be used in an amount of 175 mg/to 250 mg/L. In some cases, hexanoic acid or butyric acid can be used in an amount of 50 mg/to 250 mg/L. In some cases, hexanoic acid or butyric acid can be used in an amount of 75 mg/to 200 mg/L. In some cases, hexanoic acid or butyric acid can be used in an amount of 90 mg/to 150 mg/L. Henanoic and butyric acid can be used in similar concentrations.

In some cases, hexanoic acid can be used in an amount of 1 g/L to produce 700 mg/L of CBGA or cannabinoids with the microorganism described throughout. In some cases, hexanoic acid can be used in an amount of at least about 1 g/L, at least about 1.5 g/L, at least about 2 g/L, at least about 2.5 g/L, at least about 3 g/L, at least about 3.5 g/L, at least about 4 g/L, at least about 4.5 g/L, at least about 5 g/L, at least about 5.5 g/L, at least about 6 g/L, at least about 6.5 g/L, at least about 7 g/L, at least about 7.5 g/L, at least about 8 g/L, at least about 8.5 g/L, at least about 9 g/L, at least about 9.5 g/L, at least about 10 g/L, at least about 11 g/L, at least about 12 g/L, at least about 13 g/L, at least about 14 g/L, at least about 15 g/L, at least about 16 g/L, at least about 17 g/L, at least about 18 g/L, at least about 19 g/L, at least about 20 g/L, between about 0.1 g/L and about 20 g/L, between about 0.5 g/L and about 15 g/L, between about 1 g/L and about 10 g/L, between about 1 g/L and about 9 g/L, between about 1 g/L and about 8 g/L, between about 1 g/L and about 7 g/L, between about 2 g/L and about 6 g/L, between about 2 g/L and about 5 g/L, or between about 3 g/L and about 4 g/L. In some cases, butyric acid can be used in an amount of 1 g/L to produce 700 mg/L of CBGVA or cannabinoids with the microorganism described throughout. In some cases, butric acid can be used in an amount of at least about 1 g/L, at least about 1.5 g/L, at least about 2 g/L, at least about 2.5 g/L, at least about 3 g/L, at least about 3.5 g/L, at least about 4 g/L, at least about 4.5 g/L, at least about 5 g/L, at least about 5.5 g/L, at least about 6 g/L, at least about 6.5 g/L, at least about 7 g/L, at least about 7.5 g/L, at least about 8 g/L, at least about 8.5 g/L, at least about 9 g/L, at least about 9.5 g/L, at least about 10 g/L, at least about 11 g/L, at least about 12 g/L, at least about 13 g/L, at least about 14 g/L, at least about 15 g/L, at least about 16 g/L, at least about 17 g/L, at least about 18 g/L, at least about 19 g/L, at least about 20 g/L, between about 0.1 g/L and about 20 g/L, between about 0.5 g/L and about 15 g/L, between about 1 g/L and about 10 g/L, between about 1 g/L and about 9 g/L, between about 1 g/L and about 8 g/L, between about 1 g/L and about 7 g/L, between about 2 g/L and about 6 g/L, between about 2 g/L and about 5 g/L, or between about 3 g/L and about 4 g/L. In certain instances, the microorganism described throughout is fermented in a stirred tank fermentor.

In other cases, olivetolic acid can be used to ferment CBGA or cannabinoids with the microorganism described throughout. For example, in some cases, the media can comprise a combination of olivetolic acid, yeast extract, peptone, and glucose. In certain cases, the media can comprise 10 g/L of yeast extract, 20 g/L peptone, 20 g/L glucose and 40 mg/L hexanoic acid. In some cases, olivetolic acid can be used in an amount of 1 mg/to 1 g/L. In some cases, olivetolic acid can be used in an amount of 5 mg/to 900 mg/L. In some cases, olivetolic acid can be used in an amount of 10 mg/to 800 mg/L. In some cases, olivetolic acid can be used in an amount of 15 mg/to 700 mg/L. In some cases, olivetolic acid can be used in an amount of 20 mg/to 600 mg/L. In some cases, olivetolic acid can be used in an amount of 25 mg/to 500 mg/L. In some cases, olivetolic acid can be used in an amount of 30 mg/to 400 mg/L. In some cases, olivetolic acid can be used in an amount of 35 mg/to 300 mg/L. In some cases, olivetolic acid can be used in an amount of 40 mg/to 200 mg/L. In some cases, olivetolic acid can be used in an amount of 50 mg/to 150 mg/L. In some cases, olivetolic acid can be used in an amount of 10 mg/to 100 mg/L. In some cases, olivetolic acid can be used in an amount of 20 mg/to 75 mg/L. In some cases, olivetolic acid can be used in an amount of 30 mg/to 50 mg/L.

Product Recovery

The fermentation of the microorganisms disclosed herein can produce a fermentation broth comprising a desired product (e.g., CBGA, CBGVA, THCA, CBDA or CBCA or cannabinoid) and/or one or more by-products as well as the cells/microorganisms (e.g., a genetically modified microorganism), in a nutrient medium.

In certain embodiments the CBGA, THCA, CBDA or CBCA produced in the fermentation reaction is converted to a cannabinoid, such as THC, CBD, and/or CDC. This conversion can happen directly from the fermentation broth. However, in other embodiments, the CBGA, THCA, CBDA or CBCA can be first recovered from the fermentation broth before conversion to a cannabinoid such as THC, CBD, and/or CDC. In certain embodiments the CBGVA produced in the fermentation reaction is converted to a cannabinoid, such as THCC, CBDV, and/or CDCV. This conversion can happen directly from the fermentation broth. However, in other embodiments, the CBGVA can be first recovered from the fermentation broth before conversion to a cannabinoid such as THCV, CBDV, and/or CDCV.

In some cases, the CBGA, CBGVA, THCA, CBDA or CBCA can be continuously removed from a portion of broth and recovered as purified the CBGA. In particular embodiments, the recovery of the CBGA, CBGVA, THCA, CBDA or CBCA includes passing the removed portion of the broth containing the CBGA, CBGVA, THCA, CBDA or CBCA through a separation unit to separate the microorganisms (e.g., genetically modified microorganism) from the broth, to produce a cell-free CBGA, CBGVA, THCA, CBDA or CBCA containing permeate, and returning the microorganisms to the bioreactor. Additional nutrients can be added to the media to replenish its nutrients before it is returned to the bioreactor. The cell-free CBGA, CBGVA, THCA, CBDA or CBCA permeate can then be stored or be used for subsequent conversion to cannabinoids (or other desired product).

Also, if the pH of the broth was adjusted during recovery of CBGA CBGVA, THCA, CBDA or CBCA the pH should be re-adjusted to a similar pH to that of the broth in the fermentation bioreactor, before being returned to the bioreactor.

Subsequent purification steps can involve treating the post-fermentation CBGA, CBGVA, THCA, CBDA or CBCA product using methods known in the art to recover individual product species of interest to high purity.

In one example, CBGA, CBGVA, THCA, CBDA or CBCA extracted in an organic phase can be transferred to an aqueous solution. In some cases, the organic solvent can be evaporated by heat and/or vacuum, and the resulting powder can be dissolved in an aqueous solution of suitable pH. The aqueous phase can then be removed by decantation, centrifugation, or another method. For example, when the organic solvent is ethyl acetate, the resulting powder from evaporation is dissolved in a water:acetonitrile mixture (50:50 ratio).

The same methods as described above can be applied to cannabinoids, should they be produced.

### CBGA, CBGVA or Cannabinoid Production Levels

The microorganisms and the methods herein can produce CBGA, CBGVA, THCA, CBDA, CBCA or cannabinoids at surprisingly high efficiency, more so than other known CBGA, CBGVA, THCA, CBDA, CBCA or cannabinoids fermentation processes. For example, the microorganisms and the methods disclosed herein can convert a carbon substrate (such as sugar, alcohol, and/or fatty acid) at a rate of greater than 0.01%.

The genetic modifications to the cells described throughout can be made to produce CBGA, CBGVA, THCA, CBDA, CBCA or cannabinoids over what would have been made without any genetic modifications. For example, compared to a non-genetically altered cell, the genetically modified microorganisms described throughout can produce CBGA, CBGVA, THCA, CBDA, CBCA or cannabinoids greater than 1.1 times (compared to the production levels of a non-genetically modified microorganism or non-genetically altered cell).

In some cases, the cannabinoid can be THC, CBD, CBC, or any combination thereof. In other cases, the cannabinoid can be THCV, CBDV, CBCV, or any combination thereof.

Methods of Making CBGA, THCA, CBDA or CBCA or cannabinoids

The genetically modified cells or microorganisms described throughout can be used to make CBGA, THCA, CBDA, CBCA and/or cannabinoids, e.g., THC, CBD, and CBC. A substrate capable of being converted into a CBGA or a cannabinoid can be brought in contact with one or more of the following enzymes: acyl activating enzyme (AAE1); polyketide synthase (PKS); olivetolic acid cyclase (OAC); prenyltransferase (PT); THCA synthase (THCAS); CBDA synthase (CBDAS), CBCA synthase (CBCAS), HMG-Co reductase (HMG1), and/or farnesyl pyrophosphate synthetase (ERG20).

The CBGA, THCA, CBDA, CBCA or cannabinoids (e.g., THC, CBD, CBC) produced can be recovered and isolated from the modified cells. The CBGA, THCA, CBDA, CBCA or cannabinoids in some cases can be secreted into the media of a cell culture, in which the CBGA, THCA, CBDA, CBCA or cannabinoids is extracted directly from the media. In some cases, the CBGA, THCA, CBDA, CBCA or cannabinoids can be within the cell itself, and the cells will need to be lysed in order to recover the respective CBGA, THCA, CBDA, CBCA or cannabinoids. In some instances, both cases can be true, where some CBGA, THCA, CBDA, CBCA or cannabinoids are secreted and some remains within the cells. In this case, either method or both methods can be used.

The genetically modified cells or microorganisms described throughout can be used to make CBGVA and/or cannabinoids, e.g., THCV, CBDV, and CBCV. A substrate capable of being converted into a CBGVA or a cannabinoid can be brought in contact with one or more of the following enzymes: acyl activating enzyme (AAE1); polyketide synthase (PKS); olivetolic acid cyclase (OAC); prenyltransferase (PT); THCA synthase (THCAS); CBDA synthase (CBDAS), CBCA synthase (CBCAS), HMG-Co reductase (HMG1), and/or farnesyl pyrophosphate synthetase (ERG20).

The CBGVA or cannabinoids (e.g., THCV, CBDV, CBCV) produced can be recovered and isolated from the modified cells. The CBGVA or cannabinoids in some cases can be secreted into the media of a cell culture, in which the CBGVA or cannabinoids is extracted directly from the media. In some cases, the CBGVA or cannabinoids can be within the cell itself, and the cells will need to be lysed in order to recover the respective CBGVA or cannabinoids. In some instances, both cases can be true, where some CBGVA or cannabinoids are secreted and some remains within the cells. In this case, either method or both methods can be used.

Accordingly, disclosed herein is a method of making CBGA, CBGVA, THCA, CBDA, CBCA or a cannabinoid comprising (a) contacting the genetically modified microorganism with a medium comprising a carbon source, and (b) growing the genetically modified microorganism to produce the CBGA or cannabinoid. The genetically modified microorganism can comprise any microorganism disclosed throughout. For example, the microorganism can be a genetically modified microorganism capable of converting a carbon substrate into CBGA, CBGVA, THCA, CBDA, CBCA or a cannabinoid, the genetically modified microorganism comprising a heterologous nucleic acid encoding one or more of the enzymes disclosed throughout (e.g., microorganism can comprise a nucleic acid sequence encoding for one or more of the following enzymes: acyl activating enzyme (AAE1); polyketide synthase (PKS); olivetolic acid cyclase (OAC); prenyltransferase (PT); THCA synthase (THCAS); CBDA synthase (CBDAS), CBCA synthase (CBCAS); HMG-Co reductase (HMG1); farnesyl pyrophosphate synthetase (ERG20); or any combination thereof).

The carbon source can be any carbon source that can be used by the microorganism. In some cases, the carbon source can be a sugar, alcohol, and/or fatty acid. For example, the sugar, alcohol or fatty acid can include without limitation hexanoic acid, butyric acid, glucose, fructose, xylose, sucrose, dextrins, starch, xylan, cellulose, hemicellulose, arabinose, glycerol, ethanol, butanol, methanol, or any combination thereof. In some cases, the carbon source can be hexanoic acid. In some cases, the carbon source can be olivetolic acid. In other cases, the carbon source can be a mixture of one or more different types of carbon sources.

The cannabinoid produced by the methods disclosed throughout can be any cannabinoid including but not limited to Δ⁹-tetrahydrocannabinolic acid (THCA), cannabigerolic acid (CBGA); cannabidiolic acid (CBDA), cannabichromenic acid (CBCA), Δ⁹-tetrahydrocannabinol (THC), cannabidiol (CBD), cannabichromene (CBC), Δ⁹-tetrahydrocannabivarinic acid (THCVA), cannabidivarinic acid (CBDVA), cannabichromevarinic acid (CBCVA) or any combination thereof.

In some cases, the medium does not contain any cells. In other words, this reaction is performed in the media in vitro. In some cases, the reaction does not occur within a cell. For example, the conversion of hexanoic acid to hexanoyl-CoA can occur outside of a cell. In some cases, the conversion of hexanoyl-CoA to olivetolic acid can occur outside of a cell. In some cases, the conversion of olivetolic acid to CBGA can occur outside of a cell. In some cases, the conversion of CBGA to Δ9-tetrahydrocannabinolic acid can occur outside of a cell. In some cases, the conversion of Δ9-tetrahydrocannabinolic acid to Δ9-tetrahydrocannabinol can occur outside of a cell. In some cases, the conversion of CBGA to cannabidiolic acid can occur outside of a cell. In some cases, the conversion of cannabidiolic acid to cannabidiol can occur outside of a cell. In some cases, the conversion of CBGA to cannabichromenic acid can occur outside of a cell. In some cases, the conversion of cannabichromenic acid to cannabichromene can occur outside of a cell.

In some cases, the cannabinoid, such as CBGA, CBGVA, THCA, CBDA, or CBCA can be converted outside of a cell. For example, once CBGA, CBGVA, THCA, CBDA, or CBCA is produced, it can be either isolated (from the cell or the cell media or both). Once isolated it can be converted, enzymatically or non-enzymatically into other a different product, such as another type of cannabinoid. In some cases, the CBGA, CBGVA, THCA, CBDA, or CBCA is just secreted into the media by the microorganism that synthesized it, and then the CBGA, CBGVA, THCA, CBDA, or CBCA is directly converted enzymatically or non-enzymatically into other a different product, such as another type of cannabinoid.

In some cases, this reaction is contained within a cell that is within cell culture media. In other words, the reaction is performed in vivo. For example, the conversion of hexanoic acid to hexanoyl-CoA can occur within a cell. In some cases, the conversion of hexanoyl-CoA to olivetolic acid can occur within a cell. In some cases, the conversion of olivetolic acid to CBGA can occur within a cell. In some cases, the conversion of CBGA to Δ9-tetrahydrocannabinolic acid can occur within a cell. In some cases, the conversion of Δ9-tetrahydrocannabinolic acid to Δ9-tetrahydrocannabinol can occur within a cell. In some cases, the conversion of CBGA to cannabidiolic acid can occur within a cell. In some cases, the conversion of cannabidiolic acid to cannabidiol can occur within a cell. In some cases, the conversion of CBGA to cannabichromenic acid can occur within a cell. In some cases, the conversion of cannabichromenic acid to cannabichromene can occur within a cell.

In some cases, there is a combination of the two. Some reactions along the pathway can occur within a cell, whereas some of the reactions along the pathway occur outside of a cell.

In some cases, the medium is cell culture media. In other instances, the medium is water or other liquid in which the cells (for in vivo reactions) can survive (such as saline buffered water). In other instances, the medium is water or other liquid in which the enzymes (for in vitro reactions) are active.

The CBGA, CBGVA, THCA, CBDA, CBCA or cannabinoids produced herein can be useful inter alia in the manufacture of pharmaceutical compositions. Thus, disclosed herein is a method of making a pharmaceutical composition by using the products disclosed herein. In some cases, the CBGA, CBGVA, THCA, CBDA, CBCA or cannabinoids are mixed with excipients to produce pharmaceutical compositions.

Upon completion of the methods or reactions described throughout, the amount of a particular cannabinoid, e.g., THCA, CBDA, CBCA, THC, CBD, CBC, THCVA, CBDVA, CBCVA, THCV, CBDV or CBCV, present in the reaction mixture can be at least 10% (w/w), at least 8% (w/w), at least 9% (w/w), at least 7% (w/w), at least 6% (w/w), at least, 5% (w/w), at least 4% (w/w), at least 3% (w/w), at least 2% (w/w), at least 1% (w/w), at least 0.5% (w/w), or at least 0.01% (w/w) of the total cannabinoids in the reaction mixture. Upon completion of the methods or reactions described throughout, the amount of a particular cannabinoid, e.g., THCA, CBDA, CBCA, THC, CBD, CBC, THCVA, CBDVA, CBCVA, THCV, CBDV or CBCV, present in the reaction mixture can be at least about 20 g/L, 15 g/L, 10 g/L, 8 g/L, 5 g/L, 4 g/L, 3 g/L, 2 g/L, 1 g/L, 0.5 g/L, or 0.1 g/L.

Upon completion of the methods or reactions described throughout, the amount of a particular cannabinoid, e.g., THCA, CBDA, CBCA, THC, CBD, CBC, THCVA, CBDVA, CBCVA, THCV, CBDV or CBCV, present in the reaction mixture can be about 0.001% (w/w) to about 99% (w/w), about 0.01% (w/w) to about 25% (w/w), about 0.025% (w/w) to about 20% (w/w), about 0.05% (w/w) to about 15% (w/w), about 0.075% (w/w) to about 12% (w/w), about 0.1% (w/w) to about 10% (w/w), about 0.25% (w/w) to about 5% (w/w), about 0.5% (w/w) to about 1% (w/w), about 0.1% (w/w) to about 1% (w/w), about 0.1% (w/w) to about 0.5% (w/w), about 0% (w/w) to about 1% (w/w), and any range therebetween. In some instances, the reaction mixture comprises CBGA in a weight percentage of about 0.001% (w/w) to about 99% (w/w), about 0.01% (w/w) to about 25% (w/w), about 0.025% (w/w) to about 20% (w/w), about 0.05% (w/w) to about 15% (w/w), about 0.075% (w/w) to about 12% (w/w), about 0.1% (w/w) to about 10% (w/w), about 0.25% (w/w) to about 5% (w/w), about 0.5% (w/w) to about 1% (w/w), about 0.1% (w/w) to about 1% (w/w), about 0.1% (w/w) to about 0.5% (w/w), about 0% (w/w) to about 1% (w/w), and any range therebetween, of total cannabinoids in the reaction mixture. In other instances, the reaction mixture comprises CBGVA in a weight percentage of about 0.001% (w/w) to about 99% (w/w), about 0.01% (w/w) to about 25% (w/w), about 0.025% (w/w) to about 20% (w/w), about 0.05% (w/w) to about 15% (w/w), about 0.075% (w/w) to about 12% (w/w), about 0.1% (w/w) to about 10% (w/w), about 0.25% (w/w) to about 5% (w/w), about 0.5% (w/w) to about 1% (w/w), about 0.1% (w/w) to about 1% (w/w), about 0.1% (w/w) to about 0.5% (w/w), about 0% (w/w) to about 1% (w/w), and any range therebetween, of total cannabinoids in the reaction mixture. In other instances, the reaction mixture comprises THCA in a weight percentage of about 0.001% (w/w) to about 99% (w/w), about 0.01% (w/w) to about 25% (w/w), about 0.025% (w/w) to about 20% (w/w), about 0.05% (w/w) to about 15% (w/w), about 0.075% (w/w) to about 12% (w/w), about 0.1% (w/w) to about 10% (w/w), about 0.25% (w/w) to about 5% (w/w), about 0.5% (w/w) to about 1% (w/w), about 0.1% (w/w) to about 1% (w/w), about 0.1% (w/w) to about 0.5% (w/w), about 0% (w/w) to about 1% (w/w), and any range therebetween, of total cannabinoids in the reaction mixture. In other instances, the reaction mixture comprises CBDA in a weight percentage of about 0.001% (w/w) to about 99% (w/w), about 0.01% (w/w) to about 25% (w/w), about 0.025% (w/w) to about 20% (w/w), about 0.05% (w/w) to about 15% (w/w), about 0.075% (w/w) to about 12% (w/w), about 0.1% (w/w) to about 10% (w/w), about 0.25% (w/w) to about 5% (w/w), about 0.5% (w/w) to about 1% (w/w), about 0.1% (w/w) to about 1% (w/w), about 0.1% (w/w) to about 0.5% (w/w), about 0% (w/w) to about 1% (w/w), and any range therebetween, of total cannabinoids in the reaction mixture. In other instances, the reaction mixture comprises CBCA in a weight percentage of about 0.001% (w/w) to about 99% (w/w), about 0.01% (w/w) to about 25% (w/w), about 0.025% (w/w) to about 20% (w/w), about 0.05% (w/w) to about 15% (w/w), about 0.075% (w/w) to about 12% (w/w), about 0.1% (w/w) to about 10% (w/w), about 0.25% (w/w) to about 5% (w/w), about 0.5% (w/w) to about 1% (w/w), about 0.1% (w/w) to about 1% (w/w), about 0.1% (w/w) to about 0.5% (w/w), about 0% (w/w) to about 1% (w/w), and any range therebetween, of total cannabinoids in the reaction mixture.

### Exemplary Uses of the CBGA or Cannabinoids

Preparations of CBGA, CBGVA, THCA, CBDA, CBCA or cannabinoids obtained can be used for any and all uses. The CBGA, CBGVA, THCA, CBDA, CBCA or cannabinoids can be isolated and sold as purified products. Or these purified products (e.g., CBGA) can be a feedstock to make additional cannabinoids.

The cannabinoids made can be used to manufacture medicinal compounds.

Accordingly, in one aspect, disclosed is a use of CBGA, THCA, CBDA, or CBCA as a feedstock compound in the manufacture of a cannabinoid. In another aspect, disclosed is a use of a cannabinoid in the manufacture of a medicinal composition.

### Pharmaceutical Compositions and Routes of Administration

The cannabinoids (e.g., THC, CBD, CDC, THCV, CBDV, and/or CDCV) can include pharmaceutically acceptable derivatives or prodrugs thereof. A “pharmaceutically acceptable derivative” can mean means any pharmaceutically acceptable salt, ester, salt of an ester, pro-drug or other derivative thereof.

Pharmaceutically acceptable salts of the compounds of this invention include those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate and undecanoate. Salts derived from appropriate bases include alkali metal (e.g., sodium), alkaline earth metal (e.g., magnesium), ammonium and N-(alkyl)₄⁺ salts.

For preparing pharmaceutical compositions from the compounds of the present invention, pharmaceutically acceptable carriers include either solid or liquid carriers. Solid form preparations include powders, tablets, pills, capsules, cachets, suppositories, and dispersible granules. A solid carrier can be one or more substances, which also acts as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton Pa.

In powders, the carrier is a finely divided solid, which is in a mixture with the finely divided active component. In tablets, the active component is mixed with the carrier having the necessary binding properties in suitable proportions and compacted in the shape and size desired.

Suitable solid excipients are carbohydrate or protein fillers include, but are not limited to sugars, including lactose, sucrose, mannitol, or sorbitol; starch from corn, wheat, rice, potato, or other plants; cellulose such as methyl cellulose, hydroxypropylmethyl-cellulose, or sodium carboxymethylcellulose; and gums including arabic and tragacanth; as well as proteins such as gelatin and collagen. If desired, disintegrating or solubilizing agents are added, such as the cross-linked polyvinyl pyrrolidone, agar, alginic acid, or a salt thereof, such as sodium alginate.

Liquid form preparations include solutions, suspensions, and emulsions, for example, water or water/propylene glycol solutions. For parenteral injection, liquid preparations can be formulated in solution in aqueous polyethylene glycol solution.

The pharmaceutical preparation can be a unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

### Exemplary Uses of the Cannabinoids

Preparations of cannabinoids (e.g., CBGA, THCA, CBDA, CBCA, THC, CBD, CBC, CBGVA, THCVA, CBDVA, CBCVA, THCV, CBDV, CBCV) obtained can be used for any and all uses. The cannabinoids can be isolated and sold as purified products. Or these purified products can be a feedstock to make additional types of cannabinoids. For example, purified CBGA can be used as a feedstock to make other cannabinoids such as THCA, CBDA, CBCA, THC, CBD, and CBC. In another example, purified CB GVA can be used as a feedstock to make other cannabinoids such as THCVA, CBDVA, CBCVA, THCV, CBDV, and CBCV.

The cannabinoids made in the processes described throughout can be used to manufacture medicinal compounds. Accordingly, in one aspect, disclosed is a use of cannabinoids as a feedstock compound in the manufacture of a medicinal compound. For example, the cannabinoids can be subsequently processed to prepare a pharmaceutical formulation.

### Pharmaceutical Compositions and Routes of Administration

The cannabinoids also include pharmaceutically acceptable derivatives thereof. A “pharmaceutically acceptable derivative” means any pharmaceutically acceptable salt, ester, salt of an ester, or other derivative thereof.

Pharmaceutically acceptable salts of the compounds of this invention include those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate and undecanoate. Salts derived from appropriate bases include alkali metal (e.g., sodium), alkaline earth metal (e.g., magnesium), ammonium and N-(alkyl)₄+ salts.

For preparing pharmaceutical compositions from the compounds of the present invention, pharmaceutically acceptable carriers include either solid or liquid carriers. Solid form preparations include powders, tablets, pills, capsules, cachets, suppositories, and dispersible granules. A solid carrier can be one or more substances, which also acts as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton Pa.

In powders, the carrier is a finely divided solid, which is in a mixture with the finely divided active component. In tablets, the active component is mixed with the carrier having the necessary binding properties in suitable proportions and compacted in the shape and size desired.

Suitable solid excipients are carbohydrate or protein fillers include, but are not limited to sugars, including lactose, sucrose, mannitol, or sorbitol; starch from corn, wheat, rice, potato, or other plants; cellulose such as methyl cellulose, hydroxypropylmethyl-cellulose, or sodium carboxymethylcellulose; and gums including arabic and tragacanth; as well as proteins such as gelatin and collagen. If desired, disintegrating or solubilizing agents are added, such as the cross-linked polyvinyl pyrrolidone, agar, alginic acid, or a salt thereof, such as sodium alginate.

Liquid form preparations include solutions, suspensions, and emulsions, for example, water or water/propylene glycol solutions. For parenteral injection, liquid preparations can be formulated in solution in aqueous polyethylene glycol solution.

The pharmaceutical preparation can be a unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

One particular delivery system can be through the pulmonary system. In some cases, the cannabinoid can be for into a liquid and vaporized so that it can be inhaled. See e.g., U.S. Pat. No. 9,326,967. Vaporization of cannabinoids and delivery through the pulmonary system can result in quick absorption through the circulatory system and can provide extremely fast systemic effects. Further, vaporization can mimic one of the preferred ways in which natural cannabinoids are inhaled.

Additional delivery system that can work by intravenous injections. See e.g., WO2013009928A1. Similar to vaporization, this intravenous injection can provide extremely fast systemic effects.

Oral delivery systems, such as, delivery through the gastrointestinal tract, can be used to deliver the cannabinoids. For example, the oral delivery system can be in the form of a pharmaceutical dosage unit, food, drink, or anything that can be delivered through the gastrointestinal tract.

Treatment of Disease and Symptoms of Disease

The cannabinoids can be used to treat disease, in particular to treat disease of people that are in need thereof. This includes treating one or more symptoms of the diseases. For example, the cannabinoids can be used to treat one of more of the following diseases: anorexia, multiple sclerosis, neurodegenerative disorders, such as Parkinson's disease, Huntington's disease, Tourette's syndrome, and Alzheimer's disease, epilepsy, glaucoma, osteoporosis, schizophrenia, bipolar disorder, post-traumatic stress disorder (PTSD), asthma, cardiovascular disorders, cancer, obesity, or metabolic syndrome-related disorders.

The cannabinoids can be used to treat one or more symptoms of disease, such as depression, anxiety, insomnia, emesis, pain, or inflammation.

Some of the diseases or symptom of disease can be exclusive to humans, but other diseases or symptom of disease can be shared in more than one animal, such as in all mammals.

### Recreational Uses

The cannabinoids produced by the microorganism and methods described throughout can be used for recreational use. For example, the cannabinoids, such as Δ⁹-tetrahydrocannabinolic acid (THCA), cannabidiolic acid (CBDA), cannabichromenic acid (CBCA), Δ⁹-tetrahydrocannabinol (THC), cannabidiol (CBD), cannabichromene (CBC), Δ⁹-tetrahydrocannabivarinic acid (THCVA), cannabidivarinic acid (CBDVA), cannabichromevarinic acid (CBCVA), or any combination thereof, can be used for non-medical uses.

In some cases, the cannabinoid can be formed into a liquid and vaporized so that it can be inhaled. See e.g., U.S. Pat. No. 9,326,967. Vaporization of cannabinoids and delivery through the pulmonary system can be used and can be preferred by some recreational users. For example, recreational users who do not like the smell of burning cannabis or those that are afraid of the effects of inhaling burning substances, can use this method. Further, since this method is not invasive and can be used almost anywhere, recreational users can prefer this method.

In some cases, the cannabinoid can be formed into something that can be injected, e.g., injected intravenously. This method can be used in order to deliver substances quickly and efficiently within the blood stream. For example, this liquid can be injected into the saline solution (colloquially known as “IV”) used in hospitals to keep patients hydrated. Further, intravenous injections can be used by recreational users for immediate effects. In some cases, the intravenous cannabinoid injections can be used to treat other drug addictions, such as heroin addiction.

In additional cases, the cannabinoids produced from the microorganisms and methods described throughout can be used as an additive to food or drink. For example, the cannabinoids can be used, for example, in baked goods, such as brownies or cakes. Additionally, the cannabinoids can be added to a beverage such as water, soda, beer, liquor, etc.

Other recreational ways to use the cannabinoids include but are not limited to patches; (similar to nicotine patches); topically (such as in lotions); sprays (breath freshener), or tinctures (mouth drops).

The disclosure is now described with reference to the following examples. These examples are provided for the purpose of illustration only and the disclosure should in no way be construed as being limited to these examples, but rather should be construed to encompass any and all variations which become evident as a result of the teaching provided herein.

### EXAMPLES

### Example 1—Plasmid Construction

A prenyltransferase of interest was identified. The amino acid sequence (SEQ ID NO: 1) was used by Genscript to design and synthesize the yeast codon optimized sequence coding for the prenyltransferase and used in the experiments.

Plasmids were constructed using the GeneArt Seamless Cloning and Assembly from Thermo Fisher Scientific. The RUNM000898_511.1 vector (SEQ ID NO: 3) contained the Saccharomyces cerevisiae 2μ replication origin, the URA3 gene as an auxotrophic marker and the PKS and OAC genes under the regulation of the bidirectional GAL1/GAL10 promoter. The bCBGA0098 vector (SEQ ID NO: 4) contained the Saccharomyces cerevisiae 2μ replication origin, the LEU2 gene as an auxotrophic marker and the AAE1 and PT genes under the regulation of the bidirectional GAL1/GAL10 promoter. The bCBGA0306 vector (SEQ ID NO: 25) contained the Saccharomyces cerevisiae 2μ replication origin, the LEU2 gene as an auxotrophic marker and the PT gene under the regulation of the bidirectional GAL1/GAL10 promoter.

### Example 2—yCBGA0172 Strain Construction

The parental strain for all examples was the Saccharomyces cerevisiae CEN.PK2-1C strain. Its genotype is: MATA, ura3-52; trp1-289; leu2-3,112; his3Δ 1; MAL2-8^{C}; SUC2.

A mutant ERG20 allele was integrated into the GAL80 locus of the host. First, a plasmid was constructed carrying an ERG20 allele with two mutations: F96W and N127W. Second, the ERG20 allele together with the adjacent HygMX cassette was amplified in a PCR reaction and flanking sequences of the chromosomal GAL80 coding sequence were incorporated during the PCR reaction using oligonucleotides with 5′ extensions. Third, this DNA fragment was transformed into the host strain by electroporation. Finally, the strain with integrated mutant ERG20 sequence at the GAL80 locus were identified by its hygromycin B resistance and referred to as yCBGA0172.

Plasmids RUNM000898_511.1 (SEQ ID NO: 3) and bCBGA0098 (SEQ ID NO: 4) were transformed into the yCBGA0172 strain by electroporation. Transformants were selected by their leucine and uracil prototrophy on SD/MSG minimal medium (20 g/L glucose, 1.7 g/L yeast nitrogen base w/o ammonium sulphate and amino acids, 1 g/L monosodium glutamic acid, 20 g/L agar when solid medium is to be used) supplemented with histidine and tryptophan.

In another example plasmid bCBGA0306 (SEQ ID NO: 25) and the VVN4655922 plasmid were transformed into the yCBGA0172 strain by electroporation. The plasmid VVN4655922 encodes for the Saccharomyces cerevisiae HMG1 gene truncated of the first 530 amino acids and has the Saccharomyces cerevisiae TRP1 gene as an auxotrophic selection marker. Transformants were selected by their leucine and tryptophan prototrophies on SD/MSG minimal medium supplemented with histidine and uracil.

### Example 3—Growth

Transformant colonies were picked and inoculated into separate wells of a 96-well deep well plate. Each well contained 400 μl Synthetic Defined (SD)/MSG liquid medium supplemented with histidine and tryptophan. These inoculums were grown overnight at 30° C. and shaken at 300 rpm with a 50 mm shaking diameter.

After the overnight growth the samples were centrifuged, the supernatant discarded and cells transformed with plasmids RUNM000898_511.1 and bCBGA0098 were re-suspended in 400 μl YPD-HXA (10 g/L yeast extract, 20 g/L peptone, 20 g/L glucose and 100 mg/L hexanoic acid) medium. In case of cultures transformed with plasmids bCBGA0306 and VVN4655922 the pelleted cells were re-suspended in 400 μl YPD-OLA (10 g/L yeast extract, 20 g/L peptone, 20 g/L glucose and 40 mg/L olivetolic acid) medium.

Then samples were grown for 16 hours at 30° C. and shaken at 300 rpm with a 50 mm shaking diameter and 16 μl 50% glucose was added to the samples. Samples grown in YPD-OLA medium were supplemented additionally with 20 μl of 800 mg/L olivetolic acid solution too.

Finally, samples were grown for additional 32 hours and were analyzed for CBGA titers.

### Example 4—Sample Processing and Analytics

The samples were processed by adding 400 μl acetonitrile, then shaken for 5 minutes at 30° C. at 300 rpm with 50 mm throw. The samples were then centrifuged at 400 rpm for 5 minutes. 200 μl of supernatant were transferred into a new 96 well plate.

The new 96 well plate were transferred to a Waters Acquity UPLC (Binary pump)-TQD MS and set with the following parameters:
Instrument: Waters Acquity UPLC (Binary pump)-TQD MS
Stationary phase: Agilent Eclipse Plus C18 RRHD 1.8 mm, 2.1×50 mm
Mobile phase A: water 0.1% FA
Mobil phase B: acetonitrile 0.1% FA
Gradient info (Table 1):

TABLE 1

| Time [min] | % A | % B |
|---|---|---|
| 0 | 55 | 45 |
| 0.5 | 45 | 55 |
| 0.6 | 30 | 70 |
| 2.0 | 30 | 70 |
| 2.1 | 0 | 100 |
| 2.2 | 0 | 100 |
| 2.3 | 55 | 45 |

Flow: 0.4 mL/min
Column temp: 35° C.
Detection: Acquity TQD, MRM Mode (361.2»219.1; 361.2»149.0; 361.2»237.1; 361.2»343.2)

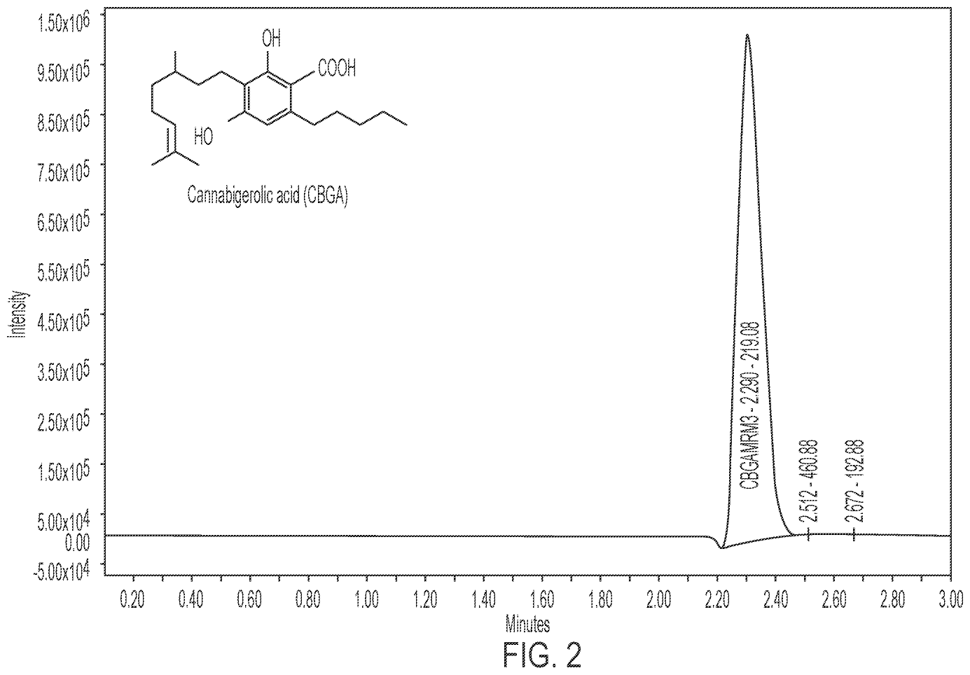

Using the strains and methods described above CBGA was produced at 200 μg/L concentration when YPD-HXA medium was used and at 15 mg/L concentration when YPD-OLA medium was used. A representative chromatogram of the sample and the CBGA standard can be seen in FIG. 2.

### Example 5—the yCBGA0189, yCBGA0197 and yCBGA0201 Strains: Testing Additional Prenyl Transferases

A modified sequence with higher prenyl transferase activity was constructed and referred to as GFP-dPT (polynucleotide sequence: SEQ ID NO: 26; amino acid sequence: SEQ ID NO: 27). The GFP-dPT gene is a fusion of two polynucleotide sequences: a gene of a modified fluorescent protein yEVenus (SEQ ID NO: 28) from the plasmid pKT90 and a truncated version of SEQ ID NO: 2 missing its first 246 nucleotides.

Plasmids were constructed using the GeneArt Seamless Cloning and Assembly from Thermo Fisher Scientific. The bCBGA0385 vector (SEQ ID NO: 29) contained the Saccharomyces cerevisiae 2μ replication origin, the LEU2 gene as an auxotrophic marker and the GFP-dPT gene under the regulation of the bidirectional GAL1/GAL10 promoter. The bCBGA0305 vector (SEQ ID NO: 30) contained the Saccharomyces cerevisiae 2μ replication origin, the TRP1 gene as an auxotrophic marker and the AAE1 gene under the regulation of the bidirectional GAL1/GAL10 promoter.

For testing the GFP-dPT activity a new parental strain was constructed: a polynucleotide fragment of the RUNM000898_511.1 vector coding for OAC, PKS and URA3 genes was transformed into the strain yCBGA0172 by electroporation. The strain with OAC, PKS and URA3 genes inserted was identified by its uracil prototrophy on SD/MSG minimal medium supplemented with histidine, tryptophan and leucine and referred to as yCBGA0189.

In an another experiment a polynucleotide fragment coding for the truncated version of HMG1 gene lacking its first 530 amino acids and a KanMX cassette was transformed into the strain yCBGA0172 by electroporation. The strain with truncated HMG1 gene inserted was identified by its G418 resistance and referred to as yCBGA0197.

The strain yCBGA0197 was transformed with a vector coding for the Saccharomyces cerevisiae HO gene and URA3 gene. The plasmid was cured and a clone with MAT alpha mating type was identified using standard laboratory methods. Finally, this MAT alpha clone was mated with yCBGA0189 and the isolated diploid strain is referred to as yCBGA0201.

Plasmids bCBGA0305 and bCBGA0385 were transformed into the yCBGA0201 strain by electroporation. Transformants were selected by their leucine and tryptophan prototrophies on SD/MSG minimal medium supplemented with histidine.

In another example, plasmid bCBGA0385 was transformed into the yCBGA0197 strain by electroporation. Transformants were selected by their leucine prototrophy on SD/MSG minimal medium supplemented with histidine, uracil and tryptophan.

Transformant colonies were picked and inoculated into separate wells of a 96-well deep well plate. Each well contained 400 μl SD/MSG liquid medium supplemented with histidine in case of strains containing both bCBGA0305 and bCBGA3085 and with histidine, uracil and tryptophan in case of strains containing bCBGA0385 plasmid alone. These inoculums were grown overnight at 30° C. and shaken at 300 rpm with a 50 mm shaking diameter.

After the overnight growth the samples were centrifuged, the supernatant discarded and cells re-suspended in 400 μl YPD-HXA (10 g/L yeast extract, 20 g/L peptone, 20 g/L glucose and 100 mg/L hexanoic acid) medium or 400 μl YPD-800LA (10 g/L yeast extract, 20 g/L peptone, 20 g/L glucose and 80 mg/L olivetolic acid) medium. In case of cultures transformed with only bCBGA0385 plasmid the YPD-800LA medium was used.

The samples were grown for 16 hours at 30° C. and shaken at 300 rpm with a 50 mm shaking diameter and 16 μl 50% glucose was added to the samples.

Finally, samples were grown for additional 32 hours and were analyzed for CBGA titers as described in “Example 4—Sample processing and analytics”.

Using the strains and methods described above CBGA was produced at 11 mg/L concentration when YPD-HXA medium was used and at 50 mg/L concentration when YPD-800LA medium was used.

### Example 6—Mutant Prenyl Transferase

Another modified sequence with increased prenyl transferase activity was constructed and referred to as ERG20mut-dPT (polynucleotide sequence: SEQ ID NO: 31; amino acid sequence: SEQ ID NO: 32). The ERG20mut-dPT gene is a fusion of two polynucleotide sequences: ERG20 gene with F96W and N127W mutations and a truncated version of SEQ ID NO: 2 missing its first 246 nucleotides.

Plasmid was constructed using the GeneArt Seamless Cloning and Assembly from Thermo Fisher Scientific. The bCBGA0559 vector (SEQ ID NO: 33) contained the Saccharomyces cerevisiae 2μ replication origin, the LEU2 gene as an auxotrophic marker and the ERG20mut-dPT gene under the regulation of the bidirectional GAL1/GAL10 promoter.

bCBGA0559 was transformed into the yCBGA0197 strain by electroporation. Transformants were selected by their leucine prototrophy on SD/MSG minimal medium supplemented with histidine, uracil and tryptophan.

Transformant colonies were picked and inoculated into separate wells of a 96-well deep well plate. Each well contained 400 μl SD/MSG liquid medium supplemented with histidine, uracil and tryptophan. These inoculums were grown overnight at 30° C. and shaken at 300 rpm with a 50 mm shaking diameter.

After the overnight growth the samples were centrifuged, the supernatant discarded and cells re-suspended in 400 μl YPD-1200LA (10 g/L yeast extract, 20 g/L peptone, 20 g/L glucose and 120 mg/L olivetolic acid) medium.

Then samples were grown for 16 hours at 30° C. and shaken at 300 rpm with a 50 mm shaking diameter and 16 μl 50% glucose was added to the samples.

Finally, samples were grown for additional 32 hours and were analyzed for CBGA titers as described in Example 4—Sample processing and analytics.

Using the strains and methods described above CBGA was produced at 90 mg/L concentration.

### Example 7—the yCBGA0237, yCBGA0253 and yCBGA0254 Strains: ERG20 Promoter Truncation

yCBGA0237 strain was constructed by deleting the HygMX and KanMX cassettes from the yCBGA0197 strain and inserting the GFP-dPT gene under the regulation of the bidirectional GAL1/GAL10 promoter into the YJL144W locus by replacing the native YJL144W open reading frame (ORF). Two more strains were constructed by deleting different fragments from the promoter of the native ERG20 allele of the yCBGA0237 strain. The yCBGA0253 strain contains a 133 nucleotide long deletion between 143 and 275 nucleotides upstream of the translational start site. The yCBGA0254 strain contains a 422 nucleotide long deletion between 69 and 490 nucleotides upstream of the translational start site.

Strains yCBGA0237, yCBGA0253 and yCBGA0254 were inoculated into separate wells of a 96-well deep well plate. Each well contained 400 μl SD/MSG liquid medium supplemented with histidine, leucine, uracil and tryptophan. These inoculums were grown overnight at 30° C. and shaken at 300 rpm with a 50 mm shaking diameter.

After the overnight growth 40 μl of the samples were transferred into 360 μl YPD-1200LA (10 g/L yeast extract, 20 g/L peptone, 20 g/L glucose and 120 mg/L olivetolic acid) medium.

Then samples were grown for 16 hours at 30° C. and shaken at 300 rpm with a 50 mm shaking diameter and 16 μl 50% glucose was added to the samples.

Finally, samples were grown for additional 32 hours and were analyzed for CBGA titers as described in Example 4—Sample processing and analytics.

Using the strains and methods described above, yCBGA0237 strain produced 53 mg/L CBGA and yCBGA0253 and yCBGA0254 reached 96 and 78 mg/L CBGA concentration, respectively.

### Example 8—Olivetolic Acid Conversion to THCA

A plasmid was constructed using the GeneArt Seamless Cloning and Assembly from Thermo Fisher Scientific. The RUNM001233_51.1 vector (SEQ ID NO: 34) contained the Saccharomyces cerevisiae 2μ replication origin, the URA3 gene as an auxotrophic marker and the THCA synthase gene under the regulation of the bidirectional GAL1/GAL10 promoter.

For testing the olivetolic acid conversion into THCA the RUNM001233_51.1 and bCBGA0385 vectors were transformed into the strain yCBGA0197 by electroporation. Transformants were selected by their leucine and uracil prototrophies on SD/MSG minimal medium supplemented with histidine and tryptophan.

Transformant colonies were picked and inoculated into separate wells of a 96-well deep well plate. Each well contained 400 μl SC-URA-LEU (6.7 g/L Yeast Nitrogen Base, 1.6 g/L Amino Acid Drop Out mix without uracil and leucine, 22 g/L glucose, buffered to pH 6.0). These inoculums were grown for 48 hours at 30° C. and shaken at 300 rpm with a 50 mm shaking diameter.

After the 48 hours growth period the samples were centrifuged, the supernatant discarded and cells re-suspended in 400 μl YPD-1200LA (10 g/L yeast extract, 20 g/L peptone, 20 g/L glucose and 120 mg/L olivetolic acid) medium.

Then samples were grown for 16 hours at 30° C. and shaken at 300 rpm with a 50 mm shaking diameter and 16 μl 50% glucose was added to the samples.

Finally, samples were grown for additional 32 hours and were analyzed for THCA and CBGA titers as described below.

Using the strains and methods described above CBGA was produced at 2 mg/L concentration while THCA was produced at 84 mg/L concentration.

### Example 9—The yCBGA0269 Strain: Hexanoic Acid Conversion to THCA

Plasmids were constructed using the GeneArt Seamless Cloning and Assembly from Thermo Fisher Scientific. The RUNM001210_96.1 vector (SEQ ID NO: 35) contained the Saccharomyces cerevisiae 2μ replication origin, the URA3 gene as an auxotrophic marker, the PKS and OAC genes under the regulation of the bidirectional GAL1/GAL10 promoter and the AAE1 gene under the regulation of the STE5 promoter. The bCBGA0409 vector (SEQ ID NO: 36) contained the Saccharomyces cerevisiae 2μ replication origin, the LEU2 gene as an auxotrophic marker, the THCA synthase and PT genes under the regulation of the bidirectional GAL1/GAL10 promoter.

The yCBGA0251 strain was constructed by inserting the ERG20mut-dPT gene under the regulation of the bidirectional GAL1/GAL10 promoter into the YMR145C locus by replacing the native YMR145C ORF of the yCBGA0237 strain. The yCBGA0269 strain was constructed by deleting a 133 nucleotide long fragment between 143 and 275 nucleotides upstream of the translational start site of the native ERG20 allele of the yCBGA0251 strain. Plasmids RUNM001210_96.1 and bCBGA0409 were transformed into the yCBGA0269 strain by electroporation. Transformants were selected by their uracil and leucine prototrophy on SD/MSG minimal medium supplemented with histidine and tryptophan.

Transformant colonies were picked and inoculated into separate wells of a 96-well deep well plate. Each well contained 400 μl SC-URA-LEU (6.7 g/L Yeast Nitrogen Base, 1.6 g/L Amino Acid Drop Out mix without uracil and leucine, 22 g/L glucose, buffered to pH 6.0). These inoculums were grown for 48 hours at 30° C. and shaken at 300 rpm with a 50 mm shaking diameter. After the 48 hours growth period 40 μl samples of these cultures were inoculated into 360 μl YPD-HXA (10 g/L yeast extract, 20 g/L peptone, 20 g/L glucose and 100 mg/L hexanoic acid) medium. Then samples were grown for 16 hours at 30° C. and shaken at 300 rpm with a 50 mm shaking diameter and 40 μg hexanoic acid dissolved in 8 μl ethanol was added to the samples. Finally, samples were grown for additional 32 hours and were analyzed for THCA and CBGA titers as described below.

Using the strains and methods described above CBGA was produced at 34 mg/L concentration while THCA was produced at 23 mg/L concentration.

### Example 10—Additional Sample Processing

The samples from examples 8 and 9 were processed by dilution with acetonitrile:water mixture (the composition of the mixture depends on the dilution factor, to reach 50% acetonitrile content for further processing), then shaken for 5 minutes at 30° C. at 300 rpm with 50 mm throw. The samples were then centrifuged at 400 rpm for 5 minutes. 200 μl of supernatant were transferred into a new 96 well plate.

The new 96 well plate were transferred to a Waters Acquity UPLC (Binary pump)-TQD MS and set with the following parameters:
Instrument: Waters Acquity UPLC (Binary pump)-TQD MS
Stationary phase: Agilent Eclipse Plus C18 RRHD 1.8 mm, 2 1×50 mm
Mobile phase A: water 0.1% FA
Mobil phase B: acetonitrile 0.1% FA
Gradient info (Table 2):

TABLE 2

| Time [min] | A [%] | B [%] |
|---|---|---|
| 0 | 55 | 45 |
| 0.5 | 45 | 55 |
| 0.6 | 30 | 70 |
| 2.0 | 30 | 70 |
| 2.1 | 5 | 95 |
| 3.1 | 5 | 95 |
| 3.2 | 55 | 45 |
| 5.5 | 55 | 45 |

Flow: 0.4 mL/min
Column temp: 35° C.
Detection: Acquity TQD, MRM Mode (361.2>>219.1; 361.2>>149.0; 361.2>>237.1; 361.2>>343.2); UV at 280 nm wavelength.

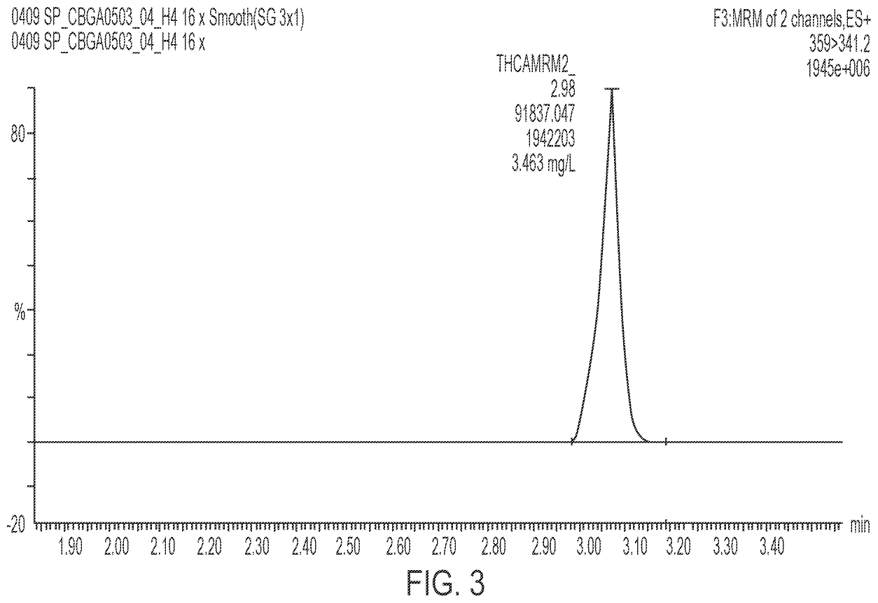

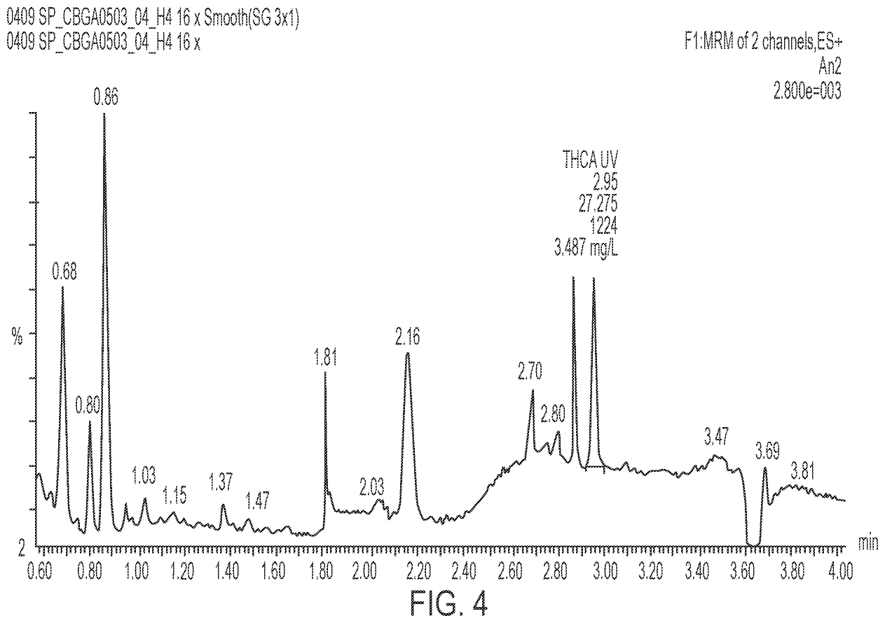

Representative chromatograms of a THCA containing sample can be seen in FIG. 3 (MRM chromatogram) and FIG. 4 (UV chromatogram).

### Example 11—the yCBGA0314 Strain: Strains with Increased Prenyl Transferase Copy Number

Increased Prenyl Transferase copy number and promoter truncation improves prenyl transferase activity. The yCBGA0314 strain was constructed and tested for the effect of PT copy number on CBGA production. The yCBGA0314 strain was constructed by inserting the ERG20mut-dPT gene under the regulation of the bidirectional GAL1/GAL10 promoter into the LPP1 locus by replacing the native LPP1 ORF of the yCBGA0269 strain. The yCBGA0314 strain and several other previously described strains are listed in Table 3 below along with their respective gene insertions/mutations:

TABLE 3

| | Mutant | | | |
|---|---|---|---|---|
| | ERG20 | | | |
| | integration; | | | |
| | truncated | GFP-dPT | ERG20mut- | |
| | HMG1 | copy | dPT copy | Promoter |
| Strain ID | integration | number | number | truncation |
| yCBGA0197 | + | 0 | 0 | 0 |
| yCBGA0237 | + | 1 | 0 | 0 |
| yCBGA0251 | + | 1 | 1 | 0 |
| yCBGA0269 | + | 1 | 1 | + |
| yCBGA0314 | + | 1 | 2 | + |

Transformant colonies were grown using the following protocol: Transformant colonies were inoculated into wells of a 96-well deep well plate. Each well contained 400 μl SC Medium (6.7 g/L Yeast Nitrogen Base, 1.6 g/L Amino Acid Drop Out mix without leucine, uracil, tryptophan and histidine, 22 g/L glucose, buffered to pH 6.0, supplemented with leucine, uracil, tryptophan and histidine). These inoculums were grown for 48 hours at 30° C. and shaken at 300 rpm with a 50 mm shaking diameter. After the 48 hours growth period, 40 μl samples of these cultures were inoculated into 360 μl YPD-2400LA (10 g/L yeast extract, 20 g/L peptone, 20 g/L glucose and 240 mg/L olivetolic acid) medium. Then samples were grown for 24 hours at 30° C. and shaken at 300 rpm with a 50 mm shaking diameter and 8 μl of 12000 mg/L OLA dissolved in EtOH was added to the samples. Finally, the samples were grown for additional 24 hours and were analyzed for cannabinoids.

The yCBGA0314 strain produced 450 mg/L CBGA using 480 mg/L olivetolic acid as substrate.

### Example 12—Preventing Hexanoic Acid Degradation and Increasing CBGA Titers

Cannabinoid production can be limited by the availability of hexanoic acid. We tested to see if the availability of hexanoic acid can be increased by knocking out several genes of the beta-oxidation pathway. In particular, we tested to see if hexanoic acid degradation could be prevented or minimized. Deletion of FAA1, FAA4, FAT1, PXA1, PXA2 and PEX11 had no obvious effect on hexanoic acid titers: after 24 hours of growth in YPD-HXA medium the hexanoic acid concentration was dropped below 5% of the original level (no heterologous cannabinoid pathway genes present). In other words, hexanoic degradation was not affected. A wild type control strain performed similarly, resulting in less than 5% final hexanoic acid concentration. However, deletion of the FOX1 gene (a.k.a. PDX1, systematic name YGL205W) increased hexanoic acid titers. The knockout of FOX1 eliminated HXA degradation almost completely: 95% of the original hexanoic concentration was still present at the end of the 24 hour long growth period.

Yeast strains having the ability to make CBGA were also knocked out for the FOX1 gene. yCBGA0326 strain was constructed by first, inserting the PKS and OAC genes under the regulation of the bidirectional GAL1/GAL10 promoter and the AAE1 gene under the regulation of the HXK1 promoter into the YGL202W locus replacing the native YGL202W ORF, second, inserting the PKS and OAC genes under the regulation of the bidirectional GAL1/GAL10 promoter and the AAE1 gene under the regulation of the HXK1 promoter into the DPP1 locus replacing the native DPP1 ORF, third, inserting the PKS and OAC genes under the regulation of the bidirectional GAL1/GAL10 promoter into the BTS1 locus replacing the native BTS1 ORF. yCBGA0373 strain was constructed by deleting the FOX1 gene, more specifically the nucleotide fragment between −73 and 3243 relative to the translational start site.

Strains yCBGA0326 and yCBGA0373 were inoculated into separate wells of a 96-well deep well plate. Each well contained 400 μl Synthetic Complete (SC) liquid medium (6.7 g/L Yeast Nitrogen Base, 1.6 g/L Amino Acid Drop Out mix without uracil and leucine, 22 g/L glucose, buffered to pH 6.0, supplemented with leucine and uracil). These inoculums were grown for 48 hours at 30° C. and shaken at 300 rpm with a 50 mm shaking diameter. After the 48 hours growth period 40 μl samples of these cultures were inoculated into 360 μl YPD-50HXA (10 g/L yeast extract, 20 g/L peptone, 20 g/L glucose and 50 mg/L hexanoic acid) medium. Then samples were grown for 24 hours at 30° C. and shaken at 300 rpm with a 50 mm shaking diameter, then 20 μg hexanoic acid dissolved in 8 μl ethanol was added to the samples. Finally, samples were grown for additional 24 hours and four replicates of both strains were analyzed for CBGA titers.

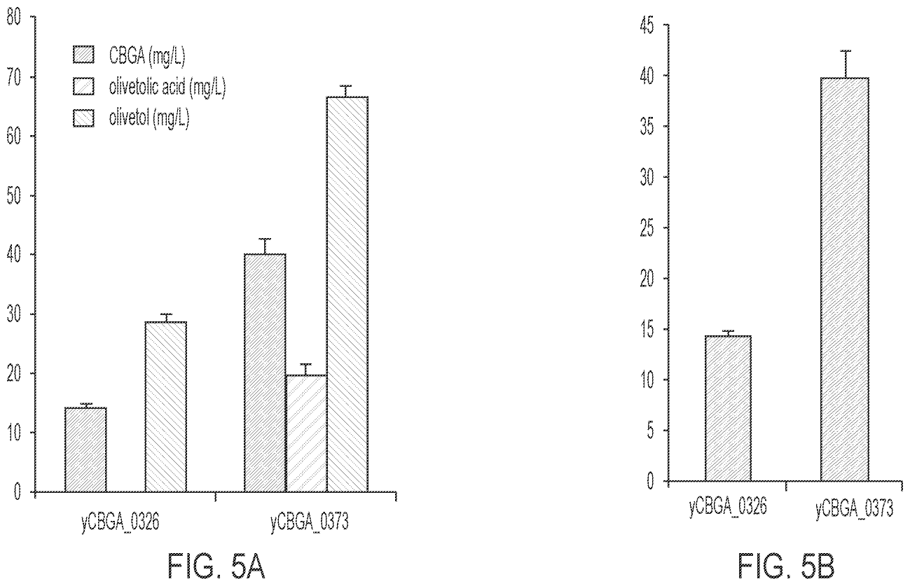

Using the methods described above, CBGA, olivetol, and olivetol acid titers were measured. As seen in FIG. 5, yCBGA0326 produced 14.3 mg/L CBGA and 28.4 mg/L olivetol while the strain yCBGA0373 deleted for the FOX1 gene produced 39.7 mg/L CBGA, 19.6 mg/L olivetolic acid and 66.1 mg/L olivetol.

### Example 13—Strain with Increased AAE1—TKS—OAC Copy Number

Increasing the copy number of genes required for olivetolic acid production increased the corresponding CBGA titer. The yCBGA0268 strain was constructed by replacing the native YGL202W ORF of the yCBGA0251 strain by inserting the PKS and OAC genes under the regulation of the bidirectional GAL1/GAL10 promoter and the AAE1 gene under the regulation of the HXK1 promoter into the YGL202W locus. The yCBGA0268 strain and several other previously described strains are listed below along with their respective gene insertions/mutations. The Table 4 illustrates how copy number of the olivetolic acid biosynthetic genes increases the final CBGA titer.

TABLE 4

| | Combined | | | | | CBGA |
|---|---|---|---|---|---|---|
| | GFP-dPT | | Copy | Copy | | titer |
| | and | Copy | num- | num- | Dele- | (mg/L); |
| | ERG20mut- | number | ber | ber | tion | hexanoic |
| | dPT copy | of | of | of | of | acid as |
| Strain ID | number | CsAAE1 | PKS | OAC | FOX1 | substrate |
| yCBGA0251 | 2 | 0 | 0 | 0 | 0 | 0 |
| yCBGA0268 | 2 | 1 | 1 | 1 | 0 | 14 |
| yCBGA0326 | 2 | 1 | 3 | 3 | 0 | 31 |
| yCBGA0373 | 2 | 1 | 3 | 3 | + | 63 |

### Example 14—the yCBGA0513 Strain: Strain with Increased Prenyl Transferase Copy Number+Increased AAE1-TKS-OAC Copy Number

The yCBGA0513 strain, a strain containing multiple copies of prenyl transferase and genes required for olivetolic acid production, was constructed. The strain produced increased levels of CBGA using hexanoic acid as its substrate. For the strain construction, the parental strain was the yCBGA0314 strain which contained 3 copies of GFP-dPT and/or ERGOmut-dPT genes in total among other host modifications as described above. Three copies of the PKS and OAC genes under the regulation of the bidirectional GAL1/GAL10 promoter and three copies of the AAE1 gene under the regulation of the STE5 promoter were inserted into the YDR508C, YRL020C and FOX1 loci replacing the native ORFs of the yCBGA0314 strain. A large number of isolates from this transformation were screened and an isolate with high CBGA productivity and the best reproducibility was identified. Next, to complement the auxotrophic mutations of this strain, URA3, HISS LEU2 and TRP1 genes on a single fragment were inserted 3′ of the genomic ARG2 locus, resulting in the final yCBGA0513 strain.

Colonies were inoculated into wells of a 96-well deep well plate. Each well contained 400 μl SC (6.7 g/L Yeast Nitrogen Base, 1.6 g/L Amino Acid Drop Out mix without leucine, uracil, tryptophan and histidine, 22 g/L glucose, buffered to pH 6.0, supplemented with leucine, uracil, tryptophan and histidine). These inoculums were grown for 48 hours at 30° C. and shaken at 300 rpm with a 50 mm shaking diameter. After the 48 hours growth period 40 μl samples of these cultures were inoculated into 360 μl YPD-HXA (10 g/L yeast extract, 20 g/L peptone, 20 g/L glucose and 100 mg/L hexanoic acid) medium. Then samples were grown for 24 hours at 30° C. and shaken at 300 rpm with a 50 mm shaking diameter and 40 μg hexanoic acid dissolved in 8 μl ethanol was added to the samples. Finally, the samples were grown for additional 24 hours and were analyzed for cannabinoids.

The yCBGA0513 strain was able to produce 140 mg/L CBGA in a standard high throughput screen. 1) The high prenyl transferase activity and optimized GPP productivity, as described for the parental yBGA0314 strain, 2) the replacement of the FOX1 gene that minimizes the degradation of hexanoic acid, and 3) and/or the integration of more than 3 copies of the genes required for olivetolic acid synthesis likely contribute to the high CBGA productivity.

To further test the potential of the yCBGA0513 strain, it was challenged in a modified screening procedure. yCBGA0513 strain was inoculated into wells of a 96-well deep well plate. Each well contained 400 μl SC liquid medium (6.7 g/L Yeast Nitrogen Base, 1.6 g/L Amino Acid Drop Out mix without leucine, uracil, tryptophan and histidine, 22 g/L glucose, buffered to pH 6.0, supplemented with leucine, uracil, tryptophan and histidine). These inoculums were grown for 48 hours at 30° C. and shaken at 300 rpm with a 50 mm shaking diameter. After the 48 hours growth period 40 μl samples of these cultures were inoculated into 360 μl YPD-HXA (10 g/L yeast extract, 20 g/L peptone, 20 g/L glucose and 100 mg/L hexanoic acid) medium. Then samples were grown for 96 hours at 30° C. and shaken at 300 rpm with a 50 mm shaking diameter and 40 μg hexanoic acid dissolved in 8 μl ethanol was added to the samples at two time-points: at 24 and 48 hour. At the end of the 96 hour growth period, the samples were analyzed for cannabinoids. The yCBGA0513 strain produced 300 mg/L CBGA using hexanoic acid as substrate.

### Example 15—the yCBGA0520, yCBGA0523 and yCBGA0526 Strains: Strains with Increased Prenyl Transferase Copy Number+Genes Required for Olivetolic Acid Production

Several additional strains containing multiple copies of prenyl transferase and genes required for olivetolic acid production were constructed utilizing yCBGA0513 as the parent strain. The yCBGA0520, yCBGA0523 and yCBGA0526 strains are able to produce high levels of CBGA using hexanoic acid as substrate. The yCBGA0520 strain was constructed by inserting the ERG13 gene (encoding Hydroxymethylglutaryl-CoA synthase) under the regulation of the ADH2 promoter, the HMG1 gene (encoding Hydroxymethylglutaryl-CoA reductase) under the regulation of the TEF1 promoter, and the ERG20mut-dPT gene with a TPI1 terminator under the regulation of the HXK1 promoter into the ATG26 locus by replacing the native ATG26 ORF of the yCBGA0513 strain. The yCBGA_0523 strain was constructed by inserting the tHMG1 gene under the regulation of the ADH2 promoter, the ERG10 gene (encoding Acetyl-CoA C-acetyltransferase) under the regulation of the TEF1 promoter, and the ERG13 gene under the regulation of the HXK1 promoter into the ATG26 locus by replacing the native ATG26 locus of the yCBGA0513 strain. The yCBGA_0526 strain was constructed by inserting the tHMG1 gene under the regulation of the ADH2 promoter, the ERG13 gene under the regulation of the TEF1 promoter, and the AAE1 gene with a TEF1 terminator under the regulation of the HXK1 promoter into the ATG26 locus by replacing the native ATG26 ORF of the yCBGA0513 strain. tHMG1 stands for the Saccharomyces cerevisiae HMG1 gene truncated of the first 530 amino acids. ERG10, ERG13, HMG1 and tHMG1 gene cassettes contain their native terminator sequences. ERG20mut-dPT and AAE1 were described earlier. The ATG26 locus of the yCBGA0513 strain and the corresponding locuses in the yCBGA0520 strain, the yCBGA0523 strain, and the yCBGA0526 strain which replaced the ATG26 locus are disclosed in FIG. 13.

The strains and their gene insertions are listed in Table 5 below:

TABLE 5

| | | | | | | | | | | |
|---|---|---|---|---|---|---|---|---|---|---|
| | Target | Pro- | | Termi- | Pro- | | Termi- | Pro- | | Termi- |
| Strain | locus | moter1 | ORF1 | nator1 | moter2 | ORF2 | nator2 | moter3 | ORF3 | nator3 |

| yCBGA_ | ATG | ADH | ERG13 | TEF1 | HMG1 | HXK | ERG20mut- | TPI1 |
|---|---|---|---|---|---|---|---|---|
| 052 | | | | | | | dpt | |
| 0 | | | | | | | | |
| yCBGA_ | | | tHMG1 | | ERG1 | | ERG13 | |
| 052 | | | | | 0 | | | |
| 3 | | | | | | | | |
| yCBGA_ | | | | | ERG1 | | AAE1 | TEF |
| 052 | | | | | 3 | | | 1 |
| 6 | | | | | | | | |

tHMG1 stands for the Saccharomyces cerevisiae HMG1 gene truncated of the first 530 amino acids. ERG10, ERG13, HMG1 and tHMG1 gene cassettes contain their native terminator sequences. ERG20mut-dPT and AAE1 were described earlier.

All 3 strains outperformed the yCBGA0513 strain in the following modified high throughput screen: Colonies were inoculated into wells of a 96-well deep well plate. Each well contains 400 μl Synthetic Complete Medium (6.7 g/L Yeast Nitrogen Base, 1.6 g/L Amino Acid Drop Out mix without leucine, uracil, tryptophan and histidine, 22 g/L glucose, buffered to pH 6.0, supplemented with leucine, uracil, tryptophan and histidine). These inoculums were grown for 48 hours at 30° C. and shaken at 300 rpm with a 50 mm shaking diameter. After the 48 hours growth period, 40 μl samples of these cultures were inoculated into 360 μl YPD-HXA (10 g/L yeast extract, 20 g/L peptone, 20 g/L glucose and 100 mg/L hexanoic acid) medium. Then samples were grown for 72 hours at 30° C. and shaken at 300 rpm with a 50 mm shaking diameter and 80 μg hexanoic acid dissolved in 8 μl ethanol was added to the samples at two time-points: at 24 and 48 hours. At the end of the 72 hour growth period, the samples were analyzed for cannabinoids. The strains and their final olivetolic acid, olivetol and CBGA titers are listed in Table 6 below:

TABLE 6

| | Olivetolic | | |
|---|---|---|---|
| | acid | Olivetol | CBGA |
| Strain | mg/L | mg/L | mg/L |
| yCBGA0513 | 94 | 185 | 199 |
| yCBGA0520 | 74 | 215 | 313 |
| yCBGA0523 | 56 | 204 | 311 |
| yCBGA0526 | 79 | 215 | 305 |

### Example 16—TKS (OS) Enzyme Evolution

To improve the performance of TKS, an enzyme evolution experiment was conducted. The wild type amino acid and nucleotide sequences of Saccharomyces cerevisiae TKS are included Table 7 below:

TABLE 7

| | | SEQ |
|---|---|---|
| Description | | ID |
| of sequence | Sequence | NO: |
| WT TKS aa | MNHLRAEGPASVLAIGTANPENILIQDEFPD | 40 |
| | YYFRVTKSEHMTQLKEKFRKICDKSMIRKRN | |
| | CFLNEEHLKQNPRLVEHEMQTLDARQDMLVV | |
| | EVPKLGKDACAKAIKEWGQPKSKITHLIFTS | |
| | ASTTDMPGADYHCAKLLGLSPSVKRVMMYQL | |
| | GCYGGGTVLRIAKDIAENNKGARVLAVCCDI | |
| | MACLFRGPSDSDLELLVGQAIFGDGAAAVIV | |
| | GAEPDESVGERPIFELVSTGQTILPNSEGTI | |
| | GGHIREAGLIFDLHKDVPMLISNNIEKCLIE | |
| | AFTPIGISDWNSIFWITHPGGKAILDKVEEK | |
| | LDLKKEKFVDSRHVLSEHGNMSSSTVLFVMD | |
| | ELRKRSLEEGKSTTGDGFEWGVLFGFGPGLT | |
| | VERVVVRSVPIKY | |
| WT TKS nt | ATGAATCATTTGAGAGCTGAAGGTCCAGCAT | 41 |
| | CAGTTTTGGCTATTGGTACTGCAAACCCAGA | |
| | AAACATCTTGATCCAAGATGAATTTCCAGAT | |
| | TATTACTTCAGAGTTACTAAGTCAGAACATA | |
| | TGACACAATTGAAGGAAAAGTTTAGAAAGAT | |
| | CTGTGATAAGTCTATGATTAGAAAAAGAAAT | |
| | TGTTTCTTGAACGAAGAACATTTGAAGCAAA | |
| | ACCCAAGATTAGTTGAACATGAAATGCAAAC | |
| | ATTGGATGCTAGACAAGATATGTTGGTTGTT | |
| | GAAGTTCCAAAGTTGGGTAAAGATGCATGTG | |
| | CTAAAGCAATTAAAGAATGGGGTCAACCAAA | |
| | GTCTAAGATCACTCATTTGATTTTTACATCA | |
| | GCATCTACTACAGATATGCCAGGTGCTGATT | |
| | ACCATTGTGCAAAGTTGTTGGGTTTGTCACC | |
| | ATCTGTTAAGAGAGTTATGATGTACCAATTA | |
| | GGTTGTTACGGTGGTGGTACTGTTTTGAGAA | |
| | TCGCTAAGGATATCGCAGAAAACAATAAGGG | |
| | TGCTAGAGTCTTGGCAGTTTGTTGTGATATC | |
| | ATGGCTTGTTTGTTTAGAGGTCCATCAGATT | |
| | CTGATTTGGAATTGTTAGTTGGTCAAGCTAT | |
| | TTTTGGTGACGGTGCTGCAGCTGTTATTGTT | |
| | GGTGCAGAACCAGATGAATCAGTTGGTGAAA | |
| | GACCAATCTTCGAATTAGTTTCAACTGGTCA | |
| | AACAATTTTGCCAAATTCTGAAGGTACAATT | |
| | GGTGGTCATATCAGAGAAGCTGGTTTGATCT | |
| | TCGATTTGCATAAAGATGTTCCAATGTTGAT | |
| | CTCTAACAACATCGAAAAGTGTTTGATCGAA | |
| | GCTTTTACTCCAATCGGTATCTCAGATTGGA | |
| | ACTCTATTTTCTGGATTACACATCCAGGTGG | |
| | TAAAGCAATCTTGGATAAGGTTGAAGAAAAA | |
| | TTGGATTTGAAGAAAGAAAAATTTGTTGATT | |
| | CAAGACATGTTTTGTCTGAACATGGTAACAT | |
| | GTCTTCATCTACTGTTTTGTTCGTTATGGAT | |
| | GAATTGAGAAAGAGATCATTAGAAGAGGGTA | |
| | AATCTACTACAGGTGACGGTTTTGAATGGGG | |
| | TGTTTTATTTGGTTTTGGTCCAGGTTTGACA | |
| | GTTGAAAGAGTTGTTGTTAGATCTGTTCCAA | |
| | TTAAATACTAA | |

Each position in TKS listed in Table 8 below was mutagenized and 20-100 yeast colonies carrying randomly changed amino acids for each marked positions were screened for elevated performance.

TABLE 8

| Position in TKS | Amino acid |
|---|---|
| 48 | Lys |
| 51 | Lys |
| 52 | Ile |
| 55 | Lys |
| 56 | Ser |
| 125 | Ala |
| 126 | Ser |
| 130 | Met |
| 156 | Gly |
| 157 | Cys |
| 185 | Asp |
| 186 | Ile |
| 187 | Met |
| 189 | Cys |
| 190 | Leu |
| 200 | Glu |
| 203 | Val |
| 207 | Ile |
| 208 | Phe |
| 209 | Gly |
| 210 | Asp |
| 248 | Ile |
| 250 | Gly |
| 257 | Leu |
| 259 | Phe |
| 261 | Leu |
| 263 | Lys |
| 264 | Asp |
| 265 | Val |
| 266 | Pro |
| 297 | His |
| 299 | Gly |
| 300 | Gly |
| 301 | Lys |
| 302 | Ala |
| 303 | Ile |
| 330 | Asn |
| 332 | Ser |
| 366 | Gly |
| 367 | Phe |
| 368 | Gly |
| 369 | Pro |
| 370 | Gly |

The amino acid position A1a125, coding for Alanine in the wild type sequence was found to be able to elevate the TKS activity when the Alanine was swapped for different amino acids. When this amino acid position of TKS contained Serine, the corresponding olivetolic acid production was 50 mg/L in contrast with the 37 mg/L measured in case of the wild type TKS gene.

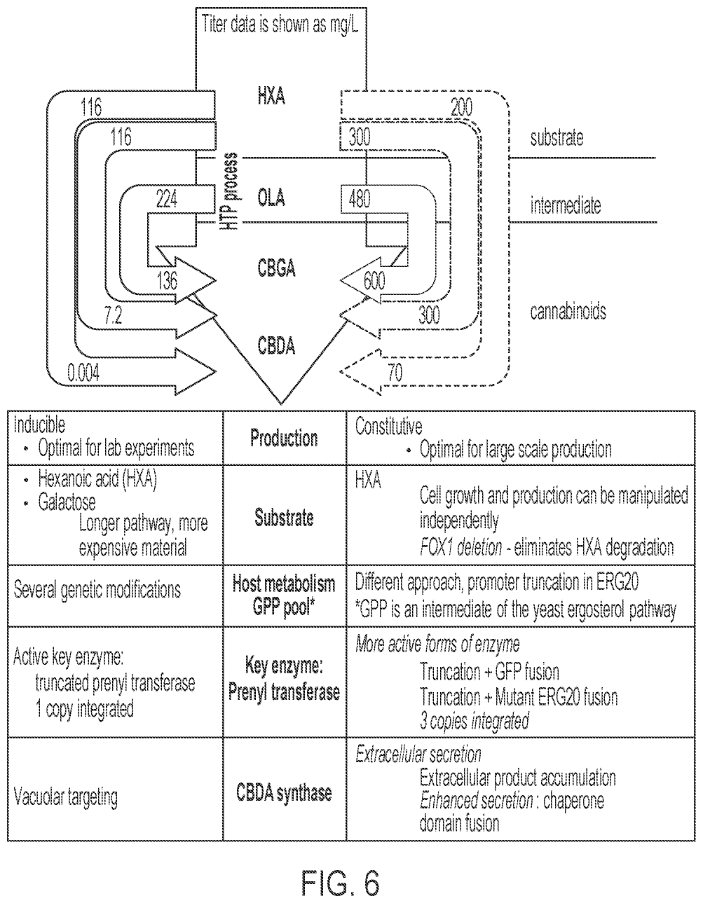

The TKS gene was inserted into a plasmid containing the Saccharomyces cerevisiae 2μ replication origin, the URA3 gene as an auxotrophic marker and the TKS gene under the regulation of the GAL10 promoter. The plasmid ID of the control plasmid with wild type TKS sequence was bCBGA1024. The ID of the plasmid with the Alanine to Serine mutation at position 125 is 0827-01-A1-1 (FIG. 6A).

The yCBGA0368 strain was used for screening these mutant plasmids. yCBGA0368 was constructed by inserting the AAE1 gene under the regulation of the STE5 promoter and the OAC gene under the regulation of the GAL1 promoter into the YKL140W locus by replacing the native YMR145C ORF of the yCBGA0215 strain (FIG. 6B). The yCBGA0215 strain was constructed by deleting the HygMX and KanMX cassettes from the yCBGA0197 strain.

### Example 17—Enhanced CBDA Synthase Secretion

The secretion of CBDAs was optimized to be able produce large amounts of CBDA in a single process using yeast cells, the wild type sequences for the Saccharomyces cerevisiae CBDA amino acid and nucleotide sequences are included in Table 9:

TABLE 9

| | | SEQ |
|---|---|---|
| Description | | ID |
| of sequence | Sequence | NO: |
| WT CBDA | ATGAAATGTTCTACATTTTCATTTTGGTTTG | 42 |
| synthase nt | TTTGTAAGATCATTTTCTTTTTCTTTTCTTT | |
| | TAATATTCAAACTTCAATCGCTAACCCAAGA | |
| | GAAAATTTCTTGAAGTGTTTCTCTCAATACA | |
| | TTCCAAATAATGCAACAAATTTGAAATTGGT | |
| | TTATACTCAAAATAATCCATTATACATGTCT | |
| | GTTTTAAATTCTACAATTCATAATTTGAGAT | |
| | TTTCTTCAGATACTACACCAAAACCATTGGT | |
| | TATTGTTACACCATCTCATGTTTCACATATC | |
| | CAAGGTACTATCTTGTGTTCTAAGAAAGTTG | |
| | GTTTGCAAATTAGAACTAGATCAGGTGGTCA | |
| | TGATTCAGAAGGCATGTCTTACATCTCACAA | |
| | GTTCCATTCGTTATCGTTGATTTGAGAAACA | |
| | TGAGATCAATTAAAATTGATGTTCATTCACA | |
| | AACAGCTTGGGTTGAAGCTGGTGCAACTTTG | |
| | GGTGAAGTTTACTACTGGGTTAACGAAAAGA | |
| | ATGAATCTTTATCATTGGCTGCTGGTTACTG | |
| | TCCAACAGTTTGTGCTGGTGGTCATTTTGGT | |
| | GGTGGTGGTTATGGTCCATTAATGAGATCCT | |
| | ATGGTTTGGCTGCTGATAACATCATCGATGC | |
| | ACATTTGGTTAACGTTCATGGTAAAGTTTTG | |
| | GATAGAAAGTCTATGGGTGAAGATTTGTTTT | |
| | GGGCTTTGAGAGGTGGTGGTGCTGAATCATT | |
| | TGGTATCATCGTTGCTTGGAAGATCAGATTG | |
| | GTTGCAGTTCCAAAATCTACTATGTTCTCAG | |
| | TTAAGAAAATTATGGAAATCCATGAATTAGT | |
| | TAAATTGGTTAATAAGTGGCAAAATATTGCT | |
| | TATAAATACGATAAAGATTTGTTATTGATGA | |
| | CTCATTTTATTACAAGAAATATTACTGATAA | |
| | CCAAGGTAAAAATAAGACAGCTATCCATACT | |
| | TACTTTTCTTCAGTTTTCTTGGGTGGTGTTG | |
| | ATTCTTTGGTTGATTTGATGAATAAGTCTTT | |
| | TCCAGAATTAGGTATTAAGAAAACTGATTGT | |
| | AGACAATTGTCTTGGATCGATACTATCATTT | |
| | TCTATTCAGGTGTTGTTAACTACGATACAGA | |
| | TAACTTCAATAAGGAAATTTTATTGGATAGA | |
| | TCAGCTGGTCAAAATGGTGCTTTTAAAATTA | |
| | AATTGGATTACGTTAAGAAACCAATTCCAGA | |
| | ATCAGTTTTCGTTCAAATTTTAGAAAAATTG | |
| | TATGAAGAAGATATTGGTGCTGGCATGTACG | |
| | CATTGTATCCATACGGTGGTATCATGGATGA | |
| | AATTTCTGAATCAGCTATTCCATTTCCACAT | |
| | AGAGCAGGTATTTTATACGAATTGTGGTACA | |
| | TTTGTTCTTGGGAAAAGCAAGAAGATAACGA | |
| | AAAACATTTGAACTGGATTAGAAACATCTAT | |
| | AACTTCATGACTCCATACGTTTCACAAAACC | |
| | CAAGATTGGCTTATTTGAACTACAGAGATTT | |
| | GGATATCGGTATTAATGATCCTAAAAATCCA | |
| | AACAACTATACACAAGCAAGAATTTGGGGTG | |
| | AAAAGTACTTCGGTAAAAATTTCGATAGATT | |
| | GGTTAAGGTTAAAACTTTGGTTGATCCAAAT | |
| | AATTTCTTTAGAAATGAACAATCTATTCCAC | |
| | CATTGCCAAGACATAGACATTGA | |
| WT CBDA | MKCSTFSFWFVCKIIFFFFSFNIQTSIANPR | 43 |
| synthase aa | ENFLKCFSQYIPNNATNLKLVYTQNNPLYMS | |
| | VLNSTIHNLRFSSDTTPKPLVIVTPSHVSHI | |
| | QGTILCSKKVGLQIRTRSGGHDSEGMSYISQ | |
| | VPFVIVDLRNMRSIKIDVHSQTAWVEAGATL | |
| | GEVYYWVNEKNESLSLAAGYCPTVCAGGHFG | |
| | GGGYGPLMRSYGLAADNIIDAHLVNVHGKVL | |
| | DRKSMGEDLFWALRGGGAESFGIIVAWKIRL | |
| | VAVPKSTMFSVKKIMEIHELVKLVNKWQNIA | |
| | YKYDKDLLLMTHFITRNITDNQGKNKTAIHT | |
| | YFSSVFLGGVDSLVDLMNKSFPELGIKKTDC | |
| | RQLSWIDTIIFYSGVVNYDTDNFNKEILLDR | |
| | SAGQNGAFKIKLDYVKKPIPESVFVQILEKL | |
| | YEEDIGAGMYALYPYGGIMDEISESAIPFPH | |
| | RAGILYELWYICSWEKQEDNEKHLNWIRNIY | |
| | NFMTPYVSQNPRLAYLNYRDLDIGINDPKNP | |
| | NNYTQARIWGEKYFGKNFDRLVKVKTLVDPN | |
| | NFFRNEQSIPPLPRHRH | |

A series of plasmids were constructed containing the Saccharomyces cerevisiae replication origin, the URA3 gene as an auxotrophic marker and the CBDA synthase gene under the regulation of the bidirectional GAL1/GAL10 promoter. In each plasmid, the predicted plant secretion signal corresponding to the first 21 amino acid of the CBDA synthase was replaced with different yeast secretion signals, see table below.

This set of plasmids were separately transformed into yCBGA0314 strains and their CBDA productivity was assayed in a high throughput screening process optimized for CBDA synthase activity. The nucleotide sequences and protein sequence of the tested signal sequences are included in Tables 10-11 below, along with the plasmid IDs, the corresponding amino acid sequences, the ORF names and CBDA titer.

TABLE 10

| Plasmid | | Signal Sequence | SEQ ID | |
|---|---|---|---|---|
| ID | ORF name | Amino Acid Sequence | NO: | Titer |
| bCBGA1827 | OST1_signal-and- | MRQVWFSWIVGLFLCFFNVSSAAPVNTT | 44 | 13 |
| | alpha_only_pro_ | TEDETAQIPAEAVIGYLDLEGDFDVAVL | | |
| | signal-d21_CBDAs | PFSNSTNNGLLFINTTIASIAAKEEGVS | | |
| | | LDKREAEA | | |
| bCBGA1829 | alpha_prepro_ | MRFPSIFTAVLFAASSALAAPVNTTTED | 45 | 7 |
| | signal-d21_CBDAs | ETAQIPAEAVIGYLDLEGDFDVAVLPFS | | |
| | | NSTNNGLLFINTTIASIAAKEEGVSLDK | | |
| | | REAEA | | |
| bCBGA1835 | AMYI_signal- | MQRPFLLAYLVLSLLFNSALG | 46 | 19 |
| | d21_CBDAs | | | |
| bCBGA1836 | BGL2_signal- | MRFSTTLATAATALFFTASQVSA | 47 | 11 |
| | d21_CBDAs | | | |
| bCBGA1837 | OST1_signal- | MRQVWFSWIVGLFLCFFNVSSA | 48 | 41 |
| | d21_CBDAs | | | |
| bCBGA1838 | SUC2_signal- | MLLQAFLFLLAGFAAKISA | 49 | 11 |
| | d21_CBDAs | | | |
| bCBGA1839 | PHO5_signal-and- | MFKSVVYSILAASLANAAPVNTTTEDET | 50 | 44 |
| | alpha_only_pro_ | AQIPAEAVIGYLDLEGDFDVAVLPFSNS | | |
| | signal-d21_CBDAs | TNNGLLFINTTIASIAAKEEGVSLDKRE | | |
| | | AEA | | |
| bCBGA1840 | AGA2_signal-and- | MQLLRCFSIFSVIASVLAAPVNTTTEDE | 51 | 10 |
| | alpha_only_pro_ | TAQIPAEAVIGYLDLEGDFDVAVLPFSN | | |
| | signal-d21_CBDAs | STNNGLLFINTTIASIAAKEEGVSLDKR | | |
| | | EAEA | | |
| bCBGA1842 | AMYI_signal-and- | MQRPFLLAYLVLSLLFNSALGAPVNTTT | 52 | 11 |
| | alpha_only_pro_ | EDETAQIPAEAVIGYLDLEGDFDVAVLP | | |
| | signal-d21_CBDAs | FSNSTNNGLLFINTTIASIAAKEEGVSL | | |
| | | DKREAEA | | |
| bCBGA1843 | BGL2_signal-and- | MRFSTTLATAATALFFTASQVSAAPVNT | 53 | 9 |
| | alpha_only_pro_ | TTEDETAQIPAEAVIGYLDLEGDFDVAV | | |
| | signal-d21_CBDAs | LPFSNSTNNGLLFINTTIASIAAKEEGV | | |
| | | SLDKREAEA | | |
| bCBGA1845 | SUC2_signal-and- | MLLQAFLFLLAGFAAKISAAPVNTTTED | 54 | 9 |
| | alpha_only_pro_ | ETAQIPAEAVIGYLDLEGDFDVAVLPFS | | |
| | signal-d21_CBDAs | NSTNNGLLFINTTIASIAAKEEGVSLDK | | |
| | | REAEA | | |
| bCBGA1846 | HSP150_delta_ | MQYKKTLVASALAATTLAAYAPSEPWST | 55 | 102 |
| | fragment-d21_ | LTPTATYSGGVTDYASTFGIAVQPISTT | | |
| | CBDAs | SSASSAATTASSKAKRAASQIGDGQVQA | | |
| | | ATTTASVSTKSTAAAVSQIGDGQIQATT | | |
| | | KTTAAAVSQIGDGQIQATTKTTSAKTTA | | |
| | | AAVSQISDGQIQATTTTLAPKSTAAAVS | | |
| | | QIGDGQVQATTTTLAPKSTAAAVSQIGD | | |
| | | GQVQATTKTTAAAVSQIGDGQVQATTKT | | |
| | | TAAAVSQIGDGQVQATTKTTAAAVSQIG | | |
| | | DGQVQATTKTTAAAVSQITDGQVQATTK | | |
| | | TTQAASQVSDGQVQATTATSASAAATST | | |
| | | DPVDAVSCKTSGT | | |
| bCBGA1847 | alpha_pre_ | MRFPSIFTAVLFAASSALAAYAPSEPWS | 56 | 94 |
| | signal-and- | TLTPTATYSGGVTDYASTFGIAVQPIST | | |
| | HSP150_delta_ | TSSASSAATTASSKAKRAASQIGDGQVQ | | |
| | fragment_wo_ | AATTTASVSTKSTAAAVSQIGDGQIQAT | | |
| | signal-d21_CBDAs | TKTTAAAVSQIGDGQVQATTKTTSAKTT | | |
| | | AAAVSQISDGQIQATTTTLAPKSTAAAV | | |
| | | SQIGDGQVQATTTTLAPKSTAAAVSQIG | | |
| | | DGQVQATTKTTAAAVSQIGDGQVQATTK | | |
| | | TTAAAVSQIGDGQVQATTKTTAAAVSQI | | |
| | | GDGQVQATTKTTAAAVSQITDGQVQATT | | |
| | | KTTQAASQVSDGQVQATTATSASAAATS | | |
| | | TDPVDAVSCKTSGT | | |
| bCBGA1851 | HSP150 secretion | MQYKKTLVASALAATTLA | 57 | 60 |
| | signal-d21_CBDAs | | | |
| bCBGA1852 | HSP150_delta_ | MQYKKTLVASALAATTLAAYAPSEPWST | 58 | 126 |
| | fragment-and- | LTPTATYSGGVTDYASTFGIAVQPISTT | | |
| | LEKREAEA- | SSASSAATTASSKAKRAASQIGDGQVQA | | |
| | d21_CBDAs | ATTTASVSTKSTAAAVSQIGDGQIQATT | | |
| | | KTTAAAVSQIGDGQIQATTKTTSAKTTA | | |
| | | AAVSQISDGQIQATTTTLAPKSTAAAVS | | |
| | | QIGDGQVQATTTTLAPKSTAAAVSQIGD | | |
| | | GQVQATTKTTAAAVSQIGDGQVQATTKT | | |
| | | TAAAVSQIGDGQVQATTKTTAAAVSQIG | | |
| | | DGQVQATTKTTAAAVSQITDGQVQATTK | | |
| | | TTQAASQVSDGQVQATTATSASAAATST | | |
| | | DPVDAVSCKTSGTLEKREAEA | | |
| bCBGA1860 | YDR055W_N term | MQLHSLIASTALLITSALA | 59 | 12 |
| | 19 AA-d21_CBDAs | | | |
| bCBGA1861 | YGR279C_N term | MRLSNLIASASLLSAATLA | 60 | 44 |
| | 19 AA-d21_CBDAs | | | |
| bCBGA1862 | YGR279C_N term | MRLSNLIASASLLSAATLAAPANHEHKD | 61 | 24 |
| | 31 AA-d21_CBDAs | KRA | | |
| bCBGA1863 | YLR300W_N term | MLSLKTLLCTLLTVSSVLA | 62 | 20 |
| | 19 AA-d21_CBDAs | | | |
| bCBGA1864 | YLR300W_N term | MLSLKTLLCTLLTVSSVLATPVPA | 63 | 54 |
| | 24 AA-d21_CBDAs | | | |
| bCBGA1865 | YLR300W_N term | MLSLKTLLCTLLTVSSVLATPVPARDPS | 64 | 58 |
| | 30 AA-d21_CBDAs | SI | | |
| bCBGA1873 | YAP_TA57- | MKLKTVRSAVLSSLFASQVLGQTTAQTN | 65 | 50 |
| | yEVenus-spacer- | SGGLDVVGLISMAMSKGEELFTGVVPIL | | |
| | d21_CBDAs | VELDGDVNGHKFSVSGEGEGDATYGKLT | | |
| | | LKLICTTGKLPVPWPTLVTTLGYGLQCF | | |
| | | ARYPDHMKQHDFFKSAMPEGYVQERTIF | | |
| | | FKDDGNYKTRAEVKFEGDTLVNRIELKG | | |
| | | IDFKEDGNILGHKLEYNYNSHNVYITAD | | |
| | | KQKNGIKANFKIRHNIEDGGVQLADHYQ | | |
| | | QNTPIGDGPVLLPDNHYLSYQSALSKDP | | |
| | | NEKRDHMVLLEFVTAAGITHGMDELYKE | | |
| | | EGEPK | | |
| bCBGA1874 | YAP_TA57-Kex2- | MKLKTVRSAVLSSLFASQVLGQTTAQTN | 66 | 51 |
| | yEVenus-spacer- | SGGLDVVGLISMAKRKRMSKGEELFTGV | | |
| | d21_CBDAs | VPILVELDGDVNGHKFSVSGEGEGDATY | | |
| | | GKLTLKLICTTGKLPVPWPTLVTTLGYG | | |
| | | LQCFARYPDHMKQHDFFKSAMPEGYVQE | | |
| | | RTIFFKDDGNYKTRAEVKFEGDTLVNRI | | |
| | | ELKGIDFKEDGNILGHKLEYNYNSHNVY | | |
| | | ITADKQKNGIKANFKIRHNIEDGGVQLA | | |
| | | DHYQQNTPIGDGPVLLPDNHYLSYQSAL | | |
| | | SKDPNEKRDHMVLLEFVTAAGITHGMDE | | |
| | | LYKEEGEPK | | |
| bCBGA1877 | YAP_TA57_Kex2_ | MKLKTVRSAVLSSLFASQVLGQTTAQTN | 67 | 49 |
| | spacer.d21_ | SGGLDVVGLISMAKREEGEPK | | |
| | CBDAs_del_KR_ | | | |
| | motif | | | |
| bCBGA1878 | YAP_TA57_Kex2_ | MKLKTVRSAVLSSLFASQVLGQTTAQTN | 68 | 50 |
| | spacer.d21_ | SGGLDVVGLISMAEEGEPK | | |
| | CBDAs_del_KEX2_ | | | |
| | motif | | | |
| bCBGA1882 | MEL1m3 signal- | MRAFLFLTACISLPGVFGVEEGEPK | 69 | 64 |
| | d21_CBDAs | | | |
| bCBGA1884 | INU1A signal- | MKLAYSLLLPLAGVSASVINYKRMAMVS | 70 | 61 |
| | noSpacer- | | | |
| | d21_CBDAs | | | |
| bCBGA1885 | INU1 signal- | MKFAYSLLLPLAGVSASVINYKRMAMVS | 71 | 60 |
| | noSpacer- | | | |
| | d21_CBDAs | | | |
| bCBGA1886 | MEL1m3 signal- | MRAFLFLTACISLPGVFGV | 72 | 25 |
| | noSpacer- | | | |
| | d21_CBDAs | | | |
| bCBGA1887 | N/A | MRAFLFLTACISLPGVFG | 73 | NA |

TABLE 11

| | | SEQ |
|---|---|---|
| Plasmid | Signal Sequence | ID |
| ID | Nucleotide Sequence | NO: |
| bCBGA1827 | ATGAGGCAGGTTTGGTTCTCTTGGATTGTGGGATTGTT | 74 |
| | CCTATGTTTTTTCAACGTGTCTTCTGCTGCTCCAGTCA | |
| | ACACTACAACAGAAGATGAAACGGCACAAATTCCGGC | |
| | TGAAGCTGTCATCGGTTACTTAGATTTAGAAGGGGAT | |
| | TTCGATGTTGCTGTTTTGCCATTTTCCAACAGCACAAA | |
| | TAACGGGTTATTGTTTATAAATACTACTATTGCCAGCA | |
| | TTGCTGCTAAAGAAGAAGGGGTATCTTTGGATAAAAG | |
| | AGAGGCTGAAGCT | |
| bCBGA1829 | ATGAGATTTCCTTCAATTTTTACTGCAGTTTTATTCGC | 75 |
| | AGCATCCTCCGCATTAGCTGCTCCAGTCAACACTACA | |
| | ACAGAAGATGAAACGGCACAAATTCCGGCTGAAGCTG | |
| | TCATCGGTTACTTAGATTTAGAAGGGGATTTCGATGTT | |
| | GCTGTTTTGCCATTTTCCAACAGCACAAATAACGGGTT | |
| | ATTGTTTATAAATACTACTATTGCCAGCATTGCTGCTA | |
| | AAGAAGAAGGGGTATCTTTGGATAAAAGAGAGGCTG | |
| | AAGCT | |
| bCBGA1835 | ATGCAAAGACCATTTCTACTCGCTTATTTGGTCCTTTC | 76 |
| | GCTTCTATTTAACTCAGCTTTGGGT | |
| bCBGA1836 | ATGCGTTTCTCTACTACACTCGCTACTGCAGCTACTGC | 77 |
| | GCTATTTTTCACAGCCTCCCAAGTTTCAGCT | |
| bCBGA1837 | ATGAGGCAGGTTTGGTTCTCTTGGATTGTGGGATTGTT | 78 |
| | CCTATGTTTTTTCAACGTGTCTTCTGCT | |
| bCBGA1838 | ATGCTTTTGCAAGCTTTCCTTTTCCTTTTGGCTGGTTT | 79 |
| | TGCAGCCAAAATATCTGCA | |
| bCBGA1839 | ATGTTTAAATCTGTTGTTTATTCAATTTTAGCCGCTTC | 80 |
| | TTTGGCCAATGCAGCTCCAGTCAACACTACAACAGAAG | |
| | ATGAAACGGCACAAATTCCGGCTGAAGCTGTCATCGG | |
| | TTACTTAGATTTAGAAGGGGATTTCGATGTTGCTGTTT | |
| | TGCCATTTTCCAACAGCACAAATAACGGGTTATTGTTT | |
| | ATAAATACTACTATTGCCAGCATTGCTGCTAAAGAAG | |
| | AAGGGGTATCTTTGGATAAAAGAGAGGCTGAAGCT | |
| bCBGA1840 | ATGCAGTTACTTCGCTGTTTTTCAATATTTTCTGTTAT | 81 |
| | TGCTTCAGTTTTAGCAGCTCCAGTCAACACTACAACAG | |
| | AAGATGAAACGGCACAAATTCCGGCTGAAGCTGTCAT | |
| | CGGTTACTTAGATTTAGAAGGGGATTTCGATGTTGCTG | |
| | TTTTGCCATTTTCCAACAGCACAAATAACGGGTTATTG | |
| | TTTATAAATACTACTATTGCCAGCATTGCTGCTAAAGA | |
| | AGAAGGGGTATCTTTGGATAAAAGAGAGGCTGAAGCT | |
| bCBGA1842 | ATGCAAAGACCATTTCTACTCGCTTATTTGGTCCTTTC | 82 |
| | GCTTCTATTTAACTCAGCTTTGGGTGCTCCAGTCAACA | |
| | CTACAACAGAAGATGAAACGGCACAAATTCCGGCTGA | |
| | AGCTGTCATCGGTTACTTAGATTTAGAAGGGGATTTCG | |
| | ATGTTGCTGTTTTGCCATTTTCCAACAGCACAAATAAC | |
| | GGGTTATTGTTTATAAATACTACTATTGCCAGCATTGC | |
| | TGCTAAAGAAGAAGGGGTATCTTTGGATAAAAGAGAG | |
| | GCTGAAGCT | |
| bCBGA1843 | ATGCGTTTCTCTACTACACTCGCTACTGCAGCTACTGC | 83 |
| | GCTATTTTTCACAGCCTCCCAAGTTTCAGCTGCTCCAG | |
| | TCAACACTACAACAGAAGATGAAACGGCACAAATTCC | |
| | GGCTGAAGCTGTCATCGGTTACTTAGATTTAGAAGGG | |
| | GATTTCGATGTTGCTGTTTTGCCATTTTCCAACAGCAC | |
| | AAATAACGGGTTATTGTTTATAAATACTACTATTGCCA | |
| | GCATTGCTGCTAAAGAAGAAGGGGTATCTTTGGATAA | |
| | AAGAGAGGCTGAAGCT | |
| bCBGA1845 | ATGCTTTTGCAAGCTTTCCTTTTCCTTTTGGCTGGTTT | 84 |
| | TGCAGCCAAAATATCTGCAGCTCCAGTCAACACTACAA | |
| | CAGAAGATGAAACGGCACAAATTCCGGCTGAAGCTGT | |
| | CATCGGTTACTTAGATTTAGAAGGGGATTTCGATGTTG | |
| | CTGTTTTGCCATTTTCCAACAGCACAAATAACGGGTTA | |
| | TTGTTTATAAATACTACTATTGCCAGCATTGCTGCTAA | |
| | AGAAGAAGGGGTATCTTTGGATAAAAGAGAGGCTGA | |
| | AGCT | |
| bCBGA1846 | ATGCAATACAAAAAGACTTTGGTTGCCTCTGCTTTGGC | 85 |
| | CGCTACTACATTGGCCGCCTATGCTCCATCTGAGCCTT | |
| | GGTCCACTTTGACTCCAACAGCCACTTACAGCGGTGG | |
| | TGTTACCGACTACGCTTCCACCTTCGGTATTGCCGTTC | |
| | AACCAATCTCCACTACATCCAGCGCATCATCTGCAGC | |
| | CACCACAGCCTCATCTAAGGCCAAGAGAGCTGCTTCC | |
| | CAAATTGGTGATGGTCAAGTCCAAGCTGCTACCACTA | |
| | CTGCTTCTGTCTCTACCAAGAGTACCGCTGCCGCCGTT | |
| | TCTCAGATCGGTGATGGTCAAATCCAAGCTACTACTA | |
| | AGACTACCGCTGCTGCTGTCTCTCAAATTGGTGATGGT | |
| | CAAATTCAAGCTACCACCAAGACTACCTCTGCTAAGA | |
| | CTACCGCCGCTGCCGTTTCTCAAATCAGTGATGGTCAA | |
| | ATCCAAGCTACCACCACTACTTTAGCCCCAAAGAGCA | |
| | CCGCTGCTGCCGTTTCTCAAATCGGTGATGGTCAAGTT | |
| | CAAGCTACCACCACTACTTTAGCCCCAAAGAGCACCG | |
| | CTGCTGCCGTTTCTCAAATCGGTGATGGTCAAGTTCAA | |
| | GCTACTACTAAGACTACCGCTGCTGCTGTCTCTCAAAT | |
| | TGGTGATGGTCAAGTTCAAGCTACCACCAAGACTACT | |
| | GCTGCCGCCGTTTCTCAAATCGGTGATGGTCAAGTTCA | |
| | AGCTACTACCAAGACTACCGCTGCTGCTGTCTCTCAAA | |
| | TCGGTGATGGTCAAGTTCAAGCAACTACCAAAACCAC | |
| | TGCCGCAGCTGTTTCCCAAATTACTGACGGTCAAGTTC | |
| | AAGCCACTACAAAAACCACTCAAGCAGCCAGCCAAGT | |
| | AAGCGATGGCCAAGTCCAAGCTACTACTGCTACTTCC | |
| | GCTTCTGCAGCCGCTACCTCCACTGACCCAGTCGATGC | |
| | TGTCTCCTGTAAGACTTCTGGTACC | |
| bCBGA1847 | ATGAGATTTCCTTCAATTTTTACTGCAGTTTTATTCGC | 86 |
| | AGCATCCTCCGCATTAGCTGCCTATGCTCCATCTGAGC | |
| | CTTGGTCCACTTTGACTCCAACAGCCACTTACAGCGGT | |
| | GGTGTTACCGACTACGCTTCCACCTTCGGTATTGCCGT | |
| | TCAACCAATCTCCACTACATCCAGCGCATCATCTGCAG | |
| | CCACCACAGCCTCATCTAAGGCCAAGAGAGCTGCTTC | |
| | CCAAATTGGTGATGGTCAAGTCCAAGCTGCTACCACT | |
| | ACTGCTTCTGTCTCTACCAAGAGTACCGCTGCCGCCGT | |
| | TTCTCAGATCGGTGATGGTCAAATCCAAGCTACTACTA | |
| | AGACTACCGCTGCTGCTGTCTCTCAAATTGGTGATGGT | |
| | CAAGTTCAAGCTACCACCAAGACTACCTCTGCTAAGA | |
| | CTACCGCCGCTGCCGTTTCTCAAATCAGTGATGGTCAA | |
| | ATCCAAGCTACCACCACTACTTTAGCCCCAAAGAGCA | |
| | CCGCTGCTGCCGTTTCTCAAATCGGTGATGGTCAAGTT | |
| | CAAGCTACCACCACTACTTTAGCCCCAAAGAGCACCG | |
| | CTGCTGCCGTTTCTCAAATCGGTGATGGTCAAGTCCAA | |
| | GCTACTACTAAGACTACCGCTGCTGCTGTCTCTCAAAT | |
| | TGGTGATGGTCAAGTTCAAGCTACCACCAAGACTACT | |
| | GCTGCCGCCGTTTCTCAAATCGGTGATGGTCAAGTTCA | |
| | AGCTACTACCAAGACTACCGCTGCTGCTGTCTCTCAAA | |
| | TCGGTGATGGTCAAGTTCAAGCAACTACCAAAACCAC | |
| | TGCCGCAGCTGTTTCCCAAATTACTGACGGTCAAGTTC | |
| | AAGCCACTACAAAAACCACTCAAGCAGCCAGCCAAGT | |
| | AAGCGATGGCCAAGTCCAAGCTACTACTGCTACTTCC | |
| | GCTTCTGCAGCCGCTACCTCCACTGACCCAGTCGATGC | |
| | TGTCTCCTGTAAGACTTCTGGTACC | |
| bCBGA1851 | ATGCAATACAAAAAGACTTTGGTTGCCTCTGCTTTGGC | 87 |
| | CGCTACTACATTGGCC | |
| bCBGA1852 | ATGCAATACAAAAAGACTTTGGTTGCCTCTGCTTTGGC | 88 |
| | CGCTACTACATTGGCCGCCTATGCTCCATCTGAGCCTT | |
| | GGTCCACTTTGACTCCAACAGCCACTTACAGCGGTGG | |
| | TGTTACCGACTACGCTTCCACCTTCGGTATTGCCGTTC | |
| | AACCAATCTCCACTACATCCAGCGCATCATCTGCAGC | |
| | CACCACAGCCTCATCTAAGGCCAAGAGAGCTGCTTCC | |
| | CAAATTGGTGATGGTCAAGTCCAAGCTGCTACCACTA | |
| | CTGCTTCTGTCTCTACCAAGAGTACCGCTGCCGCCGTT | |
| | TCTCAGATCGGTGATGGTCAAATCCAAGCTACTACTA | |
| | AGACTACCGCTGCTGCTGTCTCTCAAATTGGTGATGGT | |
| | CAAATTCAAGCTACCACCAAGACTACCTCTGCTAAGA | |
| | CTACCGCCGCTGCCGTTTCTCAAATCAGTGATGGTCAA | |
| | ATCCAAGCTACCACCACTACTTTAGCCCCAAAGAGCA | |
| | CCGCTGCTGCCGTTTCTCAAATCGGTGATGGTCAAGTT | |
| | CAAGCTACCACCACTACTTTAGCCCCAAAGAGCACCG | |
| | CTGCTGCCGTTTCTCAAATCGGTGATGGTCAAGTTCAA | |
| | GCTACTACTAAGACTACCGCTGCTGCTGTCTCTCAAAT | |
| | TGGTGATGGTCAAGTTCAAGCTACCACCAAGACTACT | |
| | GCTGCCGCCGTTTCTCAAATCGGTGATGGTCAAGTTCA | |
| | AGCTACTACCAAGACTACCGCTGCTGCTGTCTCTCAAA | |
| | TCGGTGATGGTCAAGTTCAAGCAACTACCAAAACCAC | |
| | TGCCGCAGCTGTTTCCCAAATTACTGACGGTCAAGTTC | |
| | AAGCCACTACAAAAACCACTCAAGCAGCCAGCCAAGT | |
| | AAGCGATGGCCAAGTCCAAGCTACTACTGCTACTTCC | |
| | GCTTCTGCAGCCGCTACCTCCACTGACCCAGTCGATGC | |
| | TGTCTCCTGTAAGACTTCTGGTACCTTGGAGAAAAGA | |
| | GAGGCTGAAGCA | |
| bCBGA1860 | ATGCAATTACATTCACTTATCGCTTCAACTGCGCTCTT | 89 |
| | AATAACGTCAGCTTTGGCT | |
| bCBGA1861 | ATGCGTCTCTCTAACCTAATTGCTTCTGCCTCTCTTTT | 90 |
| | ATCTGCTGCTACTCTTGCT | |
| bCBGA1862 | ATGCGTCTCTCTAACCTAATTGCTTCTGCCTCTCTTTT | 91 |
| | ATCTGCTGCTACTCTTGCTGCTCCCGCTAACCACGAAC | |
| | ACAAGGACAAGCGTGCT | |
| bCBGA1863 | ATGCTTTCGCTTAAAACGTTACTGTGTACGTTGTTGAC | 92 |
| | TGTGTCATCAGTACTCGCT | |
| bCBGA1864 | ATGCTTTCGCTTAAAACGTTACTGTGTACGTTGTTGAC | 93 |
| | TGTGTCATCAGTACTCGCTACCCCAGTCCCTGCA | |
| bCBGA1865 | ATGCTTTCGCTTAAAACGTTACTGTGTACGTTGTTGAC | 94 |
| | TGTGTCATCAGTACTCGCTACCCCAGTCCCTGCAAGAG | |
| | ACCCTTCTTCCATT | |
| bCBGA1873 | ATGAAACTGAAAACTGTAAGATCTGCGGTCCTTTCGT | 95 |
| | CACTCTTTGCATCGCAGGTTCTCGGTCAAACCACTGCC | |
| | CAGACTAATAGTGGCGGACTTGACGTGGTGGGGTTAA | |
| | TTTCTATGGCGATGTCTAAAGGTGAAGAATTATTCACT | |
| | GGTGTTGTCCCAATTTTGGTTGAATTAGATGGTGATGT | |
| | TAATGGTCACAAATTTTCTGTCTCCGGTGAAGGTGAA | |
| | GGTGATGCTACTTACGGTAAATTGACCTTAAAATTGAT | |
| | TTGTACTACTGGTAAATTGCCAGTTCCATGGCCAACCT | |
| | TAGTCACTACTTTAGGTTATGGTTTGCAATGTTTTGCT | |
| | AGATACCCAGATCATATGAAACAACATGACTTTTTCA | |
| | AGTCTGCCATGCCAGAAGGTTATGTTCAAGAAAGAAC | |
| | TATTTTTTTCAAAGATGACGGTAACTACAAGACCAGA | |
| | GCTGAAGTCAAGTTTGAAGGTGATACCTTAGTTAATA | |
| | GAATCGAATTAAAAGGTATTGATTTTAAAGAAGATGG | |
| | TAACATTTTAGGTCACAAATTGGAATACAACTATAAC | |
| | TCTCACAATGTTTACATCACTGCTGACAAACAAAAGA | |
| | ATGGTATCAAAGCTAACTTCAAAATTAGACACAACAT | |
| | TGAAGATGGTGGTGTTCAATTAGCTGACCATTATCAA | |
| | CAAAATACTCCAATTGGTGATGGTCCAGTCTTGTTACC | |
| | AGACAACCATTACTTATCCTATCAATCTGCCTTATCCA | |
| | AAGATCCAAACGAAAAGAGAGACCACATGGTCTTGTT | |
| | AGAATTTGTTACTGCTGCTGGTATTACCCATGGTATGG | |
| | ATGAATTGTACAAAGAAGAAGGTGAACCAAAA | |
| bCBGA1874 | ATGAAACTGAAAACTGTAAGATCTGCGGTCCTTTCGT | 96 |
| | CACTCTTTGCATCGCAGGTTCTCGGTCAAACCACTGCC | |
| | CAGACTAATAGTGGCGGACTTGACGTGGTGGGGTTAA | |
| | TTTCTATGGCGAAGAGGAAAAGAATGTCTAAAGGTGA | |
| | AGAATTATTCACTGGTGTTGTCCCAATTTTGGTTGAAT | |
| | TAGATGGTGATGTTAATGGTCACAAATTTTCTGTCTCC | |
| | GGTGAAGGTGAAGGTGATGCTACTTACGGTAAATTGA | |
| | CCTTAAAATTGATTTGTACTACTGGTAAATTGCCAGTT | |
| | CCATGGCCAACCTTAGTCACTACTTTAGGTTATGGTTT | |
| | GCAATGTTTTGCTAGATACCCAGATCATATGAAACAA | |
| | CATGACTTTTTCAAGTCTGCCATGCCAGAAGGTTATGT | |
| | TCAAGAAAGAACTATTTTTTTCAAAGATGACGGTAAC | |
| | TACAAGACCAGAGCTGAAGTCAAGTTTGAAGGTGATA | |
| | CCTTAGTTAATAGAATCGAATTAAAAGGTATTGATTTT | |
| | AAAGAAGATGGTAACATTTTAGGTCACAAATTGGAAT | |
| | ACAACTATAACTCTCACAATGTTTACATCACTGCTGAC | |
| | AAACAAAAGAATGGTATCAAAGCTAACTTCAAAATTA | |
| | GACACAACATTGAAGATGGTGGTGTTCAATTAGCTGA | |
| | CCATTATCAACAAAATACTCCAATTGGTGATGGTCCA | |
| | GTCTTGTTACCAGACAACCATTACTTATCCTATCAATC | |
| | TGCCTTATCCAAAGATCCAAACGAAAAGAGAGACCAC | |
| | ATGGTCTTGTTAGAATTTGTTACTGCTGCTGGTATTAC | |
| | CCATGGTATGGATGAATTGTACAAAGAAGAAGGTGAA | |
| | CCAAAA | |
| bCBGA1877 | ATGAAACTGAAAACTGTAAGATCTGCGGTCCTTTCGT | 97 |
| | CACTCTTTGCATCGCAGGTTCTCGGTCAAACCACTGCC | |
| | CAGACTAATAGTGGCGGACTTGACGTGGTGGGGTTAA | |
| | TTTCTATGGCGAAAAGAGAAGAAGGTGAACCAAAA | |
| bCBGA1878 | ATGAAACTGAAAACTGTAAGATCTGCGGTCCTTTCGT | 98 |
| | CACTCTTTGCATCGCAGGTTCTCGGTCAAACCACTGCC | |
| | CAGACTAATAGTGGCGGACTTGACGTGGTGGGGTTAA | |
| | TTTCTATGGCGGAAGAAGGTGAACCAAAA | |
| bCBGA1882 | ATGAGAGCTTTCTTGTTTCTCACCGCATGCATCAGTTT | 99 |
| | GCCAGGCGTTTTTGGGGTGGAAGAAGGTGAACCAAAA | |
| bCBGA1884 | ATGAAGTTAGCATACTCCCTCTTGCTTCCATTGGCAGG | 100 |
| | AGTCAGTGCTTCAGTTATCAATTACAAGAGAATGGCA | |
| | ATGGTATCA | |
| bCBGA1885 | ATGAAGTTCGCATACTCCCTCTTGCTTCCATTGGCAGG | 101 |
| | AGTCAGTGCTTCAGTTATCAATTACAAGAGAATGGCA | |
| | ATGGTATCA | |
| bCBGA1886 | ATGAGAGCTTTCTTGTTTCTCACCGCATGCATCAGTTT | 102 |
| | GCCAGGCGTTTTTGGGGTG | |
| bCBGA1887 | ATGAGAGCTTTCTTGTTTCTCACCGCATGCATCAGTTT | 103 |
| | GCCAGGCGTTTTTGGG | |

A high throughput screening process of the CBDAs synthase activity was conducted as follows: Colonies were inoculated into wells of a 96-well deep well plate. Each well contained 400 μl SC liquid medium (6.7 g/L Yeast Nitrogen Base, 1.6 g/L Amino Acid Drop Out mix without leucine, uracil, tryptophan and histidine, 22 g/L glucose, buffered to pH 6.0, supplemented with leucine, tryptophan and histidine). These inoculums were grown for 48 hours at 30° C. and shaken at 300 rpm with a 50 mm shaking diameter. After the 48 hours growth period 40 μl samples of these cultures were inoculated into 360 μl YPD-2400LA (10 g/L yeast extract, 20 g/L peptone, 20 g/L glucose and 240 mg/L olivetolic acid) medium. Then samples were grown for 48 hours at 30° C. and shaken at 300 rpm with a 50 mm shaking diameter and 8 μl of 12000 mg/l OLA dissolved in EtOH is added to the samples. Finally, samples were grown for additional 42 hours and are analyzed for cannabinoids. The CBDA titer in the above described screen ranged from 7 to 285 mg/L. For details, see the Table 12 below. (The titers are also included in Table 10 above.)

TABLE 12

| | | CBDA |
|---|---|---|
| plasmid ID | ORF name | (mg/L) |
| bCBGA1829 | alpha_prepro_signal-d21_CBDAs | 7 |
| bCBGA1843 | BGL2_signal-and-alpha_only_pro_signal- | 9 |
| | d21_CBDAs | |
| bCBGA1845 | SUC2_signal-and-alpha_only_pro_signal- | 9 |
| | d21_CBDAs | |
| bCBGA1840 | AGA2_signal-and-alpha_only_pro_signal- | 10 |
| | d21_CBDAs | |
| bCBGA1836 | BGL2_signal-d21_CBDAs | 11 |
| bCBGA1842 | AMYL_signal-and-alpha_only_pro_signal- | 11 |
| | d21_CBDAs | |
| bCBGA1838 | SUC2_signal-d21_CBDAs | 11 |
| bCBGA1860 | YDR055W_Nterm 19 AA-d21_CBDAs | 12 |
| bCBGA1827 | OST1_signal-and-alpha_only_pro_signal- | 13 |
| | d21_CBDAs | |
| bCBGA1835 | AMYI_signal-d21_CBDAs | 19 |
| bCBGA1863 | YLR300W_Nterm 19 AA-d21_CBDAs | 20 |
| bCBGA1862 | YGR279C_Nterm 31 AA-d21_CBDAs | 24 |
| bCBGA1886 | MEL1m3 signal-noSpacer-d21_CBDAs | 25 |
| bCBGA1826 | OST1_signal-d21_CBDAs | 27 |
| bCBGA1837 | OST1_signal-d21_CBDAs | 41 |
| bCBGA1861 | YGR279C_Nterm 19 AA-d21_CBDAs | 44 |
| bCBGA1839 | PHO5_signal-and-alpha_only_pro_signal- | 44 |
| | d21_CBDAs | |
| bCBGA1877 | YAP_TA57_Kex2_spacer.d21_CBDAs_del_KR_motif | 49 |
| bCBGA1873 | YAP_TA57-yEVenus-spacer-d21_CBDAs | 50 |
| bCBGA1878 | YAP_TA57_Kex2_spacer.d21_CBDAs_del_KEX2_motif | 50 |
| bCBGA1874 | YAP_TA57-Kex2-yEVenus-spacer-d21_CBDAs | 51 |
| bCBGA1864 | YLR300W_Nterm 24 AA-d21_CBDAs | 54 |
| bCBGA1865 | YLR300W_Nterm 30 AA-d21_CBDAs | 58 |
| bCBGA1851 | HSP150 secretion signal-d21_CBDAs | 60 |
| bCBGA1885 | INU1 signal-noSpacer-d21_CBDAs | 60 |
| bCBGA1884 | INU1A signal-noSpacer-d21_CBDAs | 61 |
| bCBGA1882 | MEL1m3 signal-d21_CBDAs | 64 |
| bCBGA1847 | alpha_pre_signal-and- | 94 |
| | HSP150_delta_fragment_wo_signal-d21_CBDAs | |
| bCBGA1875 | YAP_TA57_Kex2_spacer-yEVenus-d21_CBDAs | 101 |
| bCBGA1846 | HSP150_delta_fragment-d21_CBDAs | 102 |
| bCBGA1853 | alpha_pre_signal-and- | 114 |
| | HSP150_delta_fragment_wo_signal-and- | |
| | LEKREAEA-d21_CBDAs | |
| RUNM001233_67.1 | YAP_TA57_Kex2_spacer.d21_CBDAs | 124 |
| bCBGA1852 | HSP150_delta_fragment-and-LEKREAEA- | 126 |
| | d21_CBDAs | |
| bCBGA1831 | YAP_TA57_Kex2_spacer-d21_CBDAs | 133 |
| bCBGA1832 | PHO5_signal-d21_CBDAs | 135 |
| bCBGA1850 | PIR3 secretion signal-d21_CBDAs | 138 |
| bCBGA1883 | K28 viral signal-noSpacer-d21_CBDAs | 149 |
| bCBGA1849 | alpha_pre_signal-and- | 151 |
| | PIR3_delta_fragment_wo_singal-d21_CBDAs | |
| bCBGA1880 | INU1A signal-d21_CBDAs | 181 |
| bCBGA1881 | INU1-d21_CBDAs | 188 |
| bCBGA1855 | alpha_pre_signal-and- | 193 |
| | PIR3_delta_fragment_wo_signal-and-LEKREAEA- | |
| | d21_CBDAs | |
| bCBGA1879 | K28 viral signal-d21_CBDAs | 219 |
| bCBGA1848 | PIR3_delta_fragment-d21_CBDAs | 285 |
| bCB GA1854 | PIR3_delta_fragment-and-LEKREAEA-d21_CBDAs | 285 |

Proteomic analysis using the methods described in Example 4 confirmed that the most active sample had an elevated CBDA synthase concentration in the culture supernatant, most probably due to more effective CBDA synthase secretion. CBDA synthase supernatant concentration was over 15-fold more in strains transformed with the bCBGA1854 plasmid (SEQ ID No: 435), than strains transformed with RUNM001233_67.1. (The corresponding CBDA titers are 285 mg/L and 124 mg/L, respectively.) That is, CBDA productivity and secretion can be increased by improving CBDA synthase secretion.

The supernatant of the culture transformed with the bCBGA1854 plasmid was mixed with 100 mg/L CBGA, and after 48 hours of incubation, 30 mg/L CBDA was detected, proving that there is active CBDA synthase in the supernatant of the yeast culture.

### Example 18—Enhanced CBDA Synthase Secretion II

The secretion of CBDAs was further optimized. A second series of plasmids were constructed containing the Saccharomyces cerevisiae 2μ replication origin, the URA3 gene as an auxotrophic marker and the CBDA synthase gene under the regulation of the bidirectional GAL1/GAL10 promoter. The first 21, or in some cases 28, amino acids of the CBDA synthase in each plasmid was replaced with different yeast secretion signals. The secretion signals included various combinations of the K28 viral secretion signal, the N-terminal 233 amino acid of the PIR3 protein, denoted as PIR3 delta fragment (with or without its predicted native secretion signal), a Kex2p cleavage site and a short spacer motif. The nucleotide sequences and protein sequences of the tested signal sequences are included in Tables 13-14 below, along with the plasmid IDs, the corresponding amino acid sequences, the ORF names and CBDA titer.

TABLE 13

| | | Signal Sequence | SEQ ID | |
|---|---|---|---|---|
| Plasmid ID | ORF name | Amino Acid Sequence | NO: | Titer |
| 0253/asn001-1 | K28_signal- | MESVSSLFNIFSTIMVNYKSLVLALLSVS | 104 | 373 |
| | PIR3_delta_ | NLKYARGAYAPKDPWSTLTPSATYKGG | | |
| | fragment_wo_ | ITDYSSSFGIAIEAVATSASSVASSKAKR | | |
| | signal-Spacer- | AASQIGDGQVQAATTTAAVSKKSTAAA | | |
| | d28_CBDAs_ | VSQITDGQVQAAKSTAAAVSQITDGQV | | |
| | Onofri | QAAKSTAAAVSQITDGQVQAAKSTAAA | | |
| | | VSQITDGQVQAAKSTAAAASQISDGQVQ | | |
| | | ATTSTKAAASQITDGQIQASKTTSGASQ | | |
| | | VSDGQVQATAEVKDANDPVDVVSCNN | | |
| | | NS | | |
| 0253/asn005-1 | K28_signal- | MESVSSLFNIFSTIMVNYKSLVLALLSVS | 105 | 377 |
| | PIR3_delta_ | NLKYARGAYAPKDPWSTLTPSATYKGG | | |
| | fragment_wo_ | ITDYSSSFGIAIEAVATSASSVASSKAKR | | |
| | signal- | AASQIGDGQVQAATTTAAVSKKSTAAA | | |
| | LEKREAEA- | VSQITDGQVQAAKSTAAAVSQITDGQV | | |
| | Spacer-d28_ | QAAKSTAAAVSQITDGQVQAAKSTAAA | | |
| | CBDAs_Onofri | VSQITDGQVQAAKSTAAAASQISDGQVQ | | |
| | | ATTSTKAAASQITDGQIQASKTTSGASQ | | |
| | | VSDGQVQATAEVKDANDPVDVVSCNN | | |
| | | NSTLEKREAE | | |
| 0253/asn037-3 | K28_signal- | MESVSSLFNIFSTIMVNYKSLVLALLSVS | 106 | 366 |
| | PIR3_delta_ | NLKYARGAYAPKDPWSTLTPSATYKGG | | |
| | fragment_wo_ | ITDYSSSFGIAIEAVATSASSVASSKAKR | | |
| | signal- | AASQIGDGQVQAATTTAAVSKKSTAAA | | |
| | LEKREAEA- | VSQITDGQVQAAKSTAAAVSQITDGQV | | |
| | d21_CBDAs_ | QAAKSTAAAVSQITDGQVQAAKSTAAA | | |
| | Onofri | VSQITDGQVQAAKSTAAAASQISDGQVQ | | |
| | | ATTSTKAAASQITDGQIQASKTTSGASQ | | |
| | | VSDGQVQATAEVKDANDPVDVVSCNN | | |
| | | NSTLEKREAEA | | |
| 0253/asn049-1 | K28_signal- | MESVSSLFNIFSTIMVNYKSLVLALLSVS | 107 | 374 |
| | PIR3_delta_ | NLKYARGAYAPKDPWSTLTPSATYKGG | | |
| | fragment_wo_ | ITDYSSSFGIAIEAVATSASSVASSKAKR | | |
| | signal- | AASQIGDGQVQAATTTAAVSKKSTAAA | | |
| | Spacer-d21_ | VSQITDGQVQAAKSTAAAVSQITDGQV | | |
| | CBDAs_Onofri | QAAKSTAAAVSQITDGQVQAAKSTAAA | | |
| | | VSQITDGQVQAAKSTAAAASQISDGQVQ | | |
| | | ATTSTKAAASQITDGQIQASKTTSGASQ | | |
| | | VSDGQVQATAEVKDANDPVDVVSCNN | | |
| | | NSTEEGEPK | | |
| 0253/asn052-3 | PIR3_delta_ | MQYKKPLVVSALAATSLAAYAPKDPWS | 108 | 298 |
| | fragment- | TLTPSATYKGGITDYSSSFGIAIEAVATSA | | |
| | Spacer- | SSVASSKAKRAASQIGDGQVQAATTTA | | |
| | d21_CBDAs_ | AVSKKSTAAAVSQITDGQVQAAKSTAA | | |
| | Onofri | AVSQITDGQVQAAKSTAAAVSQITDGQ | | |
| | | VQAAKSTAAAVSQITDGQVQAAKSTAA | | |
| | | AASQISDGQVQATTSTKAAASQITDGQI | | |
| | | QASKTTSGASQVSDGQVQATAEVKDAN | | |
| | | DPVDVVSCNNNSTEEGEPK | | |
| 0253/asn053-2 | K28_signal- | MESVSSLFNIFSTIMVNYKSLVLALLSVS | 109 | 381 |
| | PIR3_delta_ | NLKYARGAYAPKDPWSTLTPSATYKGG | | |
| | fragment_ | ITDYSSSFGIAIEAVATSASSVASSKAKR | | |
| | wo_signal- | AASQIGDGQVQAATTTAAVSKKSTAAA | | |
| | LEKREAEA- | VSQITDGQVQAAKSTAAAVSQITDGQV | | |
| | Spacer- | QAAKSTAAAVSQITDGQVQAAKSTAAA | | |
| | d21_CBDAs_ | VSQITDGQVQAAKSTAAAASQISDGQVQ | | |
| | Onofri | ATTSTKAAASQITDGQIQASKTTSGASQ | | |
| | | VSDGQVQATAEVKDANDPVDVVSCNN | | |
| | | NSTLEKREAEAEEGEPK | | |
| 0253/asn056-1 | PIR3_delta_ | MESVSSLFNIFSTIMVNYKSLVLALLSVS | 110 | 298 |
| | fragment- | NLKYARGAYAPKDPWSTLTPSATYKGG | | |
| | LEKREAEA- | ITDYSSSFGIAIEAVATSASSVASSKAKR | | |
| | Spacer- | AASQIGDGQVQAATTTAAVSKKSTAAA | | |
| | d21_CBDAs_ | VSQITDGQVQAAKSTAAAVSQITDGQV | | |
| | Onofri | QAAKSTAAAVSQITDGQVQAAKSTAAA | | |
| | | VSQITDGQVQAAKSTAAAASQISDGQVQ | | |
| | | ATTSTKAAASQITDGQIQASKTTSGASQ | | |
| | | VSDGQVQATAEVKDANDPVDVVSCNN | | |
| | | NS | | |

TABLE 14

| Plasmid ID | Signal Sequence Nucleotide Sequence | SEQ ID NO: |
|---|---|---|
| 0253/asn001-1 | ATGGAATCTGTTTCTTCTTTGTTCAACATCTTCTCTAC | 111 |
| | TATCATGGTTAACTACAAGTCTTTGGTTTTGGCTTTG | |
| | TTGTCTGTTTCTAACTTGAAGTACGCTAGAGGTGCCT | |
| | ATGCTCCAAAGGACCCGTGGTCCACTTTAACTCCAT | |
| | CAGCTACTTACAAGGGTGGTATAACAGATTACTCTT | |
| | CGAGTTTCGGTATTGCTATTGAAGCCGTGGCTACCA | |
| | GTGCTTCCTCCGTCGCCTCATCTAAAGCAAAGAGAG | |
| | CCGCCTCTCAGATAGGTGATGGTCAAGTACAGGCTG | |
| | CCACTACTACTGCTGCTGTTTCTAAGAAATCCACCGC | |
| | TGCTGCTGTTTCTCAAATAACTGACGGTCAAGTTCAA | |
| | GCTGCTAAGTCTACTGCCGCTGCTGTTTCCCAAATAA | |
| | CTGACGGTCAAGTTCAAGCTGCTAAGTCTACTGCCG | |
| | CTGCCGTTTCTCAAATAACTGACGGTCAAGTTCAAG | |
| | CTGCTAAGTCTACTGCCGCTGCCGTTTCTCAAATAAC | |
| | TGATGGTCAAGTTCAAGCTGCCAAGTCTACTGCTGC | |
| | CGCTGCCTCTCAGATTTCTGACGGCCAAGTTCAGGC | |
| | CACTACCTCTACTAAGGCTGCTGCATCCCAAATTAC | |
| | AGATGGGCAGATACAAGCATCTAAAACTACCAGTGG | |
| | CGCTAGTCAAGTAAGTGATGGCCAAGTCCAGGCTAC | |
| | TGCTGAAGTGAAAGACGCTAACGATCCAGTCGATGT | |
| | TGTTTCCTGTAATAACAATAGT | |
| 0253/asn005-1 | ATGGAATCTGTTTCTTCTTTGTTCAACATCTTCTCTAC | 112 |
| | TATCATGGTTAACTACAAGTCTTTGGTTTTGGCTTTG | |
| | TTGTCTGTTTCTAACTTGAAGTACGCTAGAGGTGCCT | |
| | ATGCTCCAAAGGACCCGTGGTCCACTTTAACTCCAT | |
| | CAGCTACTTACAAGGGTGGTATAACAGATTACTCTT | |
| | CGAGTTTCGGTATTGCTATTGAAGCCGTGGCTACCA | |
| | GTGCTTCCTCCGTCGCCTCATCTAAAGCAAAGAGAG | |
| | CCGCCTCTCAGATAGGTGATGGTCAAGTACAGGCTG | |
| | CCACTACTACTGCTGCTGTTTCTAAGAAATCCACCGC | |
| | TGCTGCTGTTTCTCAAATAACTGACGGTCAAGTTCAA | |
| | GCTGCTAAGTCTACTGCCGCTGCTGTTTCCCAAATAA | |
| | CTGACGGTCAAGTTCAAGCTGCTAAGTCTACTGCCG | |
| | CTGCCGTTTCTCAAATAACTGACGGTCAAGTTCAAG | |
| | CTGCTAAGTCTACTGCCGCTGCCGTTTCTCAAATAAC | |
| | TGATGGTCAAGTTCAAGCTGCCAAGTCTACTGCTGC | |
| | CGCTGCCTCTCAGATTTCTGACGGCCAAGTTCAGGC | |
| | CACTACCTCTACTAAGGCTGCTGCATCCCAAATTAC | |
| | AGATGGGCAGATACAAGCATCTAAAACTACCAGTGG | |
| | CGCTAGTCAAGTAAGTGATGGCCAAGTCCAGGCTAC | |
| | TGCTGAAGTGAAAGACGCTAACGATCCAGTCGATGT | |
| | TGTTTCCTGTAATAACAATAGTACCTTGGAGAAAAG | |
| | AGAGGCTGAA | |
| 0253/asn037-3 | ATGGAATCTGTTTCTTCTTTGTTCAACATCTTCTCTAC | 113 |
| | TATCATGGTTAACTACAAGTCTTTGGTTTTGGCTTTG | |
| | TTGTCTGTTTCTAACTTGAAGTACGCTAGAGGTGCCT | |
| | ATGCTCCAAAGGACCCGTGGTCCACTTTAACTCCAT | |
| | CAGCTACTTACAAGGGTGGTATAACAGATTACTCTT | |
| | CGAGTTTCGGTATTGCTATTGAAGCCGTGGCTACCA | |
| | GTGCTTCCTCCGTCGCCTCATCTAAAGCAAAGAGAG | |
| | CCGCCTCTCAGATAGGTGATGGTCAAGTACAGGCTG | |
| | CCACTACTACTGCTGCTGTTTCTAAGAAATCCACCGC | |
| | TGCTGCTGTTTCTCAAATAACTGACGGTCAAGTTCAA | |
| | GCTGCTAAGTCTACTGCCGCTGCTGTTTCCCAAATAA | |
| | CTGACGGTCAAGTTCAAGCTGCTAAGTCTACTGCCG | |
| | CTGCCGTTTCTCAAATAACTGACGGTCAAGTTCAAG | |
| | CTGCTAAGTCTACTGCCGCTGCCGTTTCTCAAATAAC | |
| | TGATGGTCAAGTTCAAGCTGCCAAGTCTACTGCTGC | |
| | CGCTGCCTCTCAGATTTCTGACGGCCAAGTTCAGGC | |
| | CACTACCTCTACTAAGGCTGCTGCATCCCAAATTAC | |
| | AGATGGGCAGATACAAGCATCTAAAACTACCAGTGG | |
| | CGCTAGTCAAGTAAGTGATGGCCAAGTCCAGGCTAC | |
| | TGCTGAAGTGAAAGACGCTAACGATCCAGTCGATGT | |
| | TGTTTCCTGTAATAACAATAGTACCTTGGAGAAAAG | |
| | AGAGGCTGAAGCA | |
| 0253/asn049-1 | ATGGAATCTGTTTCTTCTTTGTTCAACATCTTCTCTAC | 114 |
| | TATCATGGTTAACTACAAGTCTTTGGTTTTGGCTTTG | |
| | TTGTCTGTTTCTAACTTGAAGTACGCTAGAGGTGCCT | |
| | ATGCTCCAAAGGACCCGTGGTCCACTTTAACTCCAT | |
| | CAGCTACTTACAAGGGTGGTATAACAGATTACTCTT | |
| | CGAGTTTCGGTATTGCTATTGAAGCCGTGGCTACCA | |
| | GTGCTTCCTCCGTCGCCTCATCTAAAGCAAAGAGAG | |
| | CCGCCTCTCAGATAGGTGATGGTCAAGTACAGGCTG | |
| | CCACTACTACTGCTGCTGTTTCTAAGAAATCCACCGC | |
| | TGCTGCTGTTTCTCAAATAACTGACGGTCAAGTTCAA | |
| | GCTGCTAAGTCTACTGCCGCTGCTGTTTCCCAAATAA | |
| | CTGACGGTCAAGTTCAAGCTGCTAAGTCTACTGCCG | |
| | CTGCCGTTTCTCAAATAACTGACGGTCAAGTTCAAG | |
| | CTGCTAAGTCTACTGCCGCTGCCGTTTCTCAAATAAC | |
| | TGATGGTCAAGTTCAAGCTGCCAAGTCTACTGCTGC | |
| | CGCTGCCTCTCAGATTTCTGACGGCCAAGTTCAGGC | |
| | CACTACCTCTACTAAGGCTGCTGCATCCCAAATTAC | |
| | AGATGGGCAGATACAAGCATCTAAAACTACCAGTGG | |
| | CGCTAGTCAAGTAAGTGATGGCCAAGTCCAGGCTAC | |
| | TGCTGAAGTGAAAGACGCTAACGATCCAGTCGATGT | |
| | TGTTTCCTGTAATAACAATAGTACCGAAGAAGGTGA | |
| | ACCAAAA | |
| 0253/asn052-3 | ATGCAATATAAAAAGCCATTAGTCGTCTCCGCTTTA | 115 |
| | GCTGCTACATCTTTAGCTGCCTATGCTCCAAAGGACC | |
| | CGTGGTCCACTTTAACTCCATCAGCTACTTACAAGG | |
| | GTGGTATAACAGATTACTCTTCGAGTTTCGGTATTGC | |
| | TATTGAAGCCGTGGCTACCAGTGCTTCCTCCGTCGCC | |
| | TCATCTAAAGCAAAGAGAGCCGCCTCTCAGATAGGT | |
| | GATGGTCAAGTACAGGCTGCCACTACTACTGCTGCT | |
| | GTTTCTAAGAAATCCACCGCTGCTGCTGTTTCTCAAA | |
| | TAACTGACGGTCAAGTTCAAGCTGCTAAGTCTACTG | |
| | CCGCTGCTGTTTCCCAAATAACTGACGGTCAAGTTC | |
| | AAGCTGCTAAGTCTACTGCCGCTGCCGTTTCTCAAAT | |
| | AACTGACGGTCAAGTTCAAGCTGCTAAGTCTACTGC | |
| | CGCTGCCGTTTCTCAAATAACTGATGGTCAAGTTCA | |
| | AGCTGCCAAGTCTACTGCTGCCGCTGCCTCTCAGATT | |
| | TCTGACGGCCAAGTTCAGGCCACTACCTCTACTAAG | |
| | GCTGCTGCATCCCAAATTACAGATGGGCAGATACAA | |
| | GCATCTAAAACTACCAGTGGCGCTAGTCAAGTAAGT | |
| | GATGGCCAAGTCCAGGCTACTGCTGAAGTGAAAGAC | |
| | GCTAACGATCCAGTCGATGTTGTTTCCTGTAATAACA | |
| | ATAGTACCGAAGAAGGTGAACCAAAA | |
| 0253/asn053-2 | ATGGAATCTGTTTCTTCTTTGTTCAACATCTTCTCTAC | 116 |
| | TATCATGGTTAACTACAAGTCTTTGGTTTTGGCTTTG | |
| | TTGTCTGTTTCTAACTTGAAGTACGCTAGAGGTGCCT | |
| | ATGCTCCAAAGGACCCGTGGTCCACTTTAACTCCAT | |
| | CAGCTACTTACAAGGGTGGTATAACAGATTACTCTT | |
| | CGAGTTTCGGTATTGCTATTGAAGCCGTGGCTACCA | |
| | GTGCTTCCTCCGTCGCCTCATCTAAAGCAAAGAGAG | |
| | CCGCCTCTCAGATAGGTGATGGTCAAGTACAGGCTG | |
| | CCACTACTACTGCTGCTGTTTCTAAGAAATCCACCGC | |
| | TGCTGCTGTTTCTCAAATAACTGACGGTCAAGTTCAA | |
| | GCTGCTAAGTCTACTGCCGCTGCTGTTTCCCAAATAA | |
| | CTGACGGTCAAGTTCAAGCTGCTAAGTCTACTGCCG | |
| | CTGCCGTTTCTCAAATAACTGACGGTCAAGTTCAAG | |
| | CTGCTAAGTCTACTGCCGCTGCCGTTTCTCAAATAAC | |
| | TGATGGTCAAGTTCAAGCTGCCAAGTCTACTGCTGC | |
| | CGCTGCCTCTCAGATTTCTGACGGCCAAGTTCAGGC | |
| | CACTACCTCTACTAAGGCTGCTGCATCCCAAATTAC | |
| | AGATGGGCAGATACAAGCATCTAAAACTACCAGTGG | |
| | CGCTAGTCAAGTAAGTGATGGCCAAGTCCAGGCTAC | |
| | TGCTGAAGTGAAAGACGCTAACGATCCAGTCGATGT | |
| | TGTTTCCTGTAATAACAATAGTACCTTGGAGAAAAG | |
| | AGAGGCTGAAGCAGAAGAAGGTGAACCAAAA | |
| 0253/asn056-1 | ATGCAATATAAAAAGCCATTAGTCGTCTCCGCTTTA | 117 |
| | GCTGCTACATCTTTAGCTGCCTATGCTCCAAAGGACC | |
| | CGTGGTCCACTTTAACTCCATCAGCTACTTACAAGG | |
| | GTGGTATAACAGATTACTCTTCGAGTTTCGGTATTGC | |
| | TATTGAAGCCGTGGCTACCAGTGCTTCCTCCGTCGCC | |
| | TCATCTAAAGCAAAGAGAGCCGCCTCTCAGATAGGT | |
| | GATGGTCAAGTACAGGCTGCCACTACTACTGCTGCT | |
| | GTTTCTAAGAAATCCACCGCTGCTGCTGTTTCTCAAA | |
| | TAACTGACGGTCAAGTTCAAGCTGCTAAGTCTACTG | |
| | CCGCTGCTGTTTCCCAAATAACTGACGGTCAAGTTC | |
| | AAGCTGCTAAGTCTACTGCCGCTGCCGTTTCTCAAAT | |
| | AACTGACGGTCAAGTTCAAGCTGCTAAGTCTACTGC | |
| | CGCTGCCGTTTCTCAAATAACTGATGGTCAAGTTCA | |
| | AGCTGCCAAGTCTACTGCTGCCGCTGCCTCTCAGATT | |
| | TCTGACGGCCAAGTTCAGGCCACTACCTCTACTAAG | |
| | GCTGCTGCATCCCAAATTACAGATGGGCAGATACAA | |
| | GCATCTAAAACTACCAGTGGCGCTAGTCAAGTAAGT | |
| | GATGGCCAAGTCCAGGCTACTGCTGAAGTGAAAGAC | |
| | GCTAACGATCCAGTCGATGTTGTTTCCTGTAATAACA | |
| | ATAGTACCTTGGAGAAAAGAGAGGCTGAAGCAGAA | |
| | GAAGGTGAACCAAAA | |

This set of plasmids were each individually transformed into the yCBGA0314 strain and their CBDA productivity was assayed in a high throughput screening process optimized for CBDAs synthase activity as described in Example 17 above. The CBDA titer in the above described screen reached up to 381 mg/L. Samples titers for several transformed strains are included in Table 15 below. (The titers are also included in Table 13 above.)

TABLE 15

| | | CBDA |
|---|---|---|
| Plasmid ID | ORF name | (mg/L) |
| 0253/asn053-2 | K28_signal-PIR3_delta_fragment_wo_signal- | 381 |
| | LEKREAEA-Spacer-d2l_CB DAs_Onofri | |
| 0253/asn005-1 | K28_signal-PIR3_delta_fragment_wo_signal- | 377 |
| | LEKREAEA-Spacer-d28_CBDAs_Onofri | |
| 0253/asn049-1 | K28_signal-PIR3_delta_fragment_wo_signal- | 374 |
| | Spacer-d21_CBDAs_Onofri | |
| 0253/asn001-1 | K28_signal-PIR3_delta_fragment_wo_signal- | 373 |
| | Spacer-d28_CBDAs_Onofri | |
| 0253/asn037-3 | K28_signal-PIR3_delta_fragment_wo_signal- | 366 |
| | LEKREAEA-d21_CBDAs_Onofri | |
| 0253/asn052-3 | PIR3_delta_fragment-Spacer- | 298 |
| | d21_CBDAs_Onofri | |
| 0253/asn056-1 | PIR3_delta_fragment-LEKREAEA-Spacer- | 298 |
| | d21_CBDAs_Onofri | |

### Example 19—the yCBGA0508 and yCBGA0509 Strains: VPS10 Deletion

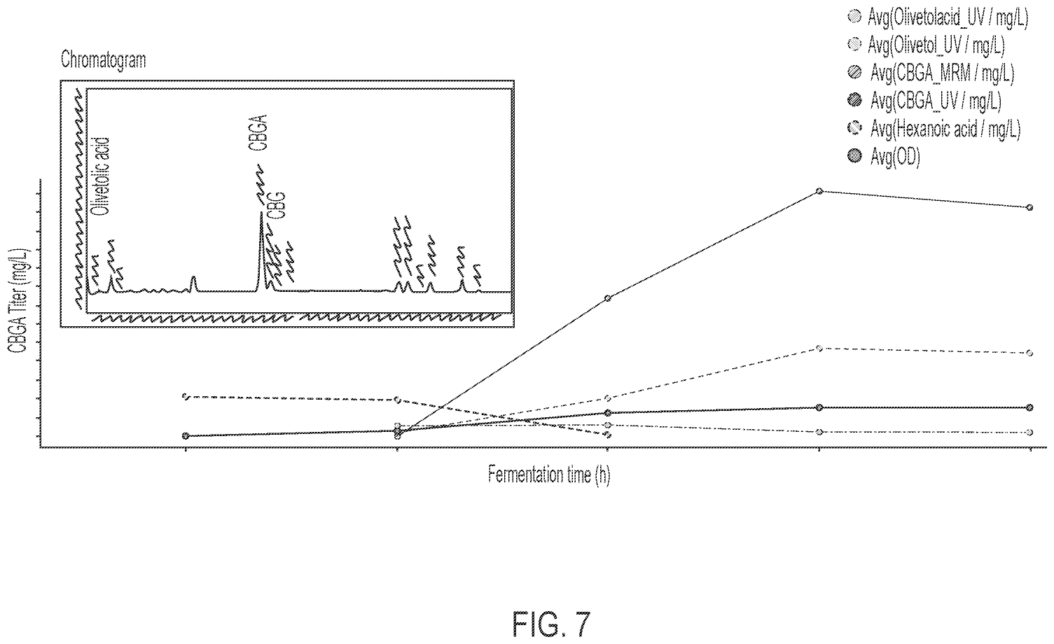

CBDA productivity was further improved by deletions in the VPS10 gene of the host. Two deletions were made: yCBGA0508 strain was constructed by deleting the whole coding sequence of the VPS locus of the yCBGA0314 strain. yCBGA0509 strain was made by deleting the majority of the 5′ half of the coding sequence of the VPS locus of the yCBGA0314 strain. (FIG. 7).

The bCBGA1854 plasmid was transformed into the strains yCBGA0314, yCBGA0508 and yCBGA0509 and their CBDA producing capacity was assayed in an improved CBDA screening process.

Improved high throughput screening process of the CBDAs synthase activity.

Colonies were inoculated into wells of a 96-well deep well plate. Each well contained 400 μl SC liquid medium (6.7 g/L Yeast Nitrogen Base, 1.6 g/L Amino Acid Drop Out mix without leucine, uracil, tryptophan and histidine, 22 g/L glucose, buffered to pH 6.0, supplemented with leucine, tryptophan and histidine). These inoculums were grown for 48 hours at 30° C. and shaken at 300 rpm with a 50 mm shaking diameter. After the 48 hours growth period 40 μl samples of these cultures were inoculated into 360 μl YPD-pH6-2400LA (10 g/L yeast extract (Ohly), 20 g/L soy peptone (Organotechnie), 20 g/L glucose, buffered to pH6 with sodium phosphate, 240 mg/L olivetolic acid) medium. Then samples were grown for 48 hours at 30° C. and shaken at 300 rpm with a 50 mm shaking diameter and 8 μl of 12000 mg/l OLA dissolved in EtOH is added to the samples. Finally, samples were grown for additional 48 hours and are analyzed for cannabinoids.

In this screen, the following CBDA titers were detected: see Table 16 below. Both types of deletion in the VPS10 gene had a positive effect on CBDA productivity.

TABLE 16

| Total olivetolic | Parental | | CBDA |
|---|---|---|---|
| acid (mg/L) | strain | Plasmid | titer (mg/L) |
| 480 | yCBGA0314 | bCBGA1854 | 232 |
| 480 | yCBGA0508 | bCBGA1854 | 296 |
| 480 | yCBGA0509 | bCBGA1854 | 286 |

### Example 20—the yCBGA0499 Strain: HXA-to-CBDA Pathway

CBDA production from a hexanoic acid substrate was tested using the yCBGA0499 strain, which contains the bCBAG1854 plasmid described in Example 19 above.

For the strain construction the parental strain was the yCBGA0314 strain. The PKS and OAC genes under the regulation of the bidirectional GAL1/GAL10 promoter and the AAE1 gene under the regulation of the STE5 promoter were inserted into the YDR508C, YRL020C and FOX1 loci replacing the native ORFs of the yCBGA0314 strain. The PKS and OAC genes (under the regulation of the bidirectional GAL1/GAL10 promoter) and the AAE1 gene (under the regulation of the STE5 promoter) were inserted into the YDR508C locus. A second copy of the PKS and OAC genes (under the regulation of the sa promoter) and the AAE1 gene (under the regulation of the STE5 promoter) were inserted into the YRL020C locus. A third copy of the PKS and OAC genes (under the regulation of the bidirectional GAL1/GAL10 promoter) and the AAE1 gene (under the regulation of the STE5 promoter) were inserted into the FOX1 locus. A large number of isolates from this transformation were screened and an isolate with high CBGA productivity was identified as yCBGA0499.

The yCBGA0499 with bCBGA1854 plasmid was assayed for CBDA productivity using the following high throughput screening process using hexanoic acid for CBDA production. Colonies were inoculated into wells of a 96-well deep well plate. Each well contained 400 μl Synthetic Complete liquid medium (6.7 g/L Yeast Nitrogen Base, 1.6 g/L Amino Acid Drop Out mix without leucine, uracil, tryptophan and histidine, 22 g/L glucose, buffered to pH 6.0, supplemented with leucine, tryptophan and histidine). These inoculums were grown for 48 hours at 30° C. and shaken at 300 rpm with a 50 mm shaking diameter. After the 48 hours growth period, 40 μl samples of these cultures were inoculated into 360 μl YPD-pH6-HXA (10 g/L yeast extract (Ohly), 20 g/L soy peptone (Organotechnie), 20 g/L glucose, buffered to pH6 with sodium phosphate, 100 mg/L hexanoic acid) medium. Then samples were grown for 48 hours at 30° C. and shaken at 300 rpm with a 50 mm shaking diameter and 40 μg hexanoic acid dissolved in 8 μl ethanol is added to the samples. Finally, the samples were grown for additional 48 hours and were analyzed for cannabinoids.

Using this screening method, the yCBGA0499 strain produced 51 mg/L CBDA in one single biological process using hexanoic acid as substrate.

### Example 21—the yCBGA0519 Strain: HXA-to-CBDA Pathway II

To produce a stable prototrophic strain for CBDA production, the yCBGA0519 strain was constructed by inserting a modified CBDA synthase gene, denoted as PIR3-CBDAs, into the yCBGA0513 strain:

For the strain construction, the parental strain was the prototrophic CBGA producer yCBGA0513 strain. The gene coding for the PIR3-CBDAs under the regulation of the bidirectional GAL1/GAL10 promoter was integrated into the YKL140W locus by replacing the native YKL140W ORF of the yCBGA0513 strain. The amino acid sequence of PIR3-CBDAs consists of the N-terminal 233 amino acid of the PIR3 protein, the LEKREAEA peptide motif (SEQ ID NO: 118) as a Kex2p cleavage site, and the CBDA synthase coding sequence lacking its first 21 amino acids. The nucleotide sequence of PIR3-CBDAs is SEQ ID NO: 301. The amino acid sequence of PIR3-CBDAs is SEQ ID NO: 302.

The yCBGA0519 strain was assayed for CBDA productivity using a modified high throughput screening process using hexanoic acid for CBDA production described in Example 20 above. The yCBGA0519 strain produced 144 mg/L CBDA in one single biological process using hexanoic acid as substrate.

### Example 22—Enhanced THCA Synthase Secretion

The secretion of THCAs was optimized to be able produce large amount of THCA in a single process using yeast cells. The wild type sequences for the Saccharomyces cerevisiae THCA synthase amino acid and nucleotide sequences are included in Table 17:

TABLE 17

| | | SEO |
|---|---|---|
| Description | | ID |
| of sequence | Sequence | NO: |
| WT THCA | ATGAACTGCTCCGCATTCTCTTTCTGGTTCGTCTGTAAAATAA | 119 |
| synthase nt | TCTTCTTCTTCTTGTCCTTCAACATCCAAATCTCCATCGCAAA | |
| | TCCACAAGAAAACTTTTTGAAGTGTTTCTCCGAATACATCCC | |
| | AAACAACCCTGCTAACCCAAAGTTTATATATACTCAACATGA | |
| | TCAATTGTACATGTCCGTTTTGAACAGTACCATCCAAAATTTG | |
| | AGATTCACTTCTGACACTACACCAAAACCTTTAGTCATTGTTA | |
| | CACCTTCCAATGTTAGTCACATTCAAGCTTCTATATTGTGCTC | |
| | TAAGAAAGTAGGTTTGCAAATCAGAACTAGATCAGGTGGTCA | |
| | TGATGCAGAAGGCATGTCTTACATCTCACAAGTTCCATTCGTT | |
| | GTAGTCGATTTGAGAAATATGCATTCCATAAAGATCGACGTT | |
| | CACAGTCAAACAGCATGGGTAGAAGCAGGTGCCACCTTGGG | |
| | TGAAGTTTACTACTGGATCAACGAAAAGAATGAAAACTTTTC | |
| | TTTCCCTGGTGGTTACTGTCCAACAGTAGGTGTCGGTGGTCAC | |
| | TTTTCTGGTGGTGGTTATGGTGCATTGATGAGAAACTACGGTT | |
| | TAGCTGCAGATAATATTATAGACGCCCATTTGGTTAACGTAG | |
| | ATGGTAAAGTTTTGGACAGAAAGTCTATGGGTGAAGATTTGT | |
| | TTTGGGCCATAAGAGGTGGTGGTGGTGAAAATTTCGGTATCA | |
| | TTGCCGCTTGGAAAATTAAGTTAGTCGCTGTTCCTTCCAAAA | |
| | GTACTATTTTCTCTGTCAAAAAGAACATGGAAATCCACGGTT | |
| | TGGTTAAGTTGTTTAATAAGTGGCAAAACATCGCTTACAAGT | |
| | ACGATAAGGACTTGGTTTTGATGACCCATTTCATCACTAAAA | |
| | ATATTACAGATAACCATGGTAAAAATAAGACCACTGTTCACG | |
| | GTTATTTTTCTTCAATTTTCCATGGTGGTGTAGATTCTTTGGTT | |
| | GATTTGATGAATAAGTCATTCCCAGAATTGGGTATTAAAAAG | |
| | ACAGATTGCAAGGAATTTTCTTGGATAGACACAACCATCTTC | |
| | TATTCAGGTGTTGTAAACTTCAACACCGCTAACTTCAAAAAG | |
| | GAAATCTTGTTGGATAGATCCGCTGGTAAAAAGACCGCTTTT | |
| | TCTATTAAATTGGACTACGTTAAGAAACCAATCCCTGAAACT | |
| | GCAATGGTCAAGATATTGGAAAAGTTGTACGAAGAAGATGT | |
| | AGGTGTCGGCATGTACGTTTTGTATCCATACGGTGGTATTATG | |
| | GAAGAAATATCTGAATCAGCCATACCATTTCCTCACAGAGCT | |
| | GGTATCATGTATGAATTATGGTACACAGCCTCATGGGAAAAG | |
| | CAAGAAGATAACGAAAAGCATATCAACTGGGTCAGATCCGT | |
| | TTACAACTTCACTACACCTTACGTTAGTCAAAACCCAAGATT | |
| | GGCATATTTGAACTACAGAGATTTGGACTTAGGTAAAACTAA | |
| | CCCTGAATCTCCAAATAACTATACACAAGCAAGAATTTGGGG | |
| | TGAAAAGTACTTTGGTAAAAATTTCAACAGATTAGTTAAAGT | |
| | AAAGACTAAAGCCGACCCTAACAACTTTTTCAGAAACGAACA | |
| | ATCCATCCCACCTTTGCCACCTCACCACCACTAA | |
| WT THCA | MNCSAFSFWFVCKIIFFFLSFNIQISIANPQENFLKCFSEYIPNNPA | 120 |
| synthase aa | NPKFIYTQHDQLYMSVLNSTIQNLRFTSDTTPKPLVIVTPSNVSHI | |
| | QASILCSKKVGLQIRTRSGGHDAEGMSYISQVPFVVVDLRNMHS | |
| | IKIDVHSQTAWVEAGATLGEVYYWINEKNENFSFPGGYCPTVG | |
| | VGGHFSGGGYGALMRNYGLAADNIIDAHLVNVDGKVLDRKSM | |
| | GEDLFWAIRGGGGENFGIIAAWKIKLVAVPSKSTIFSVKKNMEIH | |
| | GLVKLFNKWQNIAYKYDKDLVLMTHFITKNITDNHGKNKTTVH | |
| | GYFSSIFHGGVDSLVDLMNKSFPELGIKKTDCKEFSWIDTTIFYS | |
| | GVVNFNTANFKKEILLDRSAGKKTAFSIKLDYVKKPIPETAMVK | |
| | ILEKLYEEDVGVGMYVLYPYGGIMEEISESAIPFPHRAGIMYEL | |
| | WYTASWEKQEDNEKHINWVRSVYNFTTPYVSQNPRLAYLNYR | |
| | DLDLGKTNPESPNNYTQARIWGEKYFGKNFNRLVKVKTKADPN | |
| | NFFRNEQSIPPLPPHHH | |

A series of plasmids were constructed containing the Saccharomyces cerevisiae replication origin, the URA3 gene as an auxotrophic marker and the THCA synthase gene under the regulation of the bidirectional GAL1/GAL10 promoter. In each plasmid various yeast secretion signals were used, in some cases the first 28 amino acids of the THCA synthase protein predicted to be a plant secretion signal was removed. The nucleotide sequences and protein sequences of the tested signal sequences are included in Tables 18-19 below, along with the plasmid IDs, the corresponding amino acid sequences, the ORF names and THCA titer.

TABLE 18

| | | Signal Sequence Amino | SEQ ID | |
|---|---|---|---|---|
| Plasmid ID | ORF name | Acid Sequence | NO: | Titer |
| 0279/asn004-1 | PH05_signal- | MFKSVVYSILAASLANA | 121 | 36 |
| | THCAs | | | |
| 0279/asn006-2 | HSP150_delta_ | MQYKKTLVASALAATTLAAYAPSEPWS | 122 | 13 |
| | fragment-THCAs | TLTPTATYSGGVTDYASTFGIAVQPISTT | | |
| | | SSASSAATTASSKAKRAASQIGDGQVQA | | |
| | | ATTTASVSTKSTAAAVSQIGDGQIQATT | | |
| | | KTTAAAVSQIGDGQIQATTKTTSANTTA | | |
| | | AAVSQISDGQIQATTTTLAPKSTAAAVS | | |
| | | QIGDGQVQATTTTLAPKSTAAAVSQIGD | | |
| | | GQVQATTKTTAAAVSQIGDGQVQATTK | | |
| | | TTAAAVSQIGDGQVQATTKTTAAAVSQI | | |
| | | GDGQVQATTKTTAAAVSQITDGQVQAT | | |
| | | TKTTQAASQVSDGQVQATTATSASAAA | | |
| | | TSTDPVDAVSCKTSGT | | |
| 0279/asn009-3 | PIR3_delta_frag- | MQYKKPLVVSALAATSLAAYAPKDPWS | 123 | 501 |
| | ment-d28_THCAs | TLTPSATYKGGITDYSSSFGIAIEAVATSA | | |
| | | SSVASSKAKRAAYQIGDGQVQAATTTA | | |
| | | AVSKKSTAAAVSQITDGQVQAAKSTAA | | |
| | | AVSQITDGQVQAAKSTAAAVSQITDGQ | | |
| | | VQAAKSTAAAVSQITDGQVQAAKSTAA | | |
| | | AASQISDDQVQATTYTKAAASQITDGQI | | |
| | | QASKTTSGASQVSDGQVQATAEVKDAN | | |
| | | DPVDVVSCNNNST | | |
| 0279/asn010-1 | PIR3_delta_frag- | MQYKKPLVVSALAATSLAAYAPKDPWS | 124 | 4 |
| | ment-THCAs | TLTPSATYKGGITDYSSSFGIAIEAVATSA | | |
| | | SSVASSKAKRAASQIGDGQVQAATTTA | | |
| | | AVSKKSTAAAVSQITDGQVQAAKSTAA | | |
| | | AVSQITDGQVQAAKSTAAAVSQITDGQ | | |
| | | VQAAKSTAAAVSQITDGQVQAAKSTAA | | |
| | | AASQISDGQVQATTSTKAAASQITDGQI | | |
| | | QASKTTSGASQVSDGQVQATAEVKDAN | | |
| | | DPVDVVSCNNNST | | |
| 0279/asn012-3 | alpha_pre_signal- | MRFPSIFTAVLFAASSALAAYAPKDPWS | 125 | 10 |
| | and- | TLTPSATYKGGITDYSSSFGIAIEAVATSA | | |
| | PIR3_delta_frag- | SSVASSKAKRAASQIGDGQVQAATTTA | | |
| | ment_wo_singal- | AVSKKSTAAAVSQITDGQVQAAKSTAA | | |
| | THCAs | AVSQITDGQVQAAKSTAAAVSQITDGQ | | |
| | | VQAAKSTAAAVSQITDGQVQAAKSTAA | | |
| | | AASQISDGQVQATTSTKAAASQITDGQI | | |
| | | QASKTTSGASQVSDGQVQATAEVKDAN | | |
| | | DPVDVVSCNNNST | | |
| 0279/asn014-2 | PIR3 secretion | MQYKKPLVVSALAATSLA | 126 | 122 |
| | signal-THCAs | | | |
| 0279/asn015-2 | HSP150_delta_ | MQYKKTLVASALAATTLAAYAPSEPWS | 127 | 500 |
| | fragment-and- | TLTPTATYSGGVTDYASTFGIAVQQISTT | | |
| | LEKREAEA- | SSASSAATTASSKAKRAASQIGDGQVQA | | |
| | d28_THCAs | ATTTASVSTKSTAAAVSQIGDGQIQATT | | |
| | | KTTAAAVSQIGDGQIQATTKTTSAKTTA | | |
| | | AAVSQISDGQIQATTTTLAPKSTAAAVS | | |
| | | QIGDGQVQATTTTLAPKSTAAAVSQIGD | | |
| | | GQVQATTKTTAAAVSQIGDGQVQATTK | | |
| | | TTAAAVSQIGDGQVQATTKTTAAAVSQI | | |
| | | GDGQVQATTKTTAAAVSQITDGQVQAT | | |
| | | TKTTQAASQVSDGQVQATTATSASAAA | | |
| | | TSTDPVDAVSCKTSGTLEKREAEA | | |
| 0279/asn016-3 | HSP150_delta_ | MQYKKTLVASALAATTLAAYAPSEPWS | 128 | 9 |
| | fragment-and- | TLTPTATYSGGVTDYASTFGIAVQPISTT | | |
| | LEKREAEA- | SSASSAATTASSKAKRAASQIGDGQVQA | | |
| | THCAs | ATTTASVSTKSTAAAVSQIGDGQIQATT | | |
| | | KTTAAAVSQICDGQIQATTKTTSAKTTA | | |
| | | AAVSQISDGQIQATTTTLAPKSTAAAVS | | |
| | | QIGDGQVQATTTTLAPKSTAAAVSQIGD | | |
| | | GQVQATTKTTAAAVSQIGDGQVQATTK | | |
| | | TTAAAVSQIGDGQVQATTKTTAAAVSQI | | |
| | | GDGQVQATTKTTAAAVSQITDGQVQAT | | |
| | | TKTTQAASQVSDGQVQATTATSASAAA | | |
| | | TSTDPVDAVSCKTSGTLEKREAEA | | |
| 0279/asn018-1 | alpha_pre_signal- | MRFPSIFTAVLFAASSALAAYAPSEPWST | 129 | 7 |
| | and- | LTPTATYSGGVTDYASTFGIAVQPISTTS | | |
| | HSP150_delta_frag- | SASSAATTASSKAKRAASQIGDGQVQAA | | |
| | ment_wo_signal- | TTTASVSTKSTAAAVSQIGDGQIQATTK | | |
| | and-LEKREAEA- | TTAAAVSQIGDGQIQATTKTTSAKTTAA | | |
| | THCAs | AVSQISDGQIQATTTTLAPKSTAAAVSQI | | |
| | | GDGQVQATTTTLAPKSTAAAVSQIGDG | | |
| | | QVQATTKTTAAAVSQIGDGQVQATTKT | | |
| | | TAAAVSQIGDGQVQATTKTTAAAVSQIG | | |
| | | DGQVQATTKTTAAAVSQITDGQVQATT | | |
| | | KTTQAASQVSDGQVQATTATSASAAAT | | |
| | | STDPVDAVSCKTSGTLEKREAEA | | |
| 0279/asn019-2 | PIR3_delta_ | MQYKKPLVVSALAATSLAAYAPKDPWS | 130 | 491 |
| | fragment-and- | TLTPSATYKGGITDYSSSFGIAIEAVATSA | | |
| | LEKREAEA- | SSVASSKAKRAASQIGDGQVQAATTTA | | |
| | d28_THCAs | AVSKKSTAAAVSQITDGQVQAAKSTAA | | |
| | | AVSQITDGQVQAAKSTAAAVSQITDGQ | | |
| | | VQAAKSTAAAVSQITDGQVQAAKSTAA | | |
| | | AASQISDGQVQATTSTKAAASQITDGQI | | |
| | | QASKTTSGASQVSDGQVQATAEVKDAN | | |
| | | DPVDVVSCNNNSTLEKREAEA | | |
| 0279/asn020-3 | PIR3_delta_ | MQYKKPLVVSALAATSLAAYAPKDPWS | 131 | 37 |
| | fragment-and- | TLTPSATYKGGITDYSSSFGIAIEAVATSA | | |
| | LEKREAEA- | SSVASSKAKRAASQIGDGQVQAATTTA | | |
| | THCAs | AVSKKSTAAAVSQITDGQVQAAKSTAA | | |
| | | AVSQITDGQVQAAKSTSAAVSQITDGQV | | |
| | | QAAKSTAAAVSQITDGQVQAAKSTAAA | | |
| | | ASQISDGQVQATTSTKAAASQITDGQIQ | | |
| | | ASKTTSGASQVSDGQVQATAEVKDAND | | |
| | | PVDVVSCNNNSTLEKREAEAMNCSAFSF | | |
| | | WFVCKIIFFFLSFNIQISIA | | |
| 0279/asn021-2 | alpha_pre_signal- | MRFPSIFTAVLFAASSALAAYAPKDPWS | 132 | 512 |
| | and- | TLTPSATYKGGITDYSSSFGIAIEAVATSA | | |
| | PIR3_delta_frag- | SSVASSKAKRAASQIGDGQVQAATTTA | | |
| | ment_wo_signal- | AVSKKSTAAAVSQITDGQVQAAKSTAA | | |
| | and-LEKREAEA- | AVSQITDGQVQAAKSTAAAVSQITDGQ | | |
| | d28_THCAs | VQAAKSTAAAVSQITDGQVQAAKSTAA | | |
| | | AASQISDGQVQATTSTKAAASQITDGQI | | |
| | | QASKTTSGASQVSDGQVQATAEVKDAN | | |
| | | DPVDVVSCNNNSTLEKREAEA | | |
| 0279/asn024-3 | YAP_TA57_Kex2 | MKLKTVRSAVLSSLFASQVLGQTTAQT | 133 | 233 |
| | _spacer-yEVenus- | NSGGLDVVGLISMAKRKREEGEPKMSK | | |
| | THCAs | GEELFTGVVPILVELDGDVNGHKFSVSG | | |
| | | EGEGDATYGKLTLKLICTTGKLPVPWPT | | |
| | | LVTTLGYGLQCFARYPDHMKQHDFFKS | | |
| | | AMPEGYVQERTIFFKDDGNYKTRAEVK | | |
| | | FEGDTLVNRIELKGIDFKEDGNILGHKLE | | |
| | | YNYNSHNVYITADKQKNGIKANFKIRHN | | |
| | | IEDGGVQLADHYQQNTPIGDGPVLLPDN | | |
| | | HYLSYQSALSKDPNEKRDHMVLLEFVT | | |
| | | AAGITHGMDELYK | | |
| 0279/asn026-2 | K28 viral signal- | MESVSSLFNIFSTIMVNYKSLVLALLSVS | 134 | 26 |
| | THCAs | NLKYARGEEGEPK | | |
| 0279/asn028-1 | INU1A signal- | MKLAYSLLLPLAGVSASVINYKRMAMV | 135 | 56 |
| | THCAs | SEEGEPK | | |
| 0279/asn030-1 | INU1-THCAs | MKFAYSLLLPLAGVSASVINYKRMAMV | 136 | 58 |
| | | SEEGEPK | | |
| 0279/asn032-2 | K28 viral signal- | MESVSSLFNIFSTIMVNYKSLVLALLSVS | 137 | 111 |
| | noSpacer-THCAs | NLKYARG | | |
| 0279/asn034-4 | YAP_TA57_Kex2 | MKLKTVRSAVLSSLFASQVLGQTTAQT | 138 | 76 |
| | _spacer-THCAs | NSGGLDVVGLISMAKRKREEGEPK | | |

TABLE 19

| | | SEQ |
|---|---|---|
| | | ID |
| Plasmid ID | Signal Sequence Nucleotide Sequence | NO: |
| 0279/asn004-1 | ATGTTTAAATCTGTTGTTTATTCAATTTTAGCCGCTTCTTTGGC | 139 |
| | CAATGCA | |
| 0279/asn006-2 | ATGCAATACAAAAAGACTTTGGTTGCCTCTGCTTTGGCCGCT | 140 |
| | ACTACATTGGCCGCCTATGCTCCATCTGAGCCTTGGTCCACTT | |
| | TGACTCCAACAGCCACTTACAGCGGTGGTGTTACCGACTACG | |
| | CTTCCACCTTCGGTATTGCCGTTCAACCAATCTCCACTACATC | |
| | CAGCGCATCATCTGCAGCCACCACAGCCTCATCTAAGGCCAA | |
| | GAGAGCTGCTTCCCAAATTGGTGATGGTCAAGTCCAAGCTGC | |
| | TACCACTACTGCTTCTGTCTCTACCAAGAGTACCGCTGCCGCC | |
| | GTTTCTCAGATCGGTGATGGTCAAATCCAAGCTACTACTAAG | |
| | ACTACCGCTGCTGCTGTCTCTCAAATTGGTGATGGTCAAATTC | |
| | AAGCTACCACCAAGACTACCTCTGCTAATACTACCGCCGCTG | |
| | CCGTTTCTCAAATCAGTGATGGTCAAATCCAAGCTACCACCA | |
| | CTACTTTAGCCCCAAAGAGCACCGCTGCTGCCGTTTCTCAAA | |
| | TCGGTGATGGTCAAGTTCAAGCTACCACCACTACTTTAGCCC | |
| | CAAAGAGCACCGCTGCTGCCGTTTCTCAAATCGGTGATGGTC | |
| | AAGTTCAAGCTACTACTAAGACTACCGCTGCTGCTGTCTCTC | |
| | AAATTGGTGATGGTCAAGTTCAAGCTACCACCAAGACTACTG | |
| | CTGCCGCCGTTTCTCAAATCGGTGATGGTCAAGTTCAAGCTA | |
| | CTACCAAGACTACCGCTGCTGCTGTCTCTCAAATCGGTGATG | |
| | GTCAAGTTCAAGCAACTACCAAAACCACTGCCGCAGCTGTTT | |
| | CCCAAATTACTGACGGTCAAGTTCAAGCCACTACAAAAACCA | |
| | CTCAAGCAGCCAGCCAAGTAAGCGATGGCCAAGTCCAAGCT | |
| | ACTACTGCTACTTCCGCTTCTGCAGCCGCTACCTCCACTGACC | |
| | CAGTCGATGCTGTCTCCTGTAAGACTTCTGGTACC | |
| 0279/asn009-3 | ATGCAATATAAAAAGCCATTAGTCGTCTCCGCTTTAGCTGCT | 141 |
| | ACATCTTTAGCTGCCTATGCTCCAAAGGACCCGTGGTCCACTT | |
| | TAACTCCATCAGCTACTTACAAGGGTGGTATAACAGATTACT | |
| | CTTCGAGTTTCGGTATTGCTATTGAAGCCGTGGCTACCAGTGC | |
| | TTCCTCCGTCGCCTCATCTAAAGCAAAGAGAGCCGCCTATCA | |
| | GATAGGTGATGGTCAAGTACAGGCTGCCACTACTACTGCTGC | |
| | TGTTTCTAAGAAATCCACCGCTGCTGCTGTTTCTCAAATAACT | |
| | GACGGTCAAGTTCAAGCTGCTAAGTCTACTGCCGCTGCTGTT | |
| | TCCCAAATAACTGACGGTCAAGTTCAAGCTGCTAAGTCTACT | |
| | GCCGCTGCCGTTTCTCAAATAACTGACGGTCAAGTTCAAGCT | |
| | GCTAAGTCTACTGCCGCTGCCGTTTCTCAAATAACTGATGGTC | |
| | AAGTTCAAGCTGCCAAGTCTACTGCTGCCGCTGCCTCTCAGA | |
| | TTTCTGACGACCAAGTTCAGGCCACTACCTATACTAAGGCTG | |
| | CTGCATCCCAAATTACAGATGGGCAGATACAAGCATCTAAAA | |
| | CTACCAGTGGCGCTAGTCAAGTAAGTGATGGCCAAGTCCAGG | |
| | CTACTGCTGAAGTGAAAGACGCTAACGATCCAGTCGATGTTG | |
| | TTTCCTGTAATAACAATAGTACC | |
| 0279/asn010-1 | ATGCAATATAAAAAGCCATTAGTCGTCTCCGCTTTAGCTGCT | 142 |
| | ACATCTTTAGCTGCCTATGCTCCAAAGGACCCGTGGTCCACTT | |
| | TAACTCCATCAGCTACTTACAAGGGTGGTATAACAGATTACT | |
| | CTTCGAGTTTCGGTATTGCTATTGAAGCCGTGGCTACCAGTGC | |
| | TTCCTCCGTCGCCTCATCTAAAGCAAAGAGAGCCGCCTCTCA | |
| | GATAGGTGATGGTCAAGTACAGGCTGCCACTACTACTGCTGC | |
| | TGTTTCTAAGAAATCCACCGCTGCTGCTGTTTCTCAAATAACT | |
| | GACGGTCAAGTTCAAGCTGCTAAGTCTACTGCCGCTGCTGTT | |
| | TCCCAAATAACTGACGGTCAAGTTCAAGCTGCTAAGTCTACT | |
| | GCCGCTGCCGTTTCTCAAATAACTGACGGTCAAGTTCAAGCT | |
| | GCTAAGTCTACTGCCGCTGCCGTTTCTCAAATAACTGATGGTC | |
| | AAGTTCAAGCTGCCAAGTCTACTGCTGCCGCTGCCTCTCAGA | |
| | TTTCTGACGGCCAAGTTCAGGCCACTACCTCTACTAAGGCTG | |
| | CTGCATCCCAAATTACAGATGGGCAGATACAAGCATCTAAAA | |
| | CTACCAGTGGCGCTAGTCAAGTAAGTGATGGCCAAGTCCAGG | |
| | CTACTGCTGAAGTGAAAGACGCTAACGATCCAGTCGATGTTG | |
| | TTTCCTGTAATAACAATAGTACC | |
| 0279/asn012-3 | ATGAGATTTCCTTCAATTTTTACTGCAGTTTTATTCGCAGCAT | 143 |
| | CCTCCGCATTAGCTGCCTATGCTCCAAAGGACCCGTGGTCCA | |
| | CTTTAACTCCATCAGCTACTTACAAGGGTGGTATAACAGATT | |
| | ACTCTTCGAGTTTCGGTATTGCTATTGAAGCCGTGGCTACCAG | |
| | TGCTTCCTCCGTCGCCTCATCTAAAGCAAAGAGAGCCGCCTC | |
| | TCAGATAGGTGATGGTCAAGTACAGGCTGCCACTACTACTGC | |
| | TGCTGTTTCTAAGAAATCCACCGCTGCTGCTGTTTCTCAAATA | |
| | ACTGACGGTCAAGTTCAAGCTGCTAAGTCTACTGCCGCTGCT | |
| | GTTTCCCAAATAACTGACGGTCAAGTTCAAGCTGCTAAGTCT | |
| | ACTGCCGCTGCCGTTTCTCAAATAACTGACGGTCAAGTTCAA | |
| | GCTGCTAAGTCTACTGCCGCTGCCGTTTCTCAAATAACTGATG | |
| | GTCAAGTTCAAGCTGCCAAGTCTACTGCTGCCGCTGCCTCTC | |
| | AGATTTCTGACGGCCAAGTTCAGGCCACTACCTCTACTAAGG | |
| | CTGCTGCATCCCAAATTACAGATGGGCAGATACAAGCATCTA | |
| | AAACTACCAGTGGCGCTAGTCAAGTAAGTGATGGCCAAGTCC | |
| | AGGCTACTGCTGAAGTGAAAGACGCTAACGATCCAGTCGATG | |
| | TTGTTTCCTGTAATAACAATAGTACC | |
| 0279/asn014-2 | ATGCAATATAAAAAGCCATTAGTCGTCTCCGCTTTAGCTGCT | 144 |
| | ACATCTTTAGCT | |
| 0279/asn015-2 | ATGCAATACAAAAAGACTTTGGTTGCCTCTGCTTTGGCCGCT | 145 |
| | ACTACATTGGCCGCCTATGCTCCATCTGAGCCTTGGTCAACTT | |
| | TGACTCCAACAGCCACTTACAGCGGTGGTGTTACCGACTACG | |
| | CTTCCACCTTCGGTATTGCCGTTCAACAAATCTCCACTACATC | |
| | CAGCGCATCATCTGCAGCCACCACAGCCTCATCTAAGGCCAA | |
| | GAGAGCTGCTTCCCAAATTGGTGATGGTCAAGTCCAAGCTGC | |
| | TACCACTACTGCTTCTGTCTCTACCAAGAGTACCGCTGCCGCC | |
| | GTTTCTCAGATCGGTGATGGTCAAATCCAAGCTACTACTAAG | |
| | ACTACCGCTGCTGCTGTCTCTCAAATTGGTGATGGTCAAATTC | |
| | AAGCTACCACCAAGACTACCTCTGCTAAGACTACCGCCGCTG | |
| | CCGTTTCTCAAATCAGTGATGGTCAAATCCAAGCTACCACCA | |
| | CTACTTTAGCCCCAAAGAGCACCGCTGCTGCCGTTTCTCAAA | |
| | TCGGTGATGGTCAAGTTCAAGCTACCACCACTACTTTAGCCC | |
| | CAAAGAGCACCGCTGCTGCCGTTTCTCAAATCGGTGATGGTC | |
| | AAGTTCAAGCTACTACTAAGACTACCGCTGCTGCTGTCTCTC | |
| | AAATTGGTGATGGTCAAGTTCAAGCTACCACCAAGACTACTG | |
| | CTGCCGCCGTTTCTCAAATCGGTGATGGTCAAGTTCAAGCTA | |
| | CTACCAAGACTACCGCTGCTGCTGTCTCTCAAATCGGTGATG | |
| | GTCAAGTTCAAGCAACTACCAAAACCACTGCCGCAGCTGTTT | |
| | CCCAAATTACTGACGGTCAAGTTCAAGCCACTACAAAAACCA | |
| | CTCAAGCAGCCAGCCAAGTAAGCGATGGCCAAGTCCAAGCT | |
| | ACTACTGCTACTTCCGCTTCTGCAGCCGCTACCTCCACTGACC | |
| | CAGTCGATGCTGTCTCCTGTAAGACTTCTGGTACCTTGGAGA | |
| | AAAGAGAGGCTGAAGCA | |
| 0279/asn016-3 | ATGCAATACAAAAAGACTTTGGTTGCCTCTGCTTTGGCCGCT | 146 |
| | ACTACATTGGCCGCCTATGCTCCATCTGAGCCTTGGTCCACTT | |
| | TGACTCCAACAGCCACTTACAGCGGTGGTGTTACCGACTACG | |
| | CTTCCACCTTCGGTATTGCCGTTCAACCAATCTCCACTACATC | |
| | CAGCGCATCATCTGCAGCCACCACAGCCTCATCTAAGGCCAA | |
| | GAGAGCTGCTTCCCAAATTGGTGATGGTCAAGTCCAAGCTGC | |
| | TACCACTACTGCTTCTGTCTCTACCAAGAGTACCGCTGCCGCC | |
| | GTTTCTCAGATCGGTGATGGTCAAATCCAAGCTACTACTAAG | |
| | ACTACCGCTGCTGCTGTCTCTCAAATTTGTGATGGTCAAATTC | |
| | AAGCTACCACCAAGACTACCTCTGCTAAGACTACCGCCGCTG | |
| | CCGTTTCTCAAATCAGTGATGGTCAAATCCAAGCTACCACCA | |
| | CTACTTTAGCCCCAAAGAGCACCGCTGCTGCCGTTTCTCAAA | |
| | TCGGTGATGGTCAAGTTCAAGCTACCACCACTACTTTAGCCC | |
| | CAAAGAGCACCGCTGCTGCCGTTTCTCAAATCGGTGATGGTC | |
| | AAGTTCAAGCTACTACTAAGACTACCGCTGCTGCTGTCTCTC | |
| | AAATTGGTGATGGTCAAGTTCAAGCTACCACCAAGACTACTG | |
| | CTGCCGCCGTTTCTCAAATCGGTGATGGTCAAGTTCAAGCTA | |
| | CTACCAAGACTACCGCTGCTGCTGTCTCTCAAATCGGTGATG | |
| | GTCAAGTTCAAGCAACTACCAAAACCACTGCCGCAGCTGTTT | |
| | CCCAAATTACTGACGGTCAAGTTCAAGCCACTACAAAAACCA | |
| | CTCAAGCAGCCAGCCAAGTAAGCGATGGCCAAGTCCAAGCT | |
| | ACTACTGCTACTTCCGCTTCTGCAGCCGCTACCTCCACTGACC | |
| | CAGTCGATGCTGTCTCCTGTAAGACTTCTGGTACCTTGGAGA | |
| | AAAGAGAGGCTGAAGCA | |
| 0279/asn018-1 | ATGAGATTTCCTTCAATTTTTACTGCAGTTTTATTCGCAGCAT | 147 |
| | CCTCCGCATTAGCTGCCTATGCTCCATCTGAGCCTTGGTCCAC | |
| | TTTGACTCCAACAGCCACTTACAGCGGTGGTGTTACCGACTA | |
| | CGCTTCCACCTTCGGTATTGCCGTTCAACCAATCTCCACTACA | |
| | TCCAGCGCATCATCTGCAGCCACCACAGCCTCATCTAAGGCC | |
| | AAGAGAGCTGCTTCCCAAATTGGTGATGGTCAAGTCCAAGCT | |
| | GCTACCACTACTGCTTCTGTCTCTACCAAGAGTACCGCTGCCG | |
| | CCGTTTCTCAGATCGGTGATGGTCAAATCCAAGCTACTACTA | |
| | AGACTACCGCTGCTGCTGTCTCTCAAATTGGTGATGGTCAAA | |
| | TTCAAGCTACCACCAAGACTACCTCTGCTAAGACTACCGCCG | |
| | CTGCCGTTTCTCAAATCAGTGATGGTCAAATCCAAGCTACCA | |
| | CCACTACTTTAGCCCCAAAGAGCACCGCTGCTGCCGTTTCTC | |
| | AAATCGGTGATGGTCAAGTTCAAGCTACCACCACTACTTTAG | |
| | CCCCAAAGAGCACCGCTGCTGCCGTTTCTCAAATCGGTGATG | |
| | GTCAAGTTCAAGCTACTACTAAGACTACCGCTGCTGCTGTCT | |
| | CTCAAATTGGTGATGGTCAAGTTCAAGCTACCACCAAGACTA | |
| | CTGCTGCCGCCGTTTCTCAAATCGGTGATGGTCAAGTTCAAG | |
| | CTACTACCAAGACTACCGCTGCTGCTGTCTCTCAAATCGGTG | |
| | ATGGTCAAGTTCAAGCAACTACCAAAACCACTGCCGCAGCTG | |
| | TTTCCCAAATTACTGACGGTCAAGTTCAAGCCACTACAAAAA | |
| | CCACTCAAGCAGCCAGCCAAGTAAGCGATGGCCAAGTCCAA | |
| | GCTACTACTGCTACTTCCGCTTCTGCAGCCGCTACCTCCACTG | |
| | ACCCAGTCGATGCTGTCTCCTGTAAGACTTCTGGTACCTTGGA | |
| | GAAAAGAGAGGCTGAAGCA | |
| 0279/asn019-2 | ATGCAATATAAAAAGCCATTAGTCGTCTCCGCTTTAGCTGCT | 148 |
| | ACATCTTTAGCTGCCTATGCTCCAAAGGACCCGTGGTCCACTT | |
| | TAACTCCATCAGCTACTTACAAGGGTGGTATAACAGATTACT | |
| | CTTCGAGTTTCGGTATTGCTATTGAAGCCGTGGCTACCAGTGC | |
| | TTCCTCCGTCGCCTCATCTAAAGCAAAGAGAGCCGCCTCTCA | |
| | GATAGGTGATGGTCAAGTACAGGCTGCCACTACTACTGCTGC | |
| | TGTTTCTAAGAAATCCACCGCTGCTGCTGTTTCTCAAATAACT | |
| | GACGGTCAAGTTCAAGCTGCTAAGTCTACTGCCGCTGCTGTT | |
| | TCCCAAATAACTGACGGTCAAGTTCAAGCTGCTAAGTCTACT | |
| | GCCGCTGCCGTTTCTCAAATAACTGACGGTCAAGTTCAAGCT | |
| | GCTAAGTCTACTGCCGCTGCCGTTTCTCAAATAACTGATGGTC | |
| | AAGTTCAAGCTGCCAAGTCTACTGCTGCCGCTGCCTCTCAGA | |
| | TTTCTGACGGCCAAGTTCAGGCCACTACCTCTACTAAGGCTG | |
| | CTGCATCCCAAATTACAGATGGGCAGATACAAGCATCTAAAA | |
| | CTACCAGTGGCGCTAGTCAAGTAAGTGATGGCCAAGTCCAGG | |
| | CTACTGCTGAAGTGAAAGACGCTAACGATCCAGTCGATGTTG | |
| | TTTCCTGTAATAACAATAGTACCTTGGAGAAAAGAGAGGCTG | |
| | AAGCA | |
| 0279/asn020-3 | ATGCAATATAAAAAGCCATTAGTCGTCTCCGCTTTAGCTGCT | 149 |
| | ACATCTTTAGCTGCCTATGCTCCAAAGGACCCGTGGTCCACTT | |
| | TAACTCCATCAGCTACTTACAAGGGTGGTATAACAGATTACT | |
| | CTTCGAGTTTCGGTATTGCTATTGAAGCCGTGGCTACCAGTGC | |
| | TTCCTCCGTCGCCTCATCTAAAGCAAAGAGAGCCGCCTCTCA | |
| | GATAGGTGATGGTCAAGTACAGGCTGCCACTACTACTGCTGC | |
| | TGTTTCTAAGAAATCCACCGCTGCTGCTGTTTCTCAAATAACT | |
| | GACGGTCAAGTTCAAGCTGCTAAGTCTACTGCCGCTGCTGTT | |
| | TCCCAAATAACTGACGGTCAAGTTCAAGCTGCTAAGTCTACT | |
| | TCCGCTGCCGTTTCTCAAATAACTGACGGTCAAGTTCAAGCT | |
| | GCTAAGTCTACTGCCGCTGCCGTTTCTCAAATAACTGATGGTC | |
| | AAGTTCAAGCTGCCAAGTCTACTGCTGCCGCTGCCTCTCAGA | |
| | TTTCTGACGGCCAAGTTCAGGCCACTACCTCTACTAAGGCTG | |
| | CTGCATCCCAAATTACAGATGGGCAGATACAAGCATCTAAAA | |
| | CTACCAGTGGCGCTAGTCAAGTAAGTGATGGCCAAGTCCAGG | |
| | CTACTGCTGAAGTGAAAGACGCTAACGATCCAGTCGATGTTG | |
| | TTTCCTGTAATAACAATAGTACCTTGGAGAAAAGAGAGGCTG | |
| | AAGCAATGAACTGCTCCGCATTCTCTTTCTGGTTCGTCTGTAA | |
| | AATAATCTTCTTCTTCTTGTCCTTCAACATCCAAATCTCCATC | |
| | GCA | |

This set of plasmids was transformed into the yCBGA0314 strain and their CBDA productivity was assayed in the following high throughput screening process optimized for the CBDAs synthase activity. Colonies were inoculated into wells of a 96-well deep well plate. Each well contains 400 μl SC liquid medium (6.7 g/L Yeast Nitrogen Base, 1.6 g/L Amino Acid Drop Out mix without leucine, uracil, tryptophan and histidine, 22 g/L glucose, buffered to pH 6.0, supplemented with leucine, tryptophan and histidine). These inoculums were grown for 48 hours at 30° C. and shaken at 300 rpm with a 50 mm shaking diameter. After the 48 hours growth period 40 μl samples of these cultures were inoculated into 360 μl YPD-2400LA (10 g/L yeast extract, 20 g/L peptone, 20 g/L glucose and 240 mg/L olivetolic acid) medium. Then samples were grown for 48 hours at 30° C. and shaken at 300 rpm with a 50 mm shaking diameter and 8 μl of 12000 mg/l OLA dissolved in EtOH is added to the samples. Finally, samples were grown for additional 42 hours and are analyzed for cannabinoids.

The THCA titer in the above described screen ranged from 4 to 512 mg/L. Sample titers for several transformed strains are included in Table 20 below. (The titers are also included in Table 18 above.)

TABLE 20

| | | THCA |
|---|---|---|
| plasmid ID | ORF name | (mg/L) |
| 0279/asn021- | alpha_pre_signal-and- | 512 |
| 2 | PIR3_delta_fragment_wo_signal-and- | |
| | LEKREAEA-d28_THCAs | |
| 0279/asn009- | PIR3_delta_fragment-d28_THCAs | 501 |
| 3 | | |
| 0279/asn015- | HSP150_delta_fragment-and-LEKREAEA- | 500 |
| 2 | d28_THCAs | |
| 0279/asn011- | alpha_pre_signal-and- | 500 |
| 2 | PIR3_delta_fragment_wo_singal-d28_THCAs | |
| 0279/asn009- | PIR3_delta_fragment-d28_THCAs | 498 |
| 1 | | |
| 0279/asn019- | PIR3_delta_fragment-and-LEKREAEA- | 491 |
| 2 | d28_THCAs | |
| 0279/asn031- | K28 viral signal-noSpacer-d28_THCAs | 485 |
| 4 | | |
| 0279/asn025- | K28 viral signal-d28_THCAs | 479 |
| 3 | | |
| 0279/asn003- | PHO5_signal-d28_THCAs | 473 |
| 2 | | |
| 0279/asn013- | PIR3 secretion signal-d28_THCAs | 459 |
| 1 | | |
| 0279/asn029- | INU1-d28_THCAs | 440 |
| 2 | | |
| 0279/asn027- | INU1A signal-d28_THCAs | 430 |
| 1 | | |
| 0279/asn024- | YAP_TA57_Kex2_spacer-yEVenus-THCAs | 233 |
| 3 | | |
| 0279/asn033- | YAP_TA57_Kex2_spacer-d28_THCAs | 212 |
| 4 | | |
| 0279/asn001- | YAP_TA57_Kex2_spacer-d28_THCAs | 209 |
| 3 | | |
| 0279/asn023- | YAP_TA57_Kex2_spacer-yEVenus-d28_THCAs | 166 |
| 2 | | |
| 0279/asn014- | PIR3 secretion signal-THCAs | 122 |
| 2 | | |
| 0279/asn032- | K28 viral signal-noSpacer-THCAs | 111 |
| 2 | | |
| 0279/asn002- | YAP_TA57_Kex2_spacer-THCAs | 80 |
| 2 | | |
| 0279/asn034- | YAP_TA57_Kex2_spacer-THCAs | 76 |
| 4 | | |
| 0279/asn030- | INU1-THCAs | 58 |
| 1 | | |
| 0279/asn028- | INU1A signal-THCAs | 56 |
| 1 | | |
| 0279/asn020- | PIR3_delta_fragment-and-LEKREAEA-THCAs | 37 |
| 3 | | |
| 0279/asn004- | PHO5_signal-THCAs | 36 |
| 1 | | |
| 0279/asn026- | K28 viral signal-THCAs | 26 |
| 2 | | |
| 0279/asn006- | HSP150_delta_fragment-THCAs | 13 |
| 2 | | |
| 0279/asn012- | alpha_pre_signal-and- | 10 |
| 3 | PIR3_delta_fragment_wo_singal-THCAs | |
| 0279/asn012- | alpha_pre_signal-and- | 10 |
| 1 | PIR3_delta_fragment_wo_singal-THCAs | |
| 0279/asn016- | HSP150_delta_fragment-and-LEKREAEA-THCAs | 9 |
| 3 | | |
| 0279/asn018- | alpha_pre_signal-and- | 7 |
| 1 | HSP150_delta_fragment_wo_signal-and- | |
| | LEKREAEA-THCAs | |
| 0279/asn010- | PIR3_delta_fragment-THCAs | 4 |
| 1 | | |

### Example 23—THCA Synthase Mutagenesis

One amino acid position was mutagenized in the THCA synthase. The mutant genes were screened in yeast. Clones with increased THCA titer were identified.

The parental plasmid for the mutagenesis was the RUNM001233_63.1 plasmid, containing the Saccharomyces cerevisiae 2μ replication origin, the URA3 gene as an auxotrophic marker and a modified THCA synthase gene under the regulation of the bidirectional GAL1/GAL10 promoter. The nucleotide sequence of RUNM001233_63.1 is SEQ ID NO: 303. The modified THCA synthase gene contained a chimeric secretion signal and the THCA synthase gene lacking its predicted 28 amino acid long plant secretion signal.

The mutagenized position is the 469^{th} amino acid position of this modified THCA synthase protein, corresponding to the 446^{th} amino acid position of the unmodified THCA synthase protein. This position contains threonine in the natural THCA synthase. We have constructed large number of plasmids where the above-mentioned codon was randomly mutated allowing the incorporation of codons of any of the 20 amino acids.

The RUNM001233_63.1 plasmid and large number of its mutagenized variants were transformed into the yCBGA0517 strain and screened for THCA production. For the strain construction the parental strain was the yCBGA0314 strain. The PKS and OAC genes under the regulation of the bidirectional GAL1/GAL10 promoter and the AAE1 gene under the regulation of the STE5 promoter were inserted into the YDR508C, YRL020C and FOX1 loci replacing the native ORFs of the yCBGA0314 strain. A large number of isolates from this transformation was screened and few of them with high CBGA productivity and best reproducibility were identified. One of these isolates was prototrophic for uracil. Next, its URA3 gene was swapped with HIS3 gene resulting in the strain yCBGA0517, prototrophic for histidine and auxotrophic for uracil, leucine and tryptophan.

Colonies were screened using the following high throughput screening process of the THCA synthase activity: Colonies were inoculated into wells of a 96-well deep well plate. Each well contains 400 μl Synthetic Complete liquid medium (6.7 g/L Yeast Nitrogen Base, 1.6 g/L Amino Acid Drop Out mix without leucine, uracil, tryptophan and histidine, 22 g/L glucose, buffered to pH 6.0, supplemented with leucine, tryptophan and histidine). These inoculums were grown for 48 hours at 30° C. and shaken at 300 rpm with a 50 mm shaking diameter. After the 48 hours growth period 40 μl samples of these cultures are inoculated into 360 μl YPD-2400LA (10 g/L yeast extract, 20 g/L peptone, 20 g/L glucose and 240 mg/L olivetolic acid) medium. Then samples are grown for 48 hours at 30° C. and shaken at 300 rpm with a 50 mm shaking diameter and 8 μl of 12000 mg/l OLA dissolved in EtOH is added to the samples. Finally, samples were grown for additional 42 hours and are analyzed for cannabinoids.

The plasmid sequence of the best producer clones was determined. In all cases, the plasmids contained mutations only in the region of the mutagenized codon.

When Alanine, Valine or Isoleucine is coded for in the above-mentioned position of the modified THCA synthase gene, increased THCA titer was observed, as summarized in the Table 21 below:

TABLE 21

| | | | Amino acid | |
|---|---|---|---|---|
| Original | Position in | Position in | present in | THCA |
| amino | unmodified | modified | the marked | titer |
| acid | THCAs | THCAs | position | (mg/L) |
| Threonine | 446 | 469 | Threonine | 289 |
| Threonine | 446 | 469 | Alanine | 477 |
| Threonine | 446 | 469 | Valine | 565 |
| Threonine | 446 | 469 | Isoleucine | 445 |

### Example 24—CBDA Synthase Mutagenesis

Eighty-four (84) amino acid positions in the native Cannabis sativa CBDA synthase gene were mutagenized. The mutant genes were screened in yeast. Clones were identified with increased CBDA titer.

The parental plasmid for the mutagenesis was the 0285/asn080-2 plasmid, containing the Saccharomyces cerevisiae 2μ replication origin, the Hygromycin B resistance gene as a dominant marker and one of the modified CBDA synthase genes disclosed in this example under the regulation of the bidirectional GAL1/GAL10 promoter. The modified CBDA synthase gene contained the PIR3 delta fragment as secretion signal and the CBDA synthase gene lacking its predicted 21 amino acid long plant secretion signal. The modified CBDA coding sequences used for the 0285/asn080-2 parental plasmid are included in Table 22 below.

TABLE 22

| | |
|---|---|
| CBDAs | ATGCAATATAAAAAGCCATTAGTCGTCTCCGCTTTAGCTGCTACATCTTT |
| DNA | AGCTGCCTATGCTCCAAAGGACCCGTGGTCCACTTTAACTCCATCAGCTA |
| sequence | CTTACAAGGGTGGTATAACAGATTACTCTTCGAGTTTCGGTATTGCTATT |
| including | GAAGCCGTGGCTACCAGTGCTTCCTCCGTCGCCTCATCTAAAGCAAAGA |
| the signal | GAGCCGCCTCTCAGATAGGTGATGGTCAAGTACAGGCTGCCACTACTAC |
| sequence | TGCTGCTGTTTCTAAGAAATCCACCGCTGCTGCTGTTTCTCAAATAACTG |
| | ACGGTCAAGTTCAAGCTGCTAAGTCTACTGCCGCTGCTGTTTCCCAAATA |
| | ACTGACGGTCAAGTTCAAGCTGCTAAGTCTACTGCCGCTGCCGTTTCTCA |
| | AATAACTGACGGTCAAGTTCAAGCTGCTAAGTCTACTGCCGCTGCCGTTT |
| | CTCAAATAACTGATGGTCAAGTTCAAGCTGCCAAGTCTACTGCTGCCGC |
| | TGCCTCTCAGATTTCTGACGGCCAAGTTCAGGCCACTACCTCTACTAAGG |
| | CTGCTGCATCCCAAATTACAGATGGGCAGATACAAGCATCTAAAACTAC |
| | CAGTGGCGCTAGTCAAGTAAGTGATGGCCAAGTCCAGGCTACTGCTGAA |
| | GTGAAAGACGCTAACGATCCAGTCGATGTTGTTTCCTGTAATAACAATA |
| | GTACCAATATTCAAACTTCAATCGCTAACCCAAGAGAAAATTTCTTGAA |
| | GTGTTTCTCTCAATACATTCCAAATAATGCAACAAATTTGAAATTGGTTT |
| | ATACTCAAAATAATCCATTATACATGTCTGTTTTAAATTCTACAATTCAT |
| | AATTTGAGATTTTCTTCAGATACTACACCAAAACCATTGGTTATTGTTAC |
| | ACCATCTCATGTTTCACATATCCAAGGTACTATCTTGTGTTCTAAGAAAG |
| | TTGGTTTGCAAATTAGAACTAGATCAGGTGGTCATGATTCAGAAGGCAT |
| | GTCTTACATCTCACAAGTTCCATTCGTTATCGTTGATTTGAGAAACATGA |
| | GATCAATTAAAATTGATGTTCATTCACAAACAGCTTGGGTTGAAGCTGG |
| | TGCAACTTTGGGTGAAGTTTACTACTGGGTTAACGAAAAGAATGAATCT |
| | TTATCATTGGCTGCTGGTTACTGTCCAACAGTTTGTGCTGGTGGTCATTT |
| | TGGTGGTGGTGGTTATGGTCCATTAATGAGATCCTATGGTTTGGCTGCTG |
| | ATAACATCATCGATGCACATTTGGTTAACGTTCATGGTAAAGTTTTGGAT |
| | AGAAAGTCTATGGGTGAAGATTTGTTTTGGGCTTTGAGAGGTGGTGGTG |
| | CTGAATCATTTGGTATCATCGTTGCTTGGAAGATCAGATTGGTTGCAGTT |
| | CCAAAATCTACTATGTTCTCAGTTAAGAAAATTATGGAAATCCATGAAT |
| | TAGTTAAATTGGTTAATAAGTGGCAAAATATTGCTTATAAATACGATAA |
| | AGATTTGTTATTGATGACTCATTTTATTACAAGAAATATTACTGATAACC |
| | AAGGTAAAAATAAGACAGCTATCCATACTTACTTTTCTTCAGTTTTCTTG |
| | GGTGGTGTTGATTCTTTGGTTGATTTGATGAATAAGTCTTTTCCAGAATT |
| | AGGTATTAAGAAAACTGATTGTAGACAATTGTCTTGGATCGATACTATC |
| | ATTTTCTATTCAGGTGTTGTTAACTACGATACAGATAACTTCAATAAGGA |
| | AATTTTATTGGATAGATCAGCTGGTCAAAATGGTGCTTTTAAAATTAAAT |
| | TGGATTACGTTAAGAAACCAATTCCAGAATCAGTTTTCGTTCAAATTTTA |
| | GAAAAATTGTATGAAGAAGATATTGGTGCTGGCATGTACGCATTGTATC |
| | CATACGGTGGTATCATGGATGAAATTTCTGAATCAGCTATTCCATTTCCA |
| | CATAGAGCAGGTATTTTATACGAATTGTGGTACATTTGTTCTTGGGAAAA |
| | GCAAGAAGATAACGAAAAACATTTGAACTGGATTAGAAACATCTATAA |
| | CTTCATGACTCCATACGTTTCACAAAACCCAAGATTGGCTTATTTGAACT |
| | ACAGAGATTTGGATATCGGTATTAATGATCCTAAAAATCCAAACAACTA |
| | TACACAAGCAAGAATTTGGGGTGAAAAGTACTTCGGTAAAAATTTCGAT |
| | AGATTGGTTAAGGTTAAAACTTTGGTTGATCCAAATAATTTCTTTAGAAA |
| | TGAACAATCTATTCCACCATTGCCAAGACATAGACATTGA |
| | (SEQ ID NO: 150) |
| CBDAs | MQYKKPLVVSALAATSLAAYAPKDPWSTLTPSATYKGGITDYSSSFGIAIEA |
| amino | VATSASSVASSKAKRAASQIGDGQVQAATTTAAVSKKSTAAAVSQITDGQV |
| acid | QAAKSTAAAVSQITDGQVQAAKSTAAAVSQITDGQVQAAKSTAAAVSQIT |
| sequence | DGQVQAAKSTAAAASQISDGQVQATTSTKAAASQITDGQIQASKTTSGASQ |
| including | VSDGQVQATAEVKDANDPVDVVSCNNNSTNIQTSIANPRENFLKCFSQYIP |
| the signal | NNATNLKLVYTQNNPLYMSVLNSTIHNLRFSSDTTPKPLVIVTPSHVSHIQG |
| sequence | TILCSKKVGLQIRTRSGGHDSEGMSYISQVPFVIVDLRNMRSIKIDVHSQTA |
| | WVEAGATLGEVYYWVNEKNESLSLAAGYCPTVCAGGHFGGGGYGPLMRS |
| | YGLAADNIIDAHLVNVHGKVLDRKSMGEDLFWALRGGGAESFGIIVAWKI |
| | RLVAVPKSTMFSVKKIMEIHELVKLVNKWQNIAYKYDKDLLLMTHFITRNI |
| | TDNQGKNKTAIHTYFSSVFLGGVDSLVDLMNKSFPELGIKKTDCRQLSWID |
| | TIIFYSGVVNYDTDNFNKEILLDRSAGQNGAFKIKLDYVKKPIPESVFVQILE |
| | KLYEEDIGAGMYALYPYGGIMDEISESAIPFPHRAGILYELWYICSWEKQED |
| | NEKHLNWIRNIYNFMTPYVSQNPRLAYLNYRDLDIGINDPKNPNNYTQARI |
| | WGEKYFGKNFDRLVKVKTLVDPNNFFRNEQSIPPLPRHRH |
| | (SEQ ID NO: 151) |
| CBDAs | NIQTSIANPRENFLKCFSQYIPNNATNLKLVYTQNNPLYMSVLNSTIHNLRFS |
| amino | SDTTPKPLVIVTPSHVSHIQGTILCSKKVGLQIRTRSGGHDSEGMSYISQVPF |
| acid | VIVDLRNMRSIKIDVHSQTAWVEAGATLGEVYYWVNEKNESLSLAAGYCP |
| sequence | TVCAGGHFGGGGYGPLMRSYGLAADNIIDAHLVNVHGKVLDRKSMGEDL |
| without | FWALRGGGAESFGIIVAWKIRLVAVPKSTMFSVKKIMEIHELVKLVNKWQN |
| the signal | IAYKYDKDLLLMTHFITRNITDNQGKNKTAIHTYFSSVFLGGVDSLVDLMN |
| sequence | KSFPELGIKKTDCRQLSWIDTIIFYSGVVNYDTDNFNKEILLDRSAGQNGAF |
| | KIKLDYVKKPIPESVFVQILEKLYEEDIGAGMYALYPYGGIMDEISESAIPFPH |
| | RAGILYELWYICSWEKQEDNEKHLNWIRNIYNFMTPYVSQNPRLAYLNYR |
| | DLDIGINDPKNPNNYTQARIWGEKYFGKNFDRLVKVKTLVDPNNFFRNEQS |
| | IPPLPRHRH |
| | (SEQ ID NO: 152) |

The eighty-four (84) amino acid positions encoded by the wild-type CBDA synthase gene were selected for mutagenesis based on the fact that they contained amino acid differences with THCA synthase when aligned sequentially. The wild-type CBDA synthase specific amino acid was replaced at each of these positions for the corresponding amino acid found in the THCA synthase protein. 0285/asn080-2 plasmids containing the mutants were transformed into the yCBGA0513 strain and screened for CBDA production.

Several double combinations of these mutations were also introduced into the 0285/asn080-2 plasmid, and along with their corresponding single mutant plasmid counterparts, were transformed into the yCBGA0523 strain and screened for CBDA production.

The CBDA mutant (and wild type) coding sequences used for the 0285/asn080-2 parental plasmid are included in Table 22 in Example 24.

CBDA synthase activity in the strains was screened using the following high throughput screening process. Colonies were inoculated into wells of a 96-well deep well plate. Each well contained 400 μl SC liquid medium (6.7 g/L Yeast Nitrogen Base, 1.6 g/L Amino Acid Drop Out mix without leucine, uracil, tryptophan and histidine, 22 g/L glucose, buffered to pH 6.0, supplemented with leucine, tryptophan, histidine and Hygromycin B). These inoculums were grown for 48 hours at 30° C. and shaken at 300 rpm with 50 mm shaking diameter. After a 48 hour growth period, 40 μl samples of these cultures were inoculated into 360 μl YPD-2400LA (10 g/L yeast extract, 20 g/L peptone, 20 g/L glucose and 240 mg/L olivetolic acid) medium. Then samples were grown for 48 hours at 30° C. and shaken at 300 rpm with 50 mm shaking diameter and 8 μl of 12000 mg/l OLA dissolved in EtOH is added to the samples. Finally, samples were grown for additional 42 hours and were analyzed for cannabinoids.

The CBDA titer in the above described screen ranged from 10.5 to 408.6 mg/L. Sample titers for the transformed strains, along with each strain's respective amino acid mutations and amino acid sequences, are included in Tables 23-26 below. (All mutation sites refer to the mutation site on wild-type CBDA's coding sequence without the wild-type signal sequence.)

TABLE 23
yCBGA_0513 mutant strains

| | mutation site | | | |
|---|---|---|---|---|
| | on CBDAs | | | |
| | coding | | | |
| | sequence | | | Average |
| | without the | native | new | titer |
| | signal | amino | amino | of CBDA |
| Constructs | sequence | acid | acid | mg/L |
| PIR3_CBDAs_S449N | 216 | S | N | 408.6 |
| PIR3_CBDAs_G307A | 74 | G | A | 407 |
| PIR3_CBDAs_H425D | 192 | H | D | 381.1 |
| PIR3_CBDAs_P464PS | 231 | P | PS | 378.7 |
| PIR3_CBDAs_N269D | 36 | N | D | 376.9 |
| PIR3_CBDAs_V454A | 221 | V | A | 376.6 |
| PIR3_CBDAs_M468I | 235 | M | I | 367.5 |
| PIR3_CBDAs_I700L | 467 | I | L | 357 |
| PIR3_CBDAs_Y571F | 338 | Y | F | 352 |
| PIR3_CBDAs_L442I | 209 | L | I | 350.9 |
| PIR3_CBDAs_S328A | 95 | S | A | 343.8 |
| PIR3_CBDAs_I620V | 387 | I | V | 341.6 |
| PIR3_CBDAs_I676V | 443 | I | V | 340.3 |
| PIR3_CBDA5_Q252E | 19 | Q | E | 325.6 |
| PIR3_CBDAs_C392G | 159 | C | G | 323.9 |
| PIR3_CBDAs_R459K | 226 | R | K | 320.8 |
| PIR3_CBDAs_I341V | 108 | I | V | 319.6 |
| PIR3_CBDAs_A626V | 393 | A | V | 319.2 |
| PIR3_CBDAs_L261P | 28 | L | P | 318.2 |
| PIR3_CBDAs_G590T | 357 | G | T | 317.5 |
| PIR3_CBDAs_C658A | 425 | C | A | 310.5 |
| PIR3_CBDAs_V264I | 31 | V | I | 310.3 |
| PIR3_CBDAs_A622V | 389 | A | V | 309.2 |
| PIR3_CBDAs_N675S | 442 | N | S | 308.7 |
| PIR3_CBDAs_R243Q | 10 | R | Q | 306.3 |
| PIR3_CBDAs_L651M | 418 | L | M | 303 |
| PIR3_CBDAs_H281Q | 48 | H | Q | 301 |
| PIR3_CBDAs_R554K | 321 | R | K | 299 |
| PIR3_CBDAs_K706E | 473 | K | E | 297.7 |
| PIR3_CBDAs_Q555E | 322 | Q | E | 294.3 |
| PIR3_CBDAs_R755H | 522 | R | H | 294.2 |
| PIR3_CBDAs_R753P | 520 | R | P | 292.5 |
| PIR3_CBDAs_V736A | 503 | V | A | 290.7 |
| PIR3_CBDAs_A393V | 160 | A | V | 285.6 |
| PIR3_CBDAs_S286T | 53 | S | T | 285.5 |
| PIR3_CBDAs_D572N | 339 | D | N | 285.4 |
| wild type control | wild type | N/A | N/A | 283.7 |
| construct | | | | |
| PIR3_CBDAs_Q610K | 377 | Q | K | 277.8 |
| PIR3_CBDAs_G398S | 165 | G | S | 276.9 |
| PIR3_CBDAs_L499V | 266 | L | V | 274.1 |
| PIR3_CBDAs_Q513H | 280 | Q | H | 274 |
| PIR3_CBDAs_D574A | 341 | D | A | 270.7 |
| PIR3_CBDAs_L735K | 502 | L | K | 269.3 |
| PIR3_CBDAs_N577K | 344 | N | K | 265.7 |
| PIR3_CBDAs_Q588K | 355 | Q | K | 264.1 |
| PIR3_CBDAs_L383F | 150 | L | F | 261.3 |
| PIR3_CBDAs_T259A | 26 | T | A | 260 |
| PIR3_CBDAs_D635E | 402 | D | E | 256.4 |
| PIR3_CBDAs_V527I | 294 | V | I | 253.2 |
| PIR3_CBDAs_V374I | 141 | V | I | 252.8 |
| PIR3_CBDAs_R348H | 115 | R | H | 250.4 |
| PIR3_CBDAs_S380N | 147 | S | N | 248.9 |
| PIR3_CBDAs_S606T | 373 | S | T | 248.6 |
| PIR3_CBDAs_A258P | 25 | A | P | 243.2 |
| PIR3_CBDAs_R507K | 274 | R | K | 243 |
| PIR3_CBDAs_A384P | 151 | A | P | 237.4 |
| PIR3_CBDAs_L670I | 437 | L | I | 227 |
| PIR3_CBDAs_S408N | 175 | S | N | 224.3 |
| PIR3_CBDAs_T522G | 289 | T | G | 220.4 |
| PIR3_CBDAs_N268H | 35 | N | H | 215.2 |
| PIR3_CBDAs_V607A | 374 | V | A | 213.7 |
| PIR3_CBDAs_N707S | 474 | N | S | 210.9 |
| PIR3_CBDAs_I702K | 469 | I | K | 203.1 |
| PIR3_CBDAs_I520V | 287 | I | V | 199.5 |
| PIR3_CBDAs_H301N | 68 | H | N | 195.1 |
| PIR3_CBDAs_F608M | 375 | F | M | 186.4 |
| PIR3_CBDAs_A519T | 286 | A | T | 181.3 |
| PIR3_CBDAs_D727N | 494 | D | N | 174.8 |
| PIR3_CBDAs_N589K | 356 | N | K | 172.2 |
| PIR3_CBDAs_D704N | 471 | D | N | 162 |
| PIR3_CBDAs_T308S | 75 | T | S | 161.7 |
| PIR3_CBDAs_I657T | 424 | I | T | 160.2 |
| PIR3_CBDAs_V484F | 251 | V | F | 160 |
| PIR3_CBDAs_I673V | 440 | I | V | 157.8 |
| PIR3_CBDAs_A385G | 152 | A | G | 145.6 |
| PIR3_CBDAs_I474N | 241 | I | N | 141.2 |
| PIR3_CBDAs_A447G | 214 | A | G | 127.5 |
| PIR3_CBDAs_L529H | 296 | L | H | 112.1 |
| PIR3_CBDAs_M680T | 447 | M | T | 100.3 |
| PIR3_CBDAs_P270Q | 37 | P | Q | 96.2 |
| PIR3_CBDAs_N703T | 470 | N | T | 96.1 |
| PIR3_CBDAs_L556F | 323 | L | F | 92.9 |
| PIR3_CBDAs_L381F | 148 | L | F | 76.8 |
| PIR3_CBDAs_E479G | 246 | E | G | 75.3 |
| PIR3_CBDAs_I562T | 329 | I | T | 10.5 |

TABLE 24
yCBGA_0513 mutant strains

| | | SEQ |
|---|---|---|
| | | ID |
| Constructs | CBDA amino acid sequence | NO: |
| PIR3_CB | NIQTSIANPRENFLKCFSQYIPNNATNLKLVYTQNNPLYMSVLNS | 153 |
| DAs_S449 | TIHNLRFSSDTTPKPLVIVTPSHVSHIQGTILCSKKVGLQIRTRSGG | |
| N | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAENFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 154 |
| DAs_G30 | TIHNLRFSSDTTPKPLVIVTPSHVSHIQATILCSKKVGLQIRTRSGG | |
| 7A | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 155 |
| DAs_H42 | TIHNLRFSSDTTPKPLVIVTPSHVSHIQGTILCSKKVGLQIRTRSGG | |
| 5D | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVDGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 156 |
| DAs_P464 | TIHNLRFSSDTTPKPLVIVTPSHVSHIQGTILCSKKVGLQIRTRSGG | |
| PS | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPSKSTMFSVKKIMEIHELVKLVNKWQNIAYKYDK | |
| | DLLLMTHFITRNITDNQGKNKTAIHTYFSSVFLGGVDSLVDLMN | |
| | KSFPELGIKKTDCRQLSWIDTIIFYSGVVNYDTDNFNKEILLDRSA | |
| | GQNGAFKIKLDYVKKPIPESVFVQILEKLYEEDIGAGMYALYPY | |
| | GGIMDEISESAIPFPHRAGILYELWYICSWEKQEDNEKHLNWIRN | |
| | IYNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNDPLYMSVLNS | 157 |
| DAs_N26 | TIHNLRFSSDTTPKPLVIVTPSHVSHIQGTILCSKKVGLQIRTRSGG | |
| 9D | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 158 |
| DAs_V45 | TIHNLRFSSDTTPKPLVIVTPSHVSHIQGTILCSKKVGLQIRTRSGG | |
| 4A | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIA | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 159 |
| DAs_M46 | TIHNLRFSSDTTPKPLVIVTPSHVSHIQGTILCSKKVGLQIRTRSGG | |
| 81 | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTIFSVKKIMEIHELVKLVNKWQNIAYKYDKDL | |
| | LLMTHFITRNITDNQGKNKTAIHTYFSSVFLGGVDSLVDLMNKS | |
| | FPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 160 |
| DAs_I700 | TIHNLRFSSDTTPKPLVIVTPSHVSHIQGTILCSKKVGLQIRTRSGG | |
| L | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDLGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 161 |
| DAs_Y57 | TIHNLRFSSDTTPKPLVIVTPSHVSHIQGTILCSKKVGLQIRTRSGG | |
| 1F | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNFDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 162 |
| DAs_L44 | TIHNLRFSSDTTPKPLVIVTPSHVSHIQGTILCSKKVGLQIRTRSGG | |
| 2I | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWAIRGGGAESFGIIVA | |
| | WKIRLVAVPKSTMFSVKKIMEIHELVKLVNKWQNIAYKYDKDL | |
| | LLMTHFITRNITDNQGKNKTAIHTYFSSVFLGGVDSLVDLMNKS | |
| | FPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 163 |
| DAs_S328 | TIHNLRFSSDTTPKPLVIVTPSHVSHIQGTILCSKKVGLQIRTRSGG | |
| A | HDAEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 164 |
| DAs_I620 | TIHNLRFSSDTTPKPLVIVTPSHVSHIQGTILCSKKVGLQIRTRSGG | |
| V | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDVGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 165 |
| DAs_I676 | TIHNLRFSSDTTPKPLVIVTPSHVSHIQGTILCSKKVGLQIRTRSGG | |
| V | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNV | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSEYIPNNATNLKLVYTQNNPLYMSVLNS | 166 |
| DAs_Q25 | TIHNLRFSSDTTPKPLVIVTPSHVSHIQGTILCSKKVGLQIRTRSGG | |
| 2E | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 167 |
| DAs_C39 | TIHNLRFSSDTTPKPLVIVTPSHVSHIQGTILCSKKVGLQIRTRSGG | |
| 2G | HDSEGMSYISQVPFVIVDLRNMRSIKIDVHSQTAWVEAGATLGE | |
| | VYYWVNEKNESLSLAAGYCPTVGAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 168 |
| DAs_R45 | TIHNLRFSSDTTPKPLVIVTPSHVSHIQGTILCSKKVGLQIRTRSGG | |
| 9K | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIKLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 169 |
| DAs_I341 | TIHNLRFSSDTTPKPLVIVTPSHVSHIQGTILCSKKVGLQIRTRSGG | |
| V | HDSEGMSYISQVPFVVVDLRNMRSIKIDVHSQTAWVEAGATLG | |
| | EVYYWVNEKNESLSLAAGYCPTVCAGGHFGGGGYGPLMRSYG | |
| | LAADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 170 |
| DAs_A62 | TIHNLRFSSDTTPKPLVIVTPSHVSHIQGTILCSKKVGLQIRTRSGG | |
| 6V | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYVLYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNPKLVYTQNNPLYMSVLNS | 171 |
| DAs_L26 | TIHNLRFSSDTTPKPLVIVTPSHVSHIQGTILCSKKVGLQIRTRSGG | |
| 1P | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 172 |
| DAs_G59 | TIHNLRFSSDTTPKPLVIVTPSHVSHIQGTILCSKKVGLQIRTRSGG | |
| 0T | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNTAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 173 |
| DAs_C65 | TIHNLRFSSDTTPKPLVIVTPSHVSHIQGTILCSKKVGLQIRTRSGG | |
| 8A | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYIASWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLIYTQNNPLYMSVLNS | 174 |
| DAs_V26 | TIHNLRFSSDTTPKPLVIVTPSHVSHIQGTILCSKKVGLQIRTRSGG | |
| 4I | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 175 |
| DAs_A62 | TIHNLRFSSDTTPKPLVIVTPSHVSHIQGTILCSKKVGLQIRTRSGG | |
| 2V | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGVGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 176 |
| DAs_N67 | TIHNLRFSSDTTPKPLVIVTPSHVSHIQGTILCSKKVGLQIRTRSGG | |
| 5S | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRSIY | |
| | NFMTPYVSQNPRLAYLNYRDLDIGINDPKNPNNYTQARIWGEK | |
| | YFGKNFDRLVKVKTLVDPNNFFRNEQSIPPLPRHRH | |
| PIR3_CB | NIQTSIANPQENFLKCFSQYIPNNATNLKLVYTQNNPLYMSVLNS | 177 |
| DAs_R24 | TIHNLRFSSDTTPKPLVIVTPSHVSHIQGTILCSKKVGLQIRTRSGG | |
| 3Q | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 178 |
| DAs_L65 | TIHNLRFSSDTTPKPLVIVTPSHVSHIQGTILCSKKVGLQIRTRSGG | |
| 1M | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGIMYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 179 |
| DAs_H28 | TIQNLRFSSDTTPKPLVIVTPSHVSHIQGTILCSKKVGLQIRTRSGG | |
| 1Q | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 180 |
| DAs_R55 | TIHNLRFSSDTTPKPLVIVTPSHVSHIQGTILCSKKVGLQIRTRSGG | |
| 4K | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCKQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 181 |
| DAs_K70 | TIHNLRFSSDTTPKPLVIVTPSHVSHIQGTILCSKKVGLQIRTRSGG | |
| 6E | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPENPNNYTQARIWGEK | |
| | YFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 182 |
| DAs_Q55 | TIHNLRFSSDTTPKPLVIVTPSHVSHIQGTILCSKKVGLQIRTRSGG | |
| 5E | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRELSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 183 |
| DAs_R75 | TIHNLRFSSDTTPKPLVIVTPSHVSHIQGTILCSKKVGLQIRTRSGG | |
| 5H | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHHH | |
| PIR3_CB | NIQTSIANPRENFLKCFSQYIPNNATNLKLVYTQNNPLYMSVLNS | 184 |
| DAs_R75 | TIHNLRFSSDTTPKPLVIVTPSHVSHIQGTILCSKKVGLQIRTRSGG | |
| 3P | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPPHRH | |
| PIR3_CB | NIQTSIANPRENFLKCFSQYIPNNATNLKLVYTQNNPLYMSVLNS | 185 |
| DAs_V73 | TIHNLRFSSDTTPKPLVIVTPSHVSHIQGTILCSKKVGLQIRTRSGG | |
| 6A | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLADPNNFFRNEQSIPPLPRHRH | |
| PIR3_CB | NIQTSIANPRENFLKCFSQYIPNNATNLKLVYTQNNPLYMSVLNS | 186 |
| DAs_A39 | TIHNLRFSSDTTPKPLVIVTPSHVSHIQGTILCSKKVGLQIRTRSGG | |
| 3V | HDSEGMSYISQVPFVIVDLRNMRSIKIDVHSQTAWVEAGATLGE | |
| | VYYWVNEKNESLSLAAGYCPTVCVGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 187 |
| DAs_S286 | TIHNLRFTSDTTPKPLVIVTPSHVSHIQGTILCSKKVGLQIRTRSGG | |
| T | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 188 |
| DAs_D57 | TIHNLRFSSDTTPKPLVIVTPSHVSHIQGTILCSKKVGLQIRTRSGG | |
| 2N | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYNTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| wild type | NIQTSIANPRENFLKCFSQYIPNNATNLKLVYTQNNPLYMSVLNS | 189 |
| control | TIHNLRFSSDTTPKPLVIVTPSHVSHIQGTILCSKKVGLQIRTRSGG | |
| construct | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 190 |
| DAs_Q61 | TIHNLRFSSDTTPKPLVIVTPSHVSHIQGTILCSKKVGLQIRTRSGG | |
| 0K | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVKILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 191 |
| DAs_G39 | TIHNLRFSSDTTPKPLVIVTPSHVSHIQGTILCSKKVGLQIRTRSGG | |
| 8S | HDSEGMSYISQVPFVIVDLRNMRSIKIDVHSQTAWVEAGATLGE | |
| | VYYWVNEKNESLSLAAGYCPTVCAGGHFSGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 192 |
| DAs_L49 | TIHNLRFSSDTTPKPLVIVTPSHVSHIQGTILCSKKVGLQIRTRSGG | |
| 9V | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LVLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 193 |
| DAs_Q51 | TIHNLRFSSDTTPKPLVIVTPSHVSHIQGTILCSKKVGLQIRTRSGG | |
| 3H | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNHGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 194 |
| DAs_D57 | TIHNLRFSSDTTPKPLVIVTPSHVSHIQGTILCSKKVGLQIRTRSGG | |
| 4A | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTANFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 195 |
| DAs_L73 | TIHNLRFSSDTTPKPLVIVTPSHVSHIQGTILCSKKVGLQIRTRSGG | |
| 5K | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTKVDPNNFFRNEQSIPPLPRHRH | |
| PIR3_CB | NIQTSIANPRENFLKCFSQYIPNNATNLKLVYTQNNPLYMSVLNS | 196 |
| DAs_N57 | TIHNLRFSSDTTPKPLVIVTPSHVSHIQGTILCSKKVGLQIRTRSGG | |
| 7K | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFKKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 197 |
| DAs_Q58 | TIHNLRFSSDTTPKPLVIVTPSHVSHIQGTILCSKKVGLQIRTRSGG | |
| 8K | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | KNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 198 |
| DAs_L38 | TIHNLRFSSDTTPKPLVIVTPSHVSHIQGTILCSKKVGLQIRTRSGG | |
| 3F | HDSEGMSYISQVPFVIVDLRNMRSIKIDVHSQTAWVEAGATLGE | |
| | VYYWVNEKNESLSFAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNAANLKLVYTQNNPLYMSVLNS | 199 |
| DAs_T25 | TIHNLRFSSDTTPKPLVIVTPSHVSHIQGTILCSKKVGLQIRTRSGG | |
| 9A | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 200 |
| DAs_D63 | TIHNLRFSSDTTPKPLVIVTPSHVSHIQGTILCSKKVGLQIRTRSGG | |
| 5E | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMEEISESAIPFPHRAGILYELWYICSWEKQEDNEKHLNWIRNIY | |
| | NFMTPYVSQNPRLAYLNYRDLDIGINDPKNPNNYTQARIWGEK | |
| | YFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 201 |
| DAs_V52 | TIHNLRFSSDTTPKPLVIVTPSHVSHIQGTILCSKKVGLQIRTRSGG | |
| 7I | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSIFLGGVDSLVDLMNKS | |
| | FPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 202 |
| DAs_V37 | TIHNLRFSSDTTPKPLVIVTPSHVSHIQGTILCSKKVGLQIRTRSGG | |
| 4I | HDSEGMSYISQVPFVIVDLRNMRSIKIDVHSQTAWVEAGATLGE | |
| | VYYWINEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 203 |
| DAs_R34 | TIHNLRFSSDTTPKPLVIVTPSHVSHIQGTILCSKKVGLQIRTRSGG | |
| 8H | HDSEGMSYISQVPFVIVDLRNMHSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 204 |
| DAs_S380 | TIHNLRFSSDTTPKPLVIVTPSHVSHIQGTILCSKKVGLQIRTRSGG | |
| N | HDSEGMSYISQVPFVIVDLRNMRSIKIDVHSQTAWVEAGATLGE | |
| | VYYWVNEKNENLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 205 |
| DAs_S606 | TIHNLRFSSDTTPKPLVIVTPSHVSHIQGTILCSKKVGLQIRTRSGG | |
| T | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPETVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNPTNLKLVYTQNNPLYMSVLNS | 206 |
| DAs_A25 | TIHNLRFSSDTTPKPLVIVTPSHVSHIQGTILCSKKVGLQIRTRSGG | |
| 8P | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 207 |
| DAs_R50 | TIHNLRFSSDTTPKPLVIVTPSHVSHIQGTILCSKKVGLQIRTRSGG | |
| 7K | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITKNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 208 |
| DAs_A38 | TIHNLRFSSDTTPKPLVIVTPSHVSHIQGTILCSKKVGLQIRTRSGG | |
| 4P | HDSEGMSYISQVPFVIVDLRNMRSIKIDVHSQTAWVEAGATLGE | |
| | VYYWVNEKNESLSLPAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 209 |
| DAs_L67 | TIHNLRFSSDTTPKPLVIVTPSHVSHIQGTILCSKKVGLQIRTRSGG | |
| 0I | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHINWIRNIY | |
| | NFMTPYVSQNPRLAYLNYRDLDIGINDPKNPNNYTQARIWGEK | |
| | YFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 210 |
| DAs_S408 | TIHNLRFSSDTTPKPLVIVTPSHVSHIQGTILCSKKVGLQIRTRSGG | |
| N | HDSEGMSYISQVPFVIVDLRNMRSIKIDVHSQTAWVEAGATLGE | |
| | VYYWVNEKNESLSLAAGYCPTVCAGGHFGGGGYGPLMRNYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 211 |
| DAs_T52 | TIHNLRFSSDTTPKPLVIVTPSHVSHIQGTILCSKKVGLQIRTRSGG | |
| 2G | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHGYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQHNPLYMSVLNS | 212 |
| DAs_N26 | TIHNLRFSSDTTPKPLVIVTPSHVSHIQGTILCSKKVGLQIRTRSGG | |
| 8H | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 213 |
| DAs_V60 | TIHNLRFSSDTTPKPLVIVTPSHVSHIQGTILCSKKVGLQIRTRSGG | |
| 7A | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESAFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 214 |
| DAs_N70 | TIHNLRFSSDTTPKPLVIVTPSHVSHIQGTILCSKKVGLQIRTRSGG | |
| 7S | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKSPNNYTQARIWGEK | |
| | YFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 215 |
| DAs_I702 | TIHNLRFSSDTTPKPLVIVTPSHVSHIQGTILCSKKVGLQIRTRSGG | |
| K | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGKNDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 216 |
| DAs_I520 | TIHNLRFSSDTTPKPLVIVTPSHVSHIQGTILCSKKVGLQIRTRSGG | |
| V | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAVHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 217 |
| DAs_H30 | TIHNLRFSSDTTPKPLVIVTPSNVSHIQGTILCSKKVGLQIRTRSGG | |
| 1N | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 218 |
| DAs_F608 | TIHNLRFSSDTTPKPLVIVTPSHVSHIQGTILCSKKVGLQIRTRSGG | |
| M | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVMVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 219 |
| DAs_A51 | TIHNLRFSSDTTPKPLVIVTPSHVSHIQGTILCSKKVGLQIRTRSGG | |
| 9T | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTTIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 220 |
| DAs_D72 | TIHNLRFSSDTTPKPLVIVTPSHVSHIQGTILCSKKVGLQIRTRSGG | |
| 7N | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFNRLVKVKTLVDPNNFFRNEQSIPPLPRHRH | |
| PIR3_CB | NIQTSIANPRENFLKCFSQYIPNNATNLKLVYTQNNPLYMSVLNS | 221 |
| DAs_N58 | TIHNLRFSSDTTPKPLVIVTPSHVSHIQGTILCSKKVGLQIRTRSGG | |
| 9K | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QKGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 222 |
| DAs_D70 | TIHNLRFSSDTTPKPLVIVTPSHVSHIQGTILCSKKVGLQIRTRSGG | |
| 4N | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINNPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 223 |
| DAs_T30 | TIHNLRFSSDTTPKPLVIVTPSHVSHIQGSILCSKKVGLQIRTRSGG | |
| 8S | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 224 |
| DAs_I657 | TIHNLRFSSDTTPKPLVIVTPSHVSHIQGTILCSKKVGLQIRTRSGG | |
| T | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYTCSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 225 |
| DAs_V48 | TIHNLRFSSDTTPKPLVIVTPSHVSHIQGTILCSKKVGLQIRTRSGG | |
| 4F | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLFNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 226 |
| DAs_I673 | TIHNLRFSSDTTPKPLVIVTPSHVSHIQGTILCSKKVGLQIRTRSGG | |
| V | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWVRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 227 |
| DAs_A38 | TIHNLRFSSDTTPKPLVIVTPSHVSHIQGTILCSKKVGLQIRTRSGG | |
| 5G | HDSEGMSYISQVPFVIVDLRNMRSIKIDVHSQTAWVEAGATLGE | |
| | VYYWVNEKNESLSLAGGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 228 |
| DAs_I474 | TIHNLRFSSDTTPKPLVIVTPSHVSHIQGTILCSKKVGLQIRTRSGG | |
| N | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKNMEIHELVKLVNKWQNIAYKYDK | |
| | DLLLMTHFITRNITDNQGKNKTAIHTYFSSVFLGGVDSLVDLMN | |
| | KSFPELGIKKTDCRQLSWIDTIIFYSGVVNYDTDNFNKEILLDRSA | |
| | GQNGAFKIKLDYVKKPIPESVFVQILEKLYEEDIGAGMYALYPY | |
| | GGIMDEISESAIPFPHRAGILYELWYICSWEKQEDNEKHLNWIRN | |
| | IYNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 229 |
| DAs_A44 | TIHNLRFSSDTTPKPLVIVTPSHVSHIQGTILCSKKVGLQIRTRSGG | |
| 7G | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGGESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 230 |
| DAs_L52 | TIHNLRFSSDTTPKPLVIVTPSHVSHIQGTILCSKKVGLQIRTRSGG | |
| 9H | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFHGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 231 |
| DAs_M68 | TIHNLRFSSDTTPKPLVIVTPSHVSHIQGTILCSKKVGLQIRTRSGG | |
| 0T | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFTTPYVSQNPRLAYLNYRDLDIGINDPKNPNNYTQARIWGEK | |
| | YFGKNFDRLVKVKTLVDPNNFFRNEQSIPPLPRHRH | |
| PIR3_CB | NIQTSIANPRENFLKCFSQYIPNNATNLKLVYTQNNQLYMSVLNS | 232 |
| DAs_P270 | TIHNLRFSSDTTPKPLVIVTPSHVSHIQGTILCSKKVGLQIRTRSGG | |
| Q | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 233 |
| DAs_N70 | TIHNLRFSSDTTPKPLVIVTPSHVSHIQGTILCSKKVGLQIRTRSGG | |
| 3T | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGITDPKNPNNYTQARIWGEK | |
| | YFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 234 |
| DAs_L55 | TIHNLRFSSDTTPKPLVIVTPSHVSHIQGTILCSKKVGLQIRTRSGG | |
| 6F | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQFSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 235 |
| DAs_L38 | TIHNLRFSSDTTPKPLVIVTPSHVSHIQGTILCSKKVGLQIRTRSGG | |
| 1F | HDSEGMSYISQVPFVIVDLRNMRSIKIDVHSQTAWVEAGATLGE | |
| | VYYWVNEKNESFSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 236 |
| DAs_E47 | TIHNLRFSSDTTPKPLVIVTPSHVSHIQGTILCSKKVGLQIRTRSGG | |
| 9G | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHGLVKLVNKWQNIAYKYDKD | |
| | LLLMTHFITRNITDNQGKNKTAIHTYFSSVFLGGVDSLVDLMNK | |
| | SFPELGIKKTDCRQLSWIDTIIFYSGVVNYDTDNFNKEILLDRSAG | |
| | QNGAFKIKLDYVKKPIPESVFVQILEKLYEEDIGAGMYALYPYG | |
| | GIMDEISESAIPFPHRAGILYELWYICSWEKQEDNEKHLNWIRNI | |
| | YNFMTPYVSQNPRLAYLNYRDLDIGINDPKNPNNYTQARIWGE | |
| | KYFGKNFDRLVKVKTLVDPNNFFRNEQSIPPLPRHRH | |
| PIR3_CB | NIQTSIANPRENFLKCFSQYIPNNATNLKLVYTQNNPLYMSVLNS | 237 |
| DAs_I562 | TIHNLRFSSDTTPKPLVIVTPSHVSHIQGTILCSKKVGLQIRTRSGG | |
| T | HDSEGMSYISQVPFVIVDLRNMRSIKIDVHSQTAWVEAGATLGE | |
| | VYYWVNEKNESLSLAAGYCPTVCAGGHFGGGGYGPLMRSYGL | |
| | AADNIIDAHLVNVHGKVLDRKSMGEDLFWALRGGGAESFGIIV | |
| | AWKIRLVAVPKSTMFSVKKIMEIHELVKLVNKWQNIAYKYDKD | |
| | LLLMTHFITRNITDNQGKNKTAIHTYFSSVFLGGVDSLVDLMNK | |
| | SFPELGIKKTDCRQLSWIDTTIFYSGVVNYDTDNFNKEILLDRSA | |
| | GQNGAFKIKLDYVKKPIPESVFVQILEKLYEEDIGAGMYALYPY | |
| | GGIMDEISESAIPFPHRAGILYELWYICSWEKQEDNEKHLNWIRN | |
| | IYNFMTPYVSQNPRLAYLNYRDLDIGINDPKNPNNYTQARIWGE | |
| | KYFGKNFDRLVKVKTLVDPNNFFRNEQSIPPLPRHRH | |

TABLE 25
yCBGA_0523 mutant strains

| | First | | | Second | | | |
|---|---|---|---|---|---|---|---|
| | mutation | | | mutation | | | |
| | site on | | | site on | | | |
| | CBDAs | | | CBDAs | | | |
| | coding | | | coding | | | |
| | sequence | | | sequence | | | Average |
| | without | native | new | without | native | new | titer of |
| | the signal | amino | amino | the signal | amino | amino | CBDA |
| Constructs | sequence | acid | acid | sequence | acid | acid | mg/L |
| wild type control | wild type | N/A | N/A | N/A | N/A | N/A | 104.9 |
| construct | control | | | | | | |
| | construct | | | | | | |
| PIR3_CBDAS_G307A | 74 | G | A | N/A | N/A | N/A | 244.8 |
| PIR3_CBDAS_H425D | 192 | H | D | N/A | N/A | N/A | 199.5 |
| PIR3_CBDAS_I620V | 387 | I | V | N/A | N/A | N/A | 181.3 |
| PIR3_CBDAS_I676V | 443 | I | V | N/A | N/A | N/A | 155.6 |
| PIR3_CBDAS_I700L | 467 | I | L | N/A | N/A | N/A | 141.8 |
| PIR3_CBDAS_L442I | 209 | L | I | N/A | N/A | N/A | 169.6 |
| PIR3_CBDAS_M468I | 235 | M | I | N/A | N/A | N/A | 191.4 |
| PIR3_CBDAS_N269D | 36 | N | D | N/A | N/A | N/A | 210.6 |
| PIR3_CBDAS_P464P5 | 231 | P | PS | N/A | N/A | N/A | 201.3 |
| PIR3_CBDAS_R243Q | 10 | R | Q | N/A | N/A | N/A | 162.8 |
| PIR3_CBDAS_S328A | 95 | S | A | N/A | N/A | N/A | 125.2 |
| PIR3_CBDAS_S449N | 216 | S | N | N/A | N/A | N/A | 156.5 |
| PIR3_CBDAS_V454A | 221 | V | A | N/A | N/A | N/A | 145.6 |
| PIR3_CBDAS_Y571F | 338 | Y | F | N/A | N/A | N/A | 137.2 |
| PIR3_CBDAS_G307A_ | 74 | G | A | 192 | H | D | 371.8 |
| H425D | | | | | | | |
| PIR3_CBDAS_G307A_ | 74 | G | A | 387 | I | V | 373.2 |
| 1620V | | | | | | | |
| PIR3_CBDAS_G307A_ | 74 | G | A | 443 | I | V | 173.2 |
| I676V | | | | | | | |
| PIR3_CBDAS_G307A_ | 74 | G | A | 467 | I | L | 327.4 |
| 1700L | | | | | | | |
| PIR3_CBDAS_G307A_ | 74 | G | A | 209 | L | I | 353.6 |
| L442I | | | | | | | |
| PIR3_CBDAS_G307A_ | 74 | G | A | 235 | M | I | 391.7 |
| M468I | | | | | | | |
| PIR3_CBDAS_G307A_ | 74 | G | A | 36 | N | D | 281.2 |
| N269D | | | | | | | |
| PIR3_CBDAS_G307A_ | 74 | G | A | 231 | P | PS | 395.3 |
| P464PS | | | | | | | |
| PIR3_CBDAS_G307A_ | 74 | G | A | 10 | R | Q | 270.2 |
| R243Q | | | | | | | |
| PIR3_CBDAS_G307A_ | 74 | G | A | 95 | S | A | 306.8 |
| S328A | | | | | | | |
| PIR3_CBDAS_G307A_ | 74 | G | A | 216 | S | N | 346.8 |
| S449N | | | | | | | |
| PIR3_CBDAS_G307A_ | 74 | G | A | 221 | V | A | 314.2 |
| V454A | | | | | | | |
| PIR3_CBDAS_G307A_ | 74 | G | A | 338 | Y | F | 307.5 |
| Y571F | | | | | | | |
| PIR3_CBDAS_H425D_ | 192 | H | D | 235 | M | I | 324.1 |
| M468I | | | | | | | |
| PIR3_CBDAS_H425D_ | 192 | H | D | 36 | N | D | 351.5 |
| N269D | | | | | | | |
| PIR3_CBDAS_H425D_ | 192 | H | D | 221 | V | A | 277.8 |
| V454A | | | | | | | |
| PIR3_CBDAS_N269D_ | 36 | N | D | 235 | M | I | 332.8 |
| M468I | | | | | | | |
| PIR3_CBDAS_P464P5 | 231 | P | PS | 192 | H | D | 317.6 |
| H425D | | | | | | | |
| PIR3_CBDAS_P464P5 | 231 | P | PS | 36 | N | D | 348.0 |
| N269D | | | | | | | |
| PIR3_CBDAS_P464P5 | 231 | P | PS | 235 | M | I | 21.4 |
| T468I | | | | | | | |
| PIR3 CBDAS_P464PS | 231 | P | PS | 221 | V | A | 268.0 |
| V454A | | | | | | | |
| PIR3_CBDAS_S449N_ | 216 | S | N | 192 | H | D | 292.2 |
| H425D | | | | | | | |
| PIR3_CBDAS_S449N_I | 216 | S | N | 387 | I | V | 280.7 |
| 620V | | | | | | | |
| PIR3_CBDAS_S449N_I | 216 | S | N | 443 | I | V | 202.2 |
| 676V | | | | | | | |
| PIR3_CBDAS_S449N_I | 216 | S | N | 467 | I | L | 147.3 |
| 700L | | | | | | | |
| PIR3_CBDAS_S449N_ | 216 | S | N | 209 | L | I | 248.3 |
| L442I | | | | | | | |
| PIR3_CBDAS_S449N_ | 216 | S | N | 235 | M | I | 201.7 |
| M468I | | | | | | | |
| PIR3_CBDAS_S449N_ | 216 | S | N | 36 | N | D | 312.3 |
| N269D | | | | | | | |
| PIR3_CBDAS_S449N_ | 216 | S | N | 231 | P | PS | 238.2 |
| P464PS | | | | | | | |
| PIR3_CBDAS_S449N_ | 216 | S | N | 10 | R | Q | 229.1 |
| R243Q | | | | | | | |
| PIR3_CBDAS_S449N_ | 216 | S | N | 95 | S | A | 88.1 |
| S328A | | | | | | | |
| PIR3_CBDAS_S449N_ | 216 | S | N | 221 | V | A | 202.7 |
| V454A | | | | | | | |
| PIR3_CBDAS_S449N_ | 216 | S | N | 338 | Y | F | 148.7 |
| Y571F | | | | | | | |
| PIR3_CBDAS_V454A_ | 221 | V | A | 235 | M | I | 256.8 |
| M468I | | | | | | | |
| PIR3_CBDAS_V454A_ | 221 | V | A | 36 | N | D | 275.4 |
| N269D | | | | | | | |

TABLE 26
yCBGA_0523 mutant strains

| | | SEQ ID |
|---|---|---|
| Constructs | CBDA amino acid sequence | NO: |
| wild type | NIQTSIANPRENFLKCFSQYIPNNATNLKLVYTQNNPLYMSVLNSTIHN | 238 |
| control | LRFSSDTTPKPLVIVTPSHVSHIQGTILCSKKVGLQIRTRSGGHDSEGMS | |
| construct | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVHGK | |
| | VLDRKSMGEDLFWALRGGGAESFGIIVAWKIRLVAVPKSTMFSVKKI | |
| | MEIHELVKLVNKWQNIAYKYDKDLLLMTHFITRNITDNQGKNKTAIHT | |
| | YFSSVFLGGVDSLVDLMNKSFPELGIKKTDCRQLSWIDTIIFYSGVVNY | |
| | DTDNFNKEILLDRSAGQNGAFKIKLDYVKKPIPESVFVQILEKLYEEDIG | |
| | AGMYALYPYGGIMDEISESAIPFPHRAGILYELWYICSWEKQEDNEKH | |
| | LNWIRNIYNFMTPYVSQNPRLAYLNYRDLDIGINDPKNPNNYTQARIW | |
| | GEKYFGKNFDRLVKVKTLVDPNNFFRNEQSIPPLPRHRH | |
| PIR3_ | NIQTSIANPRENFLKCFSQYIPNNATNLKLVYTQNNPLYMSVLNSTIHN | 239 |
| CBDAS_ | LRFSSDTTPKPLVIVTPSHVSHIQATILCSKKVGLQIRTRSGGHDSEGMS | |
| G307A | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVHGK | |
| | VLDRKSMGEDLFWALRGGGAESFGIIVAWKIRLVAVPKSTMFSVKKI | |
| | MEIHELVKLVNKWQNIAYKYDKDLLLMTHFITRNITDNQGKNKTAIHT | |
| | YFSSVFLGGVDSLVDLMNKSFPELGIKKTDCRQLSWIDTIIFYSGVVNY | |
| | DTDNFNKEILLDRSAGQNGAFKIKLDYVKKPIPESVFVQILEKLYEEDIG | |
| | AGMYALYPYGGIMDEISESAIPFPHRAGILYELWYICSWEKQEDNEKH | |
| | LNWIRNIYNFMTPYVSQNPRLAYLNYRDLDIGINDPKNPNNYTQARIW | |
| | GEKYFGKNFDRLVKVKTLVDPNNIFFRNEQSIPPLPRHRH | |
| PIR3_ | NIQTSIANPRENFLKCFSQYIPNNATNLKLVYTQNNPLYMSVLNSTIHN | 240 |
| CBDAS_ | LRFSSDTTPKPLVIVTPSHVSHIQGTILCSKKVGLQIRTRSGGHDSEGMS | |
| H425D | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVDGK | |
| | VLDRKSMGEDLFWALRGGGAESFGIIVAWKIRLVAVPKSTMFSVKKI | |
| | MEIHELVKLVNKWQNIAYKYDKDLLLMTHFITRNITDNQGKNKTAIHT | |
| | YFSSVFLGGVDSLVDLMNKSFPELGIKKTDCRQLSWIDTIIFYSGVVNY | |
| | DTDNFNKEILLDRSAGQNGAFKIKLDYVKKPIPESVFVQILEKLYEEDIG | |
| | AGMYALYPYGGIMDEISESAIPFPHRAGILYELWYICSWEKQEDNEKH | |
| | LNWIRNIYNFMTPYVSQNPRLAYLNYRDLDIGINDPKNPNNYTQARIW | |
| | GEKYFGKNFDRLVKVKTLVDPNNFFRNEQSIPPLPRHRH | |
| PIR3_CB | NIQTSIANPRENFLKCFSQYIPNNATNLKLVYTQNNPLYMSVLNSTIHN | 241 |
| DAS_I620 | LRFSSDTTPKPLVIVTPSHVSHIQGTILCSKKVGLQIRTRSGGHDSEGMS | |
| V | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVHGK | |
| | VLDRKSMGEDLFWALRGGGAESFGIIVAWKIRLVAVPKSTMFSVKKI | |
| | MEIHELVKLVNKWQNIAYKYDKDLLLMTHFITRNITDNQGKNKTAIHT | |
| | YFSSVFLGGVDSLVDLMNKSFPELGIKKTDCRQLSWIDTIIFYSGVVNY | |
| | DTDNFNKEILLDRSAGQNGAFKIKLDYVKKPIPESVFVQILEKLYEEDV | |
| | GAGMYALYPYGGIMDEISESAIPFPHRAGILYELWYICSWEKQEDNEK | |
| | HLNWIRNIYNFMTPYVSQNPRLAYLNYRDLDIGINDPKNPNNYTQARI | |
| | WGEKYFGKNFDRLVKVKTLVDPNNFFRNEQSIPPLPRHRH | |
| PIR3_ | NIQTSIANPRENFLKCFSQYIPNNATNLKLVYTQNNPLYMSVLNSTIHN | 242 |
| CBDAS_ | LRFSSDTTPKPLVIVTPSHVSHIQGTILCSKKVGLQIRTRSGGHDSEGMS | |
| I676V | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVHGK | |
| | VLDRKSMGEDLFWALRGGGAESFGIIVAWKIRLVAVPKSTMFSVKKI | |
| | MEIHELVKLVNKWQNIAYKYDKDLLLMTHFITRNITDNQGKNKTAIHT | |
| | YFSSVFLGGVDSLVDLMNKSFPELGIKKTDCRQLSWIDTIIFYSGVVNY | |
| | DTDNFNKEILLDRSAGQNGAFKIKLDYVKKPIPESVFVQILEKLYEEDIG | |
| | AGMYALYPYGGIMDEISESAIPFPHRAGILYELWYICSWEKQEDNEKH | |
| | LNWIRNVYNFMTPYVSQNPRLAYLNYRDLDIGINDPKNPNNYTQARI | |
| | WGEKYFGKNFDRLVKVKTLVDPNNFFRNEQSIPPLPRHRH | |
| PIR3_ | NIQTSIANPRENFLKCFSQYIPNNATNLKLVYTQNNPLYMSVLNSTIHN | 243 |
| CBDAS_ | LRFSSDTTPKPLVIVTPSHVSHIQGTILCSKKVGLQIRTRSGGHDSEGMS | |
| I700L | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVHGK | |
| | VLDRKSMGEDLFWALRGGGAESFGIIVAWKIRLVAVPKSTMFSVKKI | |
| | MEIHELVKLVNKWQNIAYKYDKDLLLMTHFITRNITDNQGKNKTAIHT | |
| | YFSSVFLGGVDSLVDLMNKSFPELGIKKTDCRQLSWIDTIIFYSGVVNY | |
| | DTDNFNKEILLDRSAGQNGAFKIKLDYVKKPIPESVFVQILEKLYEEDIG | |
| | AGMYALYPYGGIMDEISESAIPFPHRAGILYELWYICSWEKQEDNEKH | |
| | LNWIRNIYNFMTPYVSQNPRLAYLNYRDLDLGINDPKNPNNYTQARIW | |
| | GEKYFGKNFDRLVKVKTLVDPNNFFRNEQSIPPLPRHRH | |
| PIR3_ | NIQTSIANPRENFLKCFSQYIPNNATNLKLVYTQNNPLYMSVLNSTIHN | 244 |
| CBDAS_ | LRFSSDTTPKPLVIVTPSHVSHIQGTILCSKKVGLQIRTRSGGHDSEGMS | |
| L442I | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVHGK | |
| | VLDRKSMGEDLFWAIRGGGAESFGIIVAWKIRLVAVPKSTMFSVKKIM | |
| | EIHELVKLVNKWQNIAYKYDKDLLLMTHFITRNITDNQGKNKTAIHTY | |
| | FSSVFLGGVDSLVDLMNKSFPELGIKKTDCRQLSWIDTIIFYSGVVNYD | |
| | TDNFNKEILLDRSAGQNGAFKIKLDYVKKPIPESVFVQILEKLYEEDIGA | |
| | GMYALYPYGGIMDEISESAIPFPHRAGILYELWYICSWEKQEDNEKHL | |
| | NWIRNIYNFMTPYVSQNPRLAYLNYRDLDIGINDPKNPNNYTQARIWG | |
| | EKYFGKNFDRLVKVKTLVDPNNFFRNEQSIPPLPRHRH | |
| PIR3_ | NIQTSIANPRENFLKCFSQYIPNNATNLKLVYTQNNPLYMSVLNSTIHN | 245 |
| CBDAS_ | LRFSSDTTPKPLVIVTPSHVSHIQGTILCSKKVGLQIRTRSGGHDSEGMS | |
| M468I | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVHGK | |
| | VLDRKSMGEDLFWALRGGGAESFGIIVAWKIRLVAVPKSTIFSVKKIM | |
| | EIHELVKLVNKWQNIAYKYDKDLLLMTHFITRNITDNQGKNKTAIHTY | |
| | FSSVFLGGVDSLVDLMNKSFPELGIKKTDCRQLSWIDTIIFYSGVVNYD | |
| | TDNFNKEILLDRSAGQNGAFKIKLDYVKKPIPESVFVQILEKLYEEDIGA | |
| | GMYALYPYGGIMDEISESAIPFPHRAGILYELWYICSWEKQEDNEKHL | |
| | NWIRNIYNFMTPYVSQNPRLAYLNYRDLDIGINDPKNPNNYTQARIWG | |
| | EKYFGKNFDRLVKVKTLVDPNNFFRNEQSIPPLPRHRH | |
| PIR3_ | NIQTSIANPRENFLKCFSQYIPNNATNLKLVYTQNDPLYMSVLNSTIHN | 246 |
| CBDAS_ | LRFSSDTTPKPLVIVTPSHVSHIQGTILCSKKVGLQIRTRSGGHDSEGMS | |
| N269D | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVHGK | |
| | VLDRKSMGEDLFWALRGGGAESFGIIVAWKIRLVAVPKSTMFSVKKI | |
| | MEIHELVKLVNKWQNIAYKYDKDLLLMTHFITRNITDNQGKNKTAIHT | |
| | YFSSVFLGGVDSLVDLMNKSFPELGIKKTDCRQLSWIDTIIFYSGVVNY | |
| | DTDNFNKEILLDRSAGQNGAFKIKLDYVKKPIPESVFVQILEKLYEEDIG | |
| | AGMYALYPYGGIMDEISESAIPFPHRAGILYELWYICSWEKQEDNEKH | |
| | LNWIRNIYNFMTPYVSQNPRLAYLNYRDLDIGINDPKNPNNYTQARIW | |
| | GEKYFGKNFDRLVKVKTLVDPNNFFRNEQSIPPLPRHRH | |
| PIR3_ | NIQTSIANPRENFLKCFSQYIPNNATNLKLVYTQNNPLYMSVLNSTIHN | 247 |
| CBDAS_ | LRFSSDTTPKPLVIVTPSHVSHIQGTILCSKKVGLQIRTRSGGHDSEGMS | |
| P464PS | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVHGK | |
| | VLDRKSMGEDLFWALRGGGAESFGIIVAWKIRLVAVPSKSTMFSVKKI | |
| | MEIHELVKLVNKWQNIAYKYDKDLLLMTHFITRNITDNQGKNKTAIHT | |
| | YFSSVFLGGVDSLVDLMNKSFPELGIKKTDCRQLSWIDTIIFYSGVVNY | |
| | DTDNFNKEILLDRSAGQNGAFKIKLDYVKKPIPESVFVQILEKLYEEDIG | |
| | AGMYALYPYGGIMDEISESAIPFPHRAGILYELWYICSWEKQEDNEKH | |
| | LNWIRNIYNFMTPYVSQNPRLAYLNYRDLDIGINDPKNPNNYTQARIW | |
| | GEKYFGKNFDRLVKVKTLVDPNNFFRNEQSIPPLPRHRH | |
| PIR3_ | NIQTSIANPQENFLKCFSQYIPNNATNLKLVYTQNNPLYMSVLNSTIHN | 248 |
| CBDAS_ | LRFSSDTTPKPLVIVTPSHVSHIQGTILCSKKVGLQIRTRSGGHDSEGMS | |
| R243Q | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVHGK | |
| | VLDRKSMGEDLFWALRGGGAESFGIIVAWKIRLVAVPKSTMFSVKKI | |
| | MEIHELVKLVNKWQNIAYKYDKDLLLMTHFITRNITDNQGKNKTAIHT | |
| | YFSSVFLGGVDSLVDLMNKSFPELGIKKTDCRQLSWIDTIIFYSGVVNY | |
| | DTDNFNKEILLDRSAGQNGAFKIKLDYVKKPIPESVFVQILEKLYEEDIG | |
| | AGMYALYPYGGIMDEISESAIPFPHRAGILYELWYICSWEKQEDNEKH | |
| | LNWIRNIYNFMTPYVSQNPRLAYLNYRDLDIGINDPKNPNNYTQARIW | |
| | GEKYFGKNFDRLVKVKTLVDPNNFFRNEQSIPPLPRHRH | |
| PIR3_ | NIQTSIANPRENFLKCFSQYIPNNATNLKLVYTQNNPLYMSVLNSTIHN | 249 |
| CBDAS- | LRFSSDTTPKPLVIVTPSHVSHIQGTILCSKKVGLQIRTRSGGHDAEGMS | |
| S328A | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVHGK | |
| | VLDRKSMGEDLFWALRGGGAESFGIIVAWKIRLVAVPKSTMFSVKKI | |
| | MEIHELVKLVNKWQNIAYKYDKDLLLMTHFITRNITDNQGKNKTAIHT | |
| | YFSSVFLGGVDSLVDLMNKSFPELGIKKTDCRQLSWIDTIIFYSGVVNY | |
| | DTDNFNKEILLDRSAGQNGAFKIKLDYVKKPIPESVFVQILEKLYEEDIG | |
| | AGMYALYPYGGIMDEISESAIPFPHRAGILYELWYICSWEKQEDNEKH | |
| | LNWIRNIYNFMTPYVSQNPRLAYLNYRDLDIGINDPKNPNNYTQARIW | |
| | GEKYFGKNFDRLVKVKTLVDPNNFFRNEQSIPPLPRHRH | |
| PIR3_ | NIQTSIANPRENFLKCFSQYIPNNATNLKLVYTQNNPLYMSVLNSTIHN | 250 |
| CBDAS_ | LRFSSDTTPKPLVIVTPSHVSHIQGTILCSKKVGLQIRTRSGGHDSEGMS | |
| S449N | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVHGK | |
| | VLDRKSMGEDLFWALRGGGAENFGIIVAWKIRLVAVPKSTMFSVKKI | |
| | MEIHELVKLVNKWQNIAYKYDKDLLLMTHFITRNITDNQGKNKTAIHT | |
| | YFSSVFLGGVDSLVDLMNKSFPELGIKKTDCRQLSWIDTIIFYSGVVNY | |
| | DTDNFNKEILLDRSAGQNGAFKIKLDYVKKPIPESVFVQILEKLYEEDIG | |
| | AGMYALYPYGGIMDEISESAIPFPHRAGILYELWYICSWEKQEDNEKH | |
| | LNWIRNIYNFMTPYVSQNPRLAYLNYRDLDIGINDPKNPNNYTQARIW | |
| | GEKYFGKNFDRLVKVKTLVDPNNFFRNEQSIPPLPRHRH | |
| PIR3_ | NIQTSIANPRENFLKCFSQYIPNNATNLKLVYTQNNPLYMSVLNSTIHN | 251 |
| CBDAS_ | LRFSSDTTPKPLVIVTPSHVSHIQGTILCSKKVGLQIRTRSGGHDSEGMS | |
| V454A | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVHGK | |
| | VLDRKSMGEDLFWALRGGGAESFGIIAAWKIRLVAVPKSTMFSVKKI | |
| | MEIHELVKLVNKWQNIAYKYDKDLLLMTHFITRNITDNQGKNKTAIHT | |
| | YFSSVFLGGVDSLVDLMNKSFPELGIKKTDCRQLSWIDTIIFYSGVVNY | |
| | DTDNFNKEILLDRSAGQNGAFKIKLDYVKKPIPESVFVQILEKLYEEDIG | |
| | AGMYALYPYGGIMDEISESAIPFPHRAGILYELWYICSWEKQEDNEKH | |
| | LNWIRNIYNFMTPYVSQNPRLAYLNYRDLDIGINDPKNPNNYTQARIW | |
| | GEKYFGKNFDRLVKVKTLVDPNNFFRNEQSIPPLPRHRH | |
| PIR3_ | NIQTSIANPRENFLKCFSQYIPNNATNLKLVYTQNNPLYMSVLNSTIHN | 252 |
| CBDAS_ | LRFSSDTTPKPLVIVTPSHVSHIQGTILCSKKVGLQIRTRSGGHDSEGMS | |
| Y571F | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVHGK | |
| | VLDRKSMGEDLFWALRGGGAESFGIIVAWKIRLVAVPKSTMFSVKKI | |
| | MEIHELVKLVNKWQNIAYKYDKDLLLMTHFITRNITDNQGKNKTAIHT | |
| | YFSSVFLGGVDSLVDLMNKSFPELGIKKTDCRQLSWIDTIIFYSGVVNF | |
| | DTDNFNKEILLDRSAGQNGAFKIKLDYVKKPIPESVFVQILEKLYEEDIG | |
| | AGMYALYPYGGIMDEISESAIPFPHRAGILYELWYICSWEKQEDNEKH | |
| | LNWIRNIYNFMTPYVSQNPRLAYLNYRDLDIGINDPKNPNNYTQARIW | |
| | GEKYFGKNFDRLVKVKTLVDPNNFFRNEQSIPPLPRHRH | |
| PIR3_ | NIQTSIANPRENFLKCFSQYIPNNATNLKLVYTQNNPLYMSVLNSTIHN | 253 |
| CBDAS_ | LRFSSDTTPKPLVIVTPSHVSHIQATILCSKKVGLQIRTRSGGHDSEGMS | |
| G307A_ | YISQVPFVIVDLRNMRSIKIDVHSQTAWVEAGATLGEVYYWVNEKNES | |
| H425D | LSLAAGYCPTVCAGGHFGGGGYGPLMRSYGLAADNIIDAHLVNVDGK | |
| | VLDRKSMGEDLFWALRGGGAESFGIIVAWKIRLVAVPKSTMFSVKKI | |
| | MEIHELVKLVNKWQNIAYKYDKDLLLMTHFITRNITDNQGKNKTAIHT | |
| | YFSSVFLGGVDSLVDLMNKSFPELGIKKTDCRQLSWIDTIIFYSGVVNY | |
| | DTDNFNKEILLDRSAGQNGAFKIKLDYVKKPIPESVFVQILEKLYEEDIG | |
| | AGMYALYPYGGIMDEISESAIPFPHRAGILYELWYICSWEKQEDNEKH | |
| | LNWIRNIYNFMTPYVSQNPRLAYLNYRDLDIGINDPKNPNNYTQARIW | |
| | GEKYFGKNFDRLVKVKTLVDPNNFFRNEQSIPPLPRHRH | |
| PIR3_ | NIQTSIANPRENFLKCFSQYIPNNATNLKLVYTQNNPLYMSVLNSTIHN | 254 |
| CBDAS_ | LRFSSDTTPKPLVIVTPSHVSHIQATILCSKKVGLQIRTRSGGHDSEGMS | |
| G307A_I620V | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVHGK | |
| | VLDRKSMGEDLFWALRGGGAESFGIIVAWKIRLVAVPKSTMFSVKKI | |
| | MEIHELVKLVNKWQNIAYKYDKDLLLMTHFITRNITDNQGKNKTAIHT | |
| | YFSSVFLGGVDSLVDLMNKSFPELGIKKTDCRQLSWIDTIIFYSGVVNY | |
| | DTDNFNKEILLDRSAGQNGAFKIKLDYVKKPIPESVFVQILEKLYEEDV | |
| | GAGMYALYPYGGIMDEISESAIPFPHRAGILYELWYICSWEKQEDNEK | |
| | HLNWIRNIYNFMTPYVSQNPRLAYLNYRDLDIGINDPKNPNNYTQARI | |
| | WGEKYFGKNFDRLVKVKTLVDPNNFFRNEQSIPPLPRHRH | |
| PIR3_ | NIQTSIANPRENFLKCFSQYIPNNATNLKLVYTQNNPLYMSVLNSTIHN | 255 |
| CBDAS_ | LRFSSDTTPKPLVIVTPSHVSHIQATILCSKKVGLQIRTRSGGHDSEGMS | |
| G307A_I676V | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVHGK | |
| | VLDRKSMGEDLFWALRGGGAESFGIIVAWKIRLVAVPKSTMFSVKKI | |
| | MEIHELVKLVNKWQNIAYKYDKDLLLMTHFITRNITDNQGKNKTAIHT | |
| | YFSSVFLGGVDSLVDLMNKSFPELGIKKTDCRQLSWIDTIIFYSGVVNY | |
| | DTDNFNKEILLDRSAGQNGAFKIKLDYVKKPIPESVFVQILEKLYEEDIG | |
| | AGMYALYPYGGIMDEISESAIPFPHRAGILYELWYICSWEKQEDNEKH | |
| | LNWIRNVYNFMTPYVSQNPRLAYLNYRDLDIGINDPKNPNNYTQARI | |
| | WGEKYFGKNFDRLVKVKTLVDPNNFFRNEQSIPPLPRHRH | |
| PIR3_ | NIQTSIANPRENFLKCFSQYIPNNATNLKLVYTQNNPLYMSVLNSTIHN | 256 |
| CBDAS_ | LRFSSDTTPKPLVIVTPSHVSHIQATILCSKKVGLQIRTRSGGHDSEGMS | |
| G307A_I700L | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVHGK | |
| | VLDRKSMGEDLFWALRGGGAESFGIIVAWKIRLVAVPKSTMFSVKKI | |
| | MEIHELVKLVNKWQNIAYKYDKDLLLMTHFITRNITDNQGKNKTAIHT | |
| | YFSSVFLGGVDSLVDLMNKSFPELGIKKTDCRQLSWIDTIIFYSGVVNY | |
| | DTDNFNKEILLDRSAGQNGAFKIKLDYVKKPIPESVFVQILEKLYEEDIG | |
| | AGMYALYPYGGIMDEISESAIPFPHRAGILYELWYICSWEKQEDNEKH | |
| | LNWIRNIYNFMTPYVSQNPRLAYLNYRDLDLGINDPKNPNNYTQARIW | |
| | GEKYFGKNFDRLVKVKTLVDPNNFFRNEQSIPPLPRHRH | |
| PIR3_ | NIQTSIANPRENFLKCFSQYIPNNATNLKLVYTQNNPLYMSVLNSTIHN | 257 |
| CBDAS_ | LRFSSDTTPKPLVIVTPSHVSHIQATILCSKKVGLQIRTRSGGHDSEGMS | |
| G307A_L442I | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVHGK | |
| | VLDRKSMGEDLFWAIRGGGAESFGIIVAWKIRLVAVPKSTMFSVKKIM | |
| | EIHELVKLVNKWQNIAYKYDKDLLLMTHFITRNITDNQGKNKTAIHTY | |
| | FSSVFLGGVDSLVDLMNKSFPELGIKKTDCRQLSWIDTIIFYSGVVNYD | |
| | TDNFNKEILLDRSAGQNGAFKIKLDYVKKPIPESVFVQILEKLYEEDIGA | |
| | GMYALYPYGGIMDEISESAIPFPHRAGILYELWYICSWEKQEDNEKHL | |
| | NWIRNIYNFMTPYVSQNPRLAYLNYRDLDIGINDPKNPNNYTQARIWG | |
| | EKYFGKNFDRLVKVKTLVDPNNFFRNEQSIPPLPRHRH | |
| PIR3_ | NIQTSIANPRENFLKCFSQYIPNNATNLKLVYTQNNPLYMSVLNSTIHN | 258 |
| CBDAS_ | LRFSSDTTPKPLVIVTPSHVSHIQATILCSKKVGLQIRTRSGGHDSEGMS | |
| G307A_M468I | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVHGK | |
| | VLDRKSMGEDLFWALRGGGAESFGIIVAWKIRLVAVPKSTIFSVKKIM | |
| | EIHELVKLVNKWQNIAYKYDKDLLLMTHFITRNITDNQGKNKTAIHTY | |
| | FSSVFLGGVDSLVDLMNKSFPELGIKKTDCRQLSWIDTIIFYSGVVNYD | |
| | TDNFNKEILLDRSAGQNGAFKIKLDYVKKPIPESVFVQILEKLYEEDIGA | |
| | GMYALYPYGGIMDEISESAIPFPHRAGILYELWYICSWEKQEDNEKHL | |
| | NWIRNIYNFMTPYVSQNPRLAYLNYRDLDIGINDPKNPNNYTQARIWG | |
| | EKYFGKNFDRLVKVKTLVDPNNFFRNEQSIPPLPRHRH | |
| PIR3_ | NIQTSIANPRENFLKCFSQYIPNNATNLKLVYTQNDPLYMSVLNSTIHN | 259 |
| CBDAS_ | LRFSSDTTPKPLVIVTPSHVSHIQATILCSKKVGLQIRTRSGGHDSEGMS | |
| G307A_ | YISQVPFVIVDLRNMRSIKIDVHSQTAWVEAGATLGEVYYWVNEKNES | |
| N269D | LSLAAGYCPTVCAGGHFGGGGYGPLMRSYGLAADNIIDAHLVNVHGK | |
| | VLDRKSMGEDLFWALRGGGAESFGIIVAWKIRLVAVPKSTMFSVKKI | |
| | MEIHELVKLVNKWQNIAYKYDKDLLLMTHFITRNITDNQGKNKTAIHT | |
| | YFSSVFLGGVDSLVDLMNKSFPELGIKKTDCRQLSWIDTIIFYSGVVNY | |
| | DTDNFNKEILLDRSAGQNGAFKIKLDYVKKPIPESVFVQILEKLYEEDIG | |
| | AGMYALYPYGGIMDEISESAIPFPHRAGILYELWYICSWEKQEDNEKH | |
| | LNWIRNIYNFMTPYVSQNPRLAYLNYRDLDIGINDPKNPNNYTQARIW | |
| | GEKYFGKNFDRLVKVKTLVDPNNFFRNEQSIPPLPRHRH | |
| PIR3_ | NIQTSIANPRENFLKCFSQYIPNNATNLKLVYTQNNPLYMSVLNSTIHN | 260 |
| CBDAS_ | LRFSSDTTPKPLVIVTPSHVSHIQATILCSKKVGLQIRTRSGGHDSEGMS | |
| G307A_P464PS | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVHGK | |
| | VLDRKSMGEDLFWALRGGGAESFGIIVAWKIRLVAVPSKSTMFSVKKI | |
| | MEIHELVKLVNKWQNIAYKYDKDLLLMTHFITRNITDNQGKNKTAIHT | |
| | YFSSVFLGGVDSLVDLMNKSFPELGIKKTDCRQLSWIDTIIFYSGVVNY | |
| | DTDNFNKEILLDRSAGQNGAFKIKLDYVKKPIPESVFVQILEKLYEEDIG | |
| | AGMYALYPYGGIMDEISESAIPFPHRAGILYELWYICSWEKQEDNEKH | |
| | LNWIRNIYNFMTPYVSQNPRLAYLNYRDLDIGINDPKNPNNYTQARIW | |
| | GEKYFGKNFDRLVKVKTLVDPNNFFRNEQSIPPLPRHRH | |
| PIR3_ | NIQTSIANPQENFLKCFSQYIPNNATNLKLVYTQNNPLYMSVLNSTIHN | 261 |
| CBDAS_ | LRFSSDTTPKPLVIVTPSHVSHIQATILCSKKVGLQIRTRSGGHDSEGMS | |
| G307A_R243Q | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVHGK | |
| | VLDRKSMGEDLFWALRGGGAESFGIIVAWKIRLVAVPKSTMFSVKKI | |
| | MEIHELVKLVNKWQNIAYKYDKDLLLMTHFITRNITDNQGKNKTAIHT | |
| | YFSSVFLGGVDSLVDLMNKSFPELGIKKTDCRQLSWIDTIIFYSGVVNY | |
| | DTDNFNKEILLDRSAGQNGAFKIKLDYVKKPIPESVFVQILEKLYEEDIG | |
| | AGMYALYPYGGIMDEISESAIPFPHRAGILYELWYICSWEKQEDNEKH | |
| | LNWIRNIYNFMTPYVSQNPRLAYLNYRDLDIGINDPKNPNNYTQARIW | |
| | GEKYFGKNFDRLVKVKTLVDPNNFFRNEQSIPPLPRHRH | |
| PIR3_ | NIQTSIANPRENFLKCFSQYIPNNATNLKLVYTQNNPLYMSVLNSTIHN | 262 |
| CBDAS_ | LRFSSDTTPKPLVIVTPSHVSHIQATILCSKKVGLQIRTRSGGHDAEGMS | |
| G307A_S328A | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVHGK | |
| | VLDRKSMGEDLFWALRGGGAESFGIIVAWKIRLVAVPKSTMFSVKKI | |
| | MEIHELVKLVNKWQNIAYKYDKDLLLMTHFITRNITDNQGKNKTAIHT | |
| | YFSSVFLGGVDSLVDLMNKSFPELGIKKTDCRQLSWIDTIIFYSGVVNY | |
| | DTDNFNKEILLDRSAGQNGAFKIKLDYVKKPIPESVFVQILEKLYEEDIG | |
| | AGMYALYPYGGIMDEISESAIPFPHRAGILYELWYICSWEKQEDNEKH | |
| | LNWIRNIYNFMTPYVSQNPRLAYLNYRDLDIGINDPKNPNNYTQARIW | |
| | GEKYFGKNFDRLVKVKTLVDPNNFFRNEQSIPPLPRHRH | |
| PIR3_ | NIQTSIANPRENFLKCFSQYIPNNATNLKLVYTQNNPLYMSVLNSTIHN | 263 |
| CBDAS_ | LRFSSDTTPKPLVIVTPSHVSHIQATILCSKKVGLQIRTRSGGHDSEGMS | |
| G307A_S449N | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVHGK | |
| | VLDRKSMGEDLFWALRGGGAENFGIIVAWKIRLVAVPKSTMFSVKKI | |
| | MEIHELVKLVNKWQNIAYKYDKDLLLMTHFITRNITDNQGKNKTAIHT | |
| | YFSSVFLGGVDSLVDLMNKSFPELGIKKTDCRQLSWIDTIIFYSGVVNY | |
| | DTDNFNKEILLDRSAGQNGAFKIKLDYVKKPIPESVFVQILEKLYEEDIG | |
| | AGMYALYPYGGIMDEISESAIPFPHRAGILYELWYICSWEKQEDNEKH | |
| | LNWIRNIYNFMTPYVSQNPRLAYLNYRDLDIGINDPKNPNNYTQARIW | |
| | GEKYFGKNFDRLVKVKTLVDPNNFIRNEQSIPPLPRHRH | |
| PIR3_ | NIQTSIANPRENFLKCFSQYIPNNATNLKLVYTQNNPLYMSVLNSTIHN | 264 |
| CBDAS_ | LRFSSDTTPKPLVIVTPSHVSHIQATILCSKKVGLQIRTRSGGHDSEGMS | |
| G307A_V454A | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVHGK | |
| | VLDRKSMGEDLFWALRGGGAESFGIIAAWKIRLVAVPKSTMFSVKKI | |
| | MEIHELVKLVNKWQNIAYKYDKDLLLMTHFITRNITDNQGKNKTAIHT | |
| | YFSSVFLGGVDSLVDLMNKSFPELGIKKTDCRQLSWIDTIIFYSGVVNY | |
| | DTDNFNKEILLDRSAGQNGAFKIKLDYVKKPIPESVFVQILEKLYEEDIG | |
| | AGMYALYPYGGIMDEISESAIPFPHRAGILYELWYICSWEKQEDNEKH | |
| | LNWIRNIYNFMTPYVSQNPRLAYLNYRDLDIGINDPKNPNNYTQARIW | |
| | GEKYFGKNFDRLVKVKTLVDPNNFFRNEQSIPPLPRHRH | |
| PIR3_ | NIQTSIANPRENFLKCFSQYIPNNATNLKLVYTQNNPLYMSVLNSTIHN | 265 |
| CBDAS_ | LRFSSDTTPKPLVIVTPSHVSHIQATILCSKKVGLQIRTRSGGHDSEGMS | |
| G307A_Y571F | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVHGK | |
| | VLDRKSMGEDLFWALRGGGAESFGIIVAWKIRLVAVPKSTMFSVKKI | |
| | MEIHELVKLVNKWQNIAYKYDKDLLLMTHFITRNITDNQGKNKTAIHT | |
| | YFSSVFLGGVDSLVDLMNKSFPELGIKKTDCRQLSWIDTIIFYSGVVNF | |
| | DTDNFNKEILLDRSAGQNGAFKIKLDYVKKPIPESVFVQILEKLYEEDIG | |
| | AGMYALYPYGGIMDEISESAIPFPHRAGILYELWYICSWEKQEDNEKH | |
| | LNWIRNIYNFMTPYVSQNPRLAYLNYRDLDIGINDPKNPNNYTQARIW | |
| | GEKYFGKNFDRLVKVKTLVDPNNFFRNEQSIPPLPRHRH | |
| PIR3_ | NIQTSIANPRENFLKCFSQYIPNNATNLKLVYTQNNPLYMSVLNSTIHN | 266 |
| CBDAS_ | LRFSSDTTPKPLVIVTPSHVSHIQGTILCSKKVGLQIRTRSGGHDSEGMS | |
| H425D_M468I | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVDGK | |
| | VLDRKSMGEDLFWALRGGGAESFGIIVAWKIRLVAVPKSTIFSVKKIM | |
| | EIHELVKLVNKWQNIAYKYDKDLLLMTHFITRNITDNQGKNKTAIHTY | |
| | FSSVFLGGVDSLVDLMNKSFPELGIKKTDCRQLSWIDTIIFYSGVVNYD | |
| | TDNFNKEILLDRSAGQNGAFKIKLDYVKKPIPESVFVQILEKLYEEDIGA | |
| | GMYALYPYGGIMDEISESAIPFPHRAGILYELWYICSWEKQEDNEKHL | |
| | NWIRNIYNFMTPYVSQNPRLAYLNYRDLDIGINDPKNPNNYTQARIWG | |
| | EKYFGKNFDRLVKVKTLVDPNNFFRNEQSIPPLPRHRH | |
| PIR3_ | NIQTSIANPRENFLKCFSQYIPNNATNLKLVYTQNDPLYMSVLNSTIHN | 267 |
| CBDAS_ | LRFSSDTTPKPLVIVTPSHVSHIQGTILCSKKVGLQIRTRSGGHDSEGMS | |
| H425D_N269D | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVDGK | |
| | VLDRKSMGEDLFWALRGGGAESFGIIVAWKIRLVAVPKSTMFSVKKI | |
| | MEIHELVKLVNKWQNIAYKYDKDLLLMTHFITRNITDNQGKNKTAIHT | |
| | YFSSVFLGGVDSLVDLMNKSFPELGIKKTDCRQLSWIDTIIFYSGVVNY | |
| | DTDNFNKEILLDRSAGQNGAFKIKLDYVKKPIPESVFVQILEKLYEEDIG | |
| | AGMYALYPYGGIMDEISESAIPFPHRAGILYELWYICSWEKQEDNEKH | |
| | LNWIRNIYNFMTPYVSQNPRLAYLNYRDLDIGINDPKNPNNYTQARIW | |
| | GEKYFGKNFDRLVKVKTLVDPNNFFRNEQSIPPLPRHRH | |
| PIR3_ | NIQTSIANPRENFLKCFSQYIPNNATNLKLVYTQNNPLYMSVLNSTIHN | 268 |
| CBDAS_ | LRFSSDTTPKPLVIVTPSHVSHIQGTILCSKKVGLQIRTRSGGHDSEGMS | |
| H425D_V454A | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVDGK | |
| | VLDRKSMGEDLFWALRGGGAESFGIIAAWKIRLVAVPKSTMFSVKKI | |
| | MEIHELVKLVNKWQNIAYKYDKDLLLMTHFITRNITDNQGKNKTAIHT | |
| | YFSSVFLGGVDSLVDLMNKSFPELGIKKTDCRQLSWIDTIIFYSGVVNY | |
| | DTDNFNKEILLDRSAGQNGAFKIKLDYVKKPIPESVFVQILEKLYEEDIG | |
| | AGMYALYPYGGIMDEISESAIPFPHRAGILYELWYICSWEKQEDNEKH | |
| | LNWIRNIYNFMTPYVSQNPRLAYLNYRDLDIGINDPKNPNNYTQARIW | |
| | GEKYFGKNFDRLVKVKTLVDPNNFFRNEQSIPPLPRHRH | |
| PIR3_ | NIQTSIANPRENFLKCFSQYIPNNATNLKLVYTQNDPLYMSVLNSTIHN | 269 |
| CBDAS_ | LRFSSDTTPKPLVIVTPSHVSHIQGTILCSKKVGLQIRTRSGGHDSEGMS | |
| N269D_M468I | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVHGK | |
| | VLDRKSMGEDLFWALRGGGAESFGIIVAWKIRLVAVPKSTIFSVKKIM | |
| | EIHELVKLVNKWQNIAYKYDKDLLLMTHFITRNITDNQGKNKTAIHTY | |
| | FSSVFLGGVDSLVDLMNKSFPELGIKKTDCRQLSWIDTIIFYSGVVNYD | |
| | TDNFNKEILLDRSAGQNGAFKIKLDYVKKPIPESVFVQILEKLYEEDIGA | |
| | GMYALYPYGGIMDEISESAIPFPHRAGILYELWYICSWEKQEDNEKHL | |
| | NWIRNIYNFMTPYVSQNPRLAYLNYRDLDIGINDPKNPNNYTQARIWG | |
| | EKYFGKNFDRLVKVKTLVDPNNFFRNEQSIPPLPRHRH | |
| PIR3_ | NIQTSIANPRENFLKCFSQYIPNNATNLKLVYTQNNPLYMSVLNSTIHN | 270 |
| CBDAS_ | LRFSSDTTPKPLVIVTPSHVSHIQGTILCSKKVGLQIRTRSGGHDSEGMS | |
| P464PS_H425D | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVDGK | |
| | VLDRKSMGEDLFWALRGGGAESFGIIVAWKIRLVAVPSKSTMFSVKKI | |
| | MEIHELVKLVNKWQNIAYKYDKDLLLMTHFITRNITDNQGKNKTAIHT | |
| | YFSSVFLGGVDSLVDLMNKSFPELGIKKTDCRQLSWIDTIIFYSGVVNY | |
| | DTDNFNKEILLDRSAGQNGAFKIKLDYVKKPIPESVFVQILEKLYEEDIG | |
| | AGMYALYPYGGIMDEISESAIPFPHRAGILYELWYICSWEKQEDNEKH | |
| | LNWIRNIYNFMTPYVSQNPRLAYLNYRDLDIGINDPKNPNNYTQARIW | |
| | GEKYFGKNFDRLVKVKTLVDPNNFFRNEQSIPPLPRHRH | |
| PIR3_ | NIQTSIANPRENFLKCFSQYIPNNATNLKLVYTQNDPLYMSVLNSTIHN | 271 |
| CBDAS_ | LRFSSDTTPKPLVIVTPSHVSHIQGTILCSKKVGLQIRTRSGGHDSEGMS | |
| P464PS_N269D | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVHGK | |
| | VLDRKSMGEDLFWALRGGGAESFGIIVAWKIRLVAVPSKSTMFSVKKI | |
| | MEIHELVKLVNKWQNIAYKYDKDLLLMTHFITRNITDNQGKNKTAIHT | |
| | YFSSVFLGGVDSLVDLMNKSFPELGIKKTDCRQLSWIDTIIFYSGVVNY | |
| | DTDNFNKEILLDRSAGQNGAFKIKLDYVKKPIPESVFVQILEKLYEEDIG | |
| | AGMYALYPYGGIMDEISESAIPFPHRAGILYELWYICSWEKQEDNEKH | |
| | LNWIRNIYNFMTPYVSQNPRLAYLNYRDLDIGINDPKNPNNYTQARIW | |
| | GEKYFGKNFDRLVKVKTLVDPNNFFRNEQSIPPLPRHRH | |
| PIR3_ | NIQTSIANPRENFLKCFSQYIPNNATNLKLVYTQNNPLYMSVLNSTIHN | 272 |
| CBDAS_ | LRFSSDTTPKPLVIVTPSHVSHIQGTILCSKKVGLQIRTRSGGHDSEGMS | |
| P464PS_T468 | YISQVPFVIVDLRNMRSIKIDVHSQTAWVEAGATLGEVYYWVNEKNES | |
| I | LSLAAGYCPTVCAGGHFGGGGYGPLMRSYGLAADNIIDAHLVNVHGK | |
| | VLDRKSMGEDLFWALRGGGAESFGIIVAWKIRLVAVPSKSIMFSVKKI | |
| | MEIHELVKLVNKWQNIAYKYDKDLLLMTHFITRNITDNQGKNKTAIHT | |
| | YFSSVFLGGVDSLVDLMNKSFPELGIKKTDCRQLSWIDTIIFYSGVVNY | |
| | DTDNFNKEILLDRSAGQNGAFKIKLDYVKKPIPESVFVQILEKLYEEDIG | |
| | AGMYALYPYGGIMDEISESAIPFPHRAGILYELWYICSWEKQEDNEKH | |
| | LNWIRNIYNFMTPYVSQNPRLAYLNYRDLDIGINDPKNPNNYTQARIW | |
| | GEKYFGKNFDRLVKVKTLVDPNNFFRNEQSIPPLPRHRH | |
| PIR3_ | NIQTSIANPRENFLKCFSQYIPNNATNLKLVYTQNNPLYMSVLNSTIHN | 273 |
| CBDAS_ | LRFSSDTTPKPLVIVTPSHVSHIQGTILCSKKVGLQIRTRSGGHDSEGMS | |
| P464PS_V454A | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVHGK | |
| | VLDRKSMGEDLFWALRGGGAESFGIIAAWKIRLVAVPSKSTMFSVKKI | |
| | MEIHELVKLVNKWQNIAYKYDKDLLLMTHFITRNITDNQGKNKTAIHT | |
| | YFSSVFLGGVDSLVDLMNKSFPELGIKKTDCRQLSWIDTIIFYSGVVNY | |
| | DTDNFNKEILLDRSAGQNGAFKIKLDYVKKPIPESVFVQILEKLYEEDIG | |
| | AGMYALYPYGGIMDEISESAIPFPHRAGILYELWYICSWEKQEDNEKH | |
| | LNWIRNIYNFMTPYVSQNPRLAYLNYRDLDIGINDPKNPNNYTQARIW | |
| | GEKYFGKNFDRLVKVKTLVDPNNFFRNEQSIPPLPRHRH | |
| PIR3_ | NIQTSIANPRENFLKCFSQYIPNNATNLKLVYTQNNPLYMSVLNSTIHN | 274 |
| CBDAS_ | LRFSSDTTPKPLVIVTPSHVSHIQGTILCSKKVGLQIRTRSGGHDSEGMS | |
| S449N_H425D | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVDGK | |
| | VLDRKSMGEDLFWALRGGGAENFGIIVAWKIRLVAVPKSTMFSVKKI | |
| | MEIHELVKLVNKWQNIAYKYDKDLLLMTHFITRNITDNQGKNKTAIHT | |
| | YFSSVFLGGVDSLVDLMNKSFPELGIKKTDCRQLSWIDTIIFYSGVVNY | |
| | DTDNFNKEILLDRSAGQNGAFKIKLDYVKKPIPESVFVQILEKLYEEDIG | |
| | AGMYALYPYGGIMDEISESAIPFPHRAGILYELWYICSWEKQEDNEKH | |
| | LNWIRNIYNFMTPYVSQNPRLAYLNYRDLDIGINDPKNPNNYTQARIW | |
| | GEKYFGKNFDRLVKVKTLVDPNNFFRNEQSIPPLPRHRH | |
| PIR3_ | NIQTSIANPRENFLKCFSQYIPNNATNLKLVYTQNNPLYMSVLNSTIHN | 275 |
| CBDAS_ | LRFSSDTTPKPLVIVTPSHVSHIQGTILCSKKVGLQIRTRSGGHDSEGMS | |
| S449N_I620V | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVHGK | |
| | VLDRKSMGEDLFWALRGGGAENFGIIVAWKIRLVAVPKSTMFSVKKI | |
| | MEIHELVKLVNKWQNIAYKYDKDLLLMTHFITRNITDNQGKNKTAIHT | |
| | YFSSVFLGGVDSLVDLMNKSFPELGIKKTDCRQLSWIDTIIFYSGVVNY | |
| | DTDNFNKEILLDRSAGQNGAFKIKLDYVKKPIPESVFVQILEKLYEEDV | |
| | GAGMYALYPYGGIMDEISESAIPFPHRAGILYELWYICSWEKQEDNEK | |
| | HLNWIRNIYNFMTPYVSQNPRLAYLNYRDLDIGINDPKNPNNYTQARI | |
| | WGEKYFGKNFDRLVKVKTLVDPNNFFRNEQSIPPLPRHRH | |
| PIR3_ | NIQTSIANPRENFLKCFSQYIPNNATNLKLVYTQNNPLYMSVLNSTIHN | 276 |
| CBDAS_ | LRFSSDTTPKPLVIVTPSHVSHIQGTILCSKKVGLQIRTRSGGHDSEGMS | |
| S449N_I676V | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVHGK | |
| | VLDRKSMGEDLFWALRGGGAENFGIIVAWKIRLVAVPKSTMFSVKKI | |
| | MEIHELVKLVNKWQNIAYKYDKDLLLMTHFITRNITDNQGKNKTAIHT | |
| | YFSSVFLGGVDSLVDLMNKSFPELGIKKTDCRQLSWIDTIIFYSGVVNY | |
| | DTDNFNKEILLDRSAGQNGAFKIKLDYVKKPIPESVFVQILEKLYEEDIG | |
| | AGMYALYPYGGIMDEISESAIPFPHRAGILYELWYICSWEKQEDNEKH | |
| | LNWIRNVYNFMTPYVSQNPRLAYLNYRDLDIGINDPKNPNNYTQARI | |
| | WGEKYFGKNFDRLVKVKTLVDPNNFFRNEQSIPPLPRHRH | |
| PIR3_ | NIQTSIANPRENFLKCFSQYIPNNATNLKLVYTQNNPLYMSVLNSTIHN | 277 |
| CBDAS_ | LRFSSDTTPKPLVIVTPSHVSHIQGTILCSKKVGLQIRTRSGGHDSEGMS | |
| S449N_I700L | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVHGK | |
| | VLDRKSMGEDLFWALRGGGAENFGIIVAWKIRLVAVPKSTMFSVKKI | |
| | MEIHELVKLVNKWQNIAYKYDKDLLLMTHFITRNITDNQGKNKTAIHT | |
| | YFSSVFLGGVDSLVDLMNKSFPELGIKKTDCRQLSWIDTIIFYSGVVNY | |
| | DTDNFNKEILLDRSAGQNGAFKIKLDYVKKPIPESVFVQILEKLYEEDIG | |
| | AGMYALYPYGGIMDEISESAIPFPHRAGILYELWYICSWEKQEDNEKH | |
| | LNWIRNIYNFMTPYVSQNPRLAYLNYRDLDLGINDPKNPNNYTQARIW | |
| | GEKYFGKNFDRLVKVKTLVDPNNFFRNEQSIPPLPRHRH | |
| PIR3_ | NIQTSIANPRENFLKCFSQYIPNNATNLKLVYTQNNPLYMSVLNSTIHN | 278 |
| CBDAS_ | LRFSSDTTPKPLVIVTPSHVSHIQGTILCSKKVGLQIRTRSGGHDSEGMS | |
| S449N_L442I | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVHGK | |
| | VLDRKSMGEDLFWAIRGGGAENFGIIVAWKIRLVAVPKSTMFSVKKIM | |
| | EIHELVKLVNKWQNIAYKYDKDLLLMTHFITRNITDNQGKNKTAIHTY | |
| | FSSVFLGGVDSLVDLMNKSFPELGIKKTDCRQLSWIDTIIFYSGVVNYD | |
| | TDNFNKEILLDRSAGQNGAFKIKLDYVKKPIPESVFVQILEKLYEEDIGA | |
| | GMYALYPYGGIMDEISESAIPFPHRAGILYELWYICSWEKQEDNEKHL | |
| | NWIRNIYNFMTPYVSQNPRLAYLNYRDLDIGINDPKNPNNYTQARIWG | |
| | EKYFGKNFDRLVKVKTLVDPNNFFRNEQSIPPLPRHRH | |
| PIR3_ | NIQTSIANPRENFLKCFSQYIPNNATNLKLVYTQNNPLYMSVLNSTIHN | 279 |
| CBDAS_ | LRFSSDTTPKPLVIVTPSHVSHIQGTILCSKKVGLQIRTRSGGHDSEGMS | |
| S449N_M468I | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVHGK | |
| | VLDRKSMGEDLFWALRGGGAENFGIIVAWKIRLVAVPKSTIFSVKKIM | |
| | EIHELVKLVNKWQNIAYKYDKDLLLMTHFITRNITDNQGKNKTAIHTY | |
| | FSSVFLGGVDSLVDLMNKSFPELGIKKTDCRQLSWIDTIIFYSGVVNYD | |
| | TDNFNKEILLDRSAGQNGAFKIKLDYVKKPIPESVFVQILEKLYEEDIGA | |
| | GMYALYPYGGIMDEISESAIPFPHRAGILYELWYICSWEKQEDNEKHL | |
| | NWIRNIYNFMTPYVSQNPRLAYLNYRDLDIGINDPKNPNNYTQARIWG | |
| | EKYFGKNFDRLVKVKTLVDPNNFFRNEQSIPPLPRHRH | |
| PIR3_ | NIQTSIANPRENFLKCFSQYIPNNATNLKLVYTQNDPLYMSVLNSTIHN | 280 |
| CBDAS_ | LRFSSDTTPKPLVIVTPSHVSHIQGTILCSKKVGLQIRTRSGGHDSEGMS | |
| S449N_N269D | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVHGK | |
| | VLDRKSMGEDLFWALRGGGAENFGIIVAWKIRLVAVPKSTMFSVKKI | |
| | MEIHELVKLVNKWQNIAYKYDKDLLLMTHFITRNITDNQGKNKTAIHT | |
| | YFSSVFLGGVDSLVDLMNKSFPELGIKKTDCRQLSWIDTIIFYSGVVNY | |
| | DTDNFNKEILLDRSAGQNGAFKIKLDYVKKPIPESVFVQILEKLYEEDIG | |
| | AGMYALYPYGGIMDEISESAIPFPHRAGILYELWYICSWEKQEDNEKH | |
| | LNWIRNIYNFMTPYVSQNPRLAYLNYRDLDIGINDPKNPNNYTQARIW | |
| | GEKYFGKNFDRLVKVKTLVDPNNFFRNEQSIPPLPRHRH | |
| PIR3_ | NIQTSIANPRENFLKCFSQYIPNNATNLKLVYTQNNPLYMSVLNSTIHN | 281 |
| CBDAS_ | LRFSSDTTPKPLVIVTPSHVSHIQGTILCSKKVGLQIRTRSGGHDSEGMS | |
| S449N_ | YISQVPFVIVDLRNMRSIKIDVHSQTAWVEAGATLGEVYYWVNEKNES | |
| P464PS | LSLAAGYCPTVCAGGHFGGGGYGPLMRSYGLAADNIIDAHLVNVHGK | |
| | VLDRKSMGEDLFWALRGGGAENFGIIVAWKIRLVAVPSKSTMFSVKKI | |
| | MEIHELVKLVNKWQNIAYKYDKDLLLMTHFITRNITDNQGKNKTAIHT | |
| | YFSSVFLGGVDSLVDLMNKSFPELGIKKTDCRQLSWIDTIIFYSGVVNY | |
| | DTDNFNKEILLDRSAGQNGAFKIKLDYVKKPIPESVFVQILEKLYEEDIG | |
| | AGMYALYPYGGIMDEISESAIPFPHRAGILYELWYICSWEKQEDNEKH | |
| | LNWIRNIYNFMTPYVSQNPRLAYLNYRDLDIGINDPKNPNNYTQARIW | |
| | GEKYFGKNFDRLVKVKTLVDPNNFFRNEQSIPPLPRHRH | |
| PIR3_ | NIQTSIANPQENFLKCFSQYIPNNATNLKLVYTQNNPLYMSVLNSTIHN | 282 |
| CBDAS_ | LRFSSDTTPKPLVIVTPSHVSHIQGTILCSKKVGLQIRTRSGGHDSEGMS | |
| S449N_R243Q | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVHGK | |
| | VLDRKSMGEDLFWALRGGGAENFGIIVAWKIRLVAVPKSTMFSVKKI | |
| | MEIHELVKLVNKWQNIAYKYDKDLLLMTHFITRNITDNQGKNKTAIHT | |
| | YFSSVFLGGVDSLVDLMNKSFPELGIKKTDCRQLSWIDTIIFYSGVVNY | |
| | DTDNFNKEILLDRSAGQNGAFKIKLDYVKKPIPESVFVQILEKLYEEDIG | |
| | AGMYALYPYGGIMDEISESAIPFPHRAGILYELWYICSWEKQEDNEKH | |
| | LNWIRNIYNFMTPYVSQNPRLAYLNYRDLDIGINDPKNPNNYTQARIW | |
| | GEKYFGKNFDRLVKVKTLVDPNNFFRNEQSIPPLPRHRH | |
| PIR3_ | NIQTSIANPRENFLKCFSQYIPNNATNLKLVYTQNNPLYMSVLNSTIHN | 283 |
| CBDAS_ | LRFSSDTTPKPLVIVTPSHVSHIQGTILCSKKVGLQIRTRSGGHDAEGMS | |
| S449N_S328A | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVHGK | |
| | VLDRKSMGEDLFWALRGGGAENFGIIVAWKIRLVAVPKSTMFSVKKI | |
| | MEIHELVKLVNKWQNIAYKYDKDLLLMTHFITRNITDNQGKNKTAIHT | |
| | YFSSVFLGGVDSLVDLMNKSFPELGIKKTDCRQLSWIDTIIFYSGVVNY | |
| | DTDNFNKEILLDRSAGQNGAFKIKLDYVKKPIPESVFVQILEKLYEEDIG | |
| | AGMYALYPYGGIMDEISESAIPFPHRAGILYELWYICSWEKQEDNEKH | |
| | LNWIRNIYNFMTPYVSQNPRLAYLNYRDLDIGINDPKNPNNYTQARIW | |
| | GEKYFGKNFDRLVKVKTLVDPNNFFRNEQSIPPLPRHRH | |
| PIR3_ | NIQTSIANPRENFLKCFSQYIPNNATNLKLVYTQNNPLYMSVLNSTIHN | 284 |
| CBDAS_ | LRFSSDTTPKPLVIVTPSHVSHIQGTILCSKKVGLQIRTRSGGHDSEGMS | |
| S449N_V454A | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVHGK | |
| | VLDRKSMGEDLFWALRGGGAENFGIIAAWKIRLVAVPKSTMFSVKKI | |
| | MEIHELVKLVNKWQNIAYKYDKDLLLMTHFITRNITDNQGKNKTAIHT | |
| | YFSSVFLGGVDSLVDLMNKSFPELGIKKTDCRQLSWIDTIIFYSGVVNY | |
| | DTDNFNKEILLDRSAGQNGAFKIKLDYVKKPIPESVFVQILEKLYEEDIG | |
| | AGMYALYPYGGIMDEISESAIPFPHRAGILYELWYICSWEKQEDNEKH | |
| | LNWIRNIYNFMTPYVSQNPRLAYLNYRDLDIGINDPKNPNNYTQARIW | |
| | GEKYFGKNFDRLVKVKTLVDPNNFFRNEQSIPPLPRHRH | |
| PIR3_ | NIQTSIANPRENFLKCFSQYIPNNATNLKLVYTQNNPLYMSVLNSTIHN | 285 |
| CBDAS_ | LRFSSDTTPKPLVIVTPSHVSHIQGTILCSKKVGLQIRTRSGGHDSEGMS | |
| S449N_Y571F | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVHGK | |
| | VLDRKSMGEDLFWALRGGGAENFGIIVAWKIRLVAVPKSTMFSVKKI | |
| | MEIHELVKLVNKWQNIAYKYDKDLLLMTHFITRNITDNQGKNKTAIHT | |
| | YFSSVFLGGVDSLVDLMNKSFPELGIKKTDCRQLSWIDTIIFYSGVVNF | |
| | DTDNFNKEILLDRSAGQNGAFKIKLDYVKKPIPESVFVQILEKLYEEDIG | |
| | AGMYALYPYGGIMDEISESAIPFPHRAGILYELWYICSWEKQEDNEKH | |
| | LNWIRNIYNFMTPYVSQNPRLAYLNYRDLDIGINDPKNPNNYTQARIW | |
| | GEKYFGKNFDRLVKVKTLVDPNNFFRNEQSIPPLPRHRH | |
| PIR3_ | NIQTSIANPRENFLKCFSQYIPNNATNLKLVYTQNNPLYMSVLNSTIHN | 286 |
| CBDAS_ | LRFSSDTTPKPLVIVTPSHVSHIQGTILCSKKVGLQIRTRSGGHDSEGMS | |
| V454A_M468I | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVHGK | |
| | VLDRKSMGEDLFWALRGGGAESFGIIAAWKIRLVAVPKSTIFSVKKIM | |
| | EIHELVKLVNKWQNIAYKYDKDLLLMTHFITRNITDNQGKNKTAIHTY | |
| | FSSVFLGGVDSLVDLMNKSFPELGIKKTDCRQLSWIDTIIFYSGVVNYD | |
| | TDNFNKEILLDRSAGQNGAFKIKLDYVKKPIPESVFVQILEKLYEEDIGA | |
| | GMYALYPYGGIMDEISESAIPFPHRAGILYELWYICSWEKQEDNEKHL | |
| | NWIRNIYNFMTPYVSQNPRLAYLNYRDLDIGINDPKNPNNYTQARIWG | |
| | EKYFGKNFDRLVKVKTLVDPNNFFRNEQSIPPLPRHRH | |
| PIR3_ | NIQTSIANPRENFLKCFSQYIPNNATNLKLVYTQNDPLYMSVLNSTIHN | 287 |
| CBDAS_ | LRFSSDTTPKPLVIVTPSHVSHIQGTILCSKKVGLQIRTRSGGHDSEGMS | |
| V454A_N269D | YISQVPFVIVDLRNMRSIKIDVHSQTAWVEAGATLGEVYYWVNEKNES | |
| | LSLAAGYCPTVCAGGHFGGGGYGPLMRSYGLAADNIIDAHLVNVHGK | |
| | VLDRKSMGEDLFWALRGGGAESFGIIAAWKIRLVAVPKSTMFSVKKI | |
| | MEIHELVKLVNKWQNIAYKYDKDLLLMTHFITRNITDNQGKNKTAIHT | |
| | YFSSVFLGGVDSLVDLMNKSFPELGIKKTDCRQLSWIDTIIFYSGVVNY | |
| | DTDNFNKEILLDRSAGQNGAFKIKLDYVKKPIPESVFVQILEKLYEEDIG | |
| | AGMYALYPYGGIMDEISESAIPFPHRAGILYELWYICSWEKQEDNEKH | |
| | LNWIRNIYNFMTPYVSQNPRLAYLNYRDLDIGINDPKNPNNYTQARIW | |
| | GEKYFGKNFDRLVKVKTLVDPNNFFRNEQSIPPLPRHRH | |

### Example 25—CBCA Synthase Mutagenesis

Fifty-three (53) positions around the predicted active site of the Cannabis sativa native CBDA synthase enzyme were mutagenized in a random manner to increase the synthesis of CBCA. The parental plasmid for mutagenesis was the 0285/asn080-2 plasmid. Plasmids carrying mutant CBDA synthase genes were isolated and screened for CBCA synthase activity. The screen identified amino acid positions where substitutions for certain amino acids result in the formation of a highly specific CBCA synthase or CBDA synthase with elevated CBCA synthase activity. The parental plasmid and plasmids with mutant CBDA synthase gene were transformed into the yCBGA0513 strain.

Mutant CBDA synthases with CBCA activity were screened using the following high throughput screening process: Colonies were inoculated into wells of a 96-well deep well plate. Each well contains 400 μl SC liquid medium (6.7 g/L Yeast Nitrogen Base, 1.6 g/L Amino Acid Drop Out mix without leucine, uracil, tryptophan and histidine, 22 g/L glucose, buffered to pH 6.0, supplemented with leucine, tryptophan, histidine and Hygromycin B). The inoculums were grown for 48 hours at 30° C. and shaken at 300 rpm with 50 mm shaking diameter. After a 48 hour growth period, 40 μl samples of these cultures were inoculated into 360 μl YPD-2400LA (10 g/L yeast extract, 20 g/L peptone, 20 g/L glucose and 240 mg/L olivetolic acid) medium. Then samples were grown for 48 hours at 30° C. and shaken at 300 rpm with 50 mm shaking diameter and 8 μl of 12000 mg/l OLA dissolved in EtOH was added to the samples. Finally, samples were grown for an additional 42 hours and were analyzed for cannabinoids.

The CBCA titer in the above described screen ranged from 18.0 to 69.6 mg/L. Sample titers for the transformed strains, along with each strain's respective amino acid mutations and amino acid sequences, are included in Tables 27-28 below. (All mutation sites refer to the mutation site on CBDA's coding sequence without the wild-type signal sequence.)

TABLE 27
yCBGA_0513 mutant strains

| | mutation | | | | |
|---|---|---|---|---|---|
| | site on | | | | |
| | CBDAs | | | | |
| | coding | | | | |
| | sequence | | | Average | Average |
| | without the | native | new | titer of | titer of |
| | signal | amino | amino | CBDA | CBCA |
| Constructs | sequence | acid | acid | mg/L | mg/L |
| SP_CBGA1137_10_B10 | 237 | S | Q | 0.0 | 20.9 |
| SP_CBGA1137_03_H01 | 268 | M | T | 0.0 | 60.0 |
| SP_CBGA1137_11_H10 | 292 | S | N | 23.3 | 32.9 |
| SP_CBGA1136_04_F03 | 330 | I | R | 23.2 | 26.8 |
| SP_CBGA1138_12_G06 | 332 | Y | L | 26.8 | 20.9 |
| SP_CBGA1135_11_F02 | 334 | G | Q | 35.9 | 35.9 |
| SP_CBGA1139_01_D07 | 338 | Y | K | 81.9 | 69.6 |
| SP_CBGA1135_10_B02 | 359 | F | H | 26.5 | 21.5 |
| SP_CBGA1136_05_A05 | 361 | I | Y | 0.0 | 22.1 |
| 0285/asn080-2 | wild type | N/A | N/A | 134.0 | 18.0 |
| | control | | | | |
| | construct | | | | |

TABLE 28
yCBGA_0513 mutants

| | | SEQ ID |
|---|---|---|
| Constructs | CBCAs amino acid sequence without the signal sequence | NO: |
| SP_CBGA | NIQTSIANPRENFLKCFSQYIPNNATNLKLVYTQNNPLYMSVLNSTIH | 288 |
| 1137_10_ | NLRFSSDTTPKPLVIVTPSHVSHIQGTILCSKKVGLQIRTRSGGHDSEG | |
| B10 | MSYISQVPFVIVDLRNMRSIKIDVHSQTAWVEAGATLGEVYYWVNE | |
| | KNESLSLAAGYCPTVCAGGHFGGGGYGPLMRSYGLAADNIIDAHLV | |
| | NVHGKVLDRKSMGEDLFWALRGGGAESFGIIVAWKIRLVAVPKSTM | |
| | FQVKKIMEIHELVKLVNKWQNIAYKYDKDLLLMTHFITRNITDNQG | |
| | KNKTAIHTYFSSVFLGGVDSLVDLMNKSFPELGIKKTDCRQLSWIDTI | |
| | IFYSGVVNYDTDNFNKEILLDRSAGQNGAFKIKLDYVKKPIPESVFVQ | |
| | ILEKLYEEDIGAGMYALYPYGGIMDEISESAIPFPHRAGILYELWYICS | |
| | WEKQEDNEKHLNWIRNIYNFMTPYVSQNPRLAYLNYRDLDIGINDP | |
| | KNPNNYTQARIWGEKYFGKNFDRLVKVKTLVDPNNFFRNEQSIPPLP | |
| | RHRH | |
| SP_CBGA | NIQTSIANPRENFLKCFSQYIPNNATNLKLVYTQNNPLYMSVLNSTIH | 289 |
| 1137_03_ | NLRFSSDTTPKPLVIVTPSHVSHIQGTILCSKKVGLQIRTRSGGHDSEG | |
| H01 | MSYISQVPFVIVDLRNMRSIKIDVHSQTAWVEAGATLGEVYYWVNE | |
| | KNESLSLAAGYCPTVCAGGHFGGGGYGPLMRSYGLAADNIIDAHLV | |
| | NVHGKVLDRKSMGEDLFWALRGGGAESFGIIVAWKIRLVAVPKSTM | |
| | FSVKKIMEIHELVKLVNKWQNIAYKYDKDLLLTTHFITRNITDNQGK | |
| | NKTAIHTYFSSVFLGGVDSLVDLMNKSFPELGIKKTDCRQLSWIDTIIF | |
| | YSGVVNYDTDNFNKEILLDRSAGQNGAFKIKLDYVKKPIPESVFVQIL | |
| | EKLYEEDIGAGMYALYPYGGIMDEISESAIPFPHRAGILYELWYICSW | |
| | EKQEDNEKHLNWIRNIYNFMTPYVSQNPRLAYLNYRDLDIGINDPKN | |
| | PNNYTQARIWGEKYFGKNFDRLVKVKTLVDPNNFFRNEQSIPPLPRH | |
| | RH | |
| SP_CBGA | NIQTSIANPRENFLKCFSQYIPNNATNLKLVYTQNNPLYMSVLNSTIH | 290 |
| 1137_11_ | NLRFSSDTTPKPLVIVTPSHVSHIQGTILCSKKVGLQIRTRSGGHDSEG | |
| H10 | MSYISQVPFVIVDLRNMRSIKIDVHSQTAWVEAGATLGEVYYWVNE | |
| | KNESLSLAAGYCPTVCAGGHFGGGGYGPLMRSYGLAADNIIDAHLV | |
| | NVHGKVLDRKSMGEDLFWALRGGGAESFGIIVAWKIRLVAVPKSTM | |
| | FSVKKIMEIHELVKLVNKWQNIAYKYDKDLLLMTHFITRNITDNQGK | |
| | NKTAIHTYFNSVFLGGVDSLVDLMNKSFPELGIKKTDCRQLSWIDTII | |
| | FYSGVVNYDTDNFNKEILLDRSAGQNGAFKIKLDYVKKPIPESVFVQI | |
| | LEKLYEEDIGAGMYALYPYGGIMDEISESAIPFPHRAGILYELWYICS | |
| | WEKQEDNEKHLNWIRNIYNFMTPYVSQNPRLAYLNYRDLDIGINDP | |
| | KNPNNYTQARIWGEKYFGKNFDRLVKVKTLVDPNNFFRNEQSIPPLP | |
| | RHRH | |
| SP_CBGA | NIQTSIANPRENFLKCFSQYIPNNATNLKLVYTQNNPLYMSVLNSTIH | 291 |
| 1136_04_ | NLRFSSDTTPKPLVIVTPSHVSHIQGTILCSKKVGLQIRTRSGGHDSEG | |
| F03 | MSYISQVPFVIVDLRNMRSIKIDVHSQTAWVEAGATLGEVYYWVNE | |
| | KNESLSLAAGYCPTVCAGGHFGGGGYGPLMRSYGLAADNIIDAHLV | |
| | NVHGKVLDRKSMGEDLFWALRGGGAESFGIIVAWKIRLVAVPKSTM | |
| | FSVKKIMEIHELVKLVNKWQNIAYKYDKDLLLMTHFITRNITDNQGK | |
| | NKTAIHTYFSSVFLGGVDSLVDLMNKSFPELGIKKTDCRQLSWIDTIR | |
| | FYSGVVNYDTDNFNKEILLDRSAGQNGAFKIKLDYVKKPIPESVFVQI | |
| | LEKLYEEDIGAGMYALYPYGGIMDEISESAIPFPHRAGILYELWYICS | |
| | WEKQEDNEKHLNWIRNIYNFMTPYVSQNPRLAYLNYRDLDIGINDP | |
| | KNPNNYTQARIWGEKYFGKNFDRLVKVKTLVDPNNFFRNEQSIPPLP | |
| | RHRH | |
| SP_CBGA | NIQTSIANPRENFLKCFSQYIPNNATNLKLVYTQNNPLYMSVLNSTIH | 292 |
| 1138_12_ | NLRFSSDTTPKPLVIVTPSHVSHIQGTILCSKKVGLQIRTRSGGHDSEG | |
| G06 | MSYISQVPFVIVDLRNMRSIKIDVHSQTAWVEAGATLGEVYYWVNE | |
| | KNESLSLAAGYCPTVCAGGHFGGGGYGPLMRSYGLAADNIIDAHLV | |
| | NVHGKVLDRKSMGEDLFWALRGGGAESFGIIVAWKIRLVAVPKSTM | |
| | FSVKKIMEIHELVKLVNKWQNIAYKYDKDLLLMTHFITRNITDNQGK | |
| | NKTAIHTYFSSVFLGGVDSLVDLMNKSFPELGIKKTDCRQLSWIDTIIF | |
| | LSGVVNYDTDNFNKEILLDRSAGQNGAFKIKLDYVKKPIPESVFVQIL | |
| | EKLYEEDIGAGMYALYPYGGIMDEISESAIPFPHRAGILYELWYICSW | |
| | EKQEDNEKHLNWIRNIYNFMTPYVSQNPRLAYLNYRDLDIGINDPKN | |
| | PNNYTQARIWGEKYFGKNFDRLVKVKTLVDPNNFFRNEQSIPPLPRH | |
| | RH | |
| SP_CBGA | NIQTSIANPRENFLKCFSQYIPNNATNLKLVYTQNNPLYMSVLNSTIH | 293 |
| 1135_11_ | NLRFSSDTTPKPLVIVTPSHVSHIQGTILCSKKVGLQIRTRSGGHDSEG | |
| F02 | MSYISQVPFVIVDLRNMRSIKIDVHSQTAWVEAGATLGEVYYWVNE | |
| | KNESLSLAAGYCPTVCAGGHFGGGGYGPLMRSYGLAADNIIDAHLV | |
| | NVHGKVLDRKSMGEDLFWALRGGGAESFGIIVAWKIRLVAVPKSTM | |
| | FSVKKIMEIHELVKLVNKWQNIAYKYDKDLLLMTHFITRNITDNQGK | |
| | NKTAIHTYFSSVFLGGVDSLVDLMNKSFPELGIKKTDCRQLSWIDTIIF | |
| | YSQVVNYDTDNFNKEILLDRSAGQNGAFKIKLDYVKKPIPESVFVQIL | |
| | EKLYEEDIGAGMYALYPYGGIMDEISESAIPFPHRAGILYELWYICSW | |
| | EKQEDNEKHLNWIRNIYNFMTPYVSQNPRLAYLNYRDLDIGINDPKN | |
| | PNNYTQARIWGEKYFGKNFDRLVKVKTLVDPNNFFRNEQSIPPLPRH | |
| | RH | |
| SP_CBGA | NIQTSIANPRENFLKCFSQYIPNNATNLKLVYTQNNPLYMSVLNSTIH | 294 |
| 1139_01_ | NLRFSSDTTPKPLVIVTPSHVSHIQGTILCSKKVGLQIRTRSGGHDSEG | |
| D07 | MSYISQVPFVIVDLRNMRSIKIDVHSQTAWVEAGATLGEVYYWVNE | |
| | KNESLSLAAGYCPTVCAGGHFGGGGYGPLMRSYGLAADNIIDAHLV | |
| | NVHGKVLDRKSMGEDLFWALRGGGAESFGIIVAWKIRLVAVPKSTM | |
| | FSVKKIMEIHELVKLVNKWQNIAYKYDKDLLLMTHFITRNITDNQ | GK |
| | NKTAIHTYFSSVFLGGVDSLVDLMNKSFPELGIKKTDCRQLSWIDTIIF | |
| | YSGVVNKDTDNFNKEILLDRSAGQNGAFKIKLDYVKKPIPESVFVQIL | |
| | EKLYEEDIGAGMYALYPYGGIMDEISESAIPFPHRAGILYELWYICSW | |
| | EKQEDNEKHLNWIRNIYNFMTPYVSQNPRLAYLNYRDLDIGINDPKN | |
| | PNNYTQARIWGEKYFGKNFDRLVKVKTLVDPNNFFRNEQSIPPLPRH | |
| | RH | |
| SP_CBGA | NIQTSIANPRENFLKCFSQYIPNNATNLKLVYTQNNPLYMSVLNSTIH | 295 |
| 1135_10_ | NLRFSSDTTPKPLVIVTPSHVSHIQGTILCSKKVGLQIRTRSGGHDSEG | |
| B02 | MSYISQVPFVIVDLRNMRSIKIDVHSQTAWVEAGATLGEVYYWVNE | |
| | KNESLSLAAGYCPTVCAGGHFGGGGYGPLMRSYGLAADNIIDAHLV | |
| | NVHGKVLDRKSMGEDLFWALRGGGAESFGIIVAWKIRLVAVPKSTM | |
| | FSVKKIMEIHELVKLVNKWQNIAYKYDKDLLLMTHFITRNITDNQGK | |
| | NKTAIHTYFSSVFLGGVDSLVDLMNKSFPELGIKKTDCRQLSWIDTIIF | |
| | YSGVVNYDTDNFNKEILLDRSAGQNGAHKIKLDYVKKPIPESVFVQI | |
| | LEKLYEEDIGAGMYALYPYGGIMDEISESAIPFPHRAGILYELWYICS | |
| | WEKQEDNEKHLNWIRNIYNFMTPYVSQNPRLAYLNYRDLDIGINDP | |
| | KNPNNYTQARIWGEKYFGKNFDRLVKVKTLVDPNNFFRNEQSIPPLP | |
| | RHRH | |
| SP_CBGA | NIQTSIANPRENFLKCFSQYIPNNATNLKLVYTQNNPLYMSVLNSTIH | 296 |
| 1136_05_ | NLRFSSDTTPKPLVIVTPSHVSHIQGTILCSKKVGLQIRTRSGGHDSEG | |
| A05 | MSYISQVPFVIVDLRNMRSIKIDVHSQTAWVEAGATLGEVYYWVNE | |
| | KNESLSLAAGYCPTVCAGGHFGGGGYGPLMRSYGLAADNIIDAHLV | |
| | NVHGKVLDRKSMGEDLFWALRGGGAESFGIIVAWKIRLVAVPKSTM | |
| | FSVKKIMEIHELVKLVNKWQNIAYKYDKDLLLMTHFITRNITDNQGK | |
| | NKTAIHTYFSSVFLGGVDSLVDLMNKSFPELGIKKTDCRQLSWIDTIIF | |
| | YSGVVNYDTDNFNKEILLDRSAGQNGAFKYKLDYVKKPIPESVFVQI | |
| | LEKLYEEDIGAGMYALYPYGGIMDEISESAIPFPHRAGILYELWYICS | |
| | WEKQEDNEKHLNWIRNIYNFMTPYVSQNPRLAYLNYRDLDIGINDP | |
| | KNPNNYTQARIWGEKYFGKNFDRLVKVKTLVDPNNFFRNEQSIPPLP | |
| | RHRH | |
| 0285/asn0 | NIQTSIANPRENFLKCFSQYIPNNATNLKLVYTQNNPLYMSVLNSTIH | 297 |
| 80-2 | NLRFSSDTTPKPLVIVTPSHVSHIQGTILCSKKVGLQIRTRSGGHDSEG | |
| | MSYISQVPFVIVDLRNMRSIKIDVHSQTAWVEAGATLGEVYYWVNE | |
| | KNESLSLAAGYCPTVCAGGHFGGGGYGPLMRSYGLAADNIIDAHLV | |
| | NVHGKVLDRKSMGEDLFWALRGGGAESFGIIVAWKIRLVAVPKSTM | |
| | FSVKKIMEIHELVKLVNKWQNIAYKYDKDLLLMTHFITRNITDNQGK | |
| | NKTAIHTYFSSVFLGGVDSLVDLMNKSFPELGIKKTDCRQLSWIDTIIF | |
| | YSGVVNYDTDNFNKEILLDRSAGQNGAFKIKLDYVKKPIPESVFVQIL | |
| | EKLYEEDIGAGMYALYPYGGIMDEISESAIPFPHRAGILYELWYICSW | |
| | EKQEDNEKHLNWIRNIYNFMTPYVSQNPRLAYLNYRDLDIGINDPKN | |
| | PNNYTQARIWGEKYFGKNFDRLVKVKTLVDPNNFFRNEQSIPPLPRH | |
| | RH | |

### Example 26—CBCA Synthase Mutagenesis

To improve the performance of CBCA synthase, an enzyme mutagenesis experiment was conducted. The mutant CBCA C-terminal core enzyme contains 3 differences as the compared to CBDA Cannabis sativa C-terminal core enzyme, G74A, S insertion at 232, and M269T (when numbering the core sequence (without the signal sequence).

The starting CBCA N-terminal signal sequence and C-terminal coding sequence are included Table 29 below:

TABLE 29

| | length of | | |
|---|---|---|---|
| | amino acid | | SEQ ID |
| | sequence | CBCA amino acid sequence | NO: |
| N-terminal | 251 | MESVSSLFNIFSTIMVNYKSLVLALLSVSNLKYA | SEQ ID |
| signal | | RGAYAPKDPWSTLTPSATYKGGITDYSSSFGIAI | No: 434 |
| sequence | | EAVATSASSVASSKAKRAASQIGDGQVQAATTT | |
| | | AAVSKKSTAAAVSQITDGQVQAAKSTAAAVSQI | |
| | | TDGQVQAAKSTAAAVSQITDGQVQAAKSTAAA | |
| | | VSQITDGQVQAAKSTAAAASQISDGQVQATTST | |
| | | KAAASQITDGQIQASKTTSGASQVSDGQVQATA | |
| | | EVKDANDPVDVVSCNNNST | |
| C-terminal | 524 | NIQTSIANPRENFLKCFSQYIPNNATNLKLVYTQ | SEQ ID |
| core | | NNPLYMSVLNSTIHNLRFSSDTTPKPLVIVTPSH | No: 304 |
| enzyme: | | VSHIQATILCSKKVGLQIRTRSGGHDSEGMSYIS | |
| (signal | | QVPFVIVDLRNMRSIKIDVHSQTAWVEAGATLG | |
| sequence | | EVYYWVNEKNESLSLAAGYCPTVCAGGHFGGG | |
| removed) | | GYGPLMRSYGLAADNIIDAHLVNVHGKVLDRK | |
| | | SMGEDLFWALRGGGAESFGIIVAWKIRLVAVPS | |
| | | KSTMFSVKKIMEIHELVKLVNKWQNIAYKYDK | |
| | | DLLLTTHFITRNITDNQGKNKTAIHTYFSSVFLG | |
| | | GVDSLVDLMNKSFPELGIKKTDCRQLSWIDTIIF | |
| | | YSGVVNYDTDNFNKEILLDRSAGQNGAFKIKLD | |
| | | YVKKPIPESVFVQILEKLYEEDIGAGMYALYPYG | |
| | | GIMDEISESAIPFPHRAGILYELWYICSWEKQED | |
| | | NEKHLNWIRNIYNFMTPYVSQNPRLAYLNYRDL | |
| | | DIGINDPKNPNNYTQARIWGEKYFGKNFDRLVK | |
| | | VKTLVDPNNFFRNEQSIPPLPRHRH | |

Positions around the predicted active site of the Cannabis sativa CBDA enzyme were mutagenized in a random manner to increase the synthesis of CBCA. The parental plasmid for mutagenesis was the 0285/asn080-2 plasmid. Plasmids carrying mutant CBCA synthase genes were isolated and screened for CBCA synthase activity. The screen identified amino acid positions where substitutions for certain amino acids result in the formation of a highly specific CBCA synthase with elevated CBCA synthase activity. The parental plasmid and plasmids with mutant CBDA synthase gene were transformed into the yCBGA0513 or the yCBGA0523 strain.

Mutant CBCA synthases were screened using the following high throughput screening process: Colonies were inoculated into wells of a 96-well deep well plate. Each well contains 400 μl SC liquid medium (6.7 g/L Yeast Nitrogen Base, 1.6 g/L Amino Acid Drop Out mix without leucine, uracil, tryptophan and histidine, 22 g/L glucose, buffered to pH 6.0, supplemented with leucine, tryptophan, histidine and Hygromycin B). The inoculums were grown for 48 hours at 30° C. and shaken at 300 rpm with 50 mm shaking diameter. After a 48 hour growth period, 40 μl samples of these cultures were inoculated into 360 μl YPD-2400LA (10 g/L yeast extract, 20 g/L peptone, 20 g/L glucose and 240 mg/L olivetolic acid) medium. Then samples were grown for 48 hours at 30° C. and shaken at 300 rpm with 50 mm shaking diameter and 8 μl of 1200 mg/l OLA dissolved in EtOH was added to the samples. Finally, samples were grown for an additional 42 hours and were analyzed for cannabinoids.

Using the CBCA mutants of Table 30 in the above described screen resulted in improved titers as compared to yCBGA0513 or yCBGA0523 strains encoding the native Cannabis sativa CBCA. Each mutant's respective amino acid mutations and amino acid sequences, are included in Tables 30 below. (All mutation sites refer to the mutation site on CBCA's coding sequence without the signal sequence.)

TABLE 30
yCBGA_0523 mutant strains

| mutation site | | | |
|---|---|---|---|
| on CBCAs | | | |
| coding | | | |
| sequence | | | |
| without the | native | new | |
| signal | amino | amino | |
| sequence | acid | acid | |
| 1 | Asn | Asn | SEQ ID NO: 305 |
| 94 | Asp | Ser | SEQ ID NO: 306 |
| 94 | Asp | Asn | SEQ ID NO: 307 |
| 267 | Leu | Thr | SEQ ID NO: 308 |
| 461 | Leu | Arg | SEQ ID NO: 309 |
| 461 | Leu | Lys | SEQ ID NO: 310 |
| 1 | Asn | Leu | SEQ ID NO: 311 |
| 1 | Asn | Gly | SEQ ID NO: 312 |
| 54 | Ser | Arg | SEQ ID NO: 313 |
| 54 | Ser | Thr | SEQ ID NO: 314 |
| 95 | Ser | Gly | SEQ ID NO: 315 |
| 95 | Ser | Ala | SEQ ID NO: 316 |
| 82 | Val | Ala | SEQ ID NO: 317 |
| 82 | Val | Thr | SEQ ID NO: 318 |

### Example 27—Prenyl Transferase Site Directed Mutagenesis

To improve the performance of prenyl transferase (PT), a site directed mutagenesis experiment was conducted. The base gene for mutagenesis was a fusion construct: N-terminal part: yEVenus (a modified GFP protein); C-terminal part: GFP-dPT of strain 314 (SEQ ID NO: 27), a truncated Saccharomyces cerevisiae prenyl-transferase (the N-terminal 98 amino acid of the original prenyl-transferase was truncated), and the sequences are included Table 31 below:

TABLE 31

| | length of | | |
|---|---|---|---|
| | amino acid | | SEQ ID |
| | sequence | prenyltransferase amino acid sequence | NO: |
| Coding | 539 | MSKGEELFTGVVPILVELDGDVNGHKFSVSGEGE | SEQ ID |
| sequence | | GDATYGKLTLKLICTTGKLPVPWPTLVTTLGYGL | NO: 319 |
| | | QCFARYPDHMKQHDFFKSAMPEGYVQERTIFFKD | |
| | | DGNYKTRAEVKFEGDTLVNRIELKGIDFKEDGNIL | |
| | | GHKLEYNYNSHNVYITADKQKNGIKANFKIRHNI | |
| | | EDGGVQLADHYQQNTPIGDGPVLLPDNHYLSYQS | |
| | | ALSKDPNEKRDHMVLLEFVTAAGITHGMDELYK | |
| | | KILNFGHTCWKLQRPYVVKGMISIACGLFGRELF | |
| | | NNRHLFSWGLMWKAFFALVPILSFNFFAAIMNQI | |
| | | YDVDIDRINKPDLPLVSGEMSIETAWILSIIVALTG | |
| | | LIVTIKLKSAPLFVFIYIFGIFAGFAYSVPPIRWKQY | |
| | | PFTNFLITISSHVGLAFTSYSATTSALGLPFVWRPA | |
| | | FSFIIAFMTVMGMTIAFAKDISDIEGDAKYGVSTV | |
| | | ATKLGARNMTFVVSGVLLLNYLVSISIGIIWPQVF | |
| | | KSNIMILSHAILAFCLIFQTRELALANYASAPSRQF | |
| | | FEFIWLLYYAEYFVYVFI | |

Positions around the predicted active site of the native Saccharomyces cerevisiae prenyltransferase synthase enzyme were mutagenized in a random manner to increase the synthesis of prenyltransferase. The parental plasmid for mutagenesis was the 0285/asn080-2 plasmid. Plasmids carrying mutant prenyltransferase synthase genes were isolated and screened for prenyltransferase synthase activity. The screen identified amino acid positions where substitutions for certain amino acids result in the formation of a highly specific prenyltransferase synthase with elevated prenyltransferase synthase activity. The parental plasmid and plasmids with mutant CBDA synthase gene were transformed into the yCBGA0537 strain (yCBGA0537=yCBGA0523 where all PT were deleted).

Mutant prenyltransferase synthases were screened using the following high throughput screening process: Colonies were inoculated into wells of a 96-well deep well plate. Each well contains 400 μl SC liquid medium (6.7 g/L Yeast Nitrogen Base, 1.6 g/L Amino Acid Drop Out mix without leucine, uracil, tryptophan and histidine, 22 g/L glucose, buffered to pH 6.0, supplemented with leucine, tryptophan, histidine and Hygromycin B). The inoculums were grown for 48 hours at 30° C. and shaken at 300 rpm with 50 mm shaking diameter. After a 48 hour growth period, 40 μl samples of these cultures were inoculated into 360 μl YPD-2400LA (10 g/L yeast extract, 20 g/L peptone, 20 g/L glucose and 240 mg/L olivetolic acid) medium. Then samples were grown for 48 hours at 30° C. and shaken at 300 rpm with 50 mm shaking diameter and 8 μl of 12000 mg/l OLA dissolved in EtOH was added to the samples. Finally, samples were grown for an additional 42 hours and were analyzed for cannabinoids.

Using the prenyltransferase mutants of Table 32 in the above described screen resulted in similar or improved CBGA titers as compared to yCBGA0537 strains encoding the prenyltransferase. Each mutant's respective amino acid mutations and amino acid sequences, are included in Tables 32 below. (All mutation sites refer to the mutation site to the prenyltransferase in table 31 (SEQ ID 319).)

TABLE 32
yCBGA_0537 mutant strains

| | First | Last | | | | | |
|---|---|---|---|---|---|---|---|
| | position | position | | | | | |
| | of the | of the | | | | Mutants | |
| | mutation | mutation | | | | titer of | |
| | site on | site on | | Wild | | CBGA/ | |
| | PT's | PT's | Length | type | mutant | Wild Type | SEQ |
| Plasmid | coding | coding | of | amino | amino | titer of | ID |
| Constructs | sequence | sequence | mutation | acid(s) | acid(s) | CBGA | NO: |
| bCBGA_0537 | NA | NA | NA | NA | NA | 1.000 | 320 |
| 0383/asn001-1 | 239 | 241 | 3 | KIL | RVA | 0.889 | 321 |
| 0382/asn002-3 | 242 | 242 | 1 | N | D | 0.983 | 322 |
| 0382/asn001-2 | 242 | 242 | 1 | N | Q | 1.158 | 323 |
| 0383/asn002-3 | 244 | 245 | 2 | GH | LK | 0.957 | 324 |
| 0382/asn003-4 | 249 | 249 | 1 | K | R | 0.978 | 325 |
| 0382/asn009-3 | 264 | 264 | 1 | C | S | 1.316 | 326 |
| 0382/asn013-2 | 272 | 272 | 1 | F | I | 0.858 | 327 |
| 0382/asn016-4 | 275 | 275 | 1 | R | P | 0.877 | 328 |
| 0382/asn015-2 | 275 | 275 | 1 | R | K | 1.002 | 329 |
| 0382/asn019-2 | 283 | 283 | 1 | M | I | 0.894 | 330 |
| 0383/asn008-3 | 283 | 284 | 2 | MW | CF | 1.145 | 331 |
| 0382/asn020-1 | 287 | 287 | 1 | F | L | 1.071 | 332 |
| 0382/asn023-4 | 295 | 295 | 1 | s | c | 0.713 | 333 |
| 0382/asn026-1 | 298 | 298 | 1 | F | G | 1.003 | 334 |
| 0382/asn028-3 | 309 | 309 | 1 | V | I | 1.338 | 335 |
| 0382/asn029-2 | 314 | 314 | 1 | I | V | 0.729 | 336 |
| 0382/asn034-2 | 323 | 323 | 1 | S | A | 0.965 | 337 |
| 0382/asn033-3 | 323 | 323 | 1 | S | T | 1.057 | 338 |
| 0382/asn035-3 | 326 | 326 | 1 | M | I | 0.744 | 339 |
| 0382/asn036-1 | 329 | 329 | 1 | E | Q | 0.975 | 340 |
| 0382/asn037-4 | 333 | 333 | 1 | I | L | 0.789 | 341 |
| 0382/asn039-2 | 343 | 343 | 1 | L | F | 0.788 | 342 |
| 0382/asn041-2 | 348 | 348 | 1 | K | G | 0.796 | 343 |
| 0382/asn042-2 | 350 | 350 | 1 | K | N | 1.196 | 344 |
| 0382/asn044-2 | 354 | 354 | 1 | L | F | 0.870 | 345 |
| 0383/asn018-4 | 354 | 356 | 3 | LFV | VYI | 1.252 | 346 |
| 0382/asn045-3 | 357 | 357 | 1 | F | Y | 0.829 | 347 |
| 0382/asn047-2 | 360 | 360 | 1 | I | c | 1.043 | 348 |
| 0382/asn048-2 | 361 | 361 | 1 | F | L | 1.715 | 349 |
| 0382/asn049-3 | 363 | 363 | 1 | I | L | 1.098 | 350 |
| 0382/asn052-1 | 374 | 374 | 1 | I | L | 1.417 | 351 |
| 0382/asn055-3 | 378 | 378 | 1 | Q | R | 0.947 | 352 |
| 0382/asn057-3 | 382 | 382 | 1 | T | A | 0.821 | 353 |
| 0382/asn061-3 | 398 | 398 | 1 | S | V | 1.138 | 354 |
| 0382/asn062-5 | 402 | 402 | 1 | T | S | 0.787 | 355 |
| 0382/asn065-4 | 417 | 417 | 1 | S | T | 1.029 | 356 |
| 0382/asn066-1 | 421 | 421 | 1 | A | L | 0.791 | 357 |
| 0382/asn068-4 | 426 | 426 | 1 | M | F | 1.050 | 358 |
| 0382/asn069-1 | 428 | 428 | 1 | M | L | 0.819 | 359 |
| 0362/asn046-4 | 447 | 450 | 4 | VSTV | ISTI | 1.058 | 360 |
| | | | | (SEQ ID | (SEQ | | |
| | | | | NO: 430) | ID NO: | | |
| | | | | | 431) | | |
| 0382/asn075-1 | 448 | 448 | 1 | S | T | 0.972 | 361 |
| 0382/asn076-1 | 450 | 450 | 1 | V | L | 0.996 | 362 |
| 0383/asn032-3 | 460 | 462 | 3 | TFV | SWL | 1.057 | 363 |
| 0382/asn079-2 | 473 | 473 | 1 | V | A | 1.069 | 364 |
| 0382/asn080-1 | 476 | 476 | 1 | S | L | 1.088 | 365 |
| 0382/asn081-3 | 481 | 481 | 1 | W | M | 0.926 | 366 |
| 0382/asn082-4 | 484 | 484 | 1 | V | A | 0.974 | 367 |
| 0362/asn053-2 | 484 | 491 | 8 | VFKSNI | LFKSN | 1.142 | 368 |
| | | | | MI (SEQ | VMV | | |
| | | | | ID NO: | (SEQ | | |
| | | | | 432) | 433) | | |
| 0382/asn084-4 | 488 | 488 | 1 | N | S | 1.093 | 369 |
| 0382/asn085-2 | 489 | 489 | 1 | I | V | 0.994 | 370 |
| 0382/asn088-2 | 493 | 493 | 1 | S | A | 1.117 | 371 |
| 0382/asn089-3 | 495 | 495 | 1 | A | I | 0.821 | 372 |
| 0382/asn090-2 | 499 | 499 | 1 | F | S | 0.745 | 373 |
| 0382/asn091-3 | 500 | 500 | 1 | C | S | 1.092 | 374 |
| 0382/asn092-3 | 503 | 503 | 1 | F | Y | 1.421 | 375 |
| 0382/asn094-1 | 510 | 510 | 1 | L | K | 0.799 | 376 |
| 0382/asn098-2 | 520 | 520 | 1 | Q | S | 0.769 | 377 |
| 0382/asn101-3 | 525 | 525 | 1 | I | L | 1.378 | 378 |
| 0382/asn102-2 | 527 | 527 | 1 | L | I | 0.741 | 379 |

Using the prenyltransferase mutant combinations of Tables 33-41 in the above described screen resulted in similar or improved CBGA titers as compared to yCBGA0537 strains encoding the native Saccharomyces cerevisiae prenyltransferase. Each mutant's respective amino acid mutations and amino acid sequences, are included in Tables 33-41 below. (All mutation sites refer to the mutation site on prenyltransferase's coding sequence without the wild-type signal sequence.)

TABLE 33
Combination “Combination Mutant Strain 1”
yCBGA_0537 combination mutant strains with multiple plasmid
constructs

| | | Last | | | |
|---|---|---|---|---|---|
| | First | position | | | |
| | position | of the | | | |
| | of the | mutation | | | |
| | mutation | site on | | | |
| | site on | PT's | | | |
| | PT's | coding | | | |
| | coding | sequence | | | |
| | sequence | without | | | |
| | without | the | Length | Wild type | mutant |
| Plasmid | the signal | signal | of | amino | amino |
| Constructs | sequence | sequence | mutation | acid | acid |
| 0382/asn001-2 | 242 | 242 | 1 | N | Q |
| 0382/asn003-4 | 249 | 249 | 1 | K | R |
| 0383/asn008-3 | 283 | 284 | 2 | MW | CF |
| 0382/asn020-1 | 287 | 287 | 1 | F | L |

TABLE 34
Combination “Combination Mutant Strain 2”
yCBGA_0537 combination mutant strains with multiple plasmid
constructs

| | | Last | | | |
|---|---|---|---|---|---|
| | First | position | | | |
| | position | of the | | | |
| | of the | mutation | | | |
| | mutation | site on | | | |
| | site on | PT's | | | |
| | PT's | coding | | | |
| | coding | sequence | | | |
| | sequence | without | | | |
| | without | the | Length | Wild type | mutant |
| Plasmid | the signal | signal | of | amino | amino |
| Constructs | sequence | sequence | mutation | acid | acid |
| 0383/asn001-1 | 239 | 241 | 3 | KIL | RVA |
| 0382/asn001-2 | 242 | 242 | 1 | N | Q |
| 0383/asn002-3 | 244 | 245 | 2 | GH | LK |
| 0382/asn003-4 | 249 | 249 | 1 | K | R |
| 0382/asn009-3 | 264 | 264 | 1 | C | S |
| 0382/asn013-2 | 272 | 272 | 1 | F | I |
| 0382/asn015-2 | 275 | 275 | 1 | R | K |
| 0383/asn008-3 | 283 | 284 | 2 | MW | CF |
| 0382/asn020-1 | 287 | 287 | 1 | F | L |

TABLE 35
Combination “Combination Mutant Strain 3”
yCBGA_0537 combination mutant strains with multiple plasmid
constructs

| | | Last | | | |
|---|---|---|---|---|---|
| | First | position | | | |
| | position | of the | | | |
| | of the | mutation | | | |
| | mutation | site on | | | |
| | site on | PT's | | | |
| | PT's | coding | | | |
| | coding | sequence | | | |
| | sequence | without | | | |
| | without | the | Length | Wild type | mutant |
| Plasmid | the signal | signal | of | amino | amino |
| Constructs | sequence | sequence | mutation | acid | acid |
| 0382/asn026-1 | 298 | 298 | 1 | F | G |
| 0382/asn028-3 | 309 | 309 | 1 | V | I |
| 0382/asn033-3 | 323 | 323 | 1 | S | T |
| 0382/asn037-4 | 333 | 333 | 1 | I | L |

TABLE 36
Combination “Combination Mutant Strain 4”
yCBGA_0537 combination mutant strains with multiple plasmid
constructs

| | | Last | | | |
|---|---|---|---|---|---|
| | First | position | | | |
| | position | of the | | | |
| | of the | mutation | | | |
| | mutation | site on | | | |
| | site on | PT's | | | |
| | PT's | coding | | | |
| | coding | sequence | | | |
| | sequence | without | | | |
| | without | the | Length | Wild type | mutant |
| Plasmid | the signal | signal | of | amino | amino |
| Constructs | sequence | sequence | mutation | acid | acid |
| 0382/asn023-4 | 295 | 295 | 1 | S | C |
| 0382/asn026-1 | 298 | 298 | 1 | F | G |
| 0382/asn028-3 | 309 | 309 | 1 | V | I |
| 0382/asn029-2 | 314 | 314 | 1 | I | V |
| 0382/asn033-3 | 323 | 323 | 1 | S | T |
| 0382/asn035-3 | 326 | 326 | 1 | M | I |
| 0382/asn036-1 | 329 | 329 | 1 | E | Q |
| 0382/asn037-4 | 333 | 333 | 1 | I | L |
| 0382/asn039-2 | 343 | 343 | 1 | L | F |

TABLE 37
Combination “Combination Mutant Strain 5”
yCBGA_0537 combination mutant strains with multiple plasmid
constructs

| | | Last | | | |
|---|---|---|---|---|---|
| | First | position | | | |
| | position | of the | | | |
| | of the | mutation | | | |
| | mutation | site on | | | |
| | site on | PT's | | | |
| | PT's | coding | | | |
| | coding | sequence | | | |
| | sequence | without | | | |
| | without | the | Length | Wild type | mutant |
| Plasmid | the signal | signal | of | amino | amino |
| Constructs | sequence | sequence | mutation | acid | acid |
| 0383/asn018-4 | 354 | 356 | 3 | LFV | VYI |
| 0382/asn048-2 | 361 | 361 | 1 | F | L |
| 0382/asn049-3 | 363 | 363 | 1 | I | L |
| 0382/asn052-1 | 374 | 374 | 1 | I | L |

TABLE 38
Combination “Combination Mutant Strain 6”
yCBGA_0537 combination mutant strains with multiple plasmid
constructs

| | | Last | | | |
|---|---|---|---|---|---|
| | First | position | | | |
| | position | of the | | | |
| | of the | mutation | | | |
| | mutation | site on | | | |
| | site on | PT's | | | |
| | PT's | coding | | | |
| | coding | sequence | | | |
| | sequence | without | | | |
| | without | the | Length | Wild type | mutant |
| Plasmid | the signal | signal | of | amino | amino |
| Constructs | sequence | sequence | mutation | acid | acid |
| 0382/asn062-5 | 402 | 402 | 1 | T | S |
| 0382/asn065-4 | 417 | 417 | 1 | S | T |
| 0382/asn066-1 | 421 | 421 | 1 | A | L |
| 0382/asn068-4 | 426 | 426 | 1 | M | F |

TABLE 39
Combination “Combination Mutant Strain 7”
yCBGA_0537 combination mutant strains with multiple plasmid
constructs

| | | Last | | | |
|---|---|---|---|---|---|
| | First | position | | | |
| | position | of the | | | |
| | of the | mutation | | | |
| | mutation | site on | | | |
| | site on | PT's | | | |
| | PT's | coding | | | |
| | coding | sequence | | | |
| | sequence | without | | | |
| | without | the | Length | Wild type | mutant |
| Plasmid | the signal | signal | of | amino | amino |
| Constructs | sequence | sequence | mutation | acid | acid |
| 0382/asn075-1 | 448 | 448 | 1 | S | T |
| 0382/asn076-1 | 450 | 450 | 1 | V | L |
| 0383/asn032-3 | 460 | 462 | 3 | TFV | SWL |
| 0382/asn079-2 | 473 | 473 | 1 | V | A |
| 0382/asn085-2 | 489 | 489 | 1 | I | V |

TABLE 40
Combination “Combination Mutant Strain 8”
yCBGA_0537 combination mutant strains with multiple plasmid
constructs

| | | Last | | | |
|---|---|---|---|---|---|
| | First | position | | | |
| | position | of the | | | |
| | of the | mutation | | | |
| | mutation | site on | | | |
| | site on | PT's | | | |
| | PT's | coding | | | |
| | coding | sequence | | | |
| | sequence | without | Length | | |
| | without | the | of | Wild type | mutant |
| Plasmid | the signal | signal | muta- | amino | amino |
| Constructs | sequence | sequence | tion | acid | acid |
| 0362/asn046-4 | 447 | 450 | 4 | VSTV | ISTI |
| 0383/asn032-3 | 460 | 462 | 3 | TFV | SWL |
| 0382/asn079-2 | 473 | 473 | 1 | V | A |
| 0382/asn080-1 | 476 | 476 | 1 | S | L |
| 0382/asn081-3 | 481 | 481 | 1 | W | M |
| 0362/asn053-2 | 484 | 491 | 8 | VFKSNI | LFKSN |
| | | | | MI | VMV |

TABLE 41
Combination “Combination Mutant Strain 9”
yCBGA_0527 comination mutant strains with multiple plasmid
constructs

| | First | Last | | | |
|---|---|---|---|---|---|
| | position | position | | | |
| | of the | of the | | | |
| | mutantion | mutation | | | |
| | site on | site on | | | |
| | PT's | PT's | | | |
| | coding | coding | | | |
| | sequence | sequence | | | |
| | without | without | | | |
| | the | the | Length | Wild type | mutant |
| Plasmid | signal | signal | of | amino | amino |
| Constructs | sequence | sequence | mutation | acid | acid |
| 0382/asn089-3 | 495 | 495 | 1 | A | I |
| 0382/asn091-3 | 500 | 500 | 1 | C | S |
| 0382/asn092-3 | 503 | 503 | 1 | F | Y |
| 0382/asn101-3 | 525 | 525 | 1 | I | L |

1. A genetically modified microorganism comprising at least three polynucleotides that encode for:
a) an amino acid sequence that is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO: 27;
b) an amino acid sequence that is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO: 32; or
c) combinations thereof.

2. The genetically modified microorganism of claim 1 comprising:
a polynucleotide that encodes for an amino acid sequence that is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO: 27.

3. The genetically modified microorganism of claim 1 or claim 2 comprising:
a polynucleotide that encodes for an amino acid sequence that is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO: 32.

4. The genetically modified microorganism of any one of claims 1 to 3 comprising:
at least two polynucleotides that encode for an amino acid sequence that is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO: 32.

5. The genetically modified microorganism of any one of claims 1 to 4, wherein the at least three polynucleotides encode for proteins having prenyltransferase activity.

6. The genetically modified microorganism of any one of claims 1 to 5, wherein the microorganism comprises at least one polynucleotide that encodes a F96W mutant of Saccharomyces cerevisiae ERG20.

7. The genetically modified microorganism of any one of claims 1 to 6, wherein the microorganism comprises at least one polynucleotide that encodes an N127W mutant of Saccharomyces cerevisiae ERG20.

8. The genetically modified microorganism of any one of claims 1 to 7, wherein at least one of the microorganism's engodenous genes is disrupted; preferably wherein the engodenous genes is deleted.

9. The genetically modified microorganism of any one of claims 1 to 8, wherein the microorganism further comprises the polynucleotide sequence of the Saccharomyces cerevisiae GAL1/GAL10 promoter.

10. The genetically modified microorganism of claim 9, wherein the GAL1/GAL10 promoter is inserted into the microorganism's native LPP1 locus.

11. The genetically modified microorganism of claim 10, wherein the microorganism's native LPP1 open reading frame is deleted.

12. The genetically modified microorganism of any one of claims 1 to 11, wherein the microorganism further comprises at least one polynucleotide that encodes for an amino acid sequence that is substantially identical to a truncated amino acid sequence of the Saccharomyces cerevisiae HMG1, wherein the first 530 amino acids of the HMG1 are truncated.

13. The genetically modified microorganism of any one of claims 1 to 12, further comprising at least one polynucleotide encoding at least one polypeptide with acyl activating activity; polyketide synthase activity; olivetol synthase activity; tetraketide synthase activity; olivetolic acid cyclase activity; THCA synthase activity; CBDA synthase activity; CBCA synthase activity; HMG-Co reductase activity; farnesyl pyrophosphate synthetase activity; or any combination thereof.

14. The genetically modified microorganism of any one of claims 1 to 13, further comprising at least one polynucleotide encoding an acyl activating enzyme (AAE1); a polyketide synthase (PKS), such as a tetraketide synthase (TKS); an olivetolic acid cyclase (OAC); a THCA synthase (THCAS); a CBDA synthase (CBDAS); a CBCA synthase (CBCAS); a HMG-Co reductase (HMG1); a farnesyl pyrophosphate synthetase (ERG20); or any combination thereof;
preferably wherein the AAE1 is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO: 14;
preferably wherein the TKS is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO: 41.
preferably wherein the OAC is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO: 8;
preferably wherein the THCAS is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to a sequence chosen from SEQ ID NO: 10 or SEQ ID NO: 120;
preferably wherein the THCAS is a T446A mutant of SEQ ID NO: 120, a T446V mutant of SEQ ID NO: 120, or a T446I mutant of SEQ ID NO: 120, or a combination thereof;
preferably wherein the polynucleotide encodes a THCAS signal sequence substantially identical to, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to a sequence chosen from SEQ ID NO: 121 to SEQ ID NO: 138;
preferably wherein the CBDAS is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO: 12;
preferably wherein the CBCAS is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO: 18;
preferably wherein the HMG1 is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO: 20 or SEQ ID NO: 22;
preferably wherein the ERG20 is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO: 24.

15. The genetically modified microorganism of any one of claims 1 to 14, further comprising at least one polynucleotide encoding an enzyme that is capable of converting olivetolic acid to cannabigerolic acid (“CBGA”).

16. The genetically modified microorganism of any one of claims 1 to 15, further comprising at least one polynucleotide encoding an enzyme that is capable of converting butyric acid to cannabigerolic acid (“CBGVA”).

17. The genetically modified microorganism of any one of claims 1 to 16, further comprising a polynucleotide that encodes for an amino acid sequence that is substantially identical to, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO: 5.

18. The genetically modified microorganism of any one of claims 1 to 17, further comprising a polynucleotide that is substantially identical to, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO: 6.

19. The genetically modified microorganism of any one of claims 1 to 18, further comprising a polynucleotide that encodes for an amino acid sequence that is substantially identical to, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO: 7.

20. The genetically modified microorganism of any one of claims 1 to 19, further comprising a polynucleotide that is substantially identical to, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO:8.

21. The genetically modified microorganism of any one of claims 1 to 20, further comprising a polynucleotide that encodes for an amino acid sequence that is substantially identical to, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO: 13.

22. The genetically modified microorganism of any one of claims 1 to 21, further comprising a polynucleotide that is substantially identical to, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO:14.

23. The genetically modified microorganism of any one of claims 1 to 22, wherein said microorganism comprises at least two polynucleotides encoding a protein with AAE1 activity.

24. The genetically modified microorganism of any one of claims 1 to 23, wherein said microorganism comprises at least three polynucleotides encoding a protein with AAE1 activity.

25. The genetically modified microorganism of any one of claims 1 to 24, wherein said microorganism comprises at least two polynucleotides encoding a protein with TKS activity.

26. The genetically modified microorganism of any one of claims 1 to 25, wherein said microorganism comprises at least three polynucleotides encoding a protein with TKS activity.

27. The genetically modified microorganism of any one of claims 1 to 26, wherein said microorganism comprises at least two polynucleotides encoding a protein with OAC activity.

28. The genetically modified microorganism of any one of claims 1 to 27, wherein said microorganism comprises at least three polynucleotides encoding a protein with OAC activity.

29. The genetically modified microorganism of any one of claims 1 to 28, wherein said microorganism comprises at least three polynucleotides encoding a protein with AAE1 activity; at least three polynucleotides encoding a protein with TKS activity; and at least three polynucleotides encoding a protein with OAC activity.

30. The genetically modified microorganism of any one of claims 1 to 29, further comprising one or more polynucleotides encoding proteins with Hydroxymethylglutaryl-CoA synthase activity; Hydroxymethylglutaryl-CoA reductase activity; tHMG1 activity; Acetyl-CoA C-acetyltransferase activity; or any combination thereof.

31. The genetically modified microorganism of any one of claims 1 to 30, further comprising one or more polynucleotides encoding a Hydroxymethylglutaryl-CoA synthase (ERG13); a Hydroxymethylglutaryl-CoA reductase (HMG1); a tHMG1; a Acetyl-CoA C-acetyltransferase (ERG10); or any combination thereof.

32. The genetically modified microorganism of any one of claims 1 to 31, further comprising a polynucleotide encoding an ERG13; a polynucleotide encoding a HGM1 and a polynucleotide encoding an amino acid sequence that is substantially identical to, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical SEQ ID NO: 32.

33. The genetically modified microorganism of any one of claims 1 to 31, further comprising a polynucleotide encoding a tHMG1; a polynucleotide encoding an ERG10 and a polynucleotide encoding an EGR13.

34. The genetically modified microorganism of any one of claims 1 to 31, further comprising a polynucleotide encoding a tHMG1; a polynucleotide encoding an ERG13 and a polynucleotide encoding an AAE1.

35. The genetically modified microorganism of any one of claims 1 to 22, further comprising a polynucleotide encoding an enzyme with CBDA synthase activity, a polynucleotide encoding an enzyme with CBCA synthase, a polynucleotide encoding an enzyme with CBCA and CBDA synthase activity, or a combination thereof,
preferably wherein the the enzyme with CBDA synthase activity is substantially identical to, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to a sequence chosen from SEQ ID NO; 43 or SEQ ID NO: 153 to SEQ ID NO: 287;
preferably wherein the polynucleotide encodes a CBDA synthase signal sequence substantially identical to, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to a sequence chosen from SEQ ID NO: 44 to SEQ ID NO: 73 or SEQ ID NO: 104 to SEQ ID NO: 110,
preferably wherein the enzyme with CBCA synthase activity is substantially identical to, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to a sequence chosen from SEQ ID NO: 288 to SEQ ID NO: 297 or SEQ ID NO: 305 to SEQ ID NO: 318.

36. The genetically modified microorganism of any one of claims 1 to 22 and 35, further comprising the bCBGA1854 plasmid of SEQ ID No.: 435.

37. The genetically modified microorganism of any one of claims 1 to 22 and 35 to 36, further comprising a polynucleotiode encoding a protein with PKS activity, a polynucleotiode encoding a protein with OAC activity, and a polynucleotiode encoding a protein with AAE1 activity.

38. The genetically modified microorganism of any one of claims 1 to 22 and 35 to 37, further comprising a polynucleotide encoding a PIR3-CBDA of SEQ ID NO: 302.

39. The genetically modified microorganism of any one of claims 1 to 22 and 35 to 38, further comprising a signal peptide corresponding to 0253/asn053-2.

40. The genetically modified microorganism of any one of claims 1 to 22, wherein the microorganism's engodenous VPS10 gene is disrupted; preferably wherein the sequence of the disrupted gene is SEQ ID NO: 300.

41. The genetically modified microorganism of any one of claims 1 to 23 and 40, wherein the coding sequence of the microorganism's engodenous VPS10 gene is deleted.

42. The genetically modified microorganism of any one of claims 1 to 41, wherein said microorganism is capable of producing cannabigerolic acid.

43. The genetically modified microorganism of any one of claims 1 to 42, wherein said microorganism is capable of producing a cannabinoid.

44. The genetically modified microorganism of claim 43, wherein said cannabinoid is selected from Δ9-tetrahydrocannabinolic acid (THCA), cannabidiolic acid (CBDA), cannabichromenic acid (CBCA), Δ9-tetrahydrocannabinol (THC), cannabidiol (CBD), cannabichromene (CBC), Δ⁹-tetrahydrocannabivarinic acid (THCVA), cannabidivarinic acid (CBDVA), cannabichromevarinic acid (CBCVA), or any combination thereof.

45. The genetically modified microorganism of claim 8, wherein said one or more endogenous genes is from a pathway that controls beta oxidation of long chain fatty acids.

46. The genetically modified microorganism of claim 45, wherein said at least one endogenous gene is FOX1, FAA1, FAA4, FAT1, PXA1, PXA2, and/or PEX11.

47. The genetically modified microorganism of claim 45 or 46, wherein said at least one endogenous gene is FOX1.

48. The genetically modified microorganism of any one of claims 45 to 47, wherein said one or more gene is disrupted using a CRISPR/Cas system.

49. The genetically modified microorganism of any one of claims 1 to 48, wherein said microorganism is a bacterium or a yeast.

50. The genetically modified microorganism of any one of claims 1 to 49, wherein said microorganism is a yeast.

51. The genetically modified microorganism of claim 50, wherein said yeast is from the genus Saccharomyces.

52. The genetically modified microorganism of claim 51, wherein said yeast is from the species Saccharomyces cerevisiae.

53. A genetically modified microorganism comprising:
at least two polynucleotides that encode for amino acid sequences that are substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO: 27, SEQ ID NO: 32, or combinations thereof,
at least three polynucleotides that encode for a protein with acyl activating activity;
at least three polynucleotides that encode for a protein with polyketide synthase activity;
at least three polynucleotides that encode for a protein with olivetolic acid cyclase activity.

54. A genetically modified microorganism comprising a polynucleotide that encodes for a Saccharomyces cerevisiae TKS with a mutation at Ala125.

55. The genetically modified microorganism of claim 54, wherein the mutation is Ala125Ser.

56. A method of producing CBGA comprising:
(a) contacting a carbon substrate with the genetically modified microorganism of any one of claims 1 to 55;
(b) growing said genetically modified microorganism to produce CBGA.

57. The method of claim 56, further comprising (c) isolating said CBGA from the genetically modified organism.

58. The method of claim 56 or 57, wherein said carbon substrate is a sugar, alcohol, and/or fatty acid.

59. The method of any one of claims 56 to 58, wherein said carbon substrate is selected from hexanoic acid, glucose, fructose, xylose, sucrose, dextrins, starch, xylan, cellulose, hemicellulose, arabinose, glycerol, ethanol, butanol, methanol, or any combination thereof.

60. The method of any one of claims 56 to 59, wherein said carbon substrate is hexanoic acid.

61. The method of any one of claims 56 to 60, wherein said CBGA is converted to Δ⁹-tetrahydrocannabinol (THC), cannabidiol (CBD), cannabichromene (CBC), or any combination thereof.

62. The method of claim 61, wherein said CBGA conversion is completed outside the microorganism.

63. The method of claim 61 or 62, wherein said conversion is a non-enzymatic conversion.

64. The method of any one of claims 61 to 63, wherein said conversion is an enzymatic conversion.

65. A method of producing CBGVA comprising:
(a) contacting a carbon substrate with the genetically modified microorganism of any one of claims 1 to 55;
(b) growing said genetically modified microorganism to produce CBGVA.

66. The method of claim 65, further comprising (c) isolating said CBGVA from the genetically modified organism.

67. The method of claim 65 or 66, wherein said carbon substrate is a fatty acid.

68. The method of any one of claims 65 to 67, wherein said carbon substrate is butyric acid.

69. The method of any one of claims 65 to 68, wherein said CBGVA is converted to Δ⁹-tetrahydrocannabivarinic acid (THCVA), cannabidivarinic acid (CBDVA), cannabichromevarinic acid (CBCVA), or any combination thereof.

70. The method of claim 69, wherein said CBGA conversion is completed outside the microorganism.

71. The method of claim 69 or 70, wherein said conversion is a non-enzymatic conversion.

72. The method of any one of claims 69 to 71, wherein said conversion is an enzymatic conversion.

73. A method of producing a cannabinoid comprising:
(a) contacting a carbon substrate with the genetically modified microorganism of any one of claims 1 to 55;
(b) growing said genetically modified microorganism to producing a cannabinoid.

74. The method of claim 73, further comprising (c) isolating said cannabinoid from the genetically modified organism.

75. The method of claim 73 or 74, wherein said carbon substrate is selected from a sugar, alcohol, and/or fatty acid.

76. The method of any one of claims 73 to 75, wherein said carbon substrate is selected from hexanoic acid, butyric acid, glucose, fructose, xylose, sucrose, dextrins, starch, xylan, cellulose, hemicellulose, arabinose, glycerol, ethanol, butanol, methanol, or any combination thereof.

77. The method of any one of claims 73 to 76, wherein said carbon substrate is hexanoic acid.

78. The method of any one of claims 73 to 77, wherein said cannabinoid is Δ⁹-tetrahydrocannabinol (THC), cannabidiol (CBD), cannabichromene (CBC), or any combination thereof.

79. The method of any one of claims 73 to 78, wherein the microorganism produces CBGA, and wherein the CBGA is converted to a cannabinoid outside the microorganism.

80. The method of any one of claims 73 to 76, wherein said carbon substrate is butyric acid.

81. The method of any one of claims 73 to 76 and 80, wherein said cannabinoid is Δ⁹-tetrahydrocannabivarinic acid (THCVA), cannabidivarinic acid (CBDVA), cannabichromevarinic acid (CBCVA), or any combination thereof.

82. The method of any one of claims 73 to 76 and 80 to 81, wherein the microorganism produces CBGVA, and wherein the CBGVA is converted to a cannabinoid outside the microorganism.

83. The method of claim 79 or 82, wherein said conversion is a non-enzymatic conversion.

84. The method of claim 79, 82 or 83, wherein said conversion is an enzymatic conversion.

85. The genetically modified organism of claim 29 or 33, wherein said organism comprises a polynucleotide sequence encoding at least one amino acid sequence substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to a sequence chosen from SEQ ID NO: 153 to SEQ ID NO: 287;
preferably wherein the polynucleotide further encodes a CBDA synthase signal sequence substantially identical to, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to a sequence chosen from SEQ ID NO: 44 to SEQ ID NO: 73 or SEQ ID NO: 104 to SEQ ID NO: 110.

86. The genetically modified organism of claim 29 or 33, wherein said organism comprises a polynucleotide sequence encoding at least one amino acid sequence substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to a sequence chosen from SEQ ID NO: 288 to SEQ ID NO: 297 or SEQ ID NO: 305 to SEQ ID NO: 318.

87. A method of producing CBCA and/or CBDA comprising:
(a) contacting a carbon substrate with the genetically modified microorganism of claim 85 or 86;
(b) growing said genetically modified microorganism to produce said CBCA and/or CBDA.

88. The method of claim 87, further comprising (c) isolating said CBCA and/or CBDA from the genetically modified organism.

89. The method of claim 87 or 88, wherein said carbon substrate is a sugar, alcohol, and/or fatty acid.

90. The method of any one of claims 87 to 89, wherein said carbon substrate is selected from hexanoic acid, butyric acid, glucose, fructose, xylose, sucrose, dextrins, starch, xylan, cellulose, hemicellulose, arabinose, glycerol, ethanol, butanol, methanol, or any combination thereof.

91. The method of any one of claims 87 to 89, wherein said carbon substrate is hexanoic acid.

92. The method of any one of claims 87 to 89, wherein said carbon substrate is butyric acid.

93. The genetically modified organism of claim 29, wherein said organism comprises a polypeptide comprising an amino acid sequence substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO: 27.

94. A method of producing a cannabinoid comprising:
(a) contacting a carbon substrate with the genetically modified microorganism of claim 93;
(b) growing said genetically modified microorganism to produce said cannabinoid.

95. The method of claim 94, further comprising (c) isolating CBCA from the genetically modified organism.

96. The method of claim 94 or 95, wherein said carbon substrate is a sugar, alcohol, and/or fatty acid.

97. The method of any one of claims 94 to 96, wherein said carbon substrate is selected from hexanoic acid, butyric acid, glucose, fructose, xylose, sucrose, dextrins, starch, xylan, cellulose, hemicellulose, arabinose, glycerol, ethanol, butanol, methanol, or any combination thereof.

98. The method of any one of claims 94 to 96, wherein said carbon substrate is hexanoic acid.

99. The method of any one of claims 94 to 96, wherein said carbon substrate is butyric acid.

100. The genetically modified organism of claim 29, wherein said organism comprises a polynucleotide sequence encoding at least one amino acid sequence substantially identical to, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID: No. 120; preferably wherein the THCAS is a T446A mutant of SEQ ID NO: 120; a T446V mutant of SEQ ID NO: 120; or a T446I mutant of SEQ ID NO: 120, or a combination thereof.

101. A method of producing THCA comprising:
(a) contacting a carbon substrate with the genetically modified microorganism of claim 100;
(b) growing said genetically modified microorganism to producing THCA.

102. The method of claim 101, further comprising (c) isolating THCA from the genetically modified organism.

103. The method of claim 101 or 102, wherein said carbon substrate is a sugar, alcohol, and/or fatty acid.

104. The method of any one of claims 101 to 103, wherein said carbon substrate is selected from hexanoic acid, butyric acid, glucose, fructose, xylose, sucrose, dextrins, starch, xylan, cellulose, hemicellulose, arabinose, glycerol, ethanol, butanol, methanol, or any combination thereof.

105. The method of any one of claims 101 to 103, wherein said carbon substrate is hexanoic acid.

106. The method of any one of claims 101 to 103, wherein said carbon substrate is butyric acid.

107. The use of a cannabinoid produced by any one of the methods of claims 56 to 84, 87 to 92, 94 to 99, and 101-106 for the manufacture of a medicament for the treatment of a disease or a symptom of a disease.

108. The use of claim 107, wherein said a disease or a symptom of a disease is anorexia, multiple sclerosis, neurodegenerative disorders, epilepsy, glaucoma, osteoporosis, schizophrenia, bipolar disorder, post-traumatic stress disorder (PTSD), asthma, cardiovascular disorders, cancer, obesity, metabolic syndrome-related disorders, depression, anxiety, insomnia, emesis, pain, or inflammation.

109. A medicament comprising a cannabinoid made by any one of the methods of claims 56 to 84, 87 to 92, 94 to 99, and 101-106 and a pharmaceutically acceptable excipient.

110. A method of treating a disease or a symptom of a disease comprising administering a subject in need thereof the cannabinoid made by any one of the methods of claims 56 to 84, 87 to 92, 94 to 99, and 101-106.

111. The method of claim 110, wherein said a disease or a symptom of a disease is anorexia, multiple sclerosis, neurodegenerative disorders, epilepsy, glaucoma, osteoporosis, schizophrenia, bipolar disorder, post-traumatic stress disorder (PTSD), asthma, cardiovascular disorders, cancer, obesity, metabolic syndrome-related disorders, depression, anxiety, insomnia, emesis, pain, or inflammation.

112. A method of treating a disease or a symptom of a disease comprising administering a subject in need thereof the medicament of claim 109.

113. The use of a cannabinoid produced by any one of the microorganisms or methods of claims 1 to 106 for the manufacture of a medicament for recreational use.

114. The use of any one of claims 107, 108 and 113, wherein the medicament is delivered through inhalation, intravenously, oral, or topical.

115. The use of claim 114, wherein the delivery is inhalation and completed through a vaporizer.

116. The use of claim 114, wherein said delivery is intravenous and the medicament is delivered through a saline solution.

117. The use of claim 114, wherein said delivery is oral and the medicament is delivered with food.

118. The use of claim 114, wherein said delivery is oral and the medicament is delivered through drink.

119. The use of claim 114, wherein said delivery is topical and the medicament is delivered through a patch.

120. The use of claim 114, wherein said delivery is topical and the medicament is delivered through a lotion.

121. A genetically modified microorganism comprising at least one polynucleotide encoding for an amino acid sequence that is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to a sequence chosen from SEQ ID NO: 320 to SEQ ID NO: 379, a K239A+I240V+L241A combination mutant of SEQ ID NO: 320; a N242D mutant of SEQ ID NO: 320; a N24Q mutant of SEQ ID NO: 320; a G244L+H245K mutant of SEQ ID NO: 320; a K249R mutant of SEQ ID NO: 320; a C264S mutant of SEQ ID NO: 320; a F272I mutant of SEQ ID NO: 320; a R275P mutant of SEQ ID NO: 320; a R275K mutant of SEQ ID NO: 320; a M283I mutant of SEQ ID NO: 320; a M283C+W284F mutant of SEQ ID NO: 320; a F287L mutant of SEQ ID NO: 320; a S295C mutant of SEQ ID NO: 320; a F298G mutant of SEQ ID NO: 320; a F309I mutant of SEQ ID NO: 320; a I314V mutant of SEQ ID NO: 320; a S323A mutant of SEQ ID NO: 320; a S323T mutant of SEQ ID NO: 320; a M326I mutant of SEQ ID NO: 320, a E329Q mutant of SEQ ID NO: 320; a I333L mutant of SEQ ID NO: 320; a L343F mutant of SEQ ID NO: 320; a K348G mutant of SEQ ID NO: 320; a K350N mutant of SEQ ID NO: 320; a L354F mutant of SEQ ID NO: 320; a L354V+F355Y+V356I mutant of SEQ ID NO: 320; a F357Y mutant of SEQ ID NO: 320; a I360C mutant of SEQ ID NO: 320, a F361L mutant of SEQ ID NO: 320; a I363L mutant of SEQ ID NO: 320; a I374L mutant of SEQ ID NO: 320; a Q378K mutant of SEQ ID NO: 320; a T382A mutant of SEQ ID NO: 320; a S398V mutant of SEQ ID NO: 320, a S398V mutant of SEQ ID NO: 320; a T402S mutant of SEQ ID NO: 320; a S417T mutant of SEQ ID NO: 320; a A421L mutant of SEQ ID NO: 320; a M426F mutant of SEQ ID NO: 320, a M428L mutant of SEQ ID NO: 320; a V447+V450I mutant of SEQ ID NO: 320; a S448T mutant of SEQ ID NO: 320; a V450L mutant of SEQ ID NO: 320; a T460S+F461W+V462L mutant of SEQ ID NO: 320, a V473A mutant of SEQ ID NO: 320; a S476L mutant of SEQ ID NO: 320; a W481M mutant of SEQ ID NO: 320; a V484A mutant of SEQ ID NO: 320; a V484L+I489V+I491V mutant of SEQ ID NO: 320, a N488S mutant of SEQ ID NO: 320; a I489V mutant of SEQ ID NO: 320; a S493A mutant of SEQ ID NO: 320; a A495I mutant of SEQ ID NO: 320; a F499S mutant of SEQ ID NO: 320, a C500S mutant of SEQ ID NO: 320; a F503Y mutant of SEQ ID NO: 320; a L510K mutant of SEQ ID NO: 320, a Q520S mutant of SEQ ID NO: 320; a I525L mutant of SEQ ID NO: 320; a L527I mutant of SEQ ID NO: 320; or combinations thereof.

122. A method of producing a cannabinoid comprising:
(a) contacting a carbon substrate with the genetically modified microorganism of claim 121;
(b) growing said genetically modified microorganism to produce a cannabinoid; and
optionally (c) isolating the cannabinoid from the genetically modified organism.

123. A polynucleotide encoding for at least one amino acid sequence that is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to a sequence chosen from SEQ ID NO: 320 to SEQ ID NO: 379, a K239A+I240V+L241A combination mutant of SEQ ID NO: 320; a N242D mutant of SEQ ID NO: 320; a N24Q mutant of SEQ ID NO: 320; a G244L+H245K mutant of SEQ ID NO: 320; a K249R mutant of SEQ ID NO: 320; a C264S mutant of SEQ ID NO: 320; a F272I mutant of SEQ ID NO: 320; a R275P mutant of SEQ ID NO: 320; a R275K mutant of SEQ ID NO: 320; a M283I mutant of SEQ ID NO: 320; a M283C+W284F mutant of SEQ ID NO: 320; a F287L mutant of SEQ ID NO: 320; a S295C mutant of SEQ ID NO: 320; a F298G mutant of SEQ ID NO: 320; a F309I mutant of SEQ ID NO: 320; a I314V mutant of SEQ ID NO: 320; a S323A mutant of SEQ ID NO: 320; a S323T mutant of SEQ ID NO: 320; a M326I mutant of SEQ ID NO: 320, a E329Q mutant of SEQ ID NO: 320; a I333L mutant of SEQ ID NO: 320; a L343F mutant of SEQ ID NO: 320; a K348G mutant of SEQ ID NO: 320; a K350N mutant of SEQ ID NO: 320; a L354F mutant of SEQ ID NO: 320; a L354V+F355Y+V356I mutant of SEQ ID NO: 320; a F357Y mutant of SEQ ID NO: 320; a I360C mutant of SEQ ID NO: 320, a F361L mutant of SEQ ID NO: 320; a I363L mutant of SEQ ID NO: 320; a I374L mutant of SEQ ID NO: 320; a Q378K mutant of SEQ ID NO: 320; a T382A mutant of SEQ ID NO: 320; a S398V mutant of SEQ ID NO: 320, a S398V mutant of SEQ ID NO: 320; a T402S mutant of SEQ ID NO: 320; a S417T mutant of SEQ ID NO: 320; a A421L mutant of SEQ ID NO: 320; a M426F mutant of SEQ ID NO: 320, a M428L mutant of SEQ ID NO: 320; a V447+V450I mutant of SEQ ID NO: 320; a S448T mutant of SEQ ID NO: 320; a V450L mutant of SEQ ID NO: 320; a T460S+F461W+V462Lmutant of SEQ ID NO: 320, a V473A mutant of SEQ ID NO: 320; a S476L mutant of SEQ ID NO: 320; a W481M mutant of SEQ ID NO: 320; a V484A mutant of SEQ ID NO: 320; a V484L+I489V+I491V mutant of SEQ ID NO: 320, a N488S mutant of SEQ ID NO: 320; a I489V mutant of SEQ ID NO: 320; a S493A mutant of SEQ ID NO: 320; a A495I mutant of SEQ ID NO: 320; a F499S mutant of SEQ ID NO: 320, a C500S mutant of SEQ ID NO: 320; a F503Y mutant of SEQ ID NO: 320; a L510K mutant of SEQ ID NO: 320, a Q520S mutant of SEQ ID NO: 320; a I525L mutant of SEQ ID NO: 320; a L527I mutant of SEQ ID NO: 320; or combinations thereof.

124. A vector comprising the polynucleotide of claim 123.

125. A polypeptide comprising an amino acid sequence that is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to a sequence chosen from SEQ ID NO: 320 to SEQ ID NO: 379, a K239A+I240V+L241A combination mutant of SEQ ID NO: 320; a N242D mutant of SEQ ID NO: 320; a N24Q mutant of SEQ ID NO: 320; a G244L+H245K mutant of SEQ ID NO: 320; a K249R mutant of SEQ ID NO: 320; a C264S mutant of SEQ ID NO: 320; a F272I mutant of SEQ ID NO: 320; a R275P mutant of SEQ ID NO: 320; a R275K mutant of SEQ ID NO: 320; a M283I mutant of SEQ ID NO: 320; a M283C+W284F mutant of SEQ ID NO: 320; a F287L mutant of SEQ ID NO: 320; a S295C mutant of SEQ ID NO: 320; a F298G mutant of SEQ ID NO: 320; a F309I mutant of SEQ ID NO: 320; a I314V mutant of SEQ ID NO: 320; a S323A mutant of SEQ ID NO: 320; a S323T mutant of SEQ ID NO: 320; a M326I mutant of SEQ ID NO: 320, a E329Q mutant of SEQ ID NO: 320; a I333L mutant of SEQ ID NO: 320; a L343F mutant of SEQ ID NO: 320; a K348G mutant of SEQ ID NO: 320; a K350N mutant of SEQ ID NO: 320; a L354F mutant of SEQ ID NO: 320; a L354V+F355Y+V356I mutant of SEQ ID NO: 320; a F357Y mutant of SEQ ID NO: 320; a I360C mutant of SEQ ID NO: 320, a F361L mutant of SEQ ID NO: 320; a I363L mutant of SEQ ID NO: 320; a I374L mutant of SEQ ID NO: 320; a Q378K mutant of SEQ ID NO: 320; a T382A mutant of SEQ ID NO: 320; a S398V mutant of SEQ ID NO: 320, a S398V mutant of SEQ ID NO: 320; a T402S mutant of SEQ ID NO: 320; a S417T mutant of SEQ ID NO: 320; a A421L mutant of SEQ ID NO: 320; a M426F mutant of SEQ ID NO: 320, a M428L mutant of SEQ ID NO: 320; a V447+V450I mutant of SEQ ID NO: 320; a S448T mutant of SEQ ID NO: 320; a V450L mutant of SEQ ID NO: 320; a T460S+F461W+V462Lmutant of SEQ ID NO: 320, a V473A mutant of SEQ ID NO: 320; a S476L mutant of SEQ ID NO: 320; a W481M mutant of SEQ ID NO: 320; a V484A mutant of SEQ ID NO: 320; a V484L+I489V+I491V mutant of SEQ ID NO: 320, a N488S mutant of SEQ ID NO: 320; a I489V mutant of SEQ ID NO: 320; a S493A mutant of SEQ ID NO: 320; a A495I mutant of SEQ ID NO: 320; a F499S mutant of SEQ ID NO: 320, a C500S mutant of SEQ ID NO: 320; a F503Y mutant of SEQ ID NO: 320; a L510K mutant of SEQ ID NO: 320, a Q520S mutant of SEQ ID NO: 320; a I525L mutant of SEQ ID NO: 320; a L527I mutant of SEQ ID NO: 320; or combinations thereof.

126. A genetically modified microorganism comprising at least one polynucleotide encoding for an amino acid sequence that is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to a sequence chosen from SEQ ID NO: 153 to SEQ ID NO: 287, or combinations thereof;
preferably wherein the at least one polynucleotide further encodes a CBDA synthase signal sequence substantially identical to, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to a sequence chosen from SEQ ID NO: 44 to SEQ ID NO: 73 or SEQ ID NO: 104 to SEQ ID NO: 110,

127. A method of producing CBDA comprising:
(a) contacting a carbon substrate with the genetically modified microorganism of claim 126;
(b) growing said genetically modified microorganism to produce CBDA; and
optionally (c) isolating the CBDA from the genetically modified organism.

128. A polynucleotide encoding for at least one amino acid sequence that is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to a sequence chosen from SEQ ID NO: 153 to SEQ ID NO: 287, or combinations thereof;
preferably wherein the at least one polynucleotide further encodes a CBDA synthase signal sequence substantially identical to, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to a sequence chosen from SEQ ID NO: 44 to SEQ ID NO: 73 or SEQ ID NO: 104 to SEQ ID NO: 110.

129. A vector comprising the polynucleotide of claim 128.

130. A polypeptide comprising an amino acid sequence that is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to a sequence chosen from SEQ ID NO: 153 to SEQ ID NO: 287.

131. A genetically modified microorganism comprising at least one polynucleotide encoding for an amino acid sequence that is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to a sequence chosen from SEQ ID NO: 288 to SEQ ID NO: 297 or SEQ ID NO: 305 to SEQ ID NO: 318, or combinations thereof.

132. A method of producing CBCA comprising:
(a) contacting a carbon substrate with the genetically modified microorganism of claim 131;
(b) growing said genetically modified microorganism to produce CBCA; and
optionally (c) isolating the CBCA from the genetically modified organism.

133. A polynucleotide encoding for at least one amino acid sequence that is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to a sequence chosen from SEQ ID NO: 288 to SEQ ID NO: 297 or SEQ ID NO: 305 to SEQ ID NO: 318, or combinations thereof.

134. A vector comprising the polynucleotide of claim 133.

135. A polypeptide comprising an amino acid sequence that is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to a sequence chosen from SEQ ID NO: 288 to SEQ ID NO: 297 or SEQ ID NO: 305 to SEQ ID NO: 318.

136. A genetically modified microorganism comprising at least one polynucleotide encoding for an amino acid sequence that is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to a sequence chosen from THCAS is a T446A mutant of SEQ ID NO: 120; a T446V mutant of SEQ ID NO: 120; or a T446I mutant of SEQ ID NO: 120, or combinations thereof;
preferably wherein the at least one polynucleotide encodes a THCAS signal sequence substantially identical to, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to a sequence chosen from SEQ ID NO: 121 to SEQ ID NO: 138.

137. A method of producing THCA comprising:
(a) contacting a carbon substrate with the genetically modified microorganism of claim 136;
(b) growing said genetically modified microorganism to producing THCA; and
optionally (c) isolating the THCA from the genetically modified organism.

138. A polynucleotide encoding for at least one amino acid sequence that is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to a sequence chosen from THCAS is a T446A mutant of SEQ ID NO: 120; a T446V mutant of SEQ ID NO: 120; or a T446I mutant of SEQ ID NO: 120, or combinations thereof;
preferably wherein the polynucleotide further encodes a THCAS signal sequence substantially identical to, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to a sequence chosen from SEQ ID NO: 121 to SEQ ID NO: 138.

139. A vector comprising the polynucleotide of claim 138.

140. A polypeptide comprising an amino acid sequence that is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to a sequence chosen from THCAS is a T446A mutant of SEQ ID NO: 120; a T446V mutant of SEQ ID NO: 120; or a T446I mutant of SEQ ID NO: 120, or combinations thereof.

141. The method of any one of claims 122, 127, 132, or 137, wherein said carbon substrate is a sugar, alcohol, and/or fatty acid.

142. The method of any one of claim 122, 127, 132, 137, or 141, wherein said carbon substrate is selected from hexanoic acid, butyric acid, glucose, fructose, xylose, sucrose, dextrins, starch, xylan, cellulose, hemicellulose, arabinose, glycerol, ethanol, butanol, methanol, or any combination thereof.

143. The method of any one of claim 122, 127, 132, 137, 141, or 142, wherein said carbon substrate is hexanoic acid.

144. The method of any one of claim 122, 127, 132, 137, 141, or 142, wherein said carbon substrate is butyric acid.

145. The use of a cannabinoid produced by any one of the methods of claims 122, 127, 132, 137, or 141 to 144, for the manufacture of a medicament for the treatment of a disease or a symptom of a disease.

146. The use of claim 145, wherein said a disease or a symptom of a disease is anorexia, multiple sclerosis, neurodegenerative disorders, epilepsy, glaucoma, osteoporosis, schizophrenia, bipolar disorder, post-traumatic stress disorder (PTSD), asthma, cardiovascular disorders, cancer, obesity, metabolic syndrome-related disorders, depression, anxiety, insomnia, emesis, pain, or inflammation.

147. A medicament comprising a cannabinoid made by any one of the methods of claims 122, 127, 132, 137, or 141 to 144, and a pharmaceutically acceptable excipient.

148. A method of treating a disease or a symptom of a disease comprising administering a subject in need thereof the cannabinoid made by any one of the methods of claims 122, 127, 132, 137, or 141 to 144.

149. The method of claim 148, wherein said a disease or a symptom of a disease is anorexia, multiple sclerosis, neurodegenerative disorders, epilepsy, glaucoma, osteoporosis, schizophrenia, bipolar disorder, post-traumatic stress disorder (PTSD), asthma, cardiovascular disorders, cancer, obesity, metabolic syndrome-related disorders, depression, anxiety, insomnia, emesis, pain, or inflammation.

150. A method of treating a disease or a symptom of a disease comprising administering a subject in need thereof the medicament of claim 147.

151. The use of a cannabinoid produced by any one of the microorganisms or methods of claims 121, 122, 126, 127, 131, 132, 136, 137, or 141 to 144, for the manufacture of a medicament for recreational use.

152. The use of any one of claims 145, 146 and 151, wherein the medicament is delivered through inhalation, intravenously, oral, or topical.

153. The use of claim 152, wherein the delivery is inhalation and completed through a vaporizer.

154. The use of claim 152, wherein said delivery is intravenous and the medicament is delivered through a saline solution.

155. The use of claim 152, wherein said delivery is oral and the medicament is delivered with food.

156. The use of claim 152, wherein said delivery is oral and the medicament is delivered through drink.

157. The use of claim 152, wherein said delivery is topical and the medicament is delivered through a patch.

158. The use of claim 152, wherein said delivery is topical and the medicament is delivered through a lotion.

159. The genetically modified microorganism of any one of claims 121, 126, 131, and 136, wherein said microorganism is a bacterium or a yeast.

160. The genetically modified microorganism of any one of claims 121, 126, 131, 136, and 159 wherein said microorganism is a yeast.

161. The genetically modified microorganism of claim 160, wherein said yeast is from the genus Saccharomyces.

162. The genetically modified microorganism of claim 160 or 161, wherein said yeast is from the species Saccharomyces cerevisiae.

163. The genetically modified microorganism of any one of claims 121, 126, 131, 136, and 159-162 further comprising at least one polynucleotide encoding at least one polypeptide with acyl activating activity; polyketide synthase activity; olivetol synthase activity; tetraketide synthase activity; olivetolic acid cyclase activity; THCA synthase activity; CBDA synthase activity; CBCA synthase activity; HMG-Co reductase activity; farnesyl pyrophosphate synthetase activity; or any combination thereof.

164. The genetically modified microorganism of any one of claims 121, 126, 131, 136, and 159-163 further comprising at least one polynucleotide encoding an acyl activating enzyme (AAE1); a polyketide synthase (PKS), such as a tetraketide synthase (TKS); an olivetolic acid cyclase (OAC); a THCA synthase (THCAS); a CBDA synthase (CBDAS); a CBCA synthase (CBCAS); a HMG-Co reductase (HMG1); a farnesyl pyrophosphate synthetase (ERG20); or any combination thereof;
preferably wherein the AAE1 is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO: 14;
preferably wherein the TKS is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO: 41.
preferably wherein the OAC is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO: 8;
preferably wherein the THCAS is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to a sequence chosen from SEQ ID NO: 10 or SEQ ID NO: 120;
preferably wherein the THCAS is a T446A mutant of SEQ ID NO: 120; a T446V mutant of SEQ ID NO: 120; or a T446I mutant of SEQ ID NO: 120;
preferably wherein the polynucleotide encodes a THCAS signal sequence substantially identical to, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to a sequence chosen from SEQ ID NO: 121 to SEQ ID NO: 138;
preferably wherein the CBDAS is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO: 12;
preferably wherein the CBCAS is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO: 18;
preferably wherein the HMG1 is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO: 20 or SEQ ID NO: 22;
preferably wherein the ERG20 is substantially identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO: 24.

165. The genetically modified microorganism of any one of claims 121, 126, 131, 136, and 159-164, further comprising at least one polynucleotide encoding an enzyme that is capable of converting olivetolic acid to cannabigerolic acid (“CBGA”).

166. The genetically modified microorganism of any one of claims 121, 126, 131, 136, 162, and and 159-164, further comprising at least one polynucleotide encoding an enzyme that is capable of converting butyric acid to cannabigerolic acid (“CBGVA”).
